(19) United States
(12) Patent Application Publication
(10) Pub. No.: US 2024/0384440 A1
(43) Pub. Date: Nov. 21, 2024

(54) ANTIBODY LIBRARIES WITH MAXIMIZED ANTIBODY DEVELOPABILITY CHARACTERISTICS

(71) Applicant: RULES-BASED MEDICINE INC., Austin, TX (US)

(72) Inventors: Andrew Raymon Morton BRADBURY, Santa Fe, NM (US); Michael Frank ERASMUS, Santa Fe, NM (US); Andre TEIXEIRA, Santa Fe, NM (US)

(73) Assignee: RULES-BASED MEDICINE INC., Austin, TX (US)

(21) Appl. No.: 18/592,499

(22) Filed: Feb. 29, 2024

Related U.S. Application Data
(63) Continuation of application No. 17/163,170, filed on Jan. 29, 2021, now Pat. No. 11,920,258.
Application No. 17/163,170 is a continuation of application No. 16/505,358, filed on Jul. 8, 2019, now Pat. No. 10,954,508.
(60) Provisional application No. 62/695,065, filed on Jul. 8, 2018.
(60) Provisional application No. 62/822,671, filed on Mar. 22, 2019.

(51) Int. Cl. C40B 40/10; C12N 15/10; C12N 15/81; C40B 40/08; C40B 50/06

(52) U.S. Cl. CPC C40B 40/10; C12N 15/1037; C12N 15/81; C40B 40/08; C40B 50/06

(57) ABSTRACT

Antibody libraries comprising a plurality of heavy chain variable domains and/or a plurality of light chain variable domains, which comprise complementary determining regions (CDRs) found in naturally-occurring human antibodies, and methods of making such antibody libraries. The antibody libraries are free of members that comprise one or more liabilities affecting one or more features of such members. Further, the antibody libraries comprise members having heavy chain and/or light chain CDRs not found in the same naturally-occurring human antibody.

### CROSS REFERENCE TO RELATED APPLICATIONS

The present application is a continuation of, and claims the benefit of and priority to, U.S. patent application Ser. No. 17/163,170, filed Jan. 29, 2021, which issued on Mar. 5, 2024 as U.S. Pat. No. 11,920,258, which is a continuation of, and claims the benefit of and priority to, U.S. patent application Ser. No. 16/505,358, filed on Jul. 8, 2019, which issued on Mar. 23, 2021 as U.S. Pat. No. 10,954,508, which claims the benefit of, and priority to, U.S. Provisional Application No. 62/822,671, filed on Mar. 22, 2019, and U.S. Provisional Application No. 62/695,065, filed on Jul. 8, 2018, the entire contents of each of which are incorporated by reference herein.

### SEQUENCE LISTING

The instant application contains a Sequence Listing which has been filed electronically in XML format and is hereby incorporated by reference in its entirety. Said XML copy, created on Feb. 29, 2024, is named 112310-0128-7000US08_SEQ.XML and is 371,564 bytes in size.

### BACKGROUND OF THE INVENTION

There are currently three recombinant antibody platforms used to generate human antibodies for human therapeutic use: (1) the “humanization” of murine monoclonal antibodies; (2) the immunization of transgenic mice containing human antibody genes; and (3) in vitro selection from vast human antibody libraries. Immunization approaches depend upon the occurrence of appropriate in vivo immune responses, and may not yield antibodies with desired characteristics. In contrast, in vitro selection has the advantage that antibodies with specific properties can be directly selected, and once selected, can be easily improved in terms of affinity or specificity.

In general, there are two types of antibody libraries: synthetic and natural antibody libraries. Synthetic antibody libraries can be constructed by introducing randomized complementarity determining region (CDR) sequences into antibody frameworks. Such antibody libraries can have vast potential genetic diversity and improved expression via selection of well-behaved frameworks. However, synthetic antibody libraries also include many non-functional antibody members and exclude much natural diversity due to the formulaic manner used to generate diversity within a restricted set of framework scaffolds. Antibody libraries created from natural sources, known as natural antibody libraries, have the advantage that the rearranged V genes undergo quality control in the B cell, and consequently a far higher proportion of the V genes are biologically functional, even if the potential diversity is lower. Disadvantages include the challenges of obtaining large numbers of B-cells to increase diversity, and the poor expression and biophysical properties of some antibodies expressed recombinantly in E. coli, yeast or mammalian cells.

### SUMMARY OF THE INVENTION

Provided herein are antibody libraries that comprise diversified heavy chain variable domains (VH) and/or light chain variable domains (VL), which comprise complementary determining regions (CDRs) obtained from naturally-occurring antibodies (e.g., naturally-occurring human antibodies or naturally-occurring camelid antibodies). Optionally, any of the VH CDRs and/or VL CDRs excludes at least members carrying one or more liabilities that affect one or more features of an antibody carrying such. Such antibody libraries, comprising CDRs from natural antibodies such as human antibodies, would have a high number of functional members and reflect natural diversity of human antibodies. Excluding members carrying one or more liabilities as described herein would enhance the percentage of members having desired properties, for example, high yield when produced by recombinant technology, high stability, reduced aggregation capacity, reduced liabilities as described below etc. Thus, the antibody libraries described herein would maximize antibody developability characteristics.

Accordingly, one aspect of the present disclosure features an antibody heavy chain library, comprising a plurality of nucleic acids or a plurality of genetic packages comprising the nucleic acids. The plurality of nucleic acids encode a population of antibody heavy chain variable domains, which collectively (in combination) comprise a population of heavy chain CDR1s, a population of heavy chain CDR2s, and/or a population of heavy chain CDR3s located at the CDR1 region, the CDR2 region, and the CDR3 region of a common antibody heavy chain variable domain gene. The heavy chain CDR1s, CDR2s, and/or CDR3s may be derived from naturally-occurring antibodies of a mammalian species, for example, human or camelid. In some embodiments, the plurality of nucleic acids encode a population of antibody heavy chain variable domains (e.g., human antibody heavy chain variable domains), which collectively (in combination) comprise a population of heavy chain CDR1s, a population of heavy chain CDR2s, and a population of heavy chain CDR3s.

In some embodiments, the common antibody heavy chain variable domain gene may be a human antibody heavy chain variable domain gene. Examples include VH1-24, VH2-70, VH3-7, VH4-30-4, VH5-51, VH1-18, VH1-69, VH3-23, VH5-10-1, VH3-9, or VH3-11. In some instances, the human antibody heavy chain variable region gene may be derived from a therapeutic antibody, for example, abrilumab, mepolizumab, crenezumab, necitumumab, anifrolumab, evoculumab, abituzumab, adalimumab, alemtuzumab, alirocumab, bapineuzumab, benralizumab, brodalumab, canakinumab, certolizumab, clazakizumab, dacetuzumab, daclizumab, daratumumab, eculizumab, efalizumab, elotuzumab, epratuzumab, farletuzumab, fasinumab, ficlatuzumab, fletikumab, fresolimumab, fulranumab, gevokizumab, ibalizumab, lintuzumab, matuzumab, mavrilimumab, mogamulizumab, motavizumab, natalizumab, nivolumab, obinutuzumab, ofatumumab, olokizumab, omalizumab, onartuzumab, otelixizumab, otlertuzumab, palivizumab, panitumumab, panobacumab, pertuzumab, pinatuzumab, polatuzumab, radretumab, ramucirumab, reslizumab, romosozumab, sarilumab, secukinumab, sifalimumab, tabalumab, tigatuzumab, tildrakizumab, tocilizumab, tovetumab, trastuzumab, vedolizumab, veltuzumab, zalutumumab, or zanolimumab.

In some embodiments, the population of heavy chain CDR1s, the population of heavy chain CDR2s, and/or the population of heavy chain CDR3s can be free (e.g., substantially free) of members comprising one or more of the following liabilities:
(i) a glycosylation site (e.g., comprising the motif NXS, NXT, or NXC, in which X represents any naturally-occurring amino acid residue except for proline),
(ii) a deamidation site (e.g., comprising the motif of NG, NS, NT, NN, NA, NH, ND, GNF, GNY, GNT, or GNG),
(iii) an isomerization site (e.g., comprising the motif of DT, DH, DS, DG, or DD),
(iv) an unpaired cysteine,
(v) net charge greater than 1 (e.g., in LCDR1-2 and/or HCDR1-2),
(vi) a tripeptide motif containing at least two aromatic residues (e.g., HYF or HWH), which may affect viscosity;
(vii) a motif that promotes aggregation (e.g., comprising the motif of FHW);
(viii) a polyspecificity site (e.g., GG, GGG, RR, VG, W, WV, WW, WWW, YY, or WXW, X referring to any amino acid residue),
(ix) a protease sensitive site (e.g., comprising the motif DX, in which X can be P, G, S, V, Y, F, Q, K, L, or D),
(x) an integrin binding site (e.g., comprising the motif RGD, LDV, or KGD),
(xi) a lysine glycation site such as a lysine glycation site (e.g., KE, EK, or ED),
(xii) a metal catalyzed fragmentation site (e.g., comprises the motif of HS, SH, KT, HXS, or SXH, in which X represents any amino acid residue),
(xiii) a polyspecificity aggregation site (e.g., the motif of of X₁X₂X₃, wherein each of X₁, X₂, and X₃ independently is F, I, L, V, W, or Y), and/or
(xiv) a streptavidin binding motif of (e.g., comprises the motif HPQ, EPDW (SEQ ID NO: 117), PWXWL (SEQ ID NO: 118), in which X represents any amino acid residue, GDWVFI (SEQ ID NO: 119), or PWPWLG (SEQ ID NO: 120)).

Alternatively, or in addition, the population of heavy chain CDR1s, the population of heavy chain CDR2s, and/or the population of heavy chain CDR3s in the antibody library described herein is free of non-functional members.

In some embodiments, the population of antibody heavy chain CDR1 fragments, the population of antibody heavy chain CDR2 fragments, and/or the population of antibody heavy chain CDR3 fragments is free of members comprising at least two of (i)-(xiv). In some examples, at least the population of antibody heavy chain CDR1 fragments, the population of antibody heavy chain CDR2 fragments, and/or the population of antibody heavy chain CDR3 fragments is free of members comprising (i)-(ix), and optionally free of members comprising one or more of (x)-(xiv).

In some embodiments, the antibody library described herein comprise a population of heavy chain CDR1s and/or a population of heavy chain CDR2s that is free of members comprising one or more of (i)-(xiv), and a population of heavy chain CDR3s is derived from naturally-occurring human antibodies (without removal of one or more the liability (i)-(xiv) disclosed herein), for example, derived from human B lymphocytes or precursor cells thereof.

In some instances, members of the antibody library described herein comprise heavy chain CDR1, heavy chain CDR2, and/or heavy chain CDR3 that are not from the same naturally-occurring antibody. For examples, at least 50% of the members in the antibody library do not comprise heavy chain CDR1, heavy chain CDR2, and/or heavy chain CDR3 that are from the same naturally-occurring antibody.

In another aspect, the instant disclosure provides an antibody light chain library, comprising a plurality of nucleic acids or a plurality of genetic packages comprising the nucleic acids. The plurality of nucleic acids encode a population of antibody light chain variable domains (e.g., human antibody light chain variable domains), which collectively (in combination) comprise a population of light chain CDR1s, a population of light chain CDR2s, and/or a population of light chain CDR3s located at the CDR1 region, the CDR2 region, and the CDR3 region of a common antibody light chain variable domain gene (e.g., a human antibody light chain variable domain gene). The light chain CDR1s, CDR2s, and CDR3s, and optionally the common light chain variable domain gene may be derived from naturally-occurring antibodies of the same mammal species, for example, human. In some embodiments, the population of human antibody light chain variable domains collectively comprises a population of light chain CDR Is, a population of light chain CDR2s, and a population of light chain CDR3s. In some embodiments, the population of light chain CDR1s, the population of light chain CDR2s, and/or the population of light chain CDR3s is free of members comprising one or more of the liabilities described herein (e.g., (i)-(xiv) described herein).

In some embodiments, the antibody light chain library described herein may comprise members having light chain CDR1, light chain CDR2, and/or light chain CDR3 that are not from the same naturally-occurring antibody. For example, at least 50% of the members in the antibody light chain library do not comprise light chain CDR1, light chain CDR2, and/or light chain CDR3 that are from the same naturally-occurring antibody.

In some embodiments, the common antibody light chain variable domain gene used in the antibody light chain library may be a human antibody light chain variable domain gene. Examples include K1-12, K4-1, K2D-29, K3-11, K3-20, or L2-14. In some instances, the human antibody heavy chain variable region gene is derived from a therapeutic antibody such as those described herein.

Also provided herein is an antibody library that comprises (i) a first plurality of nucleic acids encoding the population of antibody heavy chain variable domains of the antibody heavy chain library described herein, and (ii) a second plurality of nucleic acids encoding the population of antibody light chain variable domains of the antibody light chain library described herein. Alternatively, the antibody library provided herein may comprise (i) a first plurality of nucleic acids encoding the population of antibody heavy chain variable domains of the antibody heavy chain library described herein, and (ii) a common light chain variable domain, which may be VK3-20.

Any of the antibody libraries disclosed herein may be of a suitable format, for example, a library of full-length antibodies, a library of antigen-binding fragments such as Fab fragments, a library of single-chain antibodies, or a library of single-domain antibodies (e.g., VHH antibodies). In some examples, the antibody library disclosed herein may be a human antibody library. In other examples, the antibody library disclosed herein may be a camelid VHH antibody library.

In another aspect, the present disclosure features a method for producing an antibody library, comprising:
providing (a) a first plurality of nucleic acids encoding a population of naturally-occurring antibody heavy chain complementary determining region 1 (CDR1) fragments, and/or (b) a second plurality of nucleic acids encoding a population of naturally-occurring antibody heavy chain complementary determining region 2 (CDR2) fragments; and
inserting the first plurality of nucleic acids and/or the second plurality of nucleic acids into the CDR1 region and/or the CDR2 region, respectively, of an antibody heavy chain variable domain gene (e.g., those described herein), thereby producing an antibody library.

The method may further comprise:
providing a third plurality of nucleic acids encoding a population of naturally-occurring heavy chain complementary determining region 3 (CDR3) fragments, and
inserting the third plurality of nucleic acids into the CDR3 region of the heavy chain variable region gene.

The heavy chain CDR1 fragments, the heavy chain CDR2 fragments, and the heavy chain CDR3 fragments may be derived from naturally-occurring antibodies of a mammalian species as disclosed herein. In some embodiments, the heavy chain CDR1, CDR2, and CDR3 fragments can be derived from the same mammalian species. Optionally, the common heavy chain variable region gene may also be derived from the same mammalian species.

In some embodiments, the antibody library comprises members in each of which the heavy chain CDR1, the heavy chain CDR2, and/or the heavy chain CDR3 are not from the same naturally-occurring antibody. For example, at least 50% of the members in the antibody library do not contain heavy chain CDR1, the heavy chain CDR2, and/or the heavy chain CDR3 from the same naturally-occurring antibody.

In some embodiments, the population of antibody heavy chain CDR1 fragments, the population of antibody heavy chain CDR2 fragments, and/or the population of antibody heavy chain CDR3 fragments is free of members comprising one or more of the liabilities described herein, e.g., (i)-(xiv) disclosed herein For example, the population of antibody heavy chain CDR1 fragments, the population of antibody heavy chain CDR2 fragments, and/or the population of antibody heavy chain CDR3 fragments may be free of members comprising at least two of (i)-(xiv). In some instances, the population of antibody heavy chain CDR1 fragments, the population of antibody heavy chain CDR2 fragments, and/or the population of antibody heavy chain CDR3 fragments is free of members comprising any of (i)-(ix), optionally further free of one or more of (x)-(xiv).

In other embodiments, the population of antibody heavy chain CDR1 fragments and/or the population of antibody heavy chain CDR2 fragments is free of members comprising one or more of the liabilities described herein (e.g., at least two of (i) to (xiv) or all of (i) to (xiv)) and the population of human antibody heavy chain CDR3 fragments are from naturally-occurring human antibodies (e.g., derived from B cells), which may not have the one or more liabilities excluded.

In some embodiments, the first plurality of nucleic acids, the second plurality of nucleic acids, and/or the third plurality of nucleic acids is produced by a process comprising: (a) obtaining amino acid sequences of the heavy chain CDR1 regions, the heavy chain CDR2 regions, and/or the heavy chain CDR3 regions of a population of naturally-occurring antibodies (e.g., naturally-occurring human antibodies);
(b) excluding from (a) the heavy chain CDR1 amino acid sequences, the heavy chain CDR2 amino acid sequences, and/or the heavy chain CDR3 amino acid sequences that comprise one or more of liabilities (i) to (xiv) to obtain liability-free heavy chain CDR1 sequences, heavy chain CDR2 sequences, and/or heavy chain CDR3 sequences; and
(c) synthesizing the first plurality of nucleic acids that encode the liability-free heavy chain CDR1 regions, the second plurality of nucleic acids that encode the liability-free heavy chain CDR2 regions, and/or the third plurality of nucleic acids that encode the liability-free heavy chain CDR3 regions.

In some instances, the process described above may further comprise (d) isolating functional members from the liability-free heavy chain CDR1, CDR2, and/or CDR3 regions.

For example, the functional members of the liability-free heavy chain CDR1, CDR2, and/or CDR3 can be isolated by expressing antibodies comprising the liability-free heavy chain CDR1, CDR2, and/or CDR3 regions in host cells in a manner that the antibodies are displayed on surface of the host cells, isolating the antibodies that display on the host cells, show improved folding, and/or show reduced binding to polyspecificity reagents and identifying the CDR1, CDR2, and/or CDR3 regions in the displayed antibodies, which are functional members of the liability-free heavy chain CDR1, CDR2, and/or CDR3 regions.

The method for producing an antibody library as described herein may further comprise:
(i) providing a fourth plurality of nucleic acids encoding a population of naturally-occurring antibody light chain complementary determining region 1 (CDR1) fragments, a fifth plurality of nucleic acids encoding a population of naturally-occurring antibody light chain complementary determining region 2 (CDR2) fragments, and/or a sixth plurality of nucleic acids encoding a population of naturally-occurring human antibody light chain complementary determining region 3 (CDR3) fragments, and
(ii) inserting the fourth plurality of nucleic acids, the fifth plurality of nucleic acids, and/or the sixth plurality of nucleic acids into the CDR1 region, the CDR2 region, and the CDR3 region, respectively, of an antibody light chain variable domain gene (e.g., those described herein).

The light chain CDR1 fragments, the light chain CDR2 fragments, and the light chain CDR3 fragments may be derived from naturally-occurring antibodies of a mammalian species as disclosed herein. In some embodiments, the light chain CDR1, CDR2, and CDR3 fragments can be derived from the same mammalian species. Optionally, the common light chain variable region gene may also be derived from the same mammalian species.

The population of naturally-occurring antibody light chain CDR1 fragments, the population of antibody light chain CDR2 fragments, and/or the population of antibody light chain CDR3 fragments may be free of members comprising one or more of the liabilities described herein (e.g., at least two or all of (i) to (xiv) described herein). The antibody library may comprise members, each of which comprises a light chain CDR1, a light chain CDR2, and/or a light chain CDR3 that are not found in the same naturally-occurring antibody. For example, at least 50% of the members in the antibody library comprise light chain CDR1, light chain CDR2, and/or light chain CDR3 that are not found in the same naturally-occurring antibody.

In some embodiments, the fourth plurality of nucleic acids, the fifth plurality of nucleic acids, and/or the sixth plurality of nucleic acids is produced by a process comprising:
(a) obtaining amino acid sequences of the light chain CDR1, CDR2, and/or CDR3 regions of a population of naturally-occurring antibodies (e.g., naturally-occurring human antibodies),
(b) excluding from (a) the light chain CDR1, CDR2, and/or CDR3 amino acid sequences that comprise one or more of (i) to (x) to obtain liability-free light chain CDR1, CDR2, and/or CDR3 sequences, and
(c) synthesizing the fourth plurality of nucleic acids, the fifth plurality of the nucleic acids, and/or the sixth plurality of nucleic acids that encode the liability-free light chain CDR1, CDR2, and/or CDR3 regions.

The above process may further comprise (d) isolating functional members from the liability-free light chain CDR1, CDR2, and/or CDR3 regions. For example, the functional members of the liability-free light chain CDR1, CDR2, and/or CDR3 are isolated by expressing antibodies comprising the liability-free light chain CDR1, CDR2, and/or CDR3 regions in host cells in a manner that the antibodies are displayed on surface of the host cells, isolating the antibodies that display on the host cells, show improved folding, and/or show reduced binding to polyspecificity reagents and identifying the CDR1, CDR2, and/or CDR3 regions in the displayed antibodies, which are functional members of the liability-free light chain CDR1, CDR2, and/or CDR3 regions.

Further, the present disclosure features a method for making an antibody light chain library, the method comprising:
(i) providing a first plurality of nucleic acids encoding a population of naturally-occurring antibody light chain complementary determining region 1 (CDR1) fragments, a second plurality of nucleic acids encoding a population of naturally-occurring antibody light chain complementary determining region 2 (CDR2) fragments, and/or a third plurality of nucleic acids encoding a population of naturally-occurring antibody light chain complementary determining region 3 (CDR3) fragments, and
(ii) inserting the first plurality of nucleic acids, the second plurality of nucleic acids, and/or the third plurality of nucleic acids into the CDR1 region, the CDR2 region, and the CDR3 region, respectively, of an antibody light chain variable domain gene (e.g., those described herein).

The light chain CDR1 fragments, the light chain CDR2 fragments, and the light chain CDR3 fragments may be derived from naturally-occurring antibodies of a mammalian species as disclosed herein. In some embodiments, the light chain CDR1, CDR2, and CDR3 fragments can be derived from the same mammalian species. Optionally, the common light chain variable region gene may also be derived from the same mammalian species.

Also within the scope of the present disclosure are antibody libraries (e.g., antibody heavy chain libraries, antibody light chain libraries, or a combination thereof) produced by any of the methods described herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The instant application contains at least one drawing executed in color.

FIG. 1 is a chart showing the percentages of therapeutic antibodies in Phase 2 or Phase 3 clinical trials or approved therapeutic antibodies that are suitable or non-suitable for use as scaffold for antibody library construction. Suitable scaffolds for each of Phase 2, Phase 3, and Approved: 0-1 flags (left bar). Non-suitable scaffolds for each of Phase 2, Phase 3, and Approved: >2 flags (right bar).

FIG. 2 is a schematic showing the seven scaffolds developed from the VH and/or VL gene of a selected therapeutic antibody. Except for LCDR2, all CDRs are as described by IMGT. LCDR2 uses the Kabat description.

FIG. 3 is a schematic illustrating an exemplary design of cloning LCDR1 (as an example) into a selected light chain scaffold. Exemplary vector nucleic acids and corresponding amino acids are shown. The nucleic acid sequences correspond to SEQ ID NO: 1 (top) and SEQ ID NO: 2 (bottom). The amino acid sequence corresponds to SEQ ID NO: 3.

FIG. 4 is a flow chart describing an exemplary process of identifying unique CDRs and informatically removing CDRs based on liabilities, occurrence rate, and/or anomalous length.

FIG. 5 illustrates exemplary HCDR2 sequences comprising liabilities, such as deamidation sites, isomerization sites, glycosylation sites, or unpaired cysteine (highlighted). The first sequence of each column shows a graphical representation of a consensus sequence, which is followed by exemplary variant sequences. The depicted sequences in the “Deamidation column” correspond to SEQ ID NOs: 4-24 (top to bottom). The depicted sequences in the “Isomerization column” correspond to SEQ ID NOs: 25-45 (top to bottom). The depicted sequences in the “Glycosylation column” correspond to SEQ ID NOs: 46-66 (top to bottom). The depicted sequences in the “Unpaired Cysteine column” correspond to SEQ ID NOs: 67-87 (top to bottom).

FIG. 6 is a diagram showing presence of cysteine residues in heavy chain CDR3.

FIG. 7 shows presence of cysteine residues in heavy chain CDR3. Sequences correspond to SEQ ID NOs: 88-102 (from top to bottom).

FIG. 8 is a diagram illustrating paired cysteine residues in CDRs.

FIG. 9 includes graphs showing the presence of liabilities in heavy chain CDR1 (left panel) and CDR2 (right panel) regions before and after bioinformatic filtration.

FIG. 10 includes graphs showing the presence of liabilities in VK CDR1 (left panel) and CDR2 (right panel) regions before and after bioinformatic filtration.

FIG. 11 includes graphs showing the presence of liabilities in Vλ CDR1 (left panel) and CDR2 (right panel) before and after bioinformatic filtration.

FIG. 12 includes graphs showing the presence of liabilities in VK CDR3 (left panel) ad Vλ CDR3 (right panel) before and after bioinformatic filtration.

FIG. 13 includes a graph showing presence of liabilities in HC CDR3 before and after bioinformatic filtration.

FIG. 14 includes charts showing the percentage of CDRs in each library that are excluded at different abundance threshold. Single thread of 4 reads is used across different libraries to exclude CDRs that arise by sequencing error.

FIG. 15 includes a chart showing the number of CDRs remaining for all pooled CDRs except heavy chain CDR3 after exclusion at different threshold reads.

FIG. 16 includes graphs showing the length distribution of CDRs in Library 1 (using scaffold derived from abrilumab). The germline length for each CDR is indicated under each graph and the CDRs outside of the highlighted region are eliminated for anomalous length.

FIG. 17 includes graphs showing the length distribution of CDRs in Library 2 (using scaffold derived from mepolizumab). The germline length for each CDR is indicated under each graph and the CDRs outside of the highlighted region are eliminated for anomalous length.

FIG. 18 includes graphs showing the length distribution of CDRs in Library 3 (using scaffold derived from crenezumab). The germline length for each CDR is indicated under each graph and the CDRs outside of the highlighted region are eliminated for anomalous length.

FIG. 19 includes graphs showing the length distribution of CDRs in Library 4 (using scaffold derived from necitumumab). The germline length for each CDR is indicated under each graph and the CDRs outside of the highlighted region are eliminated for anomalous length.

FIG. 20 includes graphs showing the length distribution of CDRs in Library 5 (using scaffold derived from anifrolumab). The germline length for each CDR is indicated under each graph and the CDRs outside of the highlighted region are eliminated for anomalous length.

FIG. 21 includes graphs showing the length distribution of CDRs in Library 6 (using scaffold derived from evoculumab). The germline length for each CDR is indicated under each graph and the CDRs outside of the highlighted region are eliminated for anomalous length.

FIGS. 22A-22F includes graphs showing natural distribution (blue) and replicated natural designed distributions before (red) and after (green) filtering for well folded sequences. Data is shows for 6 different scaffolds/germlines at each of LCDR1-3 and HCDR1-2. The results are based on sequencing the libraries illustrated in FIG. 25B-G.

FIGS. 22G-22L includes cumulative plots for the diversity at each CDR position assessed for a natural distribution (natural library—blue), and replicated natural designed distributions before (red) and after (green) filtering for well folded sequences. The results are based on sequencing the libraries illustrated in FIG. 25B-25G.

FIG. 23 is a diagram illustrating an exemplary process for selecting for functional CDRs, for example, well folded LC CDR3s.

FIG. 24 is a diagram showing an exemplary process of inserting oligonucleotides encoding LC CDR Is into a yeast display vector. Nucleic acid sequences representing part of a yeast display vector to be cleaved with BsaI and Sfil correspond to SEQ ID NOs: 103-104 (from top to bottom). Sequences of the cleaved vector (middle) correspond to SEQ ID NOs: 105-108 (labeled from top to bottom then left to right). Sequences of the amplified LCDR1 oligo (middle) correspond to SEQ ID NOs: 109-112 (labeled top to bottom then left to right). Homologous recombination produces the amplified LCDR1 inserted into the yeast display vector (bottom), which corresponds to SEQ ID NOs: 113-116 (labeled top to bottom then left to right).

FIG. 25A is a diagram illustrating an exemplary process of isolating well expressed antibody CDRs by sorting yeast displaying single CDR loop libraries after cloning, using VK chains comprising functional KCDR1 as an example.

FIGS. 25B-25G illustrate the results of applying the exemplary process of isolating displayed single CDR loop libraries for each of the CDR (HCDR1-1, LCDR1-3) libraries displayed in yeast. The X axis indicates the level of antibody display, while the Y axis indicates the number of clones at each particular display level. For all histograms the display level of the clinical candidate is shown as a dotted blue line. For LCDR1-2 and HCDR1-2, fluorescence activated cell sorting was used to sort the most fluorescent 2% of yeast, corresponding to yeast displaying the most highly expressed antibodies. The display levels for the SV5 sorted (blue plot) and the most fluorescent 2% (red plot) are shown. For LCDR3, magnetic activated cell sorting was used to sort yeast displaying the most highly expressed antibodies. The transformed yeast clones (blue plot) are compared to the magnetic activated cell sorted yeast clones (red).

FIG. 26 is a diagram illustrating the pairing between a constant VH chain and VL chains with diversity in LC CDRs to select functional antibodies.

FIG. 27 is a diagram illustrating pairing between a constant VL chain and VH chains with diversity in VH CDRs for selection of functional antibodies.

FIG. 28 is a diagram illustrating construction of an exemplary antibody library comprising VL chains having well-folded LC CDRs selected via yeast display and unmodified VH domains.

FIG. 29 is a diagram illustrating an exemplary process of amplifying and assembling VH and VL CDRs into a pre-selected VH or VL scaffold.

FIG. 30 is a diagram illustrating construction of an exemplary Fab antibody library via combining a library of VH domains and a library of VL domains.

FIG. 31 is a diagram illustrating construction of an exemplary scFv antibody library via combining a library of VH domains and a library of VL domains.

FIG. 32 is a flow plot of scFvs corresponding to the clinical candidates used as scaffolds compared to the corresponding libraries. The double peak for library 5 is thought to be due to the presence of a smaller truncated product, which was eliminated in the final phage display libraries.

FIG. 33 shows a western blot of libraries 1-3 with the pIII and the scFv-pIII bands indicated.

FIG. 34 shows binding of antibodies displayed on yeast binding to targets against which they were selected at different concentrations. Display on yeast followed two rounds of selection by phage display, and two rounds of yeast display. Library 1 and Library 2 represent two different libraries created using the same LCDR1-3 and HCDR1-2 diversity, and HCDR3 diversity from different donors.

FIG. 35 is a diagram illustrating an exemplary affinity maturation approach via VL shuffling.

FIG. 36 is a diagram illustrating further HC CDR1 and HC CDR2 shuffling, following the VL shuffling depicted in FIG. 32.

FIG. 37 includes diagrams showing clones binding to the indicated antigens at various concentrations (100 nM, 10 nM, 1 nm, or 0 nM) isolated from Library A or Library B.

FIGS. 38A-38C include diagrams showing isolation of high affinity antibodies (having binding affinity at the subnanomolar level) from the libraries disclosed herein.

FIG. 39 is a diagram showing isolation of antibodies with high binding affinity to additional antigens, PDGF, TGFBR2, and TGFBR3, from the libraries disclosed herein.

### DETAILED DESCRIPTION OF THE INVENTION

It is generally accepted that the larger or more diverse an antibody library, measured in terms of the number of different antibodies, the better the antibodies that can be selected from it. Griffiths et al., EMBOJ 13(14):3245-3260, 1994 and Perelson et al., J Theor Biol., 81(4):645-70, 1979. The diversity of most antibody libraries has been estimated by counting the number of transformants, assuming that each colony represents a different antibody.

It was generally assumed that the VH gene diversity was the same as the number of colonies obtained, e.g., ˜10⁸ for the library described in Sblattero et al., Nat Biotechnol., 18(1):75-80, 2000. However, next-generation sequencing (NGS) showed that the VH clonal diversity (unique HCDR3 amino acid sequences) was actually ˜30 fold lower (3×10⁶). D'Angelo et al., MAbs., 6(1):160-72, 2014. Notwithstanding this apparent low diversity, many antibodies have been selected from this library. See, e.g., Sblattero et al., Nat Biotechnol., 18(1):75-80, 2000; Glanville et al., Curr Opin Struct Biol., 33:146-60, 2015; Lou et al., Journal of immunological methods; 253(1-2):233-42, 2001; Kehoe et al., Mol Cell Proteomics, 5(12):2350-63, 2006; Ayriss et al., J Proteome Res. 6(3):1072-82, 2007; Velappan et al., Journal of immunological methods, 321(1-2):60-9, 2007; Lillo et al., PLoS One, 6(12):e27756, 2011; Ferrara et al., PLoS One, 7(11):e49535, 2012; Close et al., BMC Microbiol. 13:270, 2013; and Ferrara et al., MAbs, 7(1):32-41, 2015.

NGS sequencing of another natural antibody library showed an even lower measured VH diversity (2×10⁵), even though the number of donors used (654) was extremely high, and the estimated number of colonies was 3×10¹⁰. Glanville et al., Proceedings of the National Academy of Sciences of the United States of America, 106(48):20216-21, 2009. Further, Fantini et al. PLoS One. 12(5):e0177574, 2017 described three libraries with maximal diversities (numbers of colonies) 6-16×10⁶, and estimated NGS diversities of 3-9×10⁶.

While genetic diversity is essential, effective functional diversity is even more important: a high genetic diversity is of no utility if the encoded antibodies are non-functional and unable to fold properly. Indeed, a single amino acid change in an antibody can result in dramatic changes in expression levels and stability. Some publications have shown the superiority of natural antibody libraries over synthetic ones. Hugo et al., Protein Eng., 16(5):381-6, 2003; Wang et al., Proteins, 82(10):2620-30, 2014; and Chan et al., Journal of immunological methods, 373(1-2):79-88, 2011. Natural diversity has the advantage that it has been prescreened for functionality by the immune system. However, it has the disadvantage that some antibodies are poorly expressed and folded in in vitro display systems, and that diversity can be dominated by a small number of clones.

The present disclosure aims, at least in part, at constructing antibody libraries comprising natural diversity such that the members of the libraries would be prescreened by the immune system for functionality, while excluding members that contain potential liabilities, would be poorly expressed, aggregating and/or poorly folded in a common screening system (e.g., yeast display, phage display, or a folding reporter such as β-lactamase; see, e.g., Saunders et al., Nat. Che Biol., 12:94-101; 1988; and D'Angelo et al., BMC genomics 12, suppl. 1, S1-S5; 2011; or green fluorescent protein; see e.g. Waldo, et al., Nat. Biotechnol., 17:691-5; 1999; Cabantous, et al., PLOS ONE., 3:e2387; 2008; and Cabantous, et al., J Struct Funct Genomics, 6:113-9; 2005). The present disclosure thus features, in some embodiments, a method to create extremely diverse, highly functional antibody libraries by combining naturally occurring CDRs, including naturally occurring CDRs containing somatic mutations generated in vivo, within antibody scaffolds such that members of the antibody libraries are expected to be well expressed and/or folded, and lacking liabilities.

As used herein, the term “liability” refers to a motif in an antibody (e.g., located in a heavy chain or light chain CDR region) that would negatively affect one or more desired features of the antibody (e.g., stability, good expression in an expression or display system, proper folding, no or reduced aggregation, solubility, no or reduced integrin binding, no or reduced glycosylation, no or reduced deamidation, no or reduced isomerization, no unpaired cysteine, or no or reduced protease sensitivity, etc.). By virtue of being comprised of highly functional members, such an antibody library would be expected to be functionally much larger than libraries of similar genetic size, in which antibodies are present that contain any of these liabilities. In other words, the antibody libraries disclosed herein would have a much larger effective diversity.

### I. Antibody Libraries and Methods of Construction

Provided herein are antibody libraries comprising the heavy chain and/or light chain CDR populations as described herein, wherein the heavy chain CDRs and/or light chain CDRs are inserted into a pre-selected heavy chain variable domain gene and/or a pre-selected light chain variable domain gene as also described herein, as well as methods of producing such antibody libraries. The heavy chain CDR1s, CDR2s, and/or CDR3s, and the pre-selected heavy chain variable domain may be of a mammalian species, for example, human, mouse, rat, rabbit, dog, pig, or camelid such as camel or llama. In some instances, the heavy chain CDR1s, CDR2s, and CDR3s may be derived from antibodies of the same mammalian species (e.g., human or camelid). Optionally, the pre-selected heavy chain variable domain gene may be from the same mammalian species. Alternatively, the heavy chain CDR1s, CDR2s, and/or CDR3s, and optionally the pre-selected heavy chain variable domain gene may be derived from naturally-occurring antibodies of different mammalian species.

Similarly, the light chain CDR1s, CDR2s, and CDR3s, as well as the pre-selected light chain variable domain gene may be of a mammalian species such as those described herein. In some instances, the light chain CDR1s, CDR2s, and CDR3s may be derived from antibodies of the same mammalian species (e.g., human or camelid). Optionally, the pre-selected light chain variable domain gene may be from the same mammalian species. Alternatively, the light chain CDR1s, CDR2s, and/or CDR3s, and optionally the pre-selected light chain variable domain gene may be derived from naturally-occurring antibodies of different mammalian species.

In some embodiments, the heavy chain CDRs and the pre-selected variable domain gene, and the light chain CDRs and the pre-selected variable domain gene are all of the same mammal species, for example, human.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody encompasses not only intact (e.g., full-length) antibodies, but also antigen-binding fragments thereof (such as Fab, Fab′, F(ab′)2, Fv), single-chain antibody (scFv), fusion proteins comprising an antibody portion, diabodies, nanobodies, single domain antibodies (also known as nanobodies, e.g., a V_{H} only antibody such as the VhH antibodies found in camelids), or multispecific antibodies (e.g., bispecific antibodies).

A typical antibody molecule comprises a heavy chain variable region (V_{H}) and a light chain variable region (V_{L}), which are usually involved in antigen binding. The V_{H} and V_{L}

regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E.A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

Single-domain antibodies, also known as nanobodies, are also within the scope of the present disclosure. In some embodiments, a single-domain antibody contains only a heavy chain (VHH). Heavy chain only antibodies (HcAb) are naturally produced by camelids and sharks. The antigen binding portion of the HcAb is comprised of the VHH fragment. Vincke et al., Methods Mol Biol. 911:15-26 (2012).

The antibody libraries disclosed herein may contain a population of antibodies of any suitable format. In some embodiments, the antibody library discloses herein comprise a population of full-length antibodies, which may be of any suitable family (e.g., IgG, or IgA). In other embodiments, the antibody library disclosed herein comprise a population of antigen-binding fragments, for example Fab fragments. In yet other embodiments, the antibody library disclosed herein comprise a population of single-chain antibodies. Alternatively, the antibody library disclosed herein may comprise a population of single-domain antibodies such as VHH fragments.

Exemplary steps for constructing the antibody libraries described herein may include:
(1) Identifying suitable VH/VL pairs for use as variable gene scaffolds;
(2) Generating vectors based on the scaffolds containing a single insertion site for each CDR, the remaining CDRs may remain unchanged;
(3) Identifying naturally occurring CDRs by analysis of a database of naturally occurring antibody sequences (which may be obtained from sequencing members of a natural antibody library);
(4) Eliminating from the database of naturally occurring CDR sequences those sequences likely to encode liabilities;
(5) Synthesize the remaining set of CDRs as oligonucleotides;
(6) Inserting the CDRs at their appropriate sites within the previously modified scaffolds, each scaffold containing CDRs at only one site (e.g., the identified collection of HCDR1s is inserted at the HCDR1 site of the modified scaffold).

In some embodiments, the CDRs (e.g., CDR1, CDR2, CDR3, or a combination thereof) identified as described herein may be experimentally screened or selected for good folding and/or expression and screened or selected against liabilities such as poor folding, poor expression, polyreactivity or aggregation. The selected CDRs may be inserted into complete V domains within the context of the scaffolds. The resultant complete V domains could be further screened and selected for good folding and/or expression, and/or screened and selected against liabilities such as poor folding or expression, polyreactivity or aggregation. The selected VH/VL complete scaffold pairs can be assembled and cloned into an appropriate display vector (e.g., phage or yeast) for screening of antibodies having desired binding specificity.

### A. Selection of Heavy Chain and Light Chain Variable Domain Framework Scaffolds

In some embodiments, the heavy chain and/or light chain framework scaffolds used in constructing the antibody libraries described here may be derived from commercially available therapeutic antibodies (e.g., those whose marketing authorization has been approved by the US Food and Drug Administration or/or the European Medicines Agency) or therapeutic antibodies that are currently in clinical trials, for example, in phase II or phase III trials. As used herein, a therapeutic antibody refers to the antibody molecule of an approved drug product (e.g., in the US, in EP, or in other jurisdictions such as CA or JP), or an antibody molecule that has been or is currently in a clinical trial in a suitable jurisdiction, for example, in the US or in Europe.

The germline heavy chain variable domain and light chain variable domain genes used in such therapeutic antibodies can be examined for features such as aggregation, hydrophobic interaction, polyspecificity, monomericity, level of expression in mammalian host cells (e.g., in HEK cells or CHO cells), Tm of its Fab form, and purification characteristics. See Table 1. Those having desired features, for example, high expression levels in mammalian cells (e.g., ≥50 mg/L in HEK cells), high Fab Tm (e.g., >64° C.), low slope for accelerated stability (e.g., <0.09), etc. can be selected as framework scaffolds for library constructions. Additional features and selection criterion are provided in Table 1, which shows as exemplary examples those therapeutic antibodies with the best properties, as well as three additional antibodies with poor therapeutic properties. This data is derived from Jain, T. et al. Biophysical properties of the clinical-stage antibody landscape. Proceedings of the National Academy of Sciences of the United States of America 114, 944-949, doi: 10.1073/pnas.1616408114 (2017).

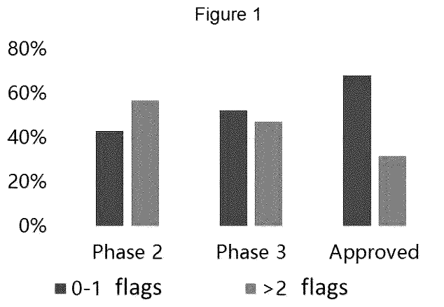

For each characteristic being evaluated, the worst 10% of the evaluated therapeutic antibodies can be assigned with a flag. See FIG. 1. In some instances, those therapeutic antibodies having less than 2 flags (e.g., having 1 flag or none) may be selected for use as the heavy chain and/or light chain framework scaffold.

In some embodiments, the heavy chain variable domain gene for use as the heavy chain framework scaffold can be VH1-24, VH2-70, VH3-7, VH4-30-4, VH5-51, VH1-18, VH1-69, VH3-23, VH5-10-1, VH3-9, or VH3-11. Alternatively, or in addition, the light chain variable domain gene for use as the light chain framework scaffold can be K1-12, K4-1, K2D-29, K3-11, K3-20, or L2-14. Such heavy chain and/or light chain framework scaffolds may be germline VH and/or VL genes. Alternatively, the heavy chain and/or light chain framework scaffolds may contain one or more mutations in one or more framework regions (e.g., FR1, FR2, FR3, or FR4) as compared with the germline gene counterpart. Such mutations may be present within the therapeutic antibody, or may be introduced to avoid specific liabilities, e.g., methionine oxidation, aggregation, integrin binding, glycosylation, deamidation, isomerization, unpaired cysteine, or protease sensitivity. In specific examples, the antibody library described herein uses the following VH and VL framework scaffold pairs: VH1-24/VK1-12, VH2-70/VK4-1, VH3-7/VK2D-29, VH4-30-4/VK3-11, VH5-51/VK3-20, or VH1-18/VL2-14.

In some specific examples, the VH and/or VL framework scaffolds used in the antibody library described herein are derived from abrilumab, mepolizumab, crenezumab, necitumumab, anifrolumab, or evoculumab, the characteristics of each of which are provided in Table 2. As used herein, “derived from” refers to the use of the VH and/or VL genes of any of these therapeutic antibodies, either with no modification, or with one or more mutations introduced into one or more of the framework regions, for example, up to 5 amino acid substitutions (e.g., up to 4, 3, 2, or 1 amino acid substitutions) in the VH gene (e.g., in one or more of the framework regions) and/or in the VL gene (e.g., in one or more of the framework regions).

In some instances, the mutations introduced into a germline VH and/or VL gene or introduced into the VH and/or VL gene of a reference therapeutic antibody (e.g., those listed in Table 2) may be conservative substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F.M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The amino acid sequences of exemplary VH and VL framework scaffolds are provided below (with CDRs that are modified indicated in boldface and mutations relative to parent therapeutic antibodies listed in Table 2 underlined):

Scaffold derived from abrilumab:
CDRs are bold and underlined
Mutations from the original antibody are italicized (all JK have been replaced for JK4).

VL:
(SEQ ID NO: 121)
DIQMTQSPSSVSASVGDRVTITCRASQGISSWLAWYQQKPGKAPKLLIYG
ASNLESGVPSRFSGSGSGTDFTLTISSLQPEDFANYYCQQANSFPWTFGG
GTKVEIK
Linker:
(SEQ ID NO: 122)
SGGSTITSYNVYYTKLSSSGT
VH:
(SEQ ID NO: 123)
QVQLVQSGAEVKKPGASVKVSCKVSGYTLSDLSIHWVRQAPGKGLEWMGG
FDPQDGETIYAQKFQGRVTMTEDTSTDTAYMELSSLKSEDTAVYYCATGS
SSSWFDPWGQGTLVTVSS

Scaffold Derived from Mepolizumab:

VL:
(SEQ ID NO: 124)
DIVMTQSPDSLAVSLGERATINCKSSQSLLNSGNQKNYLAWYQQKPGQPP
KLLIYGASTRESGVPDRFSGSGSGTDFTLTISSLQAEDVAVYYCQNVHSF
PFTFGGGTKVEIK
Linker:
(SEQ ID NO: 125)
SGGSTITSYNVYYTKLSSSGT
VH:
(SEQ ID NO: 126)
QVTLRESGPALVKPTQTLTLTCTVSGFSLSAYSVNWIRQPPGKALEWLAM
IWGDGKIVYNSALKSRLTISKDTSKNQVVLTMTNMDPVDTATYYCAGDGY
YPYAMDNWGQGTLVTVSS

Scaffold Derived from Crenezumab:

VL:
(SEQ ID NO: 127)
DIVMTQSPLSLPVTPGEPASISCRSSQSLVYSNGDTYLHWYLQKPGQSPQ
LLIYKVSNRFSGVPDRFSGSGSGTDFTLKISRVEAEDVGVYYCSQSTHVP
WTFGGGTKVEIK
Linker:
(SEQ ID NO: 128)
SGGSTITSYNVYYTKLSSSGT
VH:
(SEQ ID NO: 129)
EVQLVESGGGLVQPGGSLRLSCAASGFTFSSYGMSWVRQAPGKGLELVAS
INSNGGSTYYPDSVKGRFTISRDNAKNSLYLQMNSLRAEDTAVYYCASGD
YWGQGTTVTVSS

Scaffold Derived from Necitumumab:

VL:
(SEQ ID NO: 130)
EIVMTQSPATLSLSPGERATLSCRASQSVSSYLAWYQQKPGQAPRLLIYD
ASNRATGIPARFSGSGSGTDFTLTISSLEPEDFAVYYCHQYGSTPLTFGG
GTKVEIK
Linker:
(SEQ ID NO: 131)
SGGSTITSYNVYYTKLSSSGT
VH:
(SEQ ID NO: 132)
QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSS

Scaffold Derived from Anifrolumab:

VL:
(SEQ ID NO: 133)
EIVLTQSPGTLSLSPGERATLSCRASQSVSSSFLAWYQQKPGQAPRLLIY
GASSRATGIPDRLSGSGSGTDFTLTITRLEPEDFAVYYCQQYDSSAITFG
GGTKVEIK
Linker:
(SEQ ID NO: 134)
SGGSTITSYNVYYTKLSSSGT
VH:
(SEQ ID NO: 135)
EVQLVQSGAEVKKPGESLKISCKGSGYIFTNYWIAWVRQVPGKGLESMGI
IYPGDSDIRYSPSFQGQVTISADKSITTAYLQWSSLKASDTAIYYCARHD
IEGFDYWGRGTLVTVSS

Scaffold Derived from Evolocumab:

VL:
(SEQ ID NO: 136)
ESALTQPASVSGSPGQSITISCTGTSSDVGGYNSVSWYQQHPGKAPKLMI
YEVSNRPSGVSNRFSGSKSGNTASLTISGLQAEDEADYYCNSYTSTSMVF
GGGTKLTVL
Linker:
(SEQ ID NO: 137)
SGGSTITSYNVYYTKLSSSGT
VH:
(SEQ ID NO: 138)
EVQLVQSGAEVKKPGASVKVSCKASGYTLTSYGISWVRQAPGQGLEWMGW
VSFYNGNTNYAQKLQGRGTMTTDPSTSTAYMELRSLRSDDTAVYYCARGY
GMDVWGQGTTVTVSS

Any of the VH and/or VL framework scaffolds described herein can be used to construct a cassette that allows for cloning of one or more of pluralities of nucleic acids each encoding a diverse population of a heavy chain CDR or a light chain CDR into the corresponding framework scaffold at the corresponding CDR position.

In some instances, restriction sites can be introduced into a heavy chain scaffold flanking the CDR1 region, the CDR2 region, or the CDR3 region for cloning a plurality of nucleic acids encoding a diverse population of heavy chain CDR1s, heavy chain CDR2s, or heavy chain CDR3s, respectively. In some instances, restriction sites can be introduced into a heavy chain framework scaffold flanking at least two or the CDR1, CDR2, and CDR3 (e.g., CDR1+CDR2, CDR1+CDR3, or CDR2+CDR3) for cloning a combination of the corresponding CDR regions into the framework scaffold. In one example, restriction sites can be introduced into a heavy chain framework scaffold flanking all of the CDR1, CDR2, and CDR3 regions for cloning diverse heavy chain CDR1s, CDR2s, and CDR3s at the corresponding locations.

In some instances, restriction sites can be introduced into a light chain scaffold flanking the CDR1 region, the CDR2 region, or the CDR3 region for cloning a plurality of nucleic acids encoding a diverse population of light chain CDR1s, heavy chain CDR2s, or heavy chain CDR3s, respectively. In some instances, restriction sites can be introduced into a light chain framework scaffold flanking at least two or the CDR1, CDR2, and CDR3 (e.g., CDR1+CDR2, CDR1+CDR3, or CDR2+CDR3) for cloning a combination of the corresponding CDR regions into the framework scaffold. In one example, restriction sites can be introduced into a light chain framework scaffold flanking all of the CDR1, CDR2, and CDR3 regions for cloning diverse light chain CDR1s, CDR2s, and CDR3s at the corresponding locations.

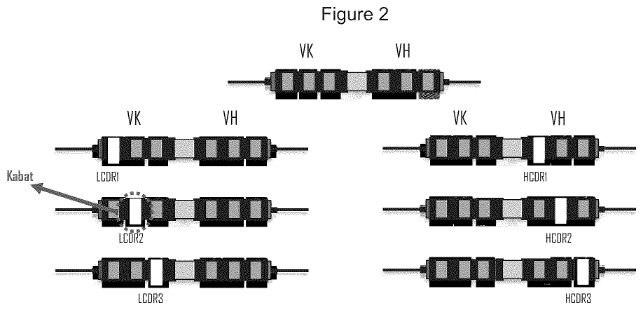

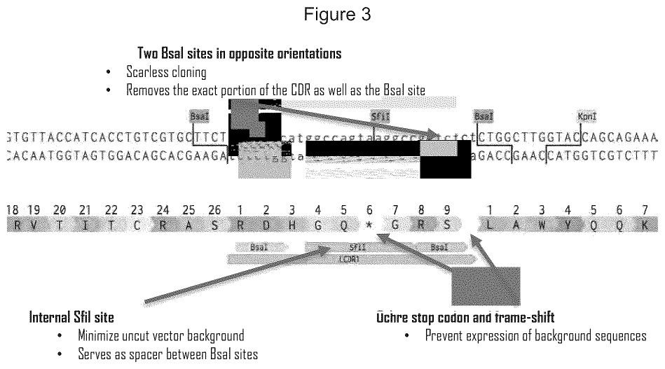

FIGS. 2 and 3 illustrate an exemplary scheme for construction of cassettes for introducing heavy chain CDR1, CDR2, or CDR3 diversities into a heavy chain scaffold and/or cassette for introducing light chain CDR1, CDR2, or CDR3 diversities into a light chain scaffold.

The resultant cassettes can be located in a suitable expression vector for producing the encoded antibodies in a suitable expression, display or folding reporter system.

### B. Heavy Chain and Light Chain CDR Populations

The heavy chain and/or light chain CDR1, CDR2, and/or CDR3 populations in the antibody libraries can be derived from naturally-occurring human antibodies. Such CDR sequences can be obtained by sequencing naturally-occurring antibodies (e.g., human antibodies) in existing natural antibody libraries and analyzing the heavy chain and light chain sequences thus obtained by conventional methods to identify heavy chain and/or light chain CDR sequences. Alternatively, or in addition, naturally-occurring antibody CDR sequences can be obtained by analyzing sequences of such antibodies in publicly available databases of naturally-occurring antibody sequences (e.g., human antibody sequences or camelid VHH antibody sequences), e.g., the NCBI database, the IMGT database, sequences from Jackson et al., J. Immunol. Methods, 324:26, 2007, and/or the sequences from Lee et al., Immunogenetics, 57:917, 2006, The Observed Antibody Space (antibodymap.org) described in Kovaltsuk, A. et al. Observed Antibody Space: A Resource for Data Mining Next-Generation Sequencing of Antibody Repertoires. Journal of Immunology, doi: 10.4049/jimmunol.1800708 (2018), and/or the iReceptor database (ireceptor.irmacs.sfu.ca) described in Corrie, B. D. et al. iReceptor: A platform for querying and analyzing antibody/B-cell and T-cell receptor repertoire data across federated repositories. Immunol Rev 284, 24-41, doi: 10.1111/imr. 12666 (2018), and/or the sequence database described in Briney, B. et al., Commonality despite exceptional diversity in the baseline human antibody repertoire. Nature, doi: 10.1038/s41586-019-0879-y (2019).

The extent of the framework region and CDRs can be precisely identified using methodology known in the art, for example, by the Kabat definition, the IMGT definition, the Chothia definition, the AbM definition, and/or the contact definition, all of which are well known in the art. See, e.g. Kabat, E.A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Lefranc, M. P. et al. IMGT unique numbering for immunoglobulin and T cell receptor variable domains and Ig superfamily V-like domains. Dev Comp Immunol 27, 55-77 (2003) and Almagro, J. Mol. Recognit. 17:132-143 (2004). See also hgmp.mrc.ac.uk, IMGT.org and bioinf.org.uk/abs.

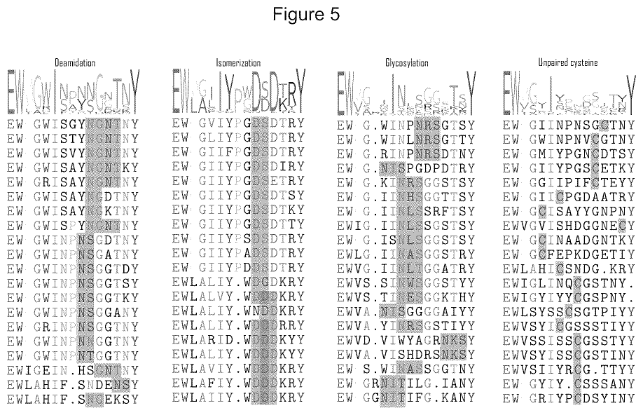

The heavy chain and/or light chain CDR1, CDR2, and/or CDR3 sequences thus obtained may be further analyzed to remove those that comprise a liability, e.g., those listed in Table 4. In some instances, heavy chain and/or light chain CDR1, CDR2, and/or CDR3 sequences comprising one of the liabilities listed in Table 4 (e.g., a glycosylation site, a deamidation site, an isomerization site, an unpaired cysteine, a net charge greater than 1 (e.g., in LCDR1-2 and/or HC CDR1-2), a tripeptide motif containing at least two aromatic residues (which may affect viscosity), a motif that promotes aggregation, (viii) a polyspecificity site such as those containing a motif of GG, GGG, RR, VG, W, WV, WW, WWW, YY, or, WXW, in which X represents any amino acid residue; a protease sensitive site (fragmentation sensitive site), or an integrin binding site) and/or FIG. 5 (using HC CDR2 as an example) can be removed such that the resultant antibody library is free (substantially free or completely free) of members comprising the excluded liability.

Alternatively or in addition, potential glycation sites such as lysine glycation sites may be removed. A glycation site refers to a site in a protein molecule that can be linked to a sugar molecule via a nonenzymatic process. Exemplary glycation sites include, but are not limited to, KE, EK, and ED. Additional liabilities include metal catalyzed fragmentation site (e.g., HS, SH, KT, HXS, or SXH, in which X represents any amino acid residue), polyspecificity aggregation site (e.g., having a motif of X₁X₂X₃, in which each of X₁, X₂, and X₃ is independently F, I, L, V, W, or Y), and streptavidin binding motif (e.g., HPQ, EPDW (SEQ ID NO: 117), PWXWL (SEQ ID NO: 118), in which X represents any amino acid residue, GDWVFI (SEQ ID NO: 119), and PWPWLG (SEQ ID NO: 120)).

Substantially free means that the number of a heavy or light chain CDR comprising the liability is less than 20% in the library, e.g., less than 15% or less than 10%.

In some examples, heavy chain and/or light chain CDR1, CDR2, and/or CDR3 sequences comprising two or more (e.g., 3, 4, 5, 6, 7, or more) of the liabilities noted above can be removed such that the resultant library is free of (substantially free of or completely free of) members comprising the excluded liabilities. In one example, heavy chain and/or light chain CDR1, CDR2, and/or CDR3 sequences comprising all of the liabilities listed in Table 4 can be removed such that the resultant library is free of (substantially free of or completely free of) members comprising any of the liabilities. Alternatively or in addition, heavy chain and/or light chain CDR1, CDR2, and/or CDR3 sequences comprising all of the liabilities listed in FIG. 5 can be removed such that the resultant library is free of (substantially free of or completely free of) members comprising any of the liabilities. In one specific example, heavy chain and/or light chain CDR1, CDR2, and/or CDR3 sequences comprising all of the liabilities disclosed herein can be removed such that the resultant library is free of (substantially free of or completely free of) members comprising any of the liabilities.

In some examples, heavy chain CDR1 and CDR2 sequences comprising one or more of liabilities, e.g., those listed in Table 4 and/or FIG. 5, can be removed, while heavy chain CDR3 sequences can be derived from naturally-occurring human antibodies without removal of members having the liabilities. Alternatively, heavy chain CDR3 sequences comprising one or more liabilities can also be removed. Alternatively, or in addition, light chain CDR1, CDR2, and CDR3 sequences comprising one or more of liabilities, e.g., those listed in Table 4 and illustrated by way of example in FIG. 5, can be removed.

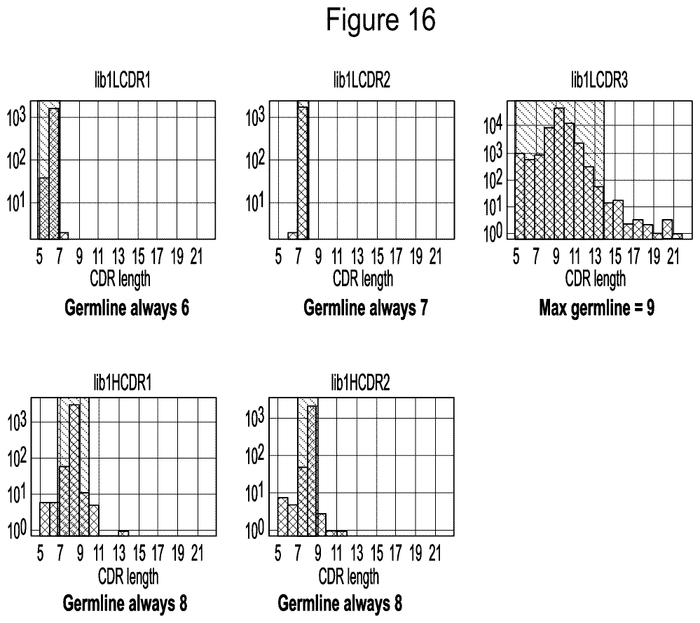

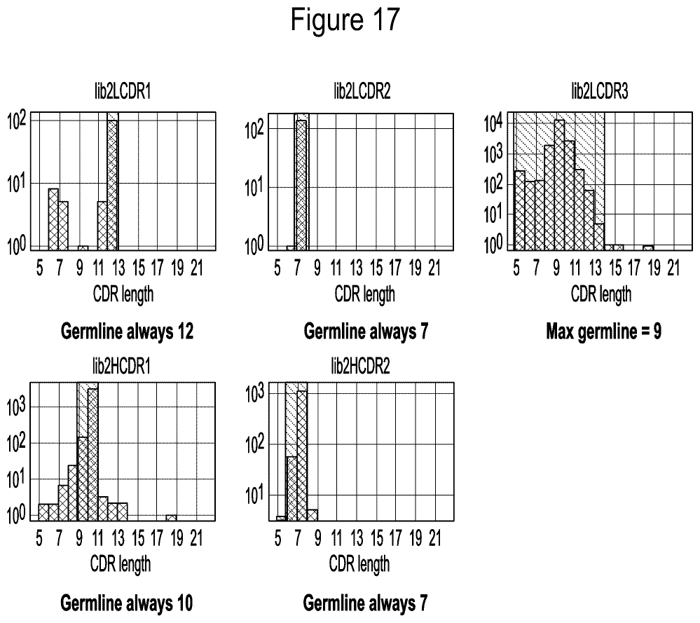

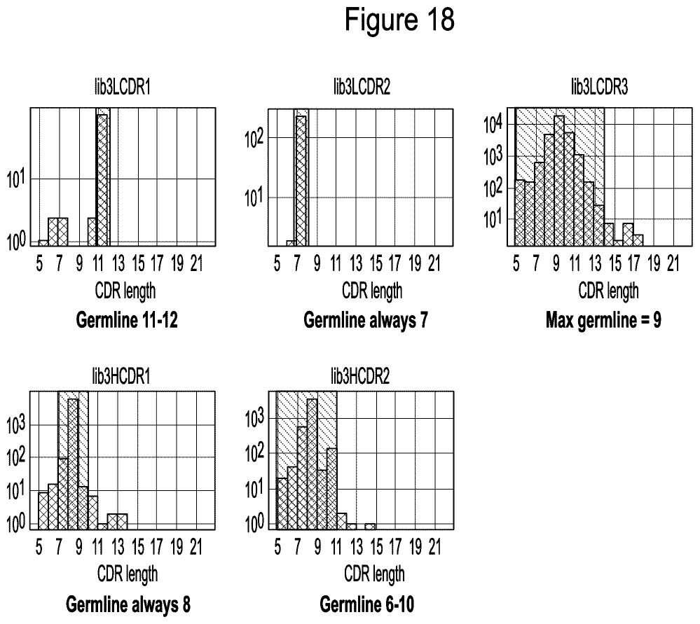

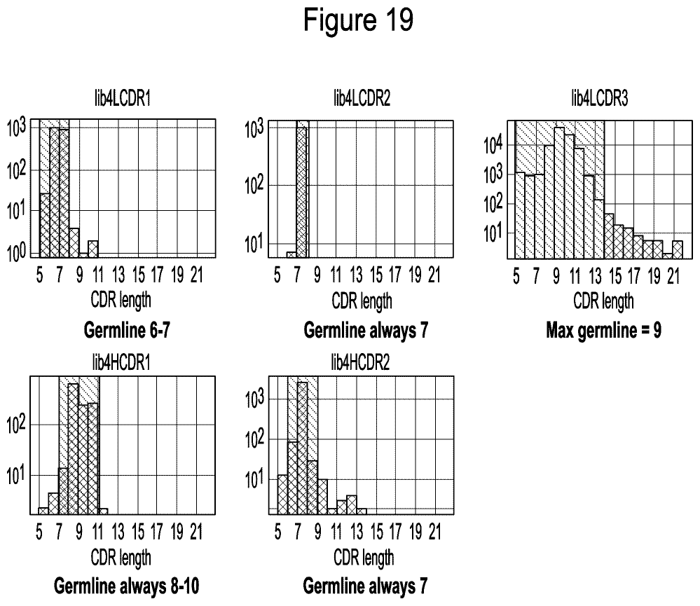

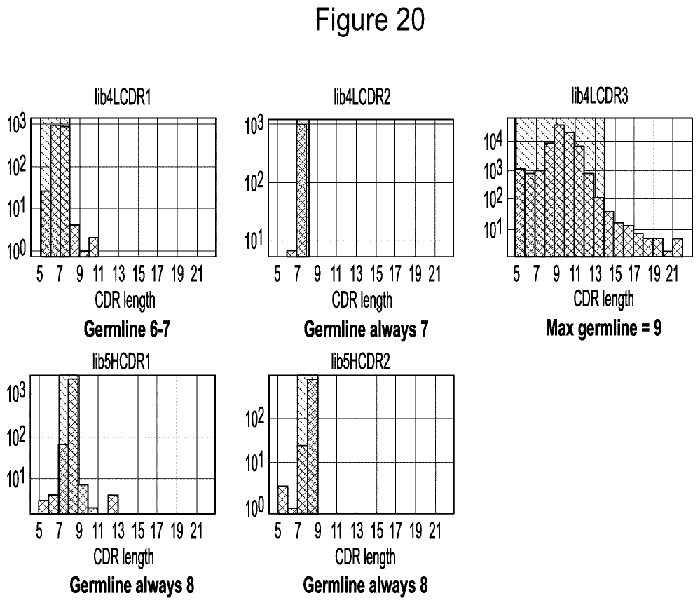

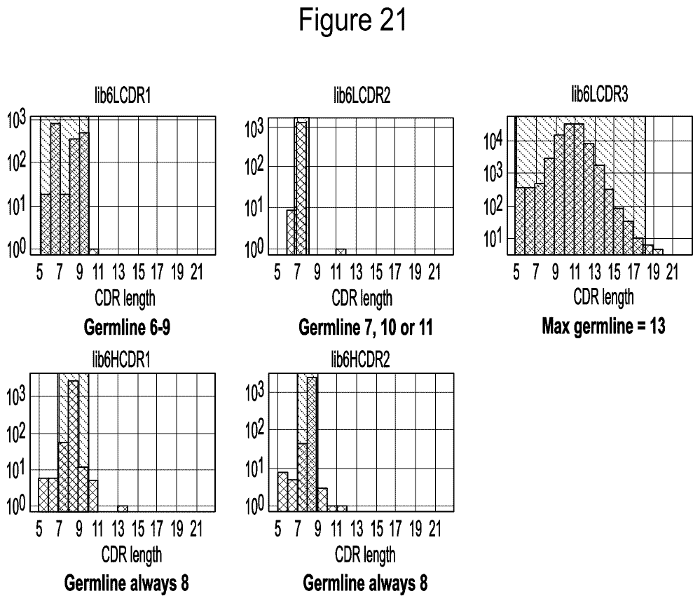

In some examples, heavy and/or light chain CDR1, CDR2, and/or CDR3 sequences having anomalous lengths can also be excluded. For example, light chain CDR1 and/or CDR2 having a length that is beyond the scope of germline length ±2-aa could be excluded. See FIGS. 16-21.

In some examples, heavy chain CDR1 and CDR2 members containing deamidation sites (e.g., NG, NS, NT, NN, GNF, GNY, GNT, GNG), isomerization sites (e.g., DG, DS, DD), aggregation site (FHW); motifs affecting viscosity (e.g., HYF and HWH), motifs indicating poor developability (e.g., net charge ≥+1 in LCDR1-2 and/or HCDR1-2), unpaired cysterine, polyspecificity site (e.g., GGG, RR, VG, VV, VVV, WW, WWW, YY, WXW, X referring to any amino acid residue, and GG), and glycosylation sites (e.g., NXS or NXT, in which X is any amino acid residue except for proline) can be excluded. In some examples, one or more of the following liabilities in heavy chain CDR1 and heavy chain CDR2 members can also be excluded: additional glycosylation sites (e.g., NXC, X being any amino acid residue except for proline), additional deamination sites (e.g., NA, NH, and/or ND), additional isomerization sites (e.g., DT and/or DH), lysine glycation sites (e.g., KE, EK, and ED), integrin binding sites (e.g., RGD, RYD, LDV, and KGD), protease sensitive sites (fragmentation site) (e.g., DP, DG, DS, DV, DY, DE, DQ, DK, DL, and DD), metal catalyzed fragmentation sites (e.g., HS, SH, KT, HXS, and SXH, in which X represents any amino acid residue), polyspecificity aggregatin sites (e.g., having a motif of X₁X₂X₃, in which each of X₁, X₂, and X₃ independently is F, I, L, V, W, or Y), and/or streptavidin binding sites (e.g., HPQ, EPDW (SEQ ID NO: 117), PWXWL (SEQ ID NO: 118), in which X represents any amino acid residue, GDWVFI (SEQ ID NO: 119), and PWPWLG (SEQ ID NO: 120)).

In some examples, the heavy chain CDR3 members having the one or more liabilities described herein can also be excluded. Alternatively, the heavy chain CDR3 members may include those derived from naturally-occurring antibodies directly without removal of the one or more liabilities described herein.

Alternatively or in addition, light chain CDR1, CDR2, and/or CDR3 members containing deamidation sites (e.g., NG, NS, NT, NN, GNF, GNY, GNT, GNG), isomerization sites (e.g., DG, DS, DD), aggregation site (FHW); motifs affecting viscosity (e.g., HYF and HWH), motifs indicating poor developability (e.g., net charge ≥+1 in LCDR1-2, HCDR1-2), unpaired cysterine, polyspecificity site (e.g., GGG, RR, VG, VV, VVV, WW, WWW, YY, WXW, X referring to any amino acid residue, and GG), and glycosylation sites (e.g., NXS or NXT, in which X is any amino acid residue except for proline) can be excluded. In some examples, one or more of the following liabilities in light chain CDR1, CDR2, and/or CDR3 members can also be excluded: additional glycosylation sites (e.g., NXC, X being any amino acid residue except for proline), additional deamidation sites (e.g., NA, NH, and/or ND), additional isomerization sites (e.g., DT and/or DH), lysine glycation sites (e.g., KE, EK, and ED), integrin binding sites (e.g., RGD, RYD, LDV, and KGD), protease sensitive sites (fragmentation site) (e.g., DP, DG, DS, DV, DY, DF, DQ, DK, DL, and DD), metal catalyzed fragmentation sites (e.g., HS, SH, KT, HXS, and SXH, in which X represents any amino acid residue), polyspecificity aggregatin sites (e.g., having a motif of X₁X₂X₃, wherein each of X₁, X₂, and X₃ independently is F, I, L, V, W, or Y), and/or streptavidin binding sites (e.g., HPQ, EPDW (SEQ ID NO: 117), PWXWL (SEQ ID NO: 118), in which X represents any amino acid residue, GDWVFI (SEQ ID NO: 119), and PWPWLG (SEQ ID NO: 120)).

In some examples, the one or more liabilities described herein may be excluded from all of the light chain CDR1, CDR2, and CDR3 members.

The resultant heavy chain and/or light chain CDR1, CDR2, and/or CDR3 sequences obtained from naturally-occurring antibodies, either excluding sequences comprising one or more liabilities or maintaining all sequences, can be used as templates to synthesis nucleic acids encoding, and replicating, the CDR sequences. Such nucleic acids can be inserted into the corresponding CDR position in the VH and/or VL scaffolds disclosed herein, and are termed “replicated natural CDRs”.

When desired, expression vectors carrying the VH and/or VL scaffolds with one or more heavy chain and or light chain CDRs inserted can be introduced into a suitable expression/display system for isolating functional members. Functional members include those having one or more superior features, for example, good expression and display in a suitable display system, improved folding, reduced aggregation or polyreactivity, and/or greater Tm. Such functional members can be identified by collecting host cells displaying antibodies produced from the expression vectors, and sequencing the corresponding heavy and/or light chain CDR sequences encoded by the expression vectors in the collected host cells.

For example, an initial antibody library may also be sorted for yeast displaying antibodies that have been stained with conformational probes that detect correct antibody folding. Traxlmayr et al., Arch Biochem Biophys. 526(2):174-80, 2012. Examples of such conformational probes include protein A (Hillson et al., The Journal of experimental medicine. 178(1):331-6, 1993; Akerstrom et al., 1994; J. Imm Methods, 177(1-2):151-63, 1994; and Roben et al., J. Immunology 154(12):6437-45, 1995) or protein L (Charbit et al., Gene, 70(1):181-9, 1988; Graille et al., Structure, 9(8):679-87, 2001; and Enever et al., Journal of molecular biology, 347(1):107-20, 2005), that are able to bind to VH3 and VK domains respectively, and derivatives of indole 3-butyric acid (Alves et al., Langmuir, 28(25):9640-8, 2012; Alves et al., Anal Chem., 84(18):7721-8, 2012; Alves et al., Bioconjug Chem., 25(7):1198-202, 2014; and Mustafaoglu et al., Biotechnol Bioeng., 112(7):1327-34, 2015) that binds to the “nucleotide binding site” found in all antibodies (Rajagopalan et al., Proceedings of the National Academy of Sciences of the United States of America, 93(12):6019-24, 1993).

The previous use of conformational probes has been shown to predict high expression and thermostability (Traxlmayr et al., 2012; Shusta et al., J Mol Biol. 292(5):949-56, 1999; Traxlmayr et al., Biochim Biophys Acta., 1824(4):542-9, 2012; Traxlmayr et al., Protein Eng Des Sel., 26(4):255-65, 2013; and Hasenhindl et al., Protein Eng Des Sel., 26(10):675-82, 2013) in yeast display. This approach selects for antibody fragments that are well expressed and well folded. Rather than positive selection for good display, each individual CDR library can be depleted of CDRs that contain liabilities. For example, adapting screens used for antibody screening (Yang et al., MAbs., 5(5):787-94, 2013; Kelly et al., MAbs, 7 (4): 770-7, 2015; Kohli et al., MAbs. 7(4):752-8, 2015; Obrezanova et al., MAbs., 7(2):352-63, 2015; Wu et al, Protein Eng Des Sel., 28(10):403-14, 2015; Yang et al., MAbs., 9 4):646-53, 2017; Xu et al., Protein Eng Des Sel., 26(10):663-70, 2013; and Kelly et al., MAbs., 9(7):1036-40, 2017) to yeast display sorting, and isolating those yeast displaying antibodies that correspond to the more “developable” phenotype selects for suitable CDRs that can then be combined to create highly functional libraries.

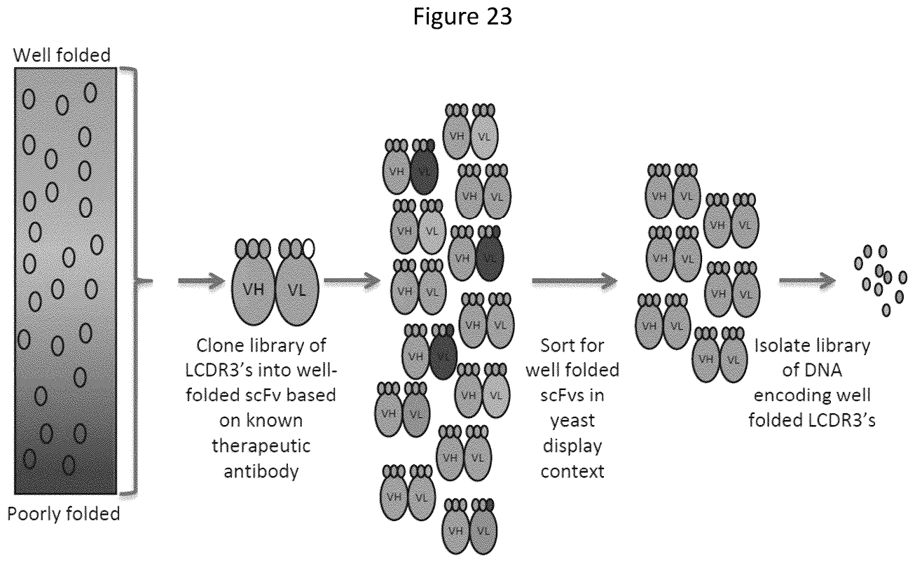

Examples of such selections include polyspecificity reagents, heparin or chaperones and only retaining those antibodies that do not bind such substances. Further stability increases can be generated by applying a heat shock step (Traxlmayr et al., 2012; Shusta et al., J Mol Biol. 292(5):949-56, 1999; Traxlmayr et al., Biochim Biophys Acta., 1824(4):542-9, 2012; Traxlmayr et al., Protein Eng Des Sel., 26(4):255-65, 2013; and Hasenhindl et al., Protein Eng Des Sel., 26(10):675-82, 2013). See also FIG. 23.

The sequences encoding functional members of the heavy and/or light CDR1, CDR2, and/or CDR3 can be used as templates for synthesizing nucleic acids coding for such functional members, or used directly. The resultant nucleic acids can then be inserted into the VH and/or VL scaffold as described herein to produce antibody libraries as also described herein. In some embodiments, the antibody library disclosed herein is substantially free of non-functional members, e.g., having less than 10% (e.g., less than 8%, less than 5%, less than 3%, less than 1%, or lower) non-functional members.

### C. Antibody Libraries

The antibody libraries described herein may comprise a plurality of nucleic acids encoding a plurality of antibody heavy chain and/or antibody light chain variable domains, which collectively comprise a common VH and/or VL framework scaffold (e.g., those described herein) with a diverse population of heavy or light chain CDR1s, a diverse population of heavy or light chain CDR2s, and/or a diverse population of heavy or light chain CDRs inserted at the corresponding CDR positions.

In some embodiments, the antibody library described herein is a heavy chain library comprising a plurality of nucleic acids encoding a plurality of antibody heavy chain variable domains. In some examples, the heavy chain library may comprise at least 10² diversity of heavy chain CDR1s (having at least 10² unique heavy chain CDR1 sequences), for example, at least 10³, 10⁴, 10⁵ diversity or at least 10⁶ diversity. Alternatively, or in addition, the heavy chain library may comprise at least 10² diversity of heavy chain CDR2s (having at least 10² unique heavy chain CDR2 sequences), for example, at least 10³, 10⁴, 10⁵ diversity or at least 10⁶ diversity. In other examples, the heavy chain library may comprise at least 10² diversity of heavy chain CDR3s (having at least 10² unique heavy chain CDR3 sequences), for example, at least 10³, 10⁴, 10⁵ diversity, at least 10⁶ diversity, at least 10⁷ diversity, or at least 10⁸ diversity.

In some examples, the heavy chain library may comprise diversity only in the heavy chain CDR1s, the heavy chain CDR2s, or the heavy chain CDR3s. In other examples, the heavy chain library may comprise diversity in at least two of the heavy chain CDR1, CDR2, and CDR3 regions (e.g., CDR1+CDR2, CDR1+CDR3, or CDR2+CDR3). In one specific example, the heavy chain library comprises diversity in all of the heavy chain CDR1, CDR2, and CDR3 regions.

In some embodiments, the heavy chain library is a secondary library generated for affinity maturation of a pre-selected antibody (the parent antibody) with binding activity to a target antigen. Such a secondary library may comprise diversity in one or two of the heavy chain CDR regions, while keeping the other CDR sequence(s) of the parent antibody. For example, the secondary library may comprise the same heavy CDR1 and CDR2 sequences as the parent antibody, and a diverse population of heavy chain CDR3 sequences. Alternatively, the secondary library may comprise the same heavy CDR3 sequence as the parent antibody and a diverse population of heavy chain CDR1 and/or CDR2 sequences.

Any of the heavy chain libraries disclosed herein may be paired with a common light chain variable region. Alternatively, it may be paired with any of the light chain antibody libraries as also described herein.

Also provided herein are antibody light chain libraries that comprise a plurality of nucleic acids encoding a plurality of antibody light chain variable domains. In some examples, the light chain library may comprise at least 10² diversity of light chain CDR1s (having at least 10² unique light chain CDR1 sequences), for example, at least 10³, 10⁴, 10⁵ diversity or at least 10⁶ diversity. Alternatively, or in addition, the light chain library may comprise at least 10² diversity of light chain CDR2s (having at least 10² unique light chain CDR2 sequences), for example, at least 10³, 10⁴, 10⁵ diversity or at least 10⁶ diversity. In other examples, the light chain library may comprise at least 102 diversity of light chain CDR3s (having at least 10² unique light chain CDR3 sequences), for example, at least 10³, 10⁴, 10⁵ diversity, at least 10⁶ diversity, at least 10⁷ diversity, or at least 10⁸ diversity.

In some examples, the light chain library may comprise diversity only in the light chain CDR1s, the light chain CDR2s, or the light chain CDR3s. In other examples, the light chain library may comprise diversity in at least two of the light chain CDR1, CDR2, and CDR3 regions (e.g., CDR1+CDR2, CDR1+CDR3, or CDR2+CDR3). In one specific example, the light chain library comprises diversity in all of the light chain CDR1, CDR2, and CDR3 regions.

In some embodiments, the light chain library is a secondary library generated for affinity maturation of a pre-selected antibody (the parent antibody) with binding activity to a target antigen. Such a secondary library may comprise diversity in one or two of the light chain CDR regions, while keeping the other CDR sequence(s) of the parent antibody. For example, the secondary library may comprise the same light CDR1 and CDR2 sequences as the parent antibody, and a diverse population of light chain CDR3 sequences. Alternatively, the secondary library may comprise the same light CDR3 sequence as the parent antibody and a diverse population of light chain CDR1 and/or CDR2 sequences.

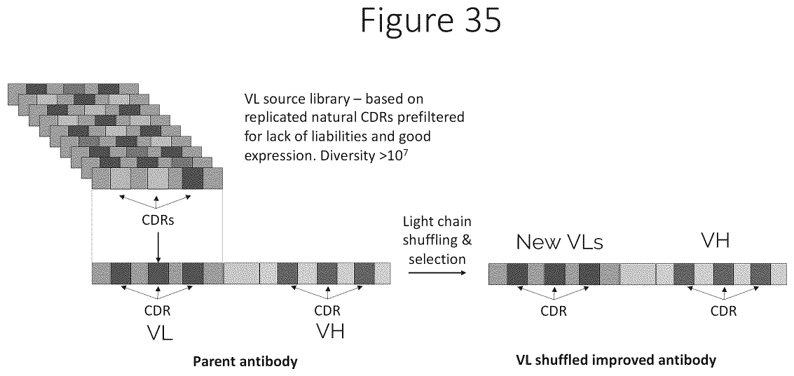

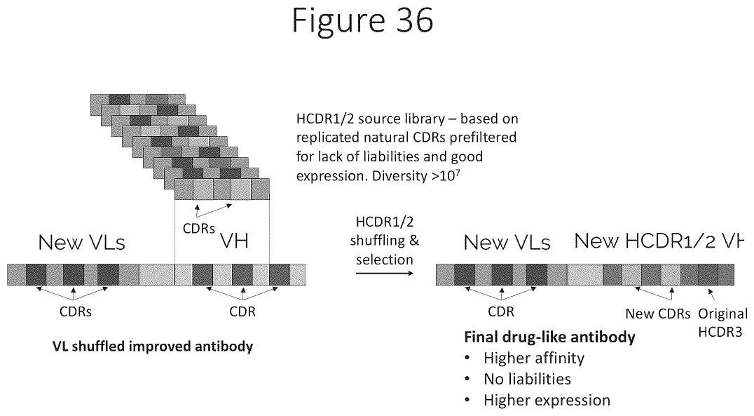

As illustrated in FIG. 35 and FIG. 36, a secondary library may be generated via VL shuffling and/or VH CDR1 and/or CDR2 shuffling.

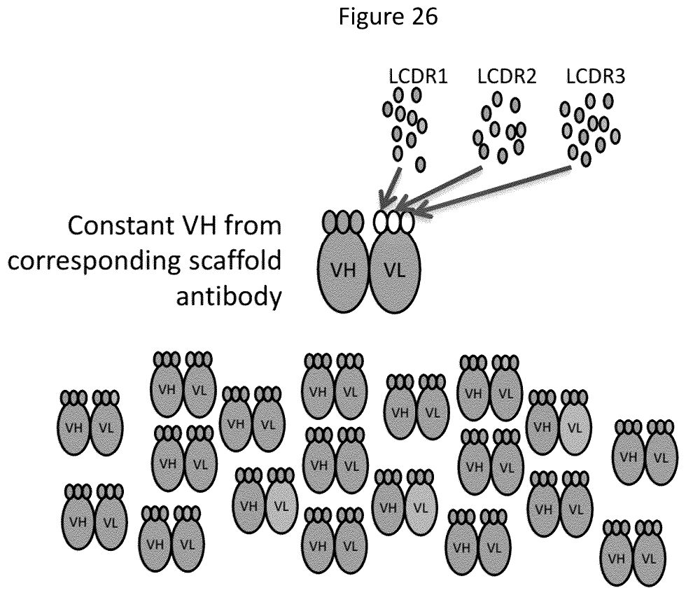

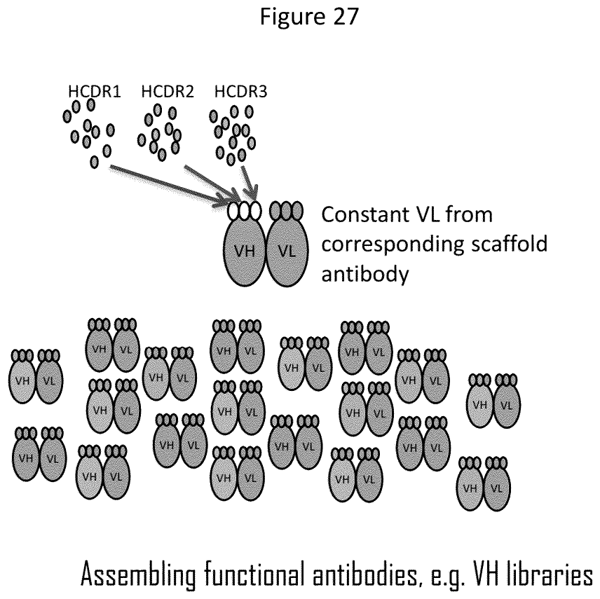

Any of the light chain libraries disclosed herein may be paired with a common heavy chain variable region. See, e.g., FIG. 26. Alternatively, it may be paired with any of the heavy chain antibody libraries as also described herein. See, e.g., FIG. 27.

### II. Antibody Library Screening

Any of the antibody libraries described herein may be used to screen for antibodies having binding specificity to an antigen of interest. Antibodies encoded by the nucleic acids in the library can be expressed and displayed using a suitable expressing/display system, for example, a cell-free display system (e.g., ribosome display), a phage display system, a prokaryotic cell-based display system (e.g., bacterial display), or a eukaryotic cell-based display system (e.g., yeast display or mammalian cell display). In certain embodiments, the antibody libraries are expressed and displayed on yeast cells. In other embodiments, the antibody libraries are expressed and displayed on phage particles (phage display). In other embodiments two or more display systems are used, e.g. phage display followed by yeast display.

The library of antibodies may be expressed/displayed in a suitable system, e.g., those described herein, in any format. Examples include intact antibodies (full-length antibodies), antigen-binding fragments thereof (e.g., Fab), or single chain antibodies (scFv).

Phage display is a protein display format using bacteriophages (e.g., phage f1, fd, and M13). In this system, at least one antibody chain (e.g., the heavy chain and/or the light chain) is typically covalently linked to a bacteriophage coat protein, for example, a gene III protein, a gene VIII protein, or a major coat protein (see, e.g., WO 00/71694). Phage display is described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20: Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; and Hoogenboom et al. (1991) Nuc Acid Res 19:4133-4137.

Bacteriophage displaying the protein component can be grown and harvested using standard phage preparatory methods, e.g., PEG precipitation from growth media. After selection of individual display phages, the nucleic acid encoding the selected protein components can be isolated from cells infected with the selected phages or from the phage themselves, after amplification. Individual colonies or plaques can be picked, the nucleic acid isolated and sequenced.

In other embodiments, a eukaryotic expression/display system, e.g., yeast cells or mammalian cells, can be used for expressing and displaying the library of antibodies as described herein. Yeast display is a protein display format, in which a protein component (e.g., an antibody component) is linked to a yeast cell wall protein (e.g., Aga1p or Aga2p) directly or indirectly. In some instances, one chain of an antibody can be covalently fused to the yeast cell wall protein for direct display. In other instances, the association between an antibody component and a yeast cell wall component can be mediated by an intermediate agent. Yeast display is described in, e.g., Cho et al., J. Immunol. Methods, 220(1-2):179-188, 1998; Boder et al., Methods Enzymol. 192(2):243-248, 2000; van den Beucken et al., FEBS Lett 546(2-3):288-294, 2003; and Boder et al., Arch Biochem Biophys 526(2):99-106, 2012.

To screen an antibody library as described herein for isolating antibodies capable of binding to a target antigen, the library of antibodies can be in contact with the target antigen under suitable conditions allowing for antibody-antigen binding. Phage particles or host cells displaying antibodies binding to the target antigen can be isolated, for example, by retention or a support member on which the target antigen is immobilized, amplified if needed, and the nucleic acids coding for the displayed antibodies can be determined. The screening process can be repeated multiple time, and display systems can be used in combination. When needed different antigens can be used for selecting antibody members having desired binding specificity or for negative selection to exclude antibody members having binding activity to a non-target antigen.

The screening of the antibodies derived from the libraries described herein can be carried out by any appropriate means. For example, binding activity can be evaluated by standard immunoassay and/or affinity chromatography. Determining the ability of candidate antibodies to bind therapeutic targets can be assayed in vitro using, e.g., a BIACORE™ instrument, which measures binding rates of an antibody to a given target antigen based on surface plasmon resonance. In vivo assays can be conducted using any of a number of animal models and then subsequently tested, as appropriate, in humans. Cell-based biological assays are also contemplated.

A lead antibody identified from antibody library screening may be subject to affinity maturation as described herein. A secondary library resulting from affinity maturation may be screened for binders having desired features, e.g., high binding affinity and/or binding specificity, following routine practice and/or disclosures provided herein.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B.D. Hames & S.J. Higgins eds. (1985»; Transcription and Translation (B.D. Hames & S.J. Higgins, eds. (1984»; Animal Cell Culture (R.I. Freshney, ed. (1986»; Immobilized Cells and Enzymes (IRL Press, (1986»; and B. Perbal, A practical Guide To Molecular Cloning (1984); F.M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Identifying Suitable VH VL Pairs for Use as Scaffolds

Suitable VH/VL pairs as scaffold for CDR insertions are crucial for creating highly diverse, highly functional antibody libraries. The usual rational for selecting scaffolds are: (i) the scaffolds are commonly used in nature (e.g., VH1-69, VH3-23); (ii) the scaffolds are chosen from known antibodies; (iii) the scaffolds are widely used by others; (iv) the scaffolds contains consensus germline sequences; and (v) the VH and VL pairs in the scaffold are considered to be stable, well expressed and non-aggregating.

Provided herein is an alternative approach for identifying suitable VH/VL pairs for use as a scaffold for antibody library construction. A recent publication describing certain approved antibodies and antibodies that are currently approved, or in Phase 2 or Phase 3 clinical trials (Jain, T. et al. Biophysical properties of the clinical-stage antibody landscape. Proceedings of the National Academy of Sciences of the United States of America 114, 944-949, doi:10.1073/pnas.1616408114 (2017)) was analyzed. The VH and VL germline genes of these antibodies were determined. Based on the developability data (e.g., aggregation, hydrophobic interaction, polyspecificity, monomericity, expression level in HEK cells, and Fab Tm) provided for each clinical antibody in the paper, the frequency and developability of these germline genes in clinical antibodies was assessed. The antibodies displaying the value in the worst 10% for each of the standard tested was flagged (highlighted in bold and italics in Table 1) and only antibodies with ≤1 flag were considered developable. In Table 1 all the clinical antibodies containing ≤1 flag are indicated in rows 2-69. In rows 70-72 examples of antibodies with >2 flags are indicated (Table 1). The antibodies chosen as exemplary examples are highlighted in bold. The correlation between the clinical development stage of the antibodies and the percentage of flags in these antibodies is shown in FIG. 1.

Six therapeutic antibodies (abrilumab, mepolizumab, crenezumab, necitumumab, anifrolumab, and evoculumab) are selected as our final scaffold choices (Highlighted in bold and italics in Table 1, and further described in Table 2). They are well expressed, showing no or minimal liabilities, containing few framework mutations and having low immunogenicity indicating the presence of suitable antibody scaffolds in these antibodies.

For each of the therapeutic antibodies listed in Table 2, seven vectors were designed and synthesized, as described in Example 2. The diagrammatic representation of vectors encoding the original scaffold, the heavy chain CDR1, heavy chain CDR2, heavy CDR3, light chain CDR1, light chain CDR2, and light chain CDR3 scaffolds derived from the six original scaffolds listed in Table 2 is shown in FIG. 2. The scaffolds represent five VH gene families (VH1-5) and five VL families (VK1-4; VA2). Table 2. Naturally occurring replicated CDRs can be inserted into these selected exemplary antibody scaffolds, which were identified as being well expressed, folded and lacking liabilities, for further testing.

TABLE 1
Analysis of Therapeutic Antibodies.

| | | | Fab Tm | SGAC-SINS | HIC | SMAC | Slope for |
|---|---|---|---|---|---|---|---|
| | | | by DSF | AS100 | Retention | Retention | Accelerated |
| | | HEK Titer | (° C.) | ((NH₄)₂SO₄ | Time | Time | Stability |
| Row | Name | (mg/L) <50 | ≤64 | mM) ≤300 | (Min) ≥11.8 | (Min) ≥13.0 | ≥0.09 |
| 2 | abituzumab | 89.6 | 75.5 | 900.0 | 9.2 | 8.7 | 0.06 |
| 3 | abrilumab | 100.2 | 71.0 | 900.0 | 9.4 | 8.7 | 0.03 |
| 4 | adalimumab | 134.9 | 71.0 | 900.0 | 8.8 | 8.7 | 0.05 |
| 5 | alemtuzumab | 144.7 | 74.5 | 1000.0 | 8.8 | 8.7 | 0.06 |
| 6 | alirocumab | 69.2 | 71.5 | 900.0 | 9.0 | 8.7 | 0.03 |
| 7 | anifrolumab | 82.0 | 62.5 | 700.0 | 8.8 | 8.6 | 0.07 |
| 8 | bapineuzumab | 151.1 | 73.0 | 1000.0 | 8.9 | 8.7 | 0.07 |
| 9 | benralizumab | 146.7 | 76.0 | 800.0 | 9.5 | 9.1 | 0.02 |
| 10 | brodalumab | 150.9 | 74.5 | 900.0 | 9.1 | 8.7 | 0.02 |
| 11 | canakinumab | 45.7 | 72.0 | 800.0 | 9.3 | 8.7 | 0.04 |
| 12 | certolizumab | 186.7 | 81.5 | 500.0 | 11.5 | 10.8 | 0.04 |
| 13 | clazakizumab | 113.5 | 69.5 | 800.0 | 9.6 | 8.9 | 0.05 |
| 14 | crenezumab | 149.3 | 72.0 | 700.0 | 10.0 | 8.7 | 0.05 |
| 15 | dacetuzumab | 128.5 | 68.0 | 1000.0 | 8.5 | 8.6 | 0.00 |
| 16 | daclizumab | 245.1 | 74.0 | 900.0 | 9.3 | 8.8 | 0.03 |
| 17 | daratumumab | 233.3 | 71.0 | 800.0 | 9.5 | 8.9 | 0.06 |
| 18 | eculizumab | 226.5 | 66.0 | 700.0 | 10.4 | 9.3 | 0.01 |
| 19 | efalizumab | 167.0 | 72.5 | 900.0 | 8.7 | 8.6 | 0.00 |
| 20 | elotuzumab | 213.2 | 83.5 | 700.0 | 10.3 | 9.3 | 0.00 |
| 21 | epratuzumab | 78.2 | 65.0 | 900.0 | 9.2 | 8.6 | 0.03 |
| 22 | evolocumab | 260.7 | 65.0 | 700.0 | 10.4 | 9.1 | 0.03 |
| 23 | farletuzumab | 220.8 | 75.5 | 800.0 | 9.5 | 9.1 | 0.01 |
| 24 | fasinumab | 110.4 | 71.0 | 900.0 | 10.0 | 8.7 | 0.07 |
| 25 | ficlatuzumab | 249.0 | 75.0 | 900.0 | 9.4 | 8.9 | 0.05 |
| 26 | fletikumab | 220.4 | 71.5 | 700.0 | 11.0 | 9.1 | 0.02 |
| 27 | fresolimumab | 166.0 | 74.0 | 700.0 | 10.9 | 9.1 | 0.06 |
| 28 | fulranumab | 142.0 | 68.5 | 900.0 | 9.3 | 9.3 | 0.07 |
| 29 | gevokizumab | 136.4 | 71.5 | 1000.0 | 8.8 | 8.6 | 0.07 |
| 30 | ibalizumab | 133.3 | 72.0 | 800.0 | 10.2 | 9.8 | 0.04 |
| 31 | lintuzumab | 230.0 | 75.5 | 700.0 | 10.9 | 9.4 | 0.05 |
| 32 | matuzumab | 224.3 | 72.0 | 900.0 | 9.8 | 8.8 | 0.02 |
| 33 | mavrilimumab | 150.5 | 68.5 | 700.0 | 10.3 | 8.7 | 0.05 |
| 34 | mepolizumab | 221.5 | 78.5 | 900.0 | 9.2 | 8.8 | 0.04 |
| 35 | mogamulizumab | 89.8 | 68.5 | 800.0 | 9.6 | 8.8 | 0.04 |
| 36 | motavizumab | 133.6 | 86.0 | 800.0 | 9.7 | 8.8 | 0.04 |
| 37 | natalizumab | 251.7 | 79.5 | 900.0 | 9.7 | 8.8 | 0.02 |
| 38 | necitumumab | 198.6 | 76.5 | 600.0 | 10.8 | 9.9 | 0.02 |
| 39 | nivolumab | 178.8 | 66.0 | 900.0 | 9.0 | 8.7 | 0.03 |
| 40 | obinutuzumab | 176.4 | 73.0 | 600.0 | 10.6 | 9.0 | 0.01 |
| 41 | ofatumumab | 249.8 | 68.0 | 800.0 | 9.7 | 9.5 | 0.03 |
| 42 | olokizumab | 115.3 | 69.0 | 700.0 | 9.9 | 9.0 | 0.04 |
| 43 | omalizumab | 150.4 | 77.5 | 800.0 | 9.5 | 8.7 | 0.05 |
| 44 | onartuzumab | 147.9 | 80.0 | 800.0 | 9.9 | 8.9 | 0.04 |
| 45 | otelixizumab | 152.1 | 75.5 | 1000.0 | 9.1 | 8.7 | 0.09 |
| 46 | otlertuzumab | 149.6 | 68.5 | 600.0 | 11.0 | 10.3 | 0.07 |
| 47 | palivizumab | 243.1 | 79.5 | 900.0 | 9.3 | 8.7 | 0.04 |
| 48 | panitumumab | 179.6 | 78.5 | 900.0 | 9.5 | 8.8 | 0.04 |
| 49 | panobacumab | 107.6 | 69.0 | 900.0 | 9.8 | 8.9 | 0.02 |
| 50 | pertuzumab | 31.4 | 78.5 | 700.0 | 10.1 | 8.9 | 0.04 |
| 51 | pinatuzumab | 130.6 | 79.0 | 800.0 | 9.2 | 8.8 | 0.07 |
| 52 | polatuzumab | 225.1 | 74.0 | 1000.0 | 8.8 | 8.7 | 0.06 |
| 53 | radretumab | 151.2 | 77.0 | 900.0 | 9.5 | 8.7 | 0.00 |
| 54 | ramucirumab | 90.7 | 66.0 | 900.0 | 9.4 | 8.7 | 0.02 |
| 55 | reslizumab | 191.6 | 75.5 | 700.0 | 9.8 | 8.9 | 0.06 |
| 56 | romosozumab | 227.7 | 76.0 | 1000.0 | 9.2 | 8.6 | 0.03 |
| 57 | sarilumab | 181.8 | 64.0 | 900.0 | 9.0 | 8.7 | 0.05 |
| 58 | secukinumab | 149.0 | 72.0 | 800.0 | 11.4 | 8.9 | 0.05 |
| 59 | sifalimumab | 158.6 | 67.0 | 800.0 | 9.7 | 8.8 | 0.01 |
| 60 | tabalumab | 121.6 | 64.0 | 700.0 | 10.8 | 9.9 | 0.06 |
| 61 | tigatuzumab | 179.0 | 64.5 | 700.0 | 10.0 | 8.7 | 0.00 |
| 62 | tildrakizumab | 181.9 | 77.5 | 600.0 | 11.1 | 9.9 | −0.01 |
| 63 | tocilizumab | 139.6 | 91.5 | 900.0 | 9.1 | 8.8 | 0.05 |
| 64 | tovetumab | 277.2 | 63.5 | 900.0 | 8.7 | 8.6 | 0.01 |
| 65 | trastuzumab | 159.5 | 78.5 | 800.0 | 9.7 | 8.8 | 0.04 |
| 66 | vedolizumab | 221.8 | 80.5 | 600.0 | 10.9 | 12.3 | 0.07 |
| 67 | veltuzumab | 225.0 | 70.0 | 700.0 | 11.1 | 9.7 | 0.04 |
| 68 | zalutumumab | 200.5 | 72.5 | 900.0 | 9.3 | 8.7 | 0.05 |
| 69 | zanolimumab | 116.4 | 80.5 | 700.0 | 9.6 | 8.8 | 0.03 |
| 70 | atezolizumab | 164.1 | 73.5 | 300.0 | 13.4 | 19.3 | 0.06 |
| 71 | belimumab | 10.5 | 60.0 | 800.0 | 10.5 | 9.3 | 0.13 |
| 72 | bevacizumab | 50.0 | 63.5 | 700.0 | 11.8 | 11.1 | 0.22 |

| | | Affinity-Capture | | | | |
|---|---|---|---|---|---|---|
| | Poly- | Self-Interaction | | | | |
| | Specificity | Nanoparticle | CIC | CSI-BLI | | |
| | Reagent | Spectroscopy | Retention | Delta | | BVP |
| | (PSR) SMP | (AC-SINS) Δλmax | Time | Response | ELISA | ELISA |
| Row | (0-1) ≥0.27 | (nm) Average ≥13.1 | (Min) >10 | (nm) ≥0.02 | >2.0 | ≥4.35 |
| 2 | 0.17 | 1.5 | 8.6 | 0.00 | 1.14 | 2.72 |
| 3 | 0.00 | −0.9 | 8.4 | −0.02 | 1.12 | 1.82 |
| 4 | 0.00 | 1.1 | 8.9 | −0.01 | 1.08 | 1.49 |
| 5 | 0.00 | −0.8 | 8.5 | −0.02 | 1.16 | 1.46 |
| 6 | 0.00 | 1.2 | 8.8 | −0.01 | 1.20 | 2.18 |
| 7 | 0.00 | −0.6 | 8.5 | −0.02 | 1.16 | 1.62 |
| 8 | 0.00 | −0.7 | 8.6 | 0.06 | 1.21 | 3.55 |
| 9 | 0.35 | 6.0 | 9.6 | −0.01 | 1.23 | 1.42 |
| 10 | 0.37 | 11.2 | 9.0 | −0.01 | 1.48 | 2.93 |
| 11 | 0.00 | 0.7 | 8.6 | 0.00 | 1.20 | 2.55 |
| 12 | 0.00 | 0.2 | 9.3 | −0.01 | 1.14 | 1.65 |
| 13 | 0.00 | 0.9 | 8.7 | −0.03 | 1.28 | 4.12 |
| 14 | 0.10 | 6.4 | 8.9 | 0.00 | 1.13 | 2.78 |
| 15 | 0.00 | 0.0 | 8.5 | −0.04 | 1.08 | 1.34 |
| 16 | 0.00 | −0.1 | 8.5 | −0.02 | 1.18 | 1.41 |
| 17 | 0.00 | 1.8 | 8.9 | 0.00 | 1.21 | 3.15 |
| 18 | 0.00 | 0.0 | 8.5 | −0.04 | 0.96 | 3.00 |
| 19 | 0.00 | 0.7 | 8.5 | −0.02 | 0.98 | 1.24 |
| 20 | 0.00 | −0.2 | 8.5 | −0.03 | 0.98 | 1.26 |
| 21 | 0.13 | 3.0 | 8.7 | −0.01 | 1.47 | 2.34 |
| 22 | 0.20 | 2.2 | 9.3 | −0.01 | 1.75 | 1.75 |
| 23 | 0.00 | −0.5 | 8.7 | −0.01 | 1.07 | 1.32 |
| 24 | 0.00 | −0.7 | 8.4 | −0.02 | 1.16 | 2.53 |
| 25 | 0.00 | −0.9 | 8.5 | −0.02 | 1.13 | 1.23 |
| 26 | 0.00 | −0.1 | 8.5 | −0.02 | 1.02 | 1.41 |
| 27 | 0.00 | −0.5 | 8.5 | −0.02 | 1.30 | 3.51 |
| 28 | 0.19 | 11.6 | 9.3 | 0.00 | 1.85 | 6.92 |
| 29 | 0.00 | −0.5 | 8.6 | −0.03 | 1.18 | 1.93 |
| 30 | 0.00 | −0.3 | 8.7 | −0.03 | 0.99 | 1.13 |
| 31 | 0.00 | 0.9 | 8.9 | −0.02 | 1.05 | 1.25 |
| 32 | 0.00 | −0.9 | 8.6 | −0.03 | 1.06 | 1.03 |
| 33 | 0.00 | −0.8 | 8.5 | −0.01 | 1.21 | 2.16 |
| 34 | 0.00 | −1.0 | 8.4 | −0.04 | 1.13 | 1.05 |
| 35 | 0.00 | −0.5 | 8.6 | −0.02 | 1.12 | 2.17 |
| 36 | 0.00 | 2.5 | 8.7 | −0.01 | 1.23 | 5.36 |
| 37 | 0.00 | 0.8 | 8.8 | −0.01 | 1.06 | 1.52 |
| 38 | 0.00 | 1.3 | 8.8 | −0.02 | 1.05 | 1.31 |
| 39 | 0.14 | 2.4 | 8.9 | −0.01 | 1.15 | 1.32 |
| 40 | 0.11 | 1.8 | 8.8 | −0.01 | 0.95 | 1.63 |
| 41 | 0.00 | 1.2 | 9.2 | −0.02 | 1.12 | 1.18 |
| 42 | 0.00 | −0.5 | 8.7 | −0.03 | 1.11 | 1.23 |
| 43 | 0.00 | −0.4 | 8.5 | −0.02 | 1.12 | 1.17 |
| 44 | 0.00 | 0.0 | 8.9 | −0.02 | 1.12 | 1.19 |
| 45 | 0.00 | 4.4 | 8.7 | −0.02 | 1.13 | 1.40 |
| 46 | 0.00 | 2.3 | 9.5 | −0.03 | 1.17 | 1.78 |
| 47 | 0.00 | −0.9 | 8.5 | −0.03 | 1.12 | 2.88 |
| 48 | 0.00 | −1.1 | 8.4 | −0.03 | 1.06 | 1.18 |
| 49 | 0.00 | −0.4 | 9.0 | −0.01 | 1.21 | 1.90 |
| 50 | 0.00 | −0.2 | 8.6 | −0.04 | 1.21 | 1.69 |
| 51 | 0.01 | 0.6 | 8.8 | −0.02 | 1.27 | 2.49 |
| 52 | 0.00 | −1.0 | 8.3 | −0.05 | 1.36 | 3.62 |
| 53 | 0.13 | 3.4 | 8.9 | 0.00 | 1.26 | 3.29 |
| 54 | 0.00 | 0.0 | 8.6 | −0.02 | 1.05 | 1.25 |
| 55 | 0.23 | 1.7 | 8.9 | 0.00 | 1.25 | 2.02 |
| 56 | 0.00 | −1.0 | 8.4 | −0.03 | 1.01 | 1.47 |
| 57 | 0.00 | 1.1 | 8.7 | −0.01 | 1.19 | 2.17 |
| 58 | 0.00 | −0.6 | 8.4 | −0.04 | 1.09 | 1.69 |
| 59 | 0.06 | 2.1 | 9.0 | −0.02 | 2.60 | 2.50 |
| 60 | 0.00 | 2.0 | 9.1 | −0.01 | 1.26 | 3.68 |
| 61 | 0.13 | 5.5 | 8.7 | −0.01 | 1.17 | 1.70 |
| 62 | 0.00 | 0.8 | 8.7 | −0.01 | 1.19 | 1.77 |
| 63 | 0.00 | 1.3 | 8.9 | 0.00 | 1.14 | 2.81 |
| 64 | 0.00 | 2.2 | 8.8 | −0.01 | 1.35 | 2.95 |
| 65 | 0.00 | 2.0 | 8.8 | −0.02 | 1.06 | 1.34 |
| 66 | 0.00 | 0.4 | 9.0 | −0.02 | 1.15 | 1.58 |
| 67 | 0.00 | 4.8 | 8.8 | −0.02 | 0.89 | 1.21 |
| 68 | 0.00 | −0.8 | 8.4 | −0.03 | 1.28 | 2.90 |
| 69 | 0.13 | 1.5 | 8.6 | −0.01 | 1.10 | 1.46 |
| 70 | 0.07 | 15.0 | 10.8 | 0.06 | 1.29 | 6.20 |
| 71 | 0.00 | 0.8 | 8.6 | −0.03 | 3.61 | 12.23 |
| 72 | 0.00 | 0.8 | 9.8 | −0.02 | 1.29 | 2.78 |

TABLE 2
Summary of Final Scaffold Choices.

| | | | | | | | | % | | |
|---|---|---|---|---|---|---|---|---|---|---|
| | | | | VH | VK | Framework | Framework | Immuno- | Fab | Phage display |
| Therapeutic | Target | Phase | Type | gene | gene | VH mutations | VL mutations | genicity* | Tm ° C. | used |
| Abrilumab | a4-β7 | Phase 2 | Human | 1-24 | 1-12 | 1 | 1 | 0 | 71.0 | Other H1-24 |
| | integrin | | | | | | | | | & K1-12 |
| Mepolizumab | IL-5 | Approved | Humanized | 2-70 | 4-1 | 4 | 0 | 6 | 78.5 | Other H2-70 |
| | | | | | | | | | | & K4-1 |
| Crenezumab | Aβ | Phase 3 | Humanized | 3-7 | 2D-29 | 2 | 3 | ND | 72.0 | None |
| Necitumumab | EGER | Approved | Human | 4-30-4 | 3-11 | 3 | 1 | 4.1 | 76.5 | For Necitumumab |
| Anifrolumab | Interferon | Phase 3 | Human | 5-51 | 3-20 | 1\hat{ } | 2 | 3.3 | 62.5 | Other H5-51 |
| | receptor | | | | | | | | | & K3-20 |
| Evoculumab | PCSK9 | Approved | Human | 1-18 | 12-14 | 2 | 1 | 0.3 | 65 | Other H1-18 |

*These identified CDRs are identical, but synthesized with different flanking regions corresponding to different VH1 genes (VH1-18 and VH1-24).
\hat{ }These identified CDRs are identical, but synthesized with different flanking regions corresponding to different VK3 genes (VK3-20 and VK3-11).

### Example 2: Creating Vectors for Experimental CDR Screening

For each of the six libraries created using the six scaffolds shown in Table 2, seven polynucleotides encoding single-chain variable fragment (scFv) corresponding to each of the scaffolds were synthesized. One of the seven synthesized polynucleotides encodes for the non-modified scFv, and the other six polynucleotides were modified to have one of the original CDRs replaced by a combination of restriction sites including two inverted BsaI sites (a type IIs enzyme that cuts outside of its recognition sequence), an additional SfiI site to ensure cleavage of the vector and serve as a spacer between the BsaI sites, a frameshift and an ochre stop codon to prevent expression of background sequence (FIGS. 2-3). Each of these modified polynucleotides encoding the scaffolds was cloned into a yeast display vector, and the presence of the stop codon in this sequence prevented the expression of the scaffold on the yeast surface until the modified CDR is replaced with a functional CDR.

### Example 3: Generating a Database of CDR Sequences and Informatic Elimination of CDRs Demonstrating Potential Liabilities

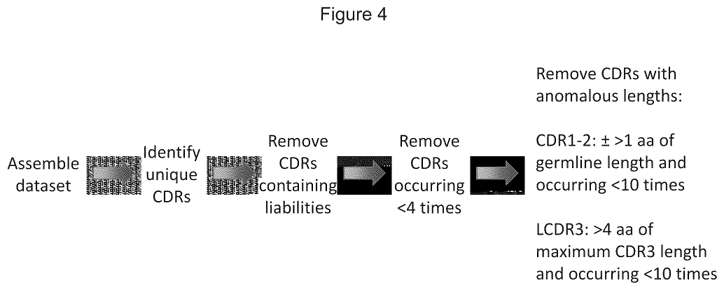

The process taken to identify suitable CDRs for use in the libraries exemplified herein is illustrated in FIG. 4. To generate a database of naturally occurring CDRs (CDRs found in naturally-occurring antibodies such as human antibodies), next generation sequencing (NGS) of the variable genes derived from a total of 40 donors was carried out, comprising a total of >140 million reads. NovaSeq analysis was applied to LCDR3 sequencing data and MiSeq analysis was applied to heavy chain and light chain CDR1 and CDR2 sequencing data. Analysis of the variable gene sequences allowed identification of the numbers of CDRs shown in Table 3.

Altogether, unique CDRs corresponding to the following heavy chain and light chain CDRs were identified showing both unique CDRs and CDRs including potential flanking scaffold oligos (in parentheses). The number of oligos is greater than the number of CDRs because of the need to synthesize some CDRs with different flanking sequences corresponding to different scaffolds.:
˜52,675 (66,020) LCDR1s;
˜19,550 (23,854) LCDR2s;
˜2,180,922 (2,617,051) LCDR3s;
˜147,741 (167,376) HCDR1s;
˜170,758 (202,170) HCDR2s; and
˜13,588,754 HCDR3s

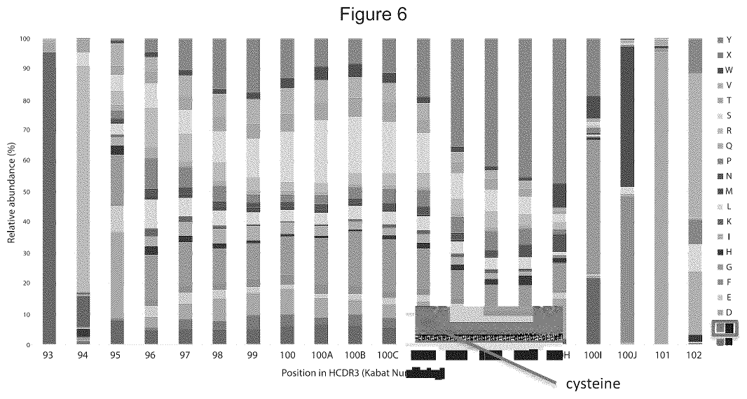

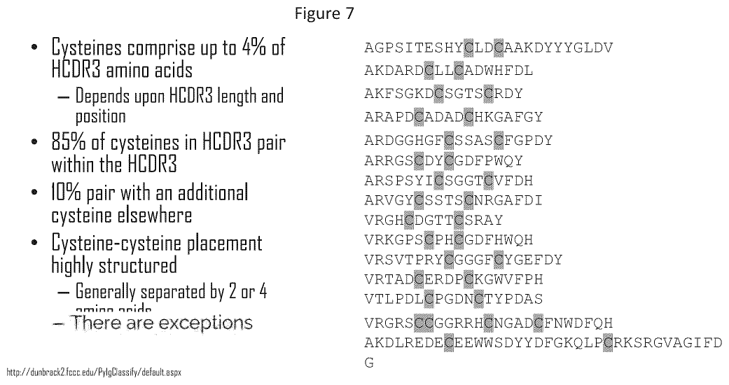

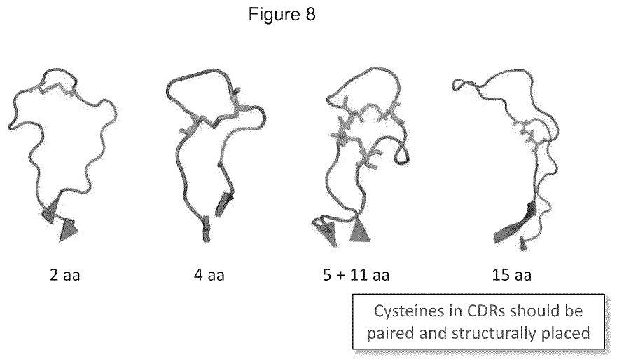

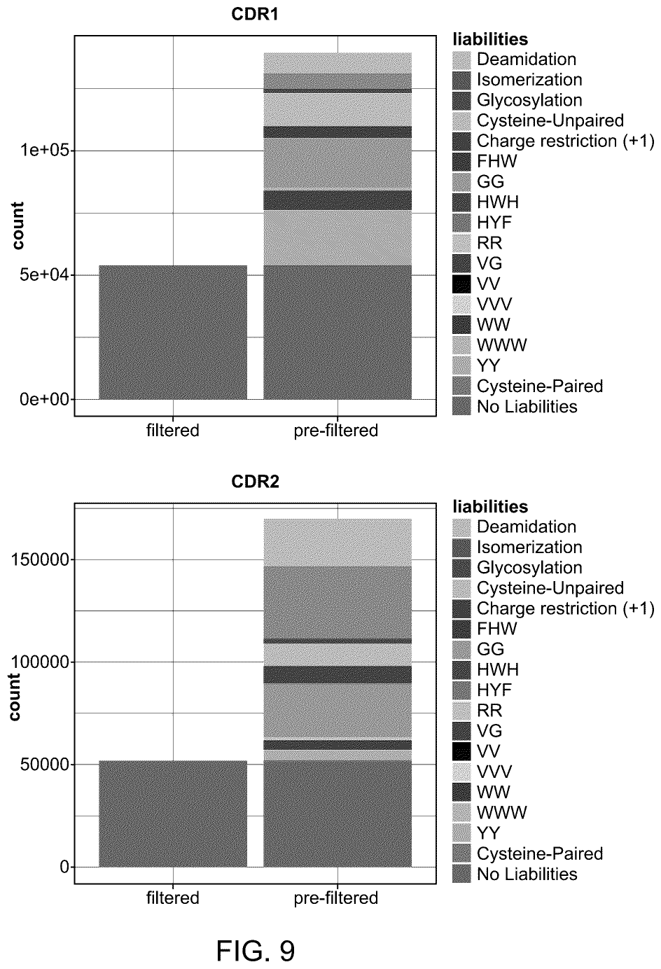

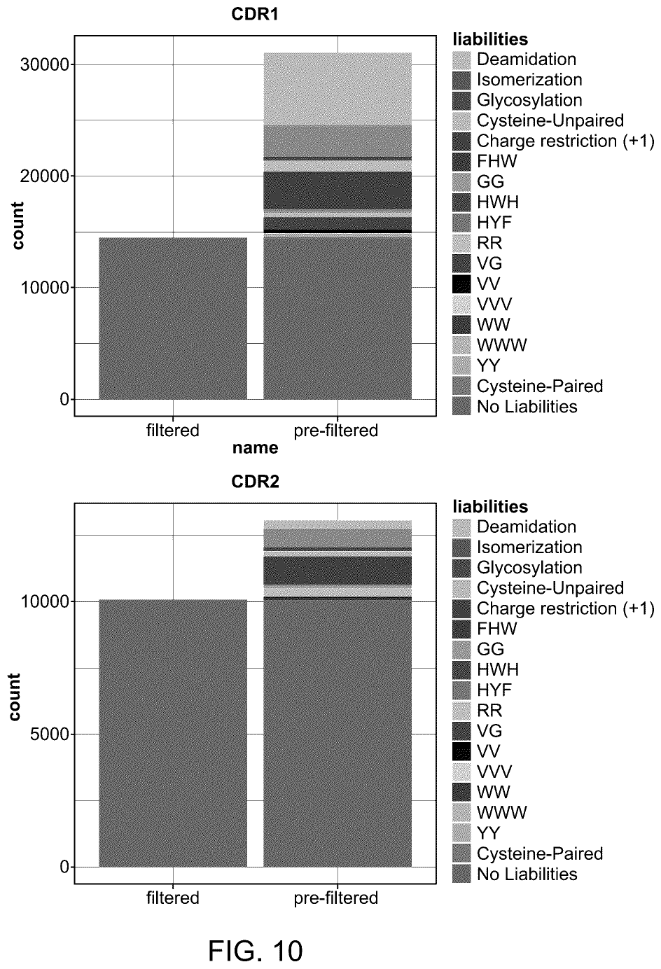

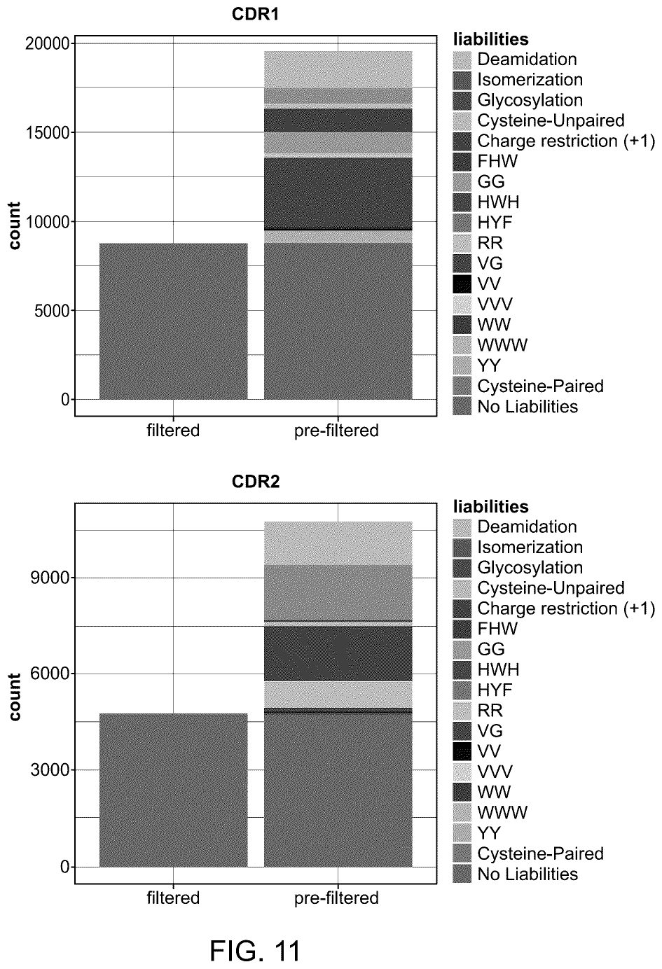

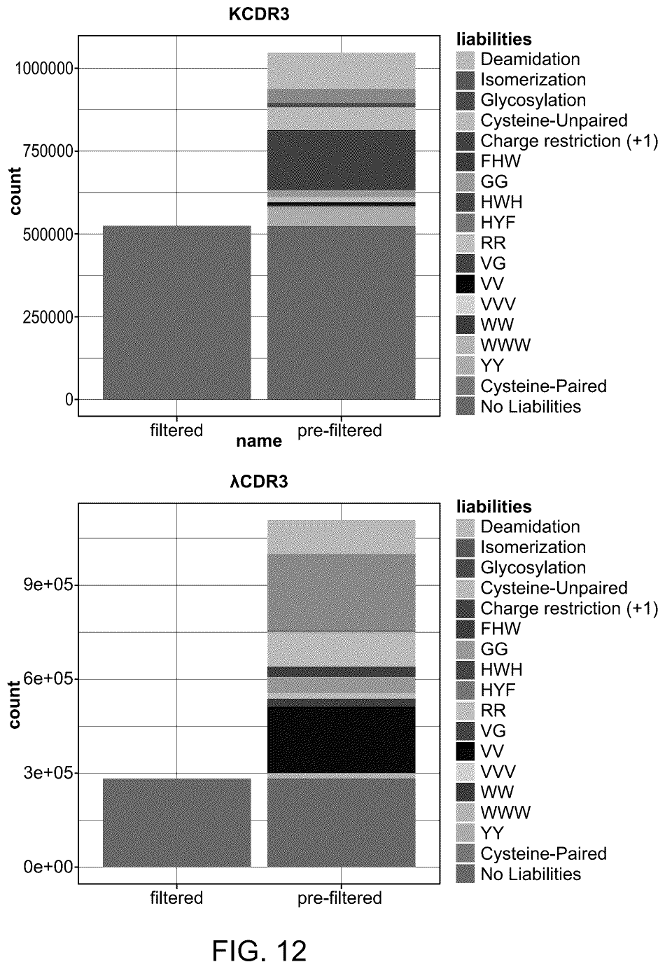

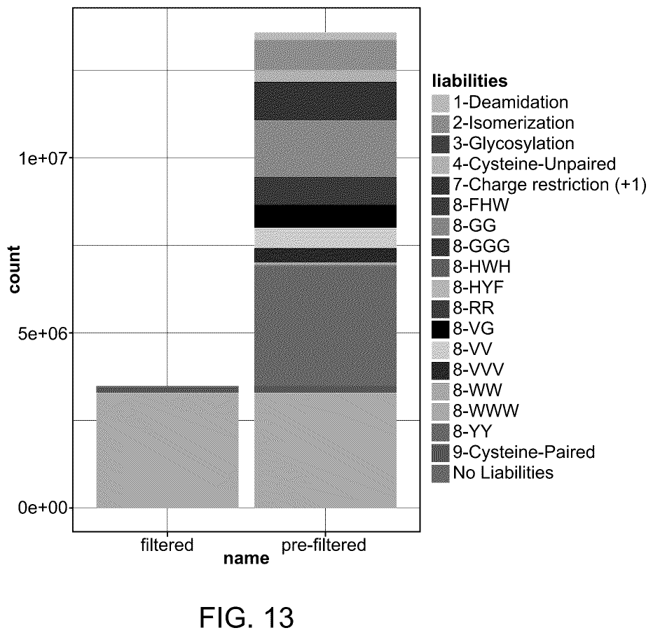

Liabilities that were reduced to short sequences were used to identify CDRs containing them. For example, HCDR2 sequences containing liabilities such as deamidation, isomerization, glycosylation or unpaired cysteines are highlighted in pink (in FIG. 5). To underscore the importance of identifying liabilities and eliminating CDRs containing liabilities from the libraries, unpaired cysteines in HCDR3 were illustrated in FIGS. 6-8 as an example. Cysteines comprise up to 4% of HCDR3 amino acid and they need to be paired and structurally positioned, otherwise the presence of unpaired cysteines or poorly positioned cysteines would introduce undesirable structure or chemical reactivity into the CDR thus rendering the CDR non-functional or non-developable. The list of exemplary liabilities identified is described in Table 4, and it is clear that additional sequence-based liabilities can be similarly screened. The list of unique CDRs previously identified in Table 3 was examined for occurrence of the listed liabilities, and all CDRs containing a liability were computationally eliminated from the list of unique CDRs. FIGS. 9-13 reflects the extent of elimination of liabilities from the different CDR populations.

TABLE 3
Unique CDRs Identified and the Remaining Unique CDRs after Elimination of liabilities.

| | | | unique LCDR1 | | unique LCDR2 | | unique LCDR3 | | unique HCDR1 | |
|---|---|---|---|---|---|---|---|---|---|---|
| Library | VH | VL | total | final | total | final | total | final | total | final |
| 1 Abrilumab | VH1-24 | VK1-12 | 8,838 | 1,717 | 6,483 | 1,406 | 385,836 | 74,091 | 19,635* | 2,860 |
| 2 Mepolizumab | VH2-70 | VK4-1 | 6,039 | 103 | 685 | 140 | 130628 | 17,917 | 31,486 | 2,296 |
| 3 Crenezumab | VH3-7 | VK2D-29 | 4,668 | 50 | 1591 | 229 | 122745 | 32,092 | 34,575 | 5,920 |
| 4 Necitumumab | VH4-30-4 | VK3-11 | 13,345 | 1,910 | 685 | 972 | 436129 | 79,038\hat{ } | 50,335 | 1,285 |
| 5 Anifrolumab | VH5-51 | VK3-20 | 13,345 | 1,910 | 685 | 972 | 436129 | 79,038\hat{ } | 11,710 | 1,979 |
| 6 Evolocumab | VH1-18 | V12-14 | 19,785 | 1,696 | 10,792 | 1,197 | 1,105,584 | 94,371 | 19,635* | 2,860 |
| | | Total | 66,020 | 5,476 | 20,921 | 3,944 | 2,617,051 | 297,509 | 167,376 | 17,200 |

| | | | | | | | Theoretical diversity | |
|---|---|---|---|---|---|---|---|---|
| | | | unique HCDR2 | | Unique HCDR3 | | No | with 1e8 |
| Library | VH | VL | total | final | total | final | HCDR3 | HCDR3's |
| 1 Abrilumab | VH1-24 | VK1-12 | 31,412 | 2,171 | 13,588,754 | 1,791,801 | 1.11E+18 | 1.11E+26 |
| 2 Mepolizumab | VH2-70 | VK4-1 | 15,550 | 1,253 | | | 7.43E+14 | 7.43E+22 |
| 3 Crenezumab | VH3-7 | VK2D-29 | 82,817 | 4,565 | | | 9.93E+15 | 9.93E+23 |
| 4 Necitumumab | VH4-30-4 | VK3-11 | 28,267 | 2,739 | | | 5.16E+17 | 5.16E+25 |
| 5 Anifrolumab | VH5-51 | VK3-20 | 12,712 | 669 | | | 1.94E+17 | 1.94E+25 |
| 6 Evolocumab | VH1-18 | V12-14 | 31,412 | 2,171 | | | 1.19E+18 | 1.19E+26 |
| | | Total | 202,170 | 13,568 | 13,588,754 | 1,791,801 | 3.00E+18 | 3.00E+26 |

Sum of final CDRs (LCDR1-3, HCDR1-2): 337, 697.
*These identified CDRs are identical, but synthesized with different flanking regions corresponding to different VH1 genes (VH1-18 and VH1-24).
\hat{ }These identified CDRs are identical, but synthesized with different flanking regions corresponding to different VK3 genes (VK3-20 and VK3-11).

TABLE 4
Liabilities to be Removed.

| Type of Liabilities | Exemplary Motif |
|---|---|
| Glycosylation - | NXS, → X = Any Amino |
| impacts stability, | Acid but Proline |
| solubility, half- | NXT, → X = Any Amino |
| life, heterogeneity, | Acid but Proline |
| and effector | NXC → X = Any Amino |
| function. | Acid but Proline |
| Deamidation - | NG, NS, NT, NN, NA, |
| Therapeutic anti- | NH, ND, GNF, GNY, |
| bodies may undergo | GNT, or GNG |
| deamidation during | |
| manufacture and | |
| storage leading to | |
| protein structural | |
| changes, aggregation, | |
| change in pharmaco- | |
| kinetics, loss of | |
| activity and | |
| immunogenicity. | |
| Isomerization - Asp | DT, DH, DG, DS, DD |
| residues can undergo | |
| isomerization and | |
| reported in CDRs. | |
| Known to increase | |
| charge heterogeneity | |
| Based on creation of | GG, GGG, RR, VG, VV, |
| synthetic library, | VVV, WW, WWW, YY, WXW |
| selection against | (X represents any |
| polyspecificity | amino acid residue) |
| (PSR) and sequencing | |
| Single cluster in | FHW |
| IL-13 human mAb | |
| HCDR3 which highly | |
| aggregating, alanine | |
| mutations increase | |
| solubility | |
| (aggregation) | |
| Two aromatic tripep- | HYF, HWH |
| tides in HCDR3 | |
| mutated improve | |
| viscosity. Compatible | |
| with idea that 3 | |
| consecutive aromatics | |
| is bad news and should | |
| be eliminated | |
| Positive charge as- | Net Charge (+1) in |
| sociated with poor | LCDR1-3, HCDR1-2 |
| developability | |
| properties. | |
| Unpaired cysteine can | Unpaired Cysteine |
| impact protein folding, | |
| function and stability. | |
| These reactive centers | |
| lead to formation of | |
| covalent aggregates | |
| and reduce protein | |
| stability | |
| Protease sensitivity | DP, DG, DS, DV, DY, |
| (fragmentation) | DF, DQ, DK, DL, DD |
| Integrin binding site | RGD, RYD, LDV, KGD |
| Lysine glycation site | KE, EK, or ED |
| Metal catalyzed | HS, SH, KT, HXS, SXH |
| fragmentation | (X represents any |
| | amino acid residue) |
| Polyspecificity, | X₁X₂X₃, in which each |
| aggregation | of X₁, X₂, and X₃ |
| | independent is F, I, |
| | L, V, W, or Y |
| Streptavidin binding | HPQ, EPDW (SEQ ID |
| motifs | NO: 117), PWXWL (SEQ |
| | ID NO: 118), GDWVFI |
| | (SEQ ID NO: 119), |
| | PWPWLG (SEQ ID NO: |
| | 120) (X represents |
| | any amino acid |
| | residue) |

### Example 4: Elimination of CDRs Arising from Sequencing Errors

Following the removal of CDRs containing potential liabilities disclosed in Example 3 above, CDRs that may have arisen as a result of sequencing errors were also computationally eliminated. Sequencing errors are more likely when the CDRs sequenced are oversampled. In general, the more copies of a particular CDR, the more likely that it is real, and not the result of a sequencing error.

The abundance of each unique CDR in the dataset was assessed after those containing liabilities had been removed. For each of the different libraries and individual CDRs, the percentage of sequences retained at different abundances (number of reads) was assessed.

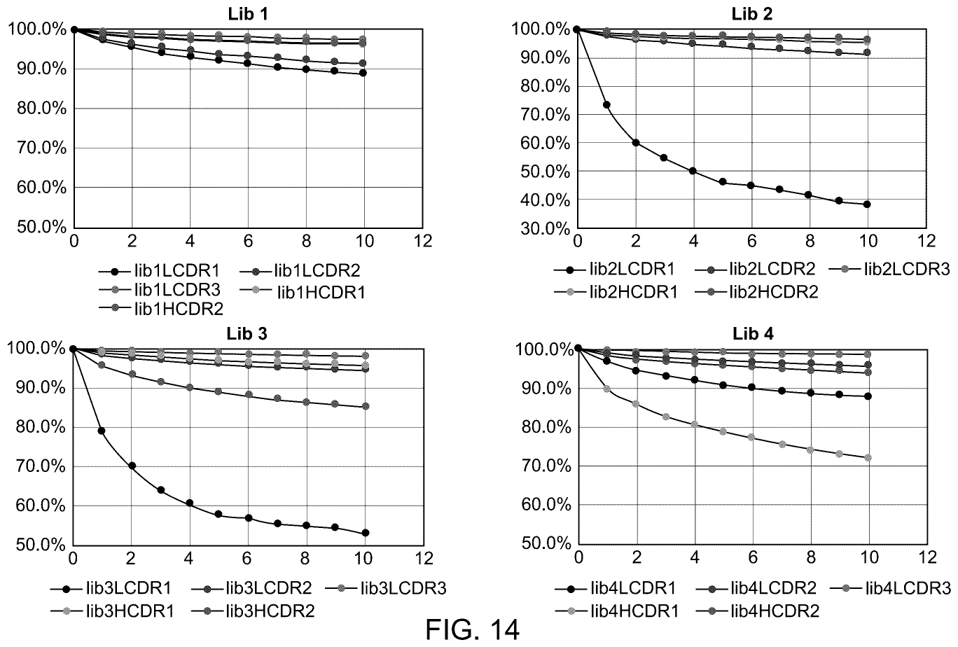

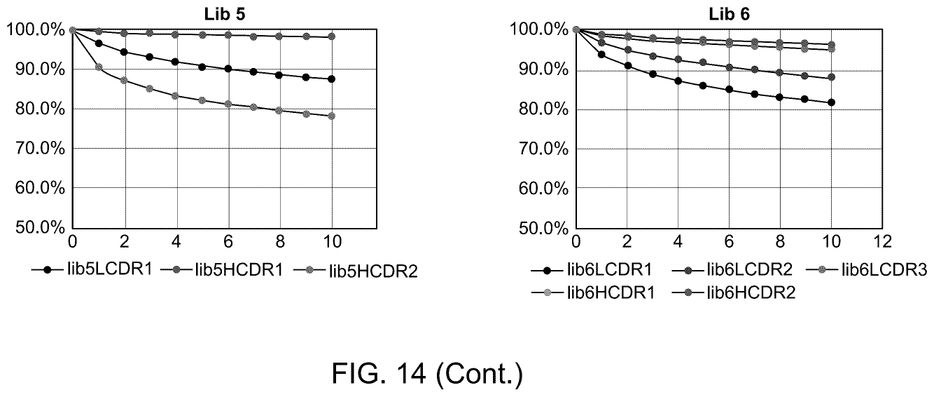

The more information retained, the more likely that rare CDRs are the result of sequencing errors, and not naturally occurring CDRs. The percentage of sequences eliminated for different CDRs except for HCDR3 at the application of different threshold numbers in each library is represented in FIG. 14. A threshold of 4 or more reads was evaluated for each unique CDR to represent the best balance between the number of unique CDRs and the retained sequence information. It is clear that different threshold numbers can be chosen depending upon the number of total reads, and the number of total unique CDRs identified. While different thresholds for each individual CDR for each library could be used, the single threshold of 4 reads was chosen to be consistent. CDRs with less than 4 reads were removed from the library.

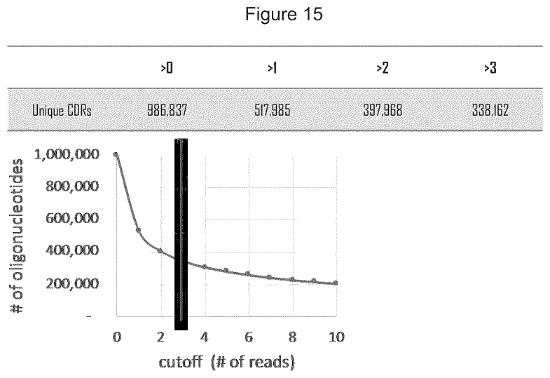

The use of different thresholds would be expected to yield different numbers of CDRs with different sequences as shown in FIG. 15.

### Example 5: Elimination of CDRs Based on Anomalous Length

The lengths of germline CDRs are conserved, although they may vary during affinity maturation. CDRs of anomalous length are expected to reduce folding and expression of antibodies that contain them. CDRs with anomalous lengths were also computationally eliminated under the rules described in FIG. 4 and CDRs with a length outside of the pink shaded area were eliminated as shown FIGS. 16-21. The effects on the number of retained CDRs are indicated in Table 5 and Table 6. It is clear that alternative rules may be applied to the identification of naturally occurring CDRs that lack liabilities, and that as new liabilities are identified, they can be similarly eliminated. Furthermore, by carrying out next generation sequencing on CDRs experimentally selected for the presence of liabilities, such as polyreactivity or aggregation behavior, additional sequence liabilities can be identified and can be eliminated in other libraries created using this approach. As the numbers of CDRs sequenced increases, it is expected that the number of both unique CDRs, as well as the number of unique CDRs lacking liabilities will increase, so increasing the potential library diversity.

After the completion of these different informatic operations, the final number of unique CDRs identified as containing no liabilities, for each scaffold for this exemplary library is indicated in Table 3 showing both unique CDRs and CDRs including flanking scaffold oligos (in parentheses):
˜3,566 (5,476) LCDR1s;
˜2,972 (3,944) LCDR2s;
˜218,471 (297,509) LCDR3s;
˜14,340 (17,200) HCDR1s;
˜11,397 (13,568) HCDR2s; and
˜1,791,801 HCDR3s

TABLE 5
Number of CDRs Retained after Defined Length Restriction.

| | No length | Length | |
|---|---|---|---|
| | restriction | Restriction | Difference |
| Library 1 | | | |
| LCDR1 | 1,719 | 1,717 | −2 |
| LCDR2 | 1,409 | 1,406 | −3 |
| LCDR3 | 74,134 | 74,091 | −43 |
| HCDR1 | 2,878 | 2,860 | −18 |
| HCDR2 | 2,189 | 2,171 | −18 |
| Total | 82,329 | 82,245 | −84 |
| Library 2 | | | |
| LCDR1 | 122 | 103 | −19 |
| LCDR2 | 141 | 140 | −1 |
| LCDR3 | 17,920 | 17,917 | −3 |
| HCDR1 | 2,335 | 2,296 | −39 |
| HCDR2 | 1,262 | 1,253 | −9 |
| Total | 21,780 | 21,709 | −71 |
| Library 3 | | | |
| LCDR1 | 57 | 50 | −7 |
| LCDR2 | 231 | 229 | −2 |
| LCDR3 | 32,111 | 32,092 | −19 |
| HCDR1 | 5,956 | 5,920 | −36 |
| HCDR2 | 4,569 | 4,565 | −4 |
| Total | 42,924 | 42,856 | −68 |
| Library 4 | | | |
| LCDR1 | 1,917 | 1,910 | −7 |
| LCDR2 | 979 | 972 | −7 |
| LCDR3 | 79,141 | 79,038 | −103 |
| HCDR1 | 1,293 | 1,285 | −8 |
| HCDR2 | 2,773 | 2,739 | −34 |
| Total | 86,103 | 85,944 | −159 |
| Library 5 | | | |
| HCDR1 | 1,999 | 1,979 | −20 |
| HCDR2 | 673 | 669 | −4 |
| Total | 24,452 | 24,357 | −95 |
| Library 6 | | | |
| LCDR1 | 1,697 | 1,696 | −1 |
| LCDR2 | 1,207 | 1,197 | −10 |
| LCDR3 | 94,383 | 94,371 | −12 |
| HCDR1 | 2,878 | 2,860 | −18 |
| HCDR2 | 2,189 | 2,171 | −18 |
| Total | 102,354 | 102,295 | −59 |

TABLE 6
Total Number of CDRs.

| | No length | Length | |
|---|---|---|---|
| | restriction | Restriction | Difference |
| Total | 338,162 | 337,697 | −465 |

### Example 6: Synthesis and Amplification of Oligonucleotides Corresponding to Final CDRs

Oligonucleotides corresponding to those identified for HCDR1-2 and LCDR1-3 after the elimination steps as described in the above Examples were synthesized (Twist, Inc., San Francisco, CA), resulting in a total of 337,697 oligonucleotides coding for the selected CDRs. The CDR coding sequence in these oligonucleotides was flanked by 5′ and 3′ sequences homologous to the framework vectors, into which the CDR coding sequences were cloned. The homologous sequences were used for both amplification and insertion of the oligonucleotides into the yeast display vectors.

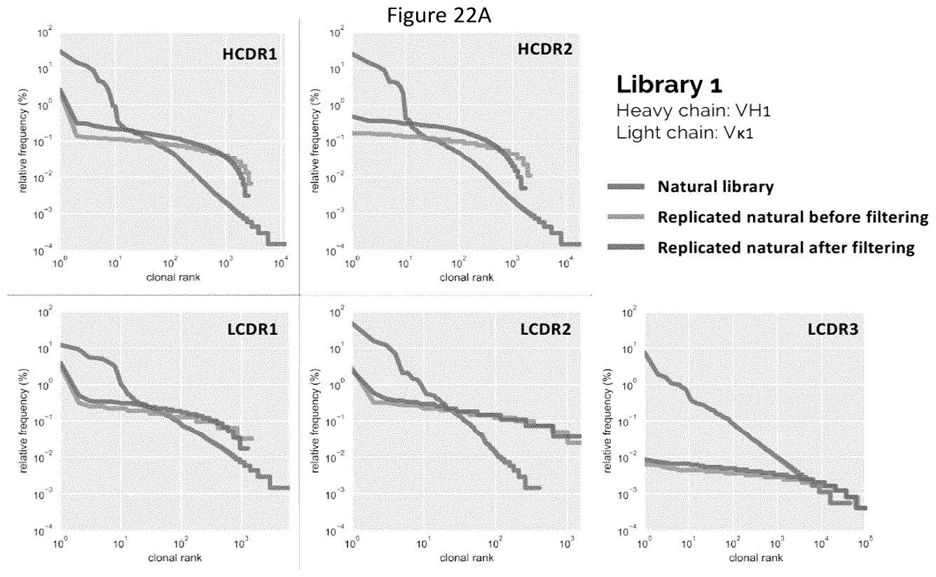

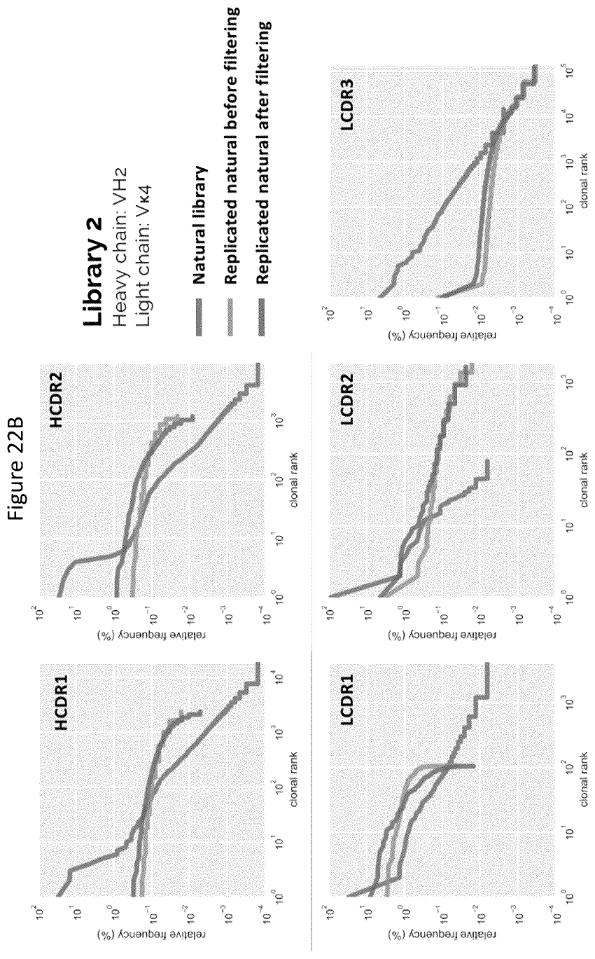

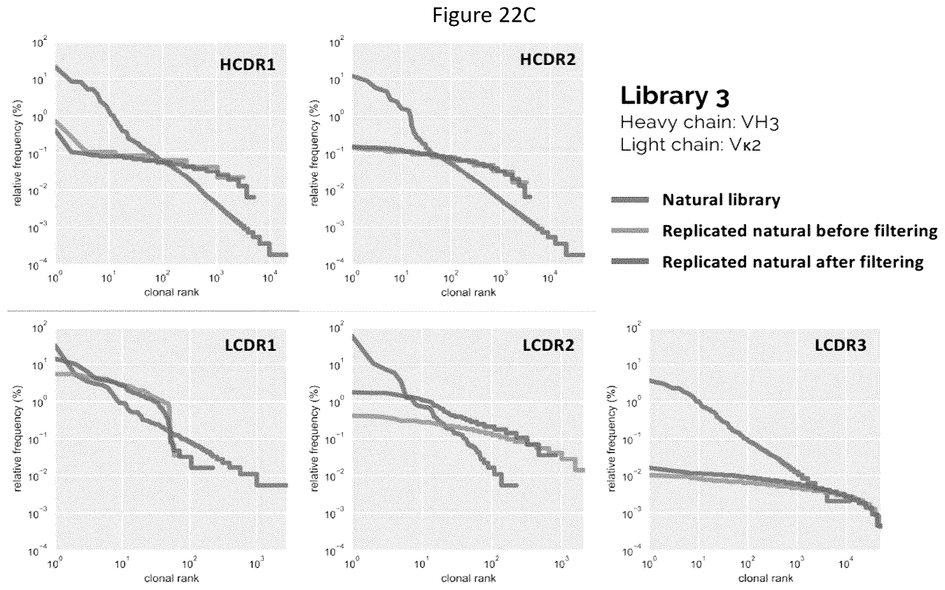

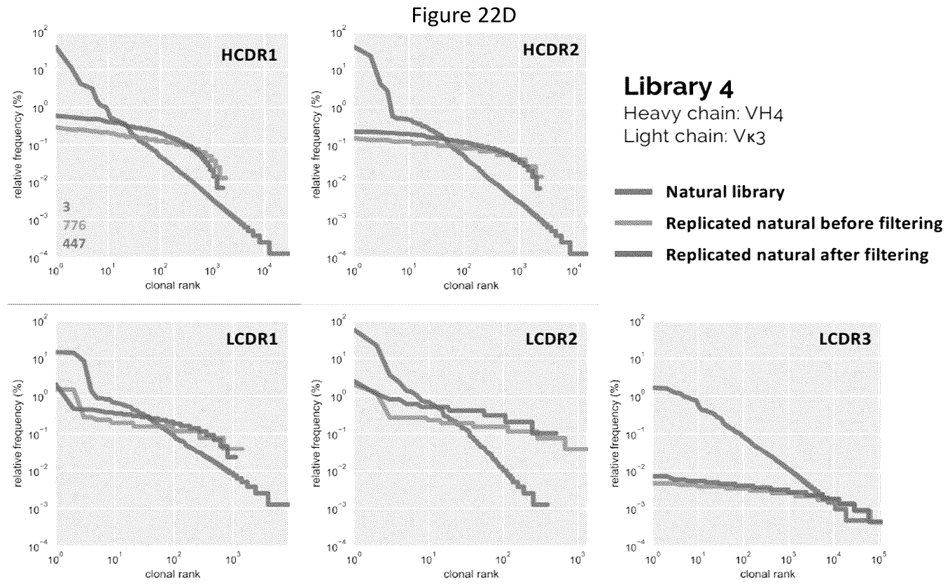

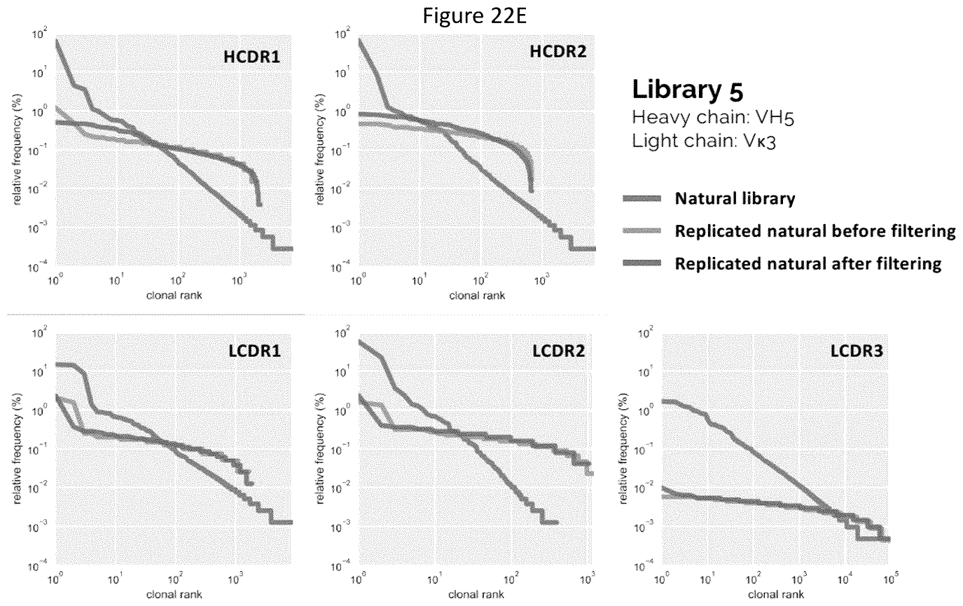

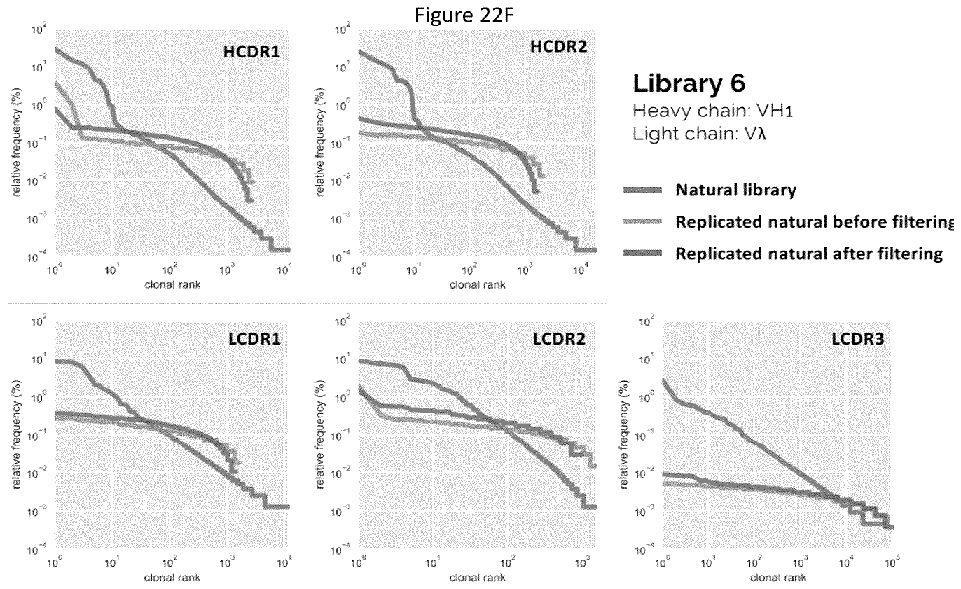

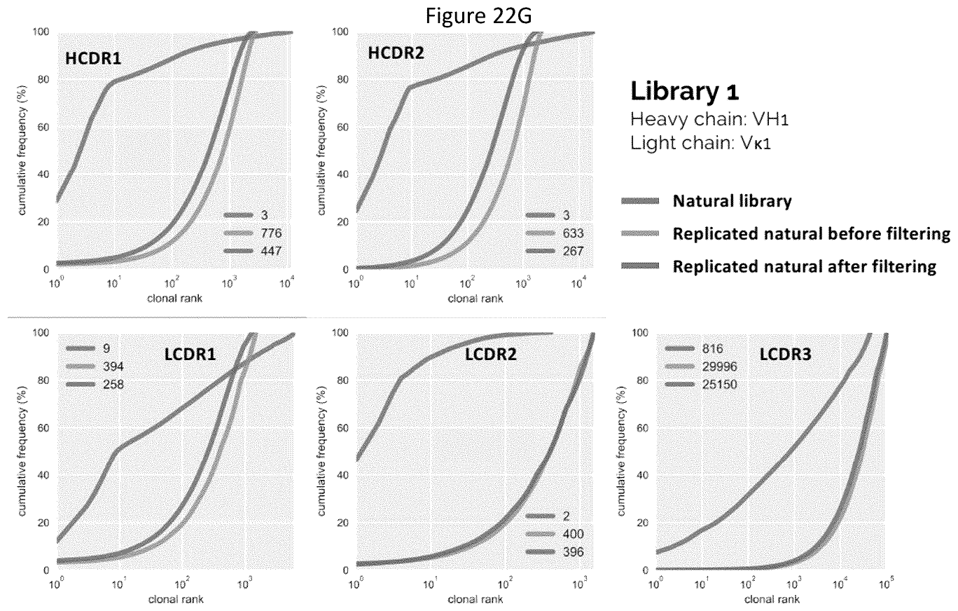

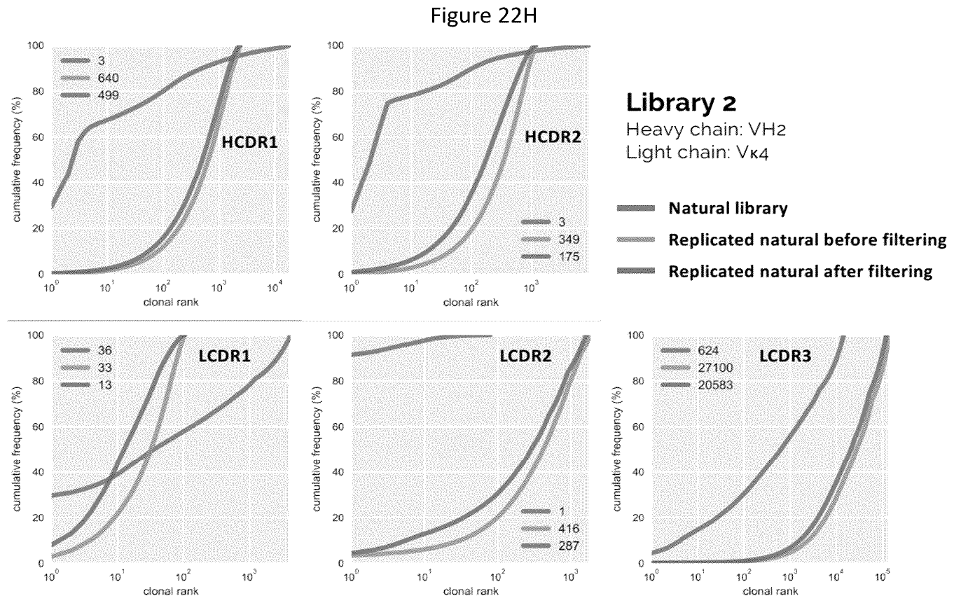

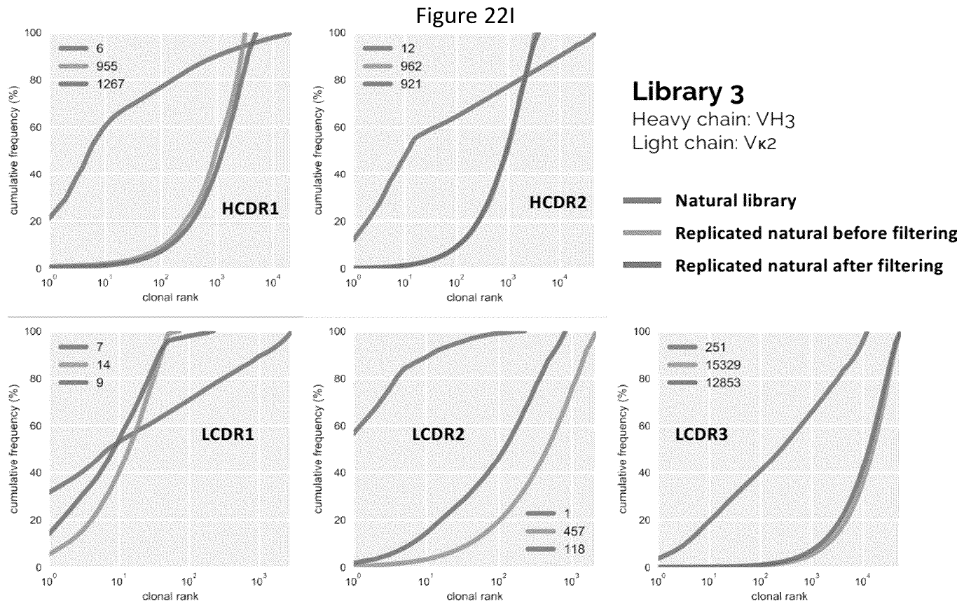

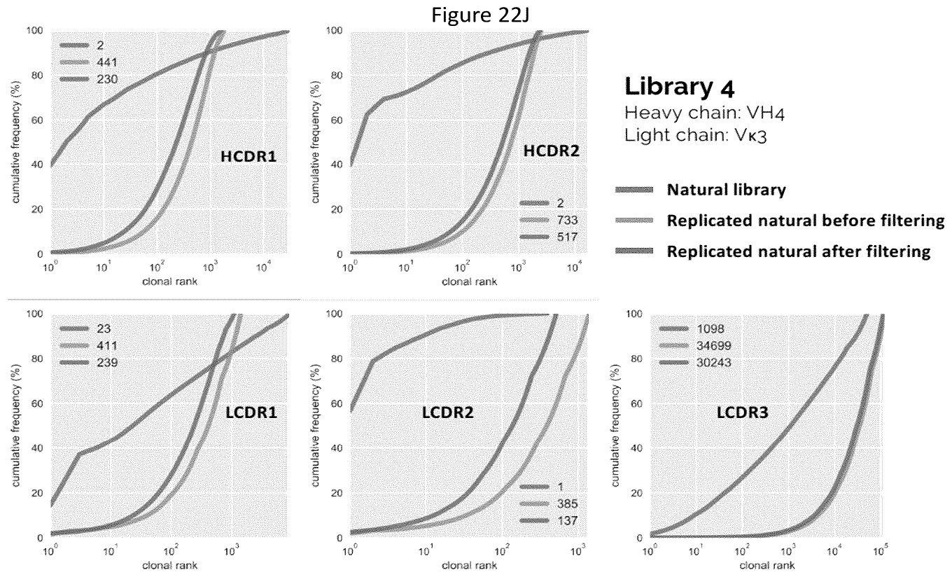

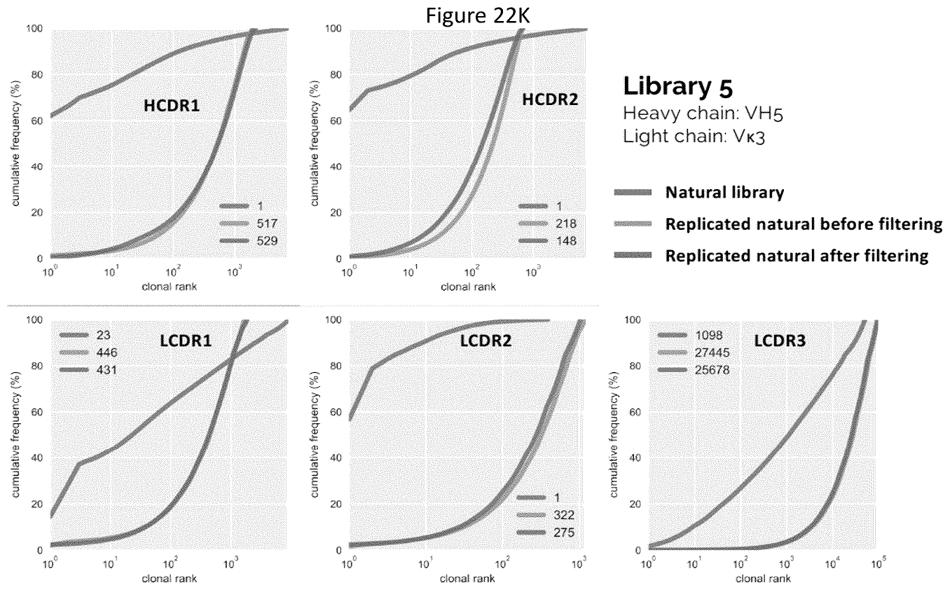

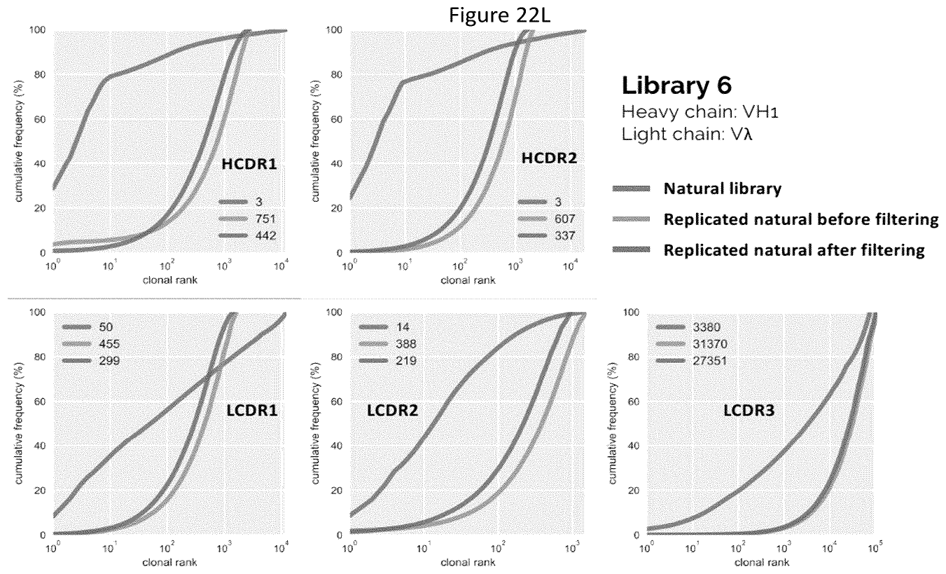

The combined pool of replicated natural CDRs amplified using primer pairs specific for each library scaffold and CDR position, cloned into the yeast display vectors described in Example 2 by homologous recombination, resulted in 30 different single CDR loop libraries (6 libraries, LCDR1-3, HCDR1-2). These were sorted for display, using a monoclonal antibody recognizing the SV5 tag by fluorescence activated cell sorting. For each of these libraries, this represents the diversity of replicated natural CDRs that are amplified using the specific primer pairs used (see below) that allow any level of display and are indicated as “Replicated natural before filtering” in FIG. 22.

FIG. 22A-F illustrates exemplary advantage of using synthetic oligonucleotides to encode HCDR1-2 and LCDR1-3 replicated natural diversity. Particularly for CDR1-2, the difference in abundance between the most and least abundant CDRs can be >300,000-fold, with the germline CDR1-2 sequences being by far the most abundant. When natural CDRs are synthesized, the distribution is always far flatter, as shown in FIGS. 22A-F, with the difference in abundance between the most and least abundant CDRs ranging from 10-200 fold, depending upon the CDR and library. The improvement in the diversity at each CDR using the approach described here is further illustrated in FIGS. 22G-L, where the cumulative distribution is indicated for each library and CDR position. The figures for each plot indicate the D50, the number of clones comprising the most abundant 50% of clones, which for all CDRs in all libraries is significantly higher for the replicated natural diversity, than for the natural diversity. In the case of HCDR3, the VDJ recombinatorial process (including addition of removal of nucleotides at the VD and DJ junctions) results in less variability in abundance between different HCDR3s.

The pool of oligonucleotides was subjected to amplification using the following primers:
For LCDR1: F-L1-LCDR1 to F-L6-LCDR1 and R-L1-LCDR1 to R-L6-LCDR1
For LCDR2: F-L1-LCDR2 to F-L6-LCDR2 and R-L1-LCDR2 to R-L6-LCDR2
For LCDR3: F-L1-LCDR3 to F-L6-LCDR3 and R-L1-LCDR3 to R-L6-LCDR3
For HCDR1: F-L1-HCDR1 to F-L1-HCDR1 and R-L1-HCDR1 to R-L1-HCDR1
For HCDR2: F-L1-HCDR2 to F-L1-HCDR2 and R-L1-HCDR2 to R-L1-HCDR2

The exemplary amplification primer sequences and assembly primer sequences are provided in Table 7 and Table 8, respectively.

TABLE 7
Exemplary Amplification Primers.

| | | | | SEQ |
|---|---|---|---|---|
| | | | Tm | ID |
| | Name | Sequence | (° C.) | NO |
| 0 | F-L1-LCDR1 | GCTTCTGTTGGTGATCGTGTT | 77.9 | 181 |
| | | ACTATTACCTGTCGTGCTTCT | | |
| 1 | F-L2-LCDR1 | GTTTCTCTGGGTGAACGTGCT | 77.9 | 182 |
| | | ACCATCAACTGCAAATCTTCT | | |
| 2 | F-L3-LCDR1 | GTTACCCCAGGTGAACCAGCT | 77.9 | 183 |
| | | TCTATTTCTTGTCGTTCTTCT | | |
| 3 | F-L4-LCDR1 | CTGTCTCCAGGTGAACGTGCC | 81.8 | 184 |
| | | ACTCTGTCTTGTCGTGCTTCT | | |
| 4 | F-L5-LCDR1 | CTGTCTCCAGGTGAACGTGCC | 81.8 | 185 |
| | | ACTCTGTCTTGTCGTGCTTCT | | |
| 5 | F-L6-LCDR1 | GGTTCTCCAGGTCAGTCTATC | 80.9 | 186 |
| | | ACCATCTCTTGTACCGGTACC | | |
| 6 | R-L1-LCDR1 | CAGTTTTGGAGCTTTACCTGG | 79.9 | 187 |
| | | TTTCTGCTGGTACCAAGCCAG | | |
| 7 | R-L2-LCDR1 | CAGTTTTGGTGGCTGACCTGG | 81.8 | 188 |
| | | TTTCTGCTGGTACCAAGCCAG | | |
| 8 | R-L3-LCDR1 | CAGCTGTGGAGACTGACCTGG | 82.8 | 189 |
| | | TTTCTGCAGGTACCAGTGCAG | | |
| 9 | R-L4-LCDR1 | CAGACGTGGAGCCTGACCTGG | 83.8 | 190 |
| | | TTTCTGCTGGTACCAAGCCAG | | |
| 10 | R-L5-LCDR1 | CAGACGTGGAGCCTGACCTGG | 83.8 | 191 |
| | | TTTCTGCTGGTACCAAGCCAG | | |
| 11 | R-L6-LCDR1 | CAGTTTTGGAGCTTTACCTGG | 79.9 | 192 |
| | | GTGCTGCTGGTACCAAGAAAC | | |
| 12 | F-L1-LCDR2 | TACCAGCAGAAACCAGGTAAA | 77.9 | 193 |
| | | GCTCCAAAACTGCTGATCTAC | | |
| 13 | F-L2-LCDR2 | TACCAGCAGAAACCAGGTCAG | 79.9 | 194 |
| | | CCACCAAAACTGCTGATCTAC | | |
| 14 | F-L3-LCDR2 | TACCTGCAGAAACCAGGTCAG | 80.9 | 195 |
| | | TCTCCACAGCTGCTGATCTAC | | |
| 15 | F-L4-LCDR2 | TACCAGCAGAAACCAGGTCAG | 81.8 | 196 |
| | | GCTCCACGTCTGCTGATCTAC | | |
| 16 | F-L5-LCDR2 | TACCAGCAGAAACCAGGTCAG | 81.8 | 197 |
| | | GCTCCACGTCTGCTGATCTAC | | |
| 17 | F-L6-LCDR2 | TACCAGCAGCACCCAGGTAAA | 78.9 | 198 |
| | | GCTCCAAAACTGATGATCTAC | | |
| 18 | R-L1-LCDR2 | ATCGGTACCAGAACCAGAACC | 79.9 | 199 |
| | | AGAGAAACGAGATGGAACACC | | |
| 19 | R-L2-LCDR2 | ATCGGTACCAGAACCAGAACC | 79.9 | 200 |
| | | AGAGAAACGATCTGGAACACC | | |
| 20 | R-L3-LCDR2 | ATCGGTACCAGAACCAGAACC | 79.9 | 201 |
| | | AGAGAAACGATCTGGAACACC | | |
| 21 | R-L4-LCDR2 | ATCGGTACCAGAACCAGAACC | 80.9 | 202 |
| | | AGAGAAACGAGCTGGGATACC | | |
| 22 | R-L5-LCDR2 | ATCGGTACCAGAACCAGAACC | 80.9 | 203 |
| | | AGACAGACGATCTGGGATACC | | |
| 23 | R-L6-LCDR2 | GGTGTTACCAGATTTAGAACC | 77.0 | 204 |
| | | AGAGAAACGGTTAGAAACACC | | |
| 24 | F-L1-LCDR3 | ATCTCTTCTCTGCAGCCAGAA | 77.0 | 205 |
| | | GATTTCGCTAACTACTACTGT | | |
| 25 | F-L2-LCDR3 | ATCTCTTCTCTGCAGGCTGAA | 77.0 | 206 |
| | | GATGTTGCTGTTTACTACTGT | | |
| 26 | F-L3-LCDR3 | ATCTCTCGTGTTGAAGCTGAA | 76.0 | 207 |
| | | GATGTTGGTGTTTACTACTGT | | |
| 27 | F-L4-LCDR3 | ATCTCTTCTCTGGAACCAGAA | 76.0 | 208 |
| | | GATTTCGCTGTTTACTACTGT | | |
| 28 | F-L5-LCDR3 | ATCACCCGTCTGGAACCAGAA | 77.9 | 209 |
| | | GATTTCGCTGTTTACTACTGT | | |
| 29 | F-L6-LCDR3 | ATCTCTGGTCTGCAGGCTGAA | 77.9 | 210 |
| | | GATGAAGCTGATTACTACTGT | | |
| 30 | R-L1-LCDR3 | CGACCCTCCGGATTTGATTTC | 79.9 | 211 |
| | | AACTTTGGTACCACCACCGAA | | |
| 31 | R-L6-LCDR3 | CGACCCTCCGGACAGAACGGT | 83.8 | 212 |
| | | CAGTTTGGTACCACCACCGAA | | |
| 32 | F-L1-HCDR1 | AAAAAACCAGGTGCTTCTGTT | 72.1 | 213 |
| | | AAAGTTTCTTGTAAAGTTTCT | | |
| 33 | F-L2-HCDR1 | GTTAAACCAACCCAGACCCTG | 80.9 | 214 |
| | | ACCCTGACCTGTACCGTTTCT | | |
| 34 | F-L3-HCDR1 | GTTCAGCCAGGTGGTTCTCTG | 81.8 | 215 |
| | | CGTCTGTCTTGTGCTGCTTCT | | |
| 35 | F-L4-HCDR1 | GTTAAACCATCTCAGACCCTG | 78.9 | 216 |
| | | TCTCTGACCTGTACCGTTTCT | | |
| 36 | F-L5-HCDR1 | AAAAAACCAGGTGAATCTCTG | 73.0 | 217 |
| | | AAAATCTCTTGTAAAGGTTCT | | |
| 37 | F-L6-HCDR1 | AAAAAACCAGGTGCTTCTGTT | 73.0 | 218 |
| | | AAAGTTTCTTGTAAAGCTTCT | | |
| 38 | R-L1-HCDR1 | CCATTCCAGACCTTTACCTGG | 81.8 | 219 |
| | | AGCCTGACGAACCCAGTGGAT | | |
| 39 | R-L2-HCDR1 | CCATTCCAGAGCTTTACCTGG | 80.9 | 220 |
| | | TGGCTGACGGATCCAGTTAAC | | |
| 40 | R-L3-HCDR1 | CAGTTCCAGACCTTTACCTGG | 80.9 | 221 |
| | | AGCCTGACGAACCCAAGACAT | | |
| 41 | R-L4-HCDR1 | CCATTCCAGACCTTTACCTGG | 81.8 | 222 |
| | | TGGCTGACGGATCCAAGACCA | | |
| 42 | R-L5-HCDR1 | AGATTCCAGACCTTTACCTGG | 79.9 | 223 |
| | | AACCTGACGAACCCAAGCGAT | | |
| 43 | R-L6-HCDR1 | CCATTCCAGACCCTGACCTGG | 82.8 | 224 |
| | | AGCCTGACGAACCCAAGAGAT | | |
| 44 | F-L1-HCDR2 | GTTCGTCAGGCTCCAGGTAAA | 81.8 | 225 |
| | | GGTCTGGAATGGATGGGTGGT | | |
| 45 | F-L2-HCDR2 | ATCCGTCAGCCACCAGGTAAA | 81.8 | 226 |
| | | GCTCTGGAATGGCTGGCTATG | | |
| 46 | F-L3-HCDR2 | GTTCGTCAGGCTCCAGGTAAA | 80.9 | 227 |
| | | GGTCTGGAACTGGTTGCTTCT | | |
| 47 | F-L4-HCDR2 | ATCCGTCAGCCACCAGGTAAA | 80.9 | 228 |
| | | GGTCTGGAATGGATCGGTTAC | | |
| 48 | F-L5-HCDR2 | GTTCGTCAGGTTCCAGGTAAA | 78.9 | 229 |
| | | GGTCTGGAATCTATGGGTATC | | |
| 49 | F-L6-HCDR2 | GTTCGTCAGGCTCCAGGTCAG | 83.8 | 230 |
| | | GGTCTGGAATGGATGGGTTGG | | |
| 50 | R-L1-HCDR2 | TTCGGTCATGGTAACACGACC | 78.9 | 231 |
| | | CTGGAATTTCTGAGCGTAGAT | | |
| 51 | R-L2-HCDR2 | AGAGATGGTCAGACGAGATTT | 77.0 | 232 |
| | | CAGAGCAGAGTTGTAAACGAT | | |
| 52 | R-L3-HCDR2 | ACGAGAGATGGTGAAACGACC | 77.0 | 233 |
| | | TTTAACAGAATCTGGGTAGTA | | |
| 53 | R-L4-HCDR2 | AACAGACATGGTAACACGAGA | 76.0 | 234 |
| | | TTTCAGAGATGGGTTGTAATC | | |
| 54 | R-L5-HCDR2 | AGCAGAGATGGTAACCTGACC | 79.9 | 235 |
| | | CTGGAAAGATGGAGAGTAACG | | |
| 55 | R-L6-HCDR2 | GGTGGTCATGGTACCACGACC | 81.8 | 236 |
| | | CTGCAGTTTCTGAGCGTAGTT | | |
| 56 | F-L1-HCDR3 | CTGTCTTCTCTGAAATCTGAG | 78.9 | 237 |
| | | GACACGGCCGTGTATTACTGT | | |
| 57 | F-L2-HCDR3 | ATGACCAACATGGATCCTGTG | 77.9 | 238 |
| | | GACACAGCCACATATTACTGT | | |
| 58 | F-L3-HCDR3 | ATGAACTCTCTGCGTGCCGAG | 80.9 | 239 |
| | | GACACGGCTGTGTATTACTGT | | |
| 59 | F-L4-HCDR3 | GTTAACTCTGTTACCGCCGCG | 80.9 | 240 |
| | | GACACGGCTGTGTATTACTGT | | |
| 60 | F-L5-HCDR3 | TGGTCTTCTCTGAAAGCCTCG | 78.9 | 241 |
| | | GACACCGCCATTTATTACTGT | | |
| 61 | F-L6-HCDR3 | CTGCGTTCTCTGCGTTCTGAC | 81.8 | 242 |
| | | GACACGGCCGTGTATTACTGT | | |
| 62 | R-JH4 | GATTGGTTTGCCGCTAGCTGA | 83.8 | 243 |
| | | GGAGACGGTGACCAGGGTTCC | | |
| 63 | R-JH6 | GATTGGTTTGCCGCTAGCTGA | 84.8 | 244 |
| | | GGAGACGGTGACCGTGGTCCC | | |

TABLE 8
Exemplary Assembly Primers.

| | | | | SEQ |
|---|---|---|---|---|
| | | | Tm | ID |
| | Name | Sequences | (° C.) | NO |
| 64 | R-L1-LCDR1- | GTAGATCAGCAGTTTTGGAGC | 61.8 | 245 |
| | ASS | TTT | | |
| 65 | R-L2-LCDR1- | GTAGATCAGCAGTTTTGGTGG | 59.4 | 246 |
| | ASS | | | |
| 66 | R-L3-LCDR1- | GTAGATCAGCAGCTGTGGAGA | 61.3 | 247 |
| | ASS | | | |
| 67 | R-L4-LCDR1- | GTAGATCAGCAGACGTGGAG | 60.5 | 248 |
| | ASS | | | |
| 68 | R-L5-LCDR1- | GTAGATCAGCAGACGTGGAG | 60.5 | 249 |
| | ASS | | | |
| 69 | R-L6-LCDR1- | GTAGATCATCAGTTTTGGAGC | 60.9 | 250 |
| | ASS | TTTA | | |
| 70 | F-L1-LCDR2- | CTGGCTTGGTACCAGCAGAAA | 61.3 | 251 |
| | ASS | | | |
| 71 | F-L2-LCDR2- | CTGGCTTGGTACCAGCAGAAA | 61.3 | 252 |
| | ASS | | | |
| 72 | F-L3-LCDR2- | CTGCACTGGTACCTGCAGAAA | 61.3 | 253 |
| | ASS | | | |
| 73 | F-L4-LCDR2- | CTGGCTTGGTACCAGCAGAAA | 61.3 | 254 |
| | ASS | | | |
| 74 | F-L5-LCDR2- | CTGGCTTGGTACCAGCAGAAA | 61.3 | 255 |
| | ASS | | | |
| 75 | F-L6-LCDR2- | GTTTCTTGGTACCAGCAGCAC | 61.3 | 256 |
| | ASS | | | |
| 76 | R-L1-LCDR2- | ACAGTAGTAGTTAGCGAAATC | 60.9 | 257 |
| | ASS | TTCT | | |
| 77 | R-L2-LCDR2- | ACAGTAGTAAACAGCAACATC | 60.9 | 258 |
| | ASS | TTCA | | |
| 78 | R-L3-LCDR2- | ACAGTAGTAAACACCAACATC | 60.9 | 259 |
| | ASS | TTCA | | |
| 79 | R-L4-LCDR2- | ACAGTAGTAAACAGCGAAATC | 60.9 | 260 |
| | ASS | TTCT | | |
| 80 | R-L5-LCDR2- | ACAGTAGTAAACAGCGAAATC | 60.9 | 261 |
| | ASS | TTCT | | |
| 81 | R-L6-LCDR2- | ACAGTAGTAATCAGCTTCATC | 60.9 | 262 |
| | ASS | TTCA | | |
| 82 | F-L1-LCDR3- | GGTGTTCCATCTCGTTTCTCT | 59.4 | 263 |
| | ASS | | | |
| 83 | F-L2-LCDR3- | GGTGTTCCAGATCGTTTCTCT | 59.4 | 264 |
| | ASS | | | |
| 84 | F-L3-LCDR3- | GGTGTTCCAGATCGTTTCTCT | 59.4 | 265 |
| | ASS | | | |
| 85 | F-L4-LCDR3- | GGTATCCCAGCTCGTTTCTCT | 61.3 | 266 |
| | ASS | | | |
| 86 | F-L5-LCDR3- | GGTATCCCAGATCGTCTGTCT | 61.3 | 267 |
| | ASS | | | |
| 87 | F-L6-LCDR3- | GGTGTTTCTAACCGTTTCTCT | 60.3 | 268 |
| | ASS | G | | |
| 90 | R-L1-HCDR1- | ACCACCCATCCATTCCAGAC | 60.5 | 269 |
| | ASS | | | |
| 91 | R-L2-HCDR1- | CATAGCCAGCCATTCCAGAG | 60.5 | 270 |
| | ASS | | | |
| 92 | R-L3-HCDR1- | AGAAGCAACCAGTTCCAGACC | 61.3 | 271 |
| | ASS | | | |
| 93 | R-L4-HCDR1- | GTAACCGATCCATTCCAGACC | 61.3 | 272 |
| | ASS | | | |
| 94 | R-L5-HCDR1- | GATACCCATAGATTCCAGACC | 61.8 | 273 |
| | ASS | TTT | | |
| 95 | R-L6-HCDR1- | CCAACCCATCCATTCCAGAC | 60.5 | 274 |
| | ASS | | | |
| 96 | F-L1-HCDR2- | ATCCACTGGGTTCGTCAGG | 59.5 | 275 |
| | ASS | | | |
| 97 | F-L2-HCDR2- | GTTAACTGGATCCGTCAGCCA | 61.3 | 276 |
| | ASS | | | |
| 98 | F-L3-HCDR2- | ATGTCTTGGGTTCGTCAGGCT | 61.3 | 277 |
| | ASS | | | |
| 99 | F-L4-HCDR2- | TGGTCTTGGATCCGTCAGC | 59.5 | 278 |
| | ASS | | | |
| 100 | F-L5-HCDR2- | ATCGCTTGGGTTCGTCAGGTT | 61.3 | 279 |
| | ASS | | | |
| 101 | F-L6-HCDR2- | ATCTCTTGGGTTCGTCAGGCT | 61.3 | 280 |
| | ASS | | | |
| 102 | R-L1-HCDR2- | ACAGTAATACACGGCCGTGTC | 61.3 | 281 |
| | ASS | | | |
| 103 | R-L2-HCDR2- | ACAGTAATATGTGGCTGTGTC | 61.1 | 282 |
| | ASS | CA | | |
| 104 | R-L3-HCDR2- | ACAGTAATACACAGCCGTGTC | 59.4 | 283 |
| | ASS | | | |
| 105 | R-L4-HCDR2- | ACAGTAATACACAGCCGTGTC | 59.4 | 284 |
| | ASS | | | |
| 106 | R-L5-HCDR2- | ACAGTAATAAATGGCGGTGTC | 60.3 | 285 |
| | ASS | C | | |
| 107 | R-L6-HCDR2- | ACAGTAATACACGGCCGTGTC | 61.3 | 286 |
| | ASS | | | |
| 108 | F-L1-HCDR3- | ATCTACGCTCAGAAATTCCAG | 60.3 | 287 |
| | ASS | G | | |
| 109 | F-L2-HCDR3- | GTTTACAACTCTGCTCTGAAA | 60.1 | 288 |
| | ASS | TCT | | |
| 110 | F-L3-HCDR3- | TACTACCCAGATTCTGTTAAA | 60.1 | 289 |
| | ASS | GGT | | |
| 111 | F-L4-HCDR3- | GATTACAACCCATCTCTGAAA | 60.1 | 290 |
| | ASS | TCT | | |
| 112 | F-L5-HCDR3- | CGTTACTCTCCATCTTTCCAG | 59.4 | 291 |
| | ASS | | | |
| 113 | F-L6-HCDR3- | AACTACGCTCAGAAACTGCAG | 59.4 | 292 |
| | ASS | | | |
| 114 | F-scfv-ASS | CGGATTGTCTTCAACCAACAC | 61.1 | 293 |
| | | AA | | |
| 115 | R-scfv-ASS | CTCCTCCTGTTGAATCCAGG | 60.5 | 294 |
| 116 | F-scfv | CAGTTAGATAAAAGAGGCGCG | 59.4 | 295 |
| 117 | R-scfv | GCCCAGCAGTGGGTTTGG | 60.7 | 296 |
| 88 | F-linker- | TCCGGAGGGTCGACCATAA | 59.5 | 297 |
| | ASS | | | |
| 89 | R-linker- | GGTACCGCTCGAGGATAACTT | 61.3 | 298 |
| | ASS | | | |

While the diversity found in HCDR1-2 and LCDR1-3 can be covered by array-based oligonucleotide synthesis relatively easily, this may not be the case for HCDR3 in some instances, where the original diversity can easily exceed 10⁸ different HCDR3s. Even after liabilities and CDRs found fewer than 4 times may be eliminated, the number of different HCDR3s can exceed 10⁷ if NovaSeq (3×10⁹ reads) is used to assess diversity. This can be addressed either by limiting synthetic HCDR3 diversity to <10⁶ sequences, which is tractable by array-based synthesis; or by combining synthetic HCDR1-2 and LCDR1-3 diversity with naturally diverse HCDR3 amplified from donor lymphocytes.

RNA from B lymphocytes from Leuko Paks from ten donors, comprising a total of >10⁹ B cells, was isolated using the Miltenyi StraightFrom LeukoPak CD19 kit. cDNA was prepared using a primer annealing in the IgM constant region. HCDR3s were amplified from the cDNA using all possible combinations of the six forward primers (F-L1-HCDR3 to F1-L6-HCDR3) and the two reverse primers (R-JH4 and RJH6) described Table 7 and Table 8. This amplification appends sequences to the 5′ and 3′ ends homologous to the framework vectors, into which the HCDR3s are to be cloned.

### Example 7: Cloning of CDRs into Single Site CDR Vectors and Selection for Functional CDRs

Although natural replicated CDRs are synthesized based on criteria that should ensure their functionality (e.g., removal of liabilities), oligonucleotide synthesis may not be 100% accurate. In addition to the problem of incorrect sequences, other unidentified liabilities causing poor expression or polyreactivity may be encoded by the synthesized oligonucleotides. Selection of the functional CDRs can be an option to address this issue.

Each of the CDRs remaining from the elimination steps described above was cloned into the appropriate yeast display scaffold vector. The coding sequences of the exemplary scaffold are provided below:

> 1 - abrilumab
(SEQ ID NO: 139)
cagttagataaaagaggcgcggcagcaagcggcgcgcatgccGATATCCAGATGACCCAGTCTCCATCTT
CTGTTTCTGCTTCTGTTGGTGATCGTGTTACTATTACCTGTCGTGCTTCTCAGGGTATCTCTTCTTGGCT
GGCTTGGTACCAGCAGAAACCAGGTAAAGCTCCAAAACTGCTGATCTACGGTGCTTCTAACCTGGAATCT
GGTGTTCCATCTCGTTTCTCTGGTTCTGGTTCTGGTACCGATTTCACCCTGACCATCTCTTCTCTGCAGC
CAGAAGATTTCGCTAACTACTACTGTCAGCAGGCTAACTCTTTCCCATGGACCTTCGGTGGTGGTACCAA
AGTTGAAATCAAATCCGGAGGGTCGACCATAACTTCGTATAATGTATACTATACGAAGTTATCCTCGAGC
GGTACCCAGGTTCAGCTGGTTCAGTCTGGTGCTGAAGTTAAAAAACCAGGTGCTTCTGTTAAAGTTTCTT
GTAAAGTTTCTGGTTACACCCTGTCTGATCTGTCTATCCACTGGGTTCGTCAGGCTCCAGGTAAAGGTCT
GGAATGGATGGGTGGTTTCGATCCACAGGATGGTGAAACCATCTACGCTCAGAAATTCCAGGGTCGTGTT
ACCATGACCGAAGATACCTCTACCGATACCGCTTACATGGAACTGTCTTCTCTGAAATCTGAGGACACGG
CCGTGTATTACTGTGCTACCGGTTCTTCTTCTTCTTGGTTCGATCCATGGGGTCAGGGAACCCTGGTCAC
CGTCTCCTCAgctagcggcaaaccaatcccaaacccactgctgggc
> 1a - abrilumab dLCDR1
(SEQ ID NO: 140)
cagttagataaaagaggcgcggcagcaagcggcgcgcatgccGATATCCAGATGACCCAGTCTCCATCTT
CTGTTTCTGCTTCTGTTGGTGATCGTGTTACTATTACCTGTCGTGCTTCTagagaccatggccagtaagg
ccggtctctCTGGCTTGGTACCAGCAGAAACCAGGTAAAGCTCCAAAACTGCTGATCTACGGTGCTTCTA
ACCTGGAATCTGGTGTTCCATCTCGTTTCTCTGGTTCTGGTTCTGGTACCGATTTCACCCTGACCATCTC
TTCTCTGCAGCCAGAAGATTTCGCTAACTACTACTGTCAGCAGGCTAACTCTTTCCCATGGACCTTCGGT
GGTGGTACCAAAGTTGAAATCAAATCCGGAGGGTCGACCATAACTTCGTATAATGTATACTATACGAAGT
TATCCTCGAGCGGTACCCAGGTTCAGCTGGTTCAGTCTGGTGCTGAAGTTAAAAAACCAGGTGCTTCTGT
TAAAGTTTCTTGTAAAGTTTCTGGTTACACCCTGTCTGATCTGTCTATCCACTGGGTTCGTCAGGCTCCA
GGTAAAGGTCTGGAATGGATGGGTGGTTTCGATCCACAGGATGGTGAAACCATCTACGCTCAGAAATTCC
AGGGTCGTGTTACCATGACCGAAGATACCTCTACCGATACCGCTTACATGGAACTGTCTTCTCTGAAATC
TGAGGACACGGCCGTGTATTACTGTGCTACCGGTTCTTCTTCTTCTTGGTTCGATCCATGGGGTCAGGGA
ACCCTGGTCACCGTCTCCTCAgctagcggcaaaccaatcccaaacccactgctgggc
> 1b - abrilumab dLCDR2
(SEQ ID NO: 141)
cagttagataaaagaggcgcggcagcaagcggcgcgcatgccGATATCCAGATGACCCAGTCTCCATCTT
CTGTTTCTGCTTCTGTTGGTGATCGTGTTACTATTACCTGTCGTGCTTCTCAGGGTATCTCTTCTTGGCT
GGCTTGGTACCAGCAGAAACCAGGTAAAGCTCCAAAACTGCTGATCTACagagaccatggccagtaaggc
cggtctctGGTGTTCCATCTCGTTTCTCTGGTTCTGGTTCTGGTACCGATTTCACCCTGACCATCTCTTC
TCTGCAGCCAGAAGATTTCGCTAACTACTACTGTCAGCAGGCTAACTCTTTCCCATGGACCTTCGGTGGT
GGTACCAAAGTTGAAATCAAATCCGGAGGGTCGACCATAACTTCGTATAATGTATACTATACGAAGTTAT
CCTCGAGCGGTACCCAGGTTCAGCTGGTTCAGTCTGGTGCTGAAGTTAAAAAACCAGGTGCTTCTGTTAA
AGTTTCTTGTAAAGTTTCTGGTTACACCCTGTCTGATCTGTCTATCCACTGGGTTCGTCAGGCTCCAGGT
AAAGGTCTGGAATGGATGGGTGGTTTCGATCCACAGGATGGTGAAACCATCTACGCTCAGAAATTCCAGG
GTCGTGTTACCATGACCGAAGATACCTCTACCGATACCGCTTACATGGAACTGTCTTCTCTGAAATCTGA
GGACACGGCCGTGTATTACTGTGCTACCGGTTCTTCTTCTTCTTGGTTCGATCCATGGGGTCAGGGAACC
CTGGTCACCGTCTCCTCAgctagcggcaaaccaatcccaaacccactgctgggc
> 1c - abrilumab dLCDR3
(SEQ ID NO: 142)
cagttagataaaagaggcgcggcagcaagcggcgcgcatgccGATATCCAGATGACCCAGTCTCCATCTT
CTGTTTCTGCTTCTGTTGGTGATCGTGTTACTATTACCTGTCGTGCTTCTCAGGGTATCTCTTCTTGGCT
GGCTTGGTACCAGCAGAAACCAGGTAAAGCTCCAAAACTGCTGATCTACGGTGCTTCTAACCTGGAATCT
GGTGTTCCATCTCGTTTCTCTGGTTCTGGTTCTGGTACCGATTTCACCCTGACCATCTCTTCTCTGCAGC
CAGAAGATTTCGCTAACTACTACTGTagagaccatggccagtaaggccggtctctTTCGGTGGTGGTACC
AAAGTTGAAATCAAATCCGGAGGGTCGACCATAACTTCGTATAATGTATACTATACGAAGTTATCCTCGA
GCGGTACCCAGGTTCAGCTGGTTCAGTCTGGTGCTGAAGTTAAAAAACCAGGTGCTTCTGTTAAAGTTTC
TTGTAAAGTTTCTGGTTACACCCTGTCTGATCTGTCTATCCACTGGGTTCGTCAGGCTCCAGGTAAAGGT
CTGGAATGGATGGGTGGTTTCGATCCACAGGATGGTGAAACCATCTACGCTCAGAAATTCCAGGGTCGTG
TTACCATGACCGAAGATACCTCTACCGATACCGCTTACATGGAACTGTCTTCTCTGAAATCTGAGGACAC
GGCCGTGTATTACTGTGCTACCGGTTCTTCTTCTTCTTGGTTCGATCCATGGGGTCAGGGAACCCTGGTC
ACCGTCTCCTCAgctagcggcaaaccaatcccaaacccactgctgggc
> 1d - abrilumab dHCDR1
(SEQ ID NO: 143)
cagttagataaaagaggcgcggcagcaagcggcgcgcatgccGATATCCAGATGACCCAGTCTCCATCTT
CTGTTTCTGCTTCTGTTGGTGATCGTGTTACTATTACCTGTCGTGCTTCTCAGGGTATCTCTTCTTGGCT
GGCTTGGTACCAGCAGAAACCAGGTAAAGCTCCAAAACTGCTGATCTACGGTGCTTCTAACCTGGAATCT
GGTGTTCCATCTCGTTTCTCTGGTTCTGGTTCTGGTACCGATTTCACCCTGACCATCTCTTCTCTGCAGC
CAGAAGATTTCGCTAACTACTACTGTCAGCAGGCTAACTCTTTCCCATGGACCTTCGGTGGTGGTACCAA
AGTTGAAATCAAATCCGGAGGGTCGACCATAACTTCGTATAATGTATACTATACGAAGTTATCCTCGAGC
GGTACCCAGGTTCAGCTGGTTCAGTCTGGTGCTGAAGTTAAAAAACCAGGTGCTTCTGTTAAAGTTTCTT
GTAAAGTTTCTagagaccatggccagtaaggccggtctctATCCACTGGGTTCGTCAGGCTCCAGGTAAA
GGTCTGGAATGGATGGGTGGTTTCGATCCACAGGATGGTGAAACCATCTACGCTCAGAAATTCCAGGGTC
GTGTTACCATGACCGAAGATACCTCTACCGATACCGCTTACATGGAACTGTCTTCTCTGAAATCTGAGGA
CACGGCCGTGTATTACTGTGCTACCGGTTCTTCTTCTTCTTGGTTCGATCCATGGGGTCAGGGAACCCTG
GTCACCGTCTCCTCAgctagcggcaaaccaatcccaaacccactgctgggc
> 1e - abrilumab dHCDR2
(SEQ ID NO: 144)
cagttagataaaagaggcgcggcagcaagcggcgcgcatgccGATATCCAGATGACCCAGTCTCCATCTT
CTGTTTCTGCTTCTGTTGGTGATCGTGTTACTATTACCTGTCGTGCTTCTCAGGGTATCTCTTCTTGGCT
GGCTTGGTACCAGCAGAAACCAGGTAAAGCTCCAAAACTGCTGATCTACGGTGCTTCTAACCTGGAATCT
GGTGTTCCATCTCGTTTCTCTGGTTCTGGTTCTGGTACCGATTTCACCCTGACCATCTCTTCTCTGCAGC
CAGAAGATTTCGCTAACTACTACTGTCAGCAGGCTAACTCTTTCCCATGGACCTTCGGTGGTGGTACCAA
AGTTGAAATCAAATCCGGAGGGTCGACCATAACTTCGTATAATGTATACTATACGAAGTTATCCTCGAGC
GGTACCCAGGTTCAGCTGGTTCAGTCTGGTGCTGAAGTTAAAAAACCAGGTGCTTCTGTTAAAGTTTCTT
GTAAAGTTTCTGGTTACACCCTGTCTGATCTGTCTATCCACTGGGTTCGTCAGGCTCCAGGTAAAGGTCT
GGAATGGATGGGTGGTagagaccatggccagtaaggccggtctctATCTACGCTCAGAAATTCCAGGGTC
GTGTTACCATGACCGAAGATACCTCTACCGATACCGCTTACATGGAACTGTCTTCTCTGAAATCTGAGGA
CACGGCCGTGTATTACTGTGCTACCGGTTCTTCTTCTTCTTGGTTCGATCCATGGGGTCAGGGAACCCTG
GTCACCGTCTCCTCAgctagcggcaaaccaatcccaaacccactgctgggc
> 1f - abrilumab dHCDR3
(SEQ ID NO: 145)
cagttagataaaagaggcgcggcagcaagcggcgcgcatgccGATATCCAGATGACCCAGTCTCCATCTT
CTGTTTCTGCTTCTGTTGGTGATCGTGTTACTATTACCTGTCGTGCTTCTCAGGGTATCTCTTCTTGGCT
GGCTTGGTACCAGCAGAAACCAGGTAAAGCTCCAAAACTGCTGATCTACGGTGCTTCTAACCTGGAATCT
GGTGTTCCATCTCGTTTCTCTGGTTCTGGTTCTGGTACCGATTTCACCCTGACCATCTCTTCTCTGCAGC
CAGAAGATTTCGCTAACTACTACTGTCAGCAGGCTAACTCTTTCCCATGGACCTTCGGTGGTGGTACCAA
AGTTGAAATCAAATCCGGAGGGTCGACCATAACTTCGTATAATGTATACTATACGAAGTTATCCTCGAGC
GGTACCCAGGTTCAGCTGGTTCAGTCTGGTGCTGAAGTTAAAAAACCAGGTGCTTCTGTTAAAGTTTCTT
GTAAAGTTTCTGGTTACACCCTGTCTGATCTGTCTATCCACTGGGTTCGTCAGGCTCCAGGTAAAGGTCT
GGAATGGATGGGTGGTTTCGATCCACAGGATGGTGAAACCATCTACGCTCAGAAATTCCAGGGTCGTGTT
ACCATGACCGAAGATACCTCTACCGATACCGCTTACATGGAACTGTCTTCTCTGAAATCTGAGGACACGG
CCGTGTATTACTGTagagaccatggccagtaaggccggtctctGGAACCCTGGTCACCGTCTCCTCAgct
agcggcaaaccaatcccaaacccactgctgggc
> 2 - mepolizumab
(SEQ ID NO: 146)
cagttagataaaagaggcgcggcagcaagcggcgcgcatgccGATATCGTTATGACCCAGTCTCCAGATT
CTCTGGCTGTTTCTCTGGGTGAACGTGCTACCATCAACTGCAAATCTTCTCAGTCTCTGCTGAACTCTGG
TAACCAGAAAAACTACCTGGCTTGGTACCAGCAGAAACCAGGTCAGCCACCAAAACTGCTGATCTACGGT
GCTTCTACCCGTGAATCTGGTGTTCCAGATCGTTTCTCTGGTTCTGGTTCTGGTACCGATTTCACCCTGA
CCATCTCTTCTCTGCAGGCTGAAGATGTTGCTGTTTACTACTGTCAGAACGTTCACTCTTTCCCATTCAC
CTTCGGTGGTGGTACCAAAGTTGAAATCAAATCCGGAGGGTCGACCATAACTTCGTATAATGTATACTAT
ACGAAGTTATCCTCGAGCGGTACCCAGGTTACCCTGCGTGAATCTGGTCCAGCTCTGGTTAAACCAACCC
AGACCCTGACCCTGACCTGTACCGTTTCTGGTTTCTCTCTGTCTGCTTACTCTGTTAACTGGATCCGTCA
GCCACCAGGTAAAGCTCTGGAATGGCTGGCTATGATCTGGGGTGATGGTAAAATCGTTTACAACTCTGCT
CTGAAATCTCGTCTGACCATCTCTAAAGATACCTCTAAAAACCAGGTTGTTCTGACCATGACCAACATGG
ATCCTGTGGACACAGCCACATATTACTGTGCTGGTGATGGTTACTACCCATACGCTATGGATAACTGGGG
TCAGGGAACCCTGGTCACCGTCTCCTCAgctagcggcaaaccaatcccaaacccactgctgggc
> 2a - mepolizumab dLCDR1
(SEQ ID NO: 147)
cagttagataaaagaggcgcggcagcaagcggcgcgcatgccGATATCGTTATGACCCAGTCTCCAGATT
CTCTGGCTGTTTCTCTGGGTGAACGTGCTACCATCAACTGCAAATCTTCTagagaccatggccagtaagg
ccggtctctCTGGCTTGGTACCAGCAGAAACCAGGTCAGCCACCAAAACTGCTGATCTACGGTGCTTCTA
CCCGTGAATCTGGTGTTCCAGATCGTTTCTCTGGTTCTGGTTCTGGTACCGATTTCACCCTGACCATCTC
TTCTCTGCAGGCTGAAGATGTTGCTGTTTACTACTGTCAGAACGTTCACTCTTTCCCATTCACCTTCGGT
GGTGGTACCAAAGTTGAAATCAAATCCGGAGGGTCGACCATAACTTCGTATAATGTATACTATACGAAGT
TATCCTCGAGCGGTACCCAGGTTACCCTGCGTGAATCTGGTCCAGCTCTGGTTAAACCAACCCAGACCCT
GACCCTGACCTGTACCGTTTCTGGTTTCTCTCTGTCTGCTTACTCTGTTAACTGGATCCGTCAGCCACCA
GGTAAAGCTCTGGAATGGCTGGCTATGATCTGGGGTGATGGTAAAATCGTTTACAACTCTGCTCTGAAAT
CTCGTCTGACCATCTCTAAAGATACCTCTAAAAACCAGGTTGTTCTGACCATGACCAACATGGATCCTGT
GGACACAGCCACATATTACTGTGCTGGTGATGGTTACTACCCATACGCTATGGATAACTGGGGTCAGGGA
ACCCTGGTCACCGTCTCCTCAgctagcggcaaaccaatcccaaacccactgctgggc
> 2b - mepolizumab dLCDR2
(SEQ ID NO: 148)
cagttagataaaagaggcgcggcagcaagcggcgcgcatgccGATATCGTTATGACCCAGTCTCCAGATT
CTCTGGCTGTTTCTCTGGGTGAACGTGCTACCATCAACTGCAAATCTTCTCAGTCTCTGCTGAACTCTGG
TAACCAGAAAAACTACCTGGCTTGGTACCAGCAGAAACCAGGTCAGCCACCAAAACTGCTGATCTACaga
gaccatggccagtaaggccggtctctGGTGTTCCAGATCGTTTCTCTGGTTCTGGTTCTGGTACCGATTT
CACCCTGACCATCTCTTCTCTGCAGGCTGAAGATGTTGCTGTTTACTACTGTCAGAACGTTCACTCTTTC
CCATTCACCTTCGGTGGTGGTACCAAAGTTGAAATCAAATCCGGAGGGTCGACCATAACTTCGTATAATG
TATACTATACGAAGTTATCCTCGAGCGGTACCCAGGTTACCCTGCGTGAATCTGGTCCAGCTCTGGTTAA
ACCAACCCAGACCCTGACCCTGACCTGTACCGTTTCTGGTTTCTCTCTGTCTGCTTACTCTGTTAACTGG
ATCCGTCAGCCACCAGGTAAAGCTCTGGAATGGCTGGCTATGATCTGGGGTGATGGTAAAATCGTTTACA
ACTCTGCTCTGAAATCTCGTCTGACCATCTCTAAAGATACCTCTAAAAACCAGGTTGTTCTGACCATGAC
CAACATGGATCCTGTGGACACAGCCACATATTACTGTGCTGGTGATGGTTACTACCCATACGCTATGGAT
AACTGGGGTCAGGGAACCCTGGTCACCGTCTCCTCAgctagcggcaaaccaatcccaaacccactgctgg
gc
> 2c - mepolizumab dLCDR3
(SEQ ID NO: 149)
cagttagataaaagaggcgcggcagcaagcggcgcgcatgccGATATCGTTATGACCCAGTCTCCAGATT
CTCTGGCTGTTTCTCTGGGTGAACGTGCTACCATCAACTGCAAATCTTCTCAGTCTCTGCTGAACTCTGG
TAACCAGAAAAACTACCTGGCTTGGTACCAGCAGAAACCAGGTCAGCCACCAAAACTGCTGATCTACGGT
GCTTCTACCCGTGAATCTGGTGTTCCAGATCGTTTCTCTGGTTCTGGTTCTGGTACCGATTTCACCCTGA
CCATCTCTTCTCTGCAGGCTGAAGATGTTGCTGTTTACTACTGTagagaccatggccagtaaggccggtc
tctTTCGGTGGTGGTACCAAAGTTGAAATCAAATCCGGAGGGTCGACCATAACTTCGTATAATGTATACT
ATACGAAGTTATCCTCGAGCGGTACCCAGGTTACCCTGCGTGAATCTGGTCCAGCTCTGGTTAAACCAAC
CCAGACCCTGACCCTGACCTGTACCGTTTCTGGTTTCTCTCTGTCTGCTTACTCTGTTAACTGGATCCGT
CAGCCACCAGGTAAAGCTCTGGAATGGCTGGCTATGATCTGGGGTGATGGTAAAATCGTTTACAACTCTG
CTCTGAAATCTCGTCTGACCATCTCTAAAGATACCTCTAAAAACCAGGTTGTTCTGACCATGACCAACAT
GGATCCTGTGGACACAGCCACATATTACTGTGCTGGTGATGGTTACTACCCATACGCTATGGATAACTGG
GGTCAGGGAACCCTGGTCACCGTCTCCTCAgctagcggcaaaccaatcccaaacccactgctgggc
> 2d - mepolizumab dHCDR1
(SEQ ID NO: 150)
cagttagataaaagaggcgcggcagcaagcggcgcgcatgccGATATCGTTATGACCCAGTCTCCAGATT
CTCTGGCTGTTTCTCTGGGTGAACGTGCTACCATCAACTGCAAATCTTCTCAGTCTCTGCTGAACTCTGG
TAACCAGAAAAACTACCTGGCTTGGTACCAGCAGAAACCAGGTCAGCCACCAAAACTGCTGATCTACGGT
TTCTACCCGTGAATCTGGTGTTCCAGATCGTTTCTCTGGTTCTGGTTCTGGTACCGATTTCACCCTGA
CCATCTCTTCTCTGCAGGCTGAAGATGTTGCTGTTTACTACTGTCAGAACGTTCACTCTTTCCCATTCAC
CTTCGGTGGTGGTACCAAAGTTGAAATCAAATCCGGAGGGTCGACCATAACTTCGTATAATGTATACTAT
ACGAAGTTATCCTCGAGCGGTACCCAGGTTACCCTGCGTGAATCTGGTCCAGCTCTGGTTAAACCAACCC
AGACCCTGACCCTGACCTGTACCGTTTCTagagaccatggccagtaaggccggtctctGTTAACTGGATC
CGTCAGCCACCAGGTAAAGCTCTGGAATGGCTGGCTATGATCTGGGGTGATGGTAAAATCGTTTACAACT
CTGCTCTGAAATCTCGTCTGACCATCTCTAAAGATACCTCTAAAAACCAGGTTGTTCTGACCATGACCAA
CATGGATCCTGTGGACACAGCCACATATTACTGTGCTGGTGATGGTTACTACCCATACGCTATGGATAAC
TGGGGTCAGGGAACCCTGGTCACCGTCTCCTCAgctagcggcaaaccaatcccaaacccactgctgggc
> 2e - mepolizumab dHCDR2
(SEQ ID NO: 151)
cagttagataaaagaggcgcggcagcaagcggcgcgcatgccGATATCGTTATGACCCAGTCTCCAGATT
CTCTGGCTGTTTCTCTGGGTGAACGTGCTACCATCAACTGCAAATCTTCTCAGTCTCTGCTGAACTCTGG
TAACCAGAAAAACTACCTGGCTTGGTACCAGCAGAAACCAGGTCAGCCACCAAAACTGCTGATCTACGGT
GCTTCTACCCGTGAATCTGGTGTTCCAGATCGTTTCTCTGGTTCTGGTTCTGGTACCGATTTCACCCTGA
CCATCTCTTCTCTGCAGGCTGAAGATGTTGCTGTTTACTACTGTCAGAACGTTCACTCTTTCCCATTCAC
CTTCGGTGGTGGTACCAAAGTTGAAATCAAATCCGGAGGGTCGACCATAACTTCGTATAATGTATACTAT
ACGAAGTTATCCTCGAGCGGTACCCAGGTTACCCTGCGTGAATCTGGTCCAGCTCTGGTTAAACCAACCC
AGACCCTGACCCTGACCTGTACCGTTTCTGGTTTCTCTCTGTCTGCTTACTCTGTTAACTGGATCCGTCA
GCCACCAGGTAAAGCTCTGGAATGGCTGGCTATGagagaccatggccagtaaggccggtctctATCGTTT
ACAACTCTGCTCTGAAATCTCGTCTGACCATCTCTAAAGATACCTCTAAAAACCAGGTTGTTCTGACCAT
GACCAACATGGATCCTGTGGACACAGCCACATATTACTGTGCTGGTGATGGTTACTACCCATACGCTATG
GATAACTGGGGTCAGGGAACCCTGGTCACCGTCTCCTCAgctagcggcaaaccaatcccaaacccactgc
tgggc
> 2f - mepolizumab dHCDR3
(SEQ ID NO: 152)
cagttagataaaagaggcgcggcagcaagcggcgcgcatgccGATATCGTTATGACCCAGTCTCCAGATT
CTCTGGCTGTTTCTCTGGGTGAACGTGCTACCATCAACTGCAAATCTTCTCAGTCTCTGCTGAACTCTGG
TAACCAGAAAAACTACCTGGCTTGGTACCAGCAGAAACCAGGTCAGCCACCAAAACTGCTGATCTACGGT
GCTTCTACCCGTGAATCTGGTGTTCCAGATCGTTTCTCTGGTTCTGGTTCTGGTACCGATTTCACCCTGA
CCATCTCTTCTCTGCAGGCTGAAGATGTTGCTGTTTACTACTGTCAGAACGTTCACTCTTTCCCATTCAC
CTTCGGTGGTGGTACCAAAGTTGAAATCAAATCCGGAGGGTCGACCATAACTTCGTATAATGTATACTAT
ACGAAGTTATCCTCGAGCGGTACCCAGGTTACCCTGCGTGAATCTGGTCCAGCTCTGGTTAAACCAACCC
AGACCCTGACCCTGACCTGTACCGTTTCTGGTTTCTCTCTGTCTGCTTACTCTGTTAACTGGATCCGTCA
GCCACCAGGTAAAGCTCTGGAATGGCTGGCTATGATCTGGGGTGATGGTAAAATCGTTTACAACTCTGCT
CTGAAATCTCGTCTGACCATCTCTAAAGATACCTCTAAAAACCAGGTTGTTCTGACCATGACCAACATGG
ATCCTGTGGACACAGCCACATATTACTGTagagaccatggccagtaaggccggtctctGGAACCCTGGTC
ACCGTCTCCTCAgctagcggcaaaccaatcccaaacccactgctgggc
> 3 - crenezumab
(SEQ ID NO: 153)
cagttagataaaagaggcgcggcagcaagcggcgcgcatgccGATATCGTTATGACCCAGTCTCCACTGT
CTCTGCCAGTTACCCCAGGTGAACCAGCTTCTATTTCTTGTCGTTCTTCTCAGTCTCTGGTTTACTCTAA
CGGTGATACCTACCTGCACTGGTACCTGCAGAAACCAGGTCAGTCTCCACAGCTGCTGATCTACAAAGTT
TCTAACCGTTTCTCTGGTGTTCCAGATCGTTTCTCTGGTTCTGGTTCTGGTACCGATTTCACCCTGAAAA
TCTCTCGTGTTGAAGCTGAAGATGTTGGTGTTTACTACTGTTCTCAGTCTACCCACGTTCCATGGACCTT
CGGTGGTGGTACCAAAGTTGAAATCAAATCCGGAGGGTCGACCATAACTTCGTATAATGTATACTATACG
AAGTTATCCTCGAGCGGTACCGAAGTTCAGCTGGTTGAATCTGGTGGTGGTCTGGTTCAGCCAGGTGGTT
CTCTGCGTCTGTCTTGTGCTGCTTCTGGTTTCACCTTCTCTTCTTACGGTATGTCTTGGGTTCGTCAGGC
TCCAGGTAAAGGTCTGGAACTGGTTGCTTCTATCAACTCTAACGGTGGTTCTACCTACTACCCAGATTCT
GTTAAAGGTCGTTTCACCATCTCTCGTGATAACGCTAAAAACTCTCTGTACCTGCAGATGAACTCTCTGC
GTGCCGAGGACACGGCTGTGTATTACTGTGCTTCTGGTGATTACTGGGGTCAGGGGACCACGGTCACCGT
CTCCTCAgctagcggcaaaccaatcccaaacccactgctgggc
> 3a - crenezumab dLCDR1
(SEQ ID NO: 154)
cagttagataaaagaggcgcggcagcaagcggcgcgcatgccGATATCGTTATGACCCAGTCTCCACTGT
CTCTGCCAGTTACCCCAGGTGAACCAGCTTCTATTTCTTGTCGTTCTTCTagagaccatggccagtaagg
ccggtctctCTGCACTGGTACCTGCAGAAACCAGGTCAGTCTCCACAGCTGCTGATCTACAAAGTTTCTA
ACCGTTTCTCTGGTGTTCCAGATCGTTTCTCTGGTTCTGGTTCTGGTACCGATTTCACCCTGAAAATCTC
TCGTGTTGAAGCTGAAGATGTTGGTGTTTACTACTGTTCTCAGTCTACCCACGTTCCATGGACCTTCGGT
GGTGGTACCAAAGTTGAAATCAAATCCGGAGGGTCGACCATAACTTCGTATAATGTATACTATACGAAGT
TATCCTCGAGCGGTACCGAAGTTCAGCTGGTTGAATCTGGTGGTGGTCTGGTTCAGCCAGGTGGTTCTCT
GCGTCTGTCTTGTGCTGCTTCTGGTTTCACCTTCTCTTCTTACGGTATGTCTTGGGTTCGTCAGGCTCCA
GGTAAAGGTCTGGAACTGGTTGCTTCTATCAACTCTAACGGTGGTTCTACCTACTACCCAGATTCTGTTA
AAGGTCGTTTCACCATCTCTCGTGATAACGCTAAAAACTCTCTGTACCTGCAGATGAACTCTCTGCGTGC
CGAGGACACGGCTGTGTATTACTGTGCTTCTGGTGATTACTGGGGTCAGGGGACCACGGTCACCGTCTCC
TCAgctagcggcaaaccaatcccaaacccactgctgggc
> 3b - crenezumab dLCDR2
(SEQ ID NO: 155)
cagttagataaaagaggcgcggcagcaagcggcgcgcatgccGATATCGTTATGACCCAGTCTCCACTGT
CTCTGCCAGTTACCCCAGGTGAACCAGCTTCTATTTCTTGTCGTTCTTCTCAGTCTCTGGTTTACTCTAA
CGGTGATACCTACCTGCACTGGTACCTGCAGAAACCAGGTCAGTCTCCACAGCTGCTGATCTACagagac
catggccagtaaggccggtctctGGTGTTCCAGATCGTTTCTCTGGTTCTGGTTCTGGTACCGATTTCAC
CCTGAAAATCTCTCGTGTTGAAGCTGAAGATGTTGGTGTTTACTACTGTTCTCAGTCTACCCACGTTCCA
TGGACCTTCGGTGGTGGTACCAAAGTTGAAATCAAATCCGGAGGGTCGACCATAACTTCGTATAATGTAT
ACTATACGAAGTTATCCTCGAGCGGTACCGAAGTTCAGCTGGTTGAATCTGGTGGTGGTCTGGTTCAGCC
AGGTGGTTCTCTGCGTCTGTCTTGTGCTGCTTCTGGTTTCACCTTCTCTTCTTACGGTATGTCTTGGGTT
CGTCAGGCTCCAGGTAAAGGTCTGGAACTGGTTGCTTCTATCAACTCTAACGGTGGTTCTACCTACTACC
CAGATTCTGTTAAAGGTCGTTTCACCATCTCTCGTGATAACGCTAAAAACTCTCTGTACCTGCAGATGAA
CTCTCTGCGTGCCGAGGACACGGCTGTGTATTACTGTGCTTCTGGTGATTACTGGGGTCAGGGGACCACG
GTCACCGTCTCCTCAgctagcggcaaaccaatcccaaacccactgctgggc
> 3c - crenezumab dLCDR3
(SEQ ID NO: 156)
cagttagataaaagaggcgcggcagcaagcggcgcgcatgccGATATCGTTATGACCCAGTCTCCACTGT
CTCTGCCAGTTACCCCAGGTGAACCAGCTTCTATTTCTTGTCGTTCTTCTCAGTCTCTGGTTTACTCTAA
CGGTGATACCTACCTGCACTGGTACCTGCAGAAACCAGGTCAGTCTCCACAGCTGCTGATCTACAAAGTT
TCTAACCGTTTCTCTGGTGTTCCAGATCGTTTCTCTGGTTCTGGTTCTGGTACCGATTTCACCCTGAAAA
TCTCTCGTGTTGAAGCTGAAGATGTTGGTGTTTACTACTGTagagaccatggccagtaaggccggtctct
TTCGGTGGTGGTACCAAAGTTGAAATCAAATCCGGAGGGTCGACCATAACTTCGTATAATGTATACTATA
CGAAGTTATCCTCGAGCGGTACCGAAGTTCAGCTGGTTGAATCTGGTGGTGGTCTGGTTCAGCCAGGTGG
TTCTCTGCGTCTGTCTTGTGCTGCTTCTGGTTTCACCTTCTCTTCTTACGGTATGTCTTGGGTTCGTCAG
GCTCCAGGTAAAGGTCTGGAACTGGTTGCTTCTATCAACTCTAACGGTGGTTCTACCTACTACCCAGATT
CTGTTAAAGGTCGTTTCACCATCTCTCGTGATAACGCTAAAAACTCTCTGTACCTGCAGATGAACTCTCT
GCGTGCCGAGGACACGGCTGTGTATTACTGTGCTTCTGGTGATTACTGGGGTCAGGGGACCACGGTCACC
GTCTCCTCAgctagcggcaaaccaatcccaaacccactgctgggc
> 3d - crenezumab dHCDR1
(SEQ ID NO: 157)
cagttagataaaagaggcgcggcagcaagcggcgcgcatgccGATATCGTTATGACCCAGTCTCCACTGT
CTCTGCCAGTTACCCCAGGTGAACCAGCTTCTATTTCTTGTCGTTCTTCTCAGTCTCTGGTTTACTCTAA
CGGTGATACCTACCTGCACTGGTACCTGCAGAAACCAGGTCAGTCTCCACAGCTGCTGATCTACAAAGTT
TCTAACCGTTTCTCTGGTGTTCCAGATCGTTTCTCTGGTTCTGGTTCTGGTACCGATTTCACCCTGAAAA
TCTCTCGTGTTGAAGCTGAAGATGTTGGTGTTTACTACTGTTCTCAGTCTACCCACGTTCCATGGACCTT
CGGTGGTGGTACCAAAGTTGAAATCAAATCCGGAGGGTCGACCATAACTTCGTATAATGTATACTATACG
AAGTTATCCTCGAGCGGTACCGAAGTTCAGCTGGTTGAATCTGGTGGTGGTCTGGTTCAGCCAGGTGGTT
CTCTGCGTCTGTCTTGTGCTGCTTCTagagaccatggccagtaaggccggtctctATGTCTTGGGTTCGT
CAGGCTCCAGGTAAAGGTCTGGAACTGGTTGCTTCTATCAACTCTAACGGTGGTTCTACCTACTACCCAG
ATTCTGTTAAAGGTCGTTTCACCATCTCTCGTGATAACGCTAAAAACTCTCTGTACCTGCAGATGAACTC
TCTGCGTGCCGAGGACACGGCTGTGTATTACTGTGCTTCTGGTGATTACTGGGGTCAGGGGACCACGGTC
ACCGTCTCCTCAgctagcggcaaaccaatcccaaacccactgctgggc
> 3e - crenezumab dHCDR2
(SEQ ID NO: 158)
cagttagataaaagaggcgcggcagcaagcggcgcgcatgccGATATCGTTATGACCCAGTCTCCACTGT
CTCTGCCAGTTACCCCAGGTGAACCAGCTTCTATTTCTTGTCGTTCTTCTCAGTCTCTGGTTTACTCTAA
CGGTGATACCTACCTGCACTGGTACCTGCAGAAACCAGGTCAGTCTCCACAGCTGCTGATCTACAAAGTT
TCTAACCGTTTCTCTGGTGTTCCAGATCGTTTCTCTGGTTCTGGTTCTGGTACCGATTTCACCCTGAAAA
TCTCTCGTGTTGAAGCTGAAGATGTTGGTGTTTACTACTGTTCTCAGTCTACCCACGTTCCATGGACCTT
CGGTGGTGGTACCAAAGTTGAAATCAAATCCGGAGGGTCGACCATAACTTCGTATAATGTATACTATACG
AAGTTATCCTCGAGCGGTACCGAAGTTCAGCTGGTTGAATCTGGTGGTGGTCTGGTTCAGCCAGGTGGTT
CTCTGCGTCTGTCTTGTGCTGCTTCTGGTTTCACCTTCTCTTCTTACGGTATGTCTTGGGTTCGTCAGGC
TCCAGGTAAAGGTCTGGAACTGGTTGCTTCTagagaccatggccagtaaggccggtctctTACTACCCAG
ATTCTGTTAAAGGTCGTTTCACCATCTCTCGTGATAACGCTAAAAACTCTCTGTACCTGCAGATGAACTC
TCTGCGTGCCGAGGACACGGCTGTGTATTACTGTGCTTCTGGTGATTACTGGGGTCAGGGGACCACGGTC
ACCGTCTCCTCAgctagcggcaaaccaatcccaaacccactgctgggc
> 3f - crenezumab dHCDR3
(SEQ ID NO: 159)
cagttagataaaagaggcgcggcagcaagcggcgcgcatgccGATATCGTTATGACCCAGTCTCCACTGT
CTCTGCCAGTTACCCCAGGTGAACCAGCTTCTATTTCTTGTCGTTCTTCTCAGTCTCTGGTTTACTCTAA
CGGTGATACCTACCTGCACTGGTACCTGCAGAAACCAGGTCAGTCTCCACAGCTGCTGATCTACAAAGTT
TCTAACCGTTTCTCTGGTGTTCCAGATCGTTTCTCTGGTTCTGGTTCTGGTACCGATTTCACCCTGAAAA
TCTCTCGTGTTGAAGCTGAAGATGTTGGTGTTTACTACTGTTCTCAGTCTACCCACGTTCCATGGACCTT
CGGTGGTGGTACCAAAGTTGAAATCAAATCCGGAGGGTCGACCATAACTTCGTATAATGTATACTATACG
AAGTTATCCTCGAGCGGTACCGAAGTTCAGCTGGTTGAATCTGGTGGTGGTCTGGTTCAGCCAGGTGGTT
CTCTGCGTCTGTCTTGTGCTGCTTCTGGTTTCACCTTCTCTTCTTACGGTATGTCTTGGGTTCGTCAGGC
TCCAGGTAAAGGTCTGGAACTGGTTGCTTCTATCAACTCTAACGGTGGTTCTACCTACTACCCAGATTCT
GTTAAAGGTCGTTTCACCATCTCTCGTGATAACGCTAAAAACTCTCTGTACCTGCAGATGAACTCTCTGC
GTGCCGAGGACACGGCTGTGTATTACTGTagagaccatggccagtaaggccggtctctGGGACCACGGTC
ACCGTCTCCTCAgctagcggcaaaccaatcccaaacccactgctgggc
> 4 - necitumumab
(SEQ ID NO: 160)
cagttagataaaagaggcgcggcagcaagcggcgcgcatgccGAAATCGTTATGACCCAGTCTCCAGCTA
CCCTGTCTCTGTCTCCAGGTGAACGTGCCACTCTGTCTTGTCGTGCTTCTCAGTCTGTTTCTTCTTACCT
GGCTTGGTACCAGCAGAAACCAGGTCAGGCTCCACGTCTGCTGATCTACGATGCTTCTAACCGTGCTACC
GGTATCCCAGCTCGTTTCTCTGGTTCTGGTTCTGGTACCGATTTCACCCTGACCATCTCTTCTCTGGAAC
CAGAAGATTTCGCTGTTTACTACTGTCACCAGTACGGTTCTACCCCACTGACCTTCGGTGGTGGTACCAA
AGTTGAAATCAAATCCGGAGGGTCGACCATAACTTCGTATAATGTATACTATACGAAGTTATCCTCGAGC
GGTACCCAGGTTCAGCTGCAGGAATCTGGTCCAGGTCTGGTTAAACCATCTCAGACCCTGTCTCTGACCT
GTACCGTTTCTGGTGGTTCTATCTCTTCTGGTGATTACTACTGGTCTTGGATCCGTCAGCCACCAGGTAA
AGGTCTGGAATGGATCGGTTACATCTACTACTCTGGTTCTACCGATTACAACCCATCTCTGAAATCTCGT
GTTACCATGTCTGTTGATACCTCTAAAAACCAGTTCTCTCTGAAAGTTAACTCTGTTACCGCCGCGGACA
CGGCTGTGTATTACTGTGCTCGTGTTTCTATCTTCGGTGTTGGTACCTTCGATTACTGGGGTCAGGGAAC
CCTGGTCACCGTCTCCTCAgctagcggcaaaccaatcccaaacccactgctgggc
> 4a - necitumumab dLCDR1
(SEQ ID NO: 161)
cagttagataaaagaggcgcggcagcaagcggcgcgcatgccGAAATCGTTATGACCCAGTCTCCAGCTA
CCCTGTCTCTGTCTCCAGGTGAACGTGCCACTCTGTCTTGTCGTGCTTCTagagaccatggccagtaagg
ccggtctctCTGGCTTGGTACCAGCAGAAACCAGGTCAGGCTCCACGTCTGCTGATCTACGATGCTTCTA
ACCGTGCTACCGGTATCCCAGCTCGTTTCTCTGGTTCTGGTTCTGGTACCGATTTCACCCTGACCATCTC
TTCTCTGGAACCAGAAGATTTCGCTGTTTACTACTGTCACCAGTACGGTTCTACCCCACTGACCTTCGGT
GGTGGTACCAAAGTTGAAATCAAATCCGGAGGGTCGACCATAACTTCGTATAATGTATACTATACGAAGT
TATCCTCGAGCGGTACCCAGGTTCAGCTGCAGGAATCTGGTCCAGGTCTGGTTAAACCATCTCAGACCCT
GTCTCTGACCTGTACCGTTTCTGGTGGTTCTATCTCTTCTGGTGATTACTACTGGTCTTGGATCCGTCAG
CCACCAGGTAAAGGTCTGGAATGGATCGGTTACATCTACTACTCTGGTTCTACCGATTACAACCCATCTC
TGAAATCTCGTGTTACCATGTCTGTTGATACCTCTAAAAACCAGTTCTCTCTGAAAGTTAACTCTGTTAC
CGCCGCGGACACGGCTGTGTATTACTGTGCTCGTGTTTCTATCTTCGGTGTTGGTACCTTCGATTACTGG
GGTCAGGGAACCCTGGTCACCGTCTCCTCAgctagcggcaaaccaatcccaaacccactgctgggc
> 4b - necitumumab dLCDR2
(SEQ ID NO: 162)
cagttagataaaagaggcgcggcagcaagcggcgcgcatgccGAAATCGTTATGACCCAGTCTCCAGCTA
CCCTGTCTCTGTCTCCAGGTGAACGTGCCACTCTGTCTTGTCGTGCTTCTCAGTCTGTTTCTTCTTACCT
GGCTTGGTACCAGCAGAAACCAGGTCAGGCTCCACGTCTGCTGATCTACagagaccatggccagtaaggc
cggtctctGGTATCCCAGCTCGTTTCTCTGGTTCTGGTTCTGGTACCGATTTCACCCTGACCATCTCTTC
TCTGGAACCAGAAGATTTCGCTGTTTACTACTGTCACCAGTACGGTTCTACCCCACTGACCTTCGGTGGT
GGTACCAAAGTTGAAATCAAATCCGGAGGGTCGACCATAACTTCGTATAATGTATACTATACGAAGTTAT
CCTCGAGCGGTACCCAGGTTCAGCTGCAGGAATCTGGTCCAGGTCTGGTTAAACCATCTCAGACCCTGTC
TCTGACCTGTACCGTTTCTGGTGGTTCTATCTCTTCTGGTGATTACTACTGGTCTTGGATCCGTCAGCCA
CCAGGTAAAGGTCTGGAATGGATCGGTTACATCTACTACTCTGGTTCTACCGATTACAACCCATCTCTGA
AATCTCGTGTTACCATGTCTGTTGATACCTCTAAAAACCAGTTCTCTCTGAAAGTTAACTCTGTTACCGC
CGCGGACACGGCTGTGTATTACTGTGCTCGTGTTTCTATCTTCGGTGTTGGTACCTTCGATTACTGGGGT
CAGGGAACCCTGGTCACCGTCTCCTCAgctagcggcaaaccaatcccaaacccactgctgggc
> 4c - necitumumab dLCDR3
(SEQ ID NO: 163)
cagttagataaaagaggcgcggcagcaagcggcgcgcatgccGAAATCGTTATGACCCAGTCTCCAGCTA
CCCTGTCTCTGTCTCCAGGTGAACGTGCCACTCTGTCTTGTCGTGCTTCTCAGTCTGTTTCTTCTTACCT
GGCTTGGTACCAGCAGAAACCAGGTCAGGCTCCACGTCTGCTGATCTACGATGCTTCTAACCGTGCTACC
GGTATCCCAGCTCGTTTCTCTGGTTCTGGTTCTGGTACCGATTTCACCCTGACCATCTCTTCTCTGGAAC
CAGAAGATTTCGCTGTTTACTACTGTagagaccatggccagtaaggccggtctctTTCGGTGGTGGTACC
AAAGTTGAAATCAAATCCGGAGGGTCGACCATAACTTCGTATAATGTATACTATACGAAGTTATCCTCGA
GCGGTACCCAGGTTCAGCTGCAGGAATCTGGTCCAGGTCTGGTTAAACCATCTCAGACCCTGTCTCTGAC
CTGTACCGTTTCTGGTGGTTCTATCTCTTCTGGTGATTACTACTGGTCTTGGATCCGTCAGCCACCAGGT
AAAGGTCTGGAATGGATCGGTTACATCTACTACTCTGGTTCTACCGATTACAACCCATCTCTGAAATCTC
GTGTTACCATGTCTGTTGATACCTCTAAAAACCAGTTCTCTCTGAAAGTTAACTCTGTTACCGCCGCGGA
CACGGCTGTGTATTACTGTGCTCGTGTTTCTATCTTCGGTGTTGGTACCTTCGATTACTGGGGTCAGGGA
ACCCTGGTCACCGTCTCCTCAgctagcggcaaaccaatcccaaacccactgctgggc
> 4d - necitumumab dHCDR1
(SEQ ID NO: 164)
cagttagataaaagaggcgcggcagcaagcggcgcgcatgccGAAATCGTTATGACCCAGTCTCCAGCTA
CCCTGTCTCTGTCTCCAGGTGAACGTGCCACTCTGTCTTGTCGTGCTTCTCAGTCTGTTTCTTCTTACCT
GGCTTGGTACCAGCAGAAACCAGGTCAGGCTCCACGTCTGCTGATCTACGATGCTTCTAACCGTGCTACC
GGTATCCCAGCTCGTTTCTCTGGTTCTGGTTCTGGTACCGATTTCACCCTGACCATCTCTTCTCTGGAAC
CAGAAGATTTCGCTGTTTACTACTGTCACCAGTACGGTTCTACCCCACTGACCTTCGGTGGTGGTACCAA
AGTTGAAATCAAATCCGGAGGGTCGACCATAACTTCGTATAATGTATACTATACGAAGTTATCCTCGAGC
GGTACCCAGGTTCAGCTGCAGGAATCTGGTCCAGGTCTGGTTAAACCATCTCAGACCCTGTCTCTGACCT
GTACCGTTTCTagagaccatggccagtaaggccggtctctTGGTCTTGGATCCGTCAGCCACCAGGTAAA
GGTCTGGAATGGATCGGTTACATCTACTACTCTGGTTCTACCGATTACAACCCATCTCTGAAATCTCGTG
TTACCATGTCTGTTGATACCTCTAAAAACCAGTTCTCTCTGAAAGTTAACTCTGTTACCGCCGCGGACAC
GGCTGTGTATTACTGTGCTCGTGTTTCTATCTTCGGTGTTGGTACCTTCGATTACTGGGGTCAGGGAACC
CTGGTCACCGTCTCCTCAgctagcggcaaaccaatcccaaacccactgctgggc
> 4e - necitumumab dHCDR2
(SEQ ID NO: 165)
cagttagataaaagaggcgcggcagcaagcggcgcgcatgccGAAATCGTTATGACCCAGTCTCCAGCTA
CCCTGTCTCTGTCTCCAGGTGAACGTGCCACTCTGTCTTGTCGTGCTTCTCAGTCTGTTTCTTCTTACCT
GGCTTGGTACCAGCAGAAACCAGGTCAGGCTCCACGTCTGCTGATCTACGATGCTTCTAACCGTGCTACC
GGTATCCCAGCTCGTTTCTCTGGTTCTGGTTCTGGTACCGATTTCACCCTGACCATCTCTTCTCTGGAAC
CAGAAGATTTCGCTGTTTACTACTGTCACCAGTACGGTTCTACCCCACTGACCTTCGGTGGTGGTACCAA
AGTTGAAATCAAATCCGGAGGGTCGACCATAACTTCGTATAATGTATACTATACGAAGTTATCCTCGAGC
GGTACCCAGGTTCAGCTGCAGGAATCTGGTCCAGGTCTGGTTAAACCATCTCAGACCCTGTCTCTGACCT
GTACCGTTTCTGGTGGTTCTATCTCTTCTGGTGATTACTACTGGTCTTGGATCCGTCAGCCACCAGGTAA
AGGTCTGGAATGGATCGGTTACagagaccatggccagtaaggccggtctctGATTACAACCCATCTCTGA
AATCTCGTGTTACCATGTCTGTTGATACCTCTAAAAACCAGTTCTCTCTGAAAGTTAACTCTGTTACCGC
CGCGGACACGGCTGTGTATTACTGTGCTCGTGTTTCTATCTTCGGTGTTGGTACCTTCGATTACTGGGGT
CAGGGAACCCTGGTCACCGTCTCCTCAgctagcggcaaaccaatcccaaacccactgctgggc
> 4f - necitumumab dHCDR3
(SEQ ID NO: 166)
cagttagataaaagaggcgcggcagcaagcggcgcgcatgccGAAATCGTTATGACCCAGTCTCCAGCTA
CCCTGTCTCTGTCTCCAGGTGAACGTGCCACTCTGTCTTGTCGTGCTTCTCAGTCTGTTTCTTCTTACCT
GGCTTGGTACCAGCAGAAACCAGGTCAGGCTCCACGTCTGCTGATCTACGATGCTTCTAACCGTGCTACC
GGTATCCCAGCTCGTTTCTCTGGTTCTGGTTCTGGTACCGATTTCACCCTGACCATCTCTTCTCTGGAAC
CAGAAGATTTCGCTGTTTACTACTGTCACCAGTACGGTTCTACCCCACTGACCTTCGGTGGTGGTACCAA
AGTTGAAATCAAATCCGGAGGGTCGACCATAACTTCGTATAATGTATACTATACGAAGTTATCCTCGAGC
GGTACCCAGGTTCAGCTGCAGGAATCTGGTCCAGGTCTGGTTAAACCATCTCAGACCCTGTCTCTGACCT
GTACCGTTTCTGGTGGTTCTATCTCTTCTGGTGATTACTACTGGTCTTGGATCCGTCAGCCACCAGGTAA
AGGTCTGGAATGGATCGGTTACATCTACTACTCTGGTTCTACCGATTACAACCCATCTCTGAAATCTCGT
GTTACCATGTCTGTTGATACCTCTAAAAACCAGTTCTCTCTGAAAGTTAACTCTGTTACCGCCGCGGACA
CGGCTGTGTATTACTGTagagaccatggccagtaaggccggtctctGGAACCCTGGTCACCGTCTCCTCA
gctagcggcaaaccaatcccaaacccactgctgggc
> 5 - anifrolumab
(SEQ ID NO: 167)
cagttagataaaagaggcgcggcagcaagcggcgcgcatgccGAAATCGTTCTGACCCAGTCTCCAGGTA
CCCTGTCTCTGTCTCCAGGTGAACGTGCCACTCTGTCTTGTCGTGCTTCTCAGTCTGTTTCTTCTTCTTT
CCTGGCTTGGTACCAGCAGAAACCAGGTCAGGCTCCACGTCTGCTGATCTACGGTGCTTCTTCTCGTGCT
ACCGGTATCCCAGATCGTCTGTCTGGTTCTGGTTCTGGTACCGATTTCACCCTGACCATCACCCGTCTGG
AACCAGAAGATTTCGCTGTTTACTACTGTCAGCAGTACGATTCTTCTGCTATCACCTTCGGTGGTGGTAC
CAAAGTTGAAATCAAATCCGGAGGGTCGACCATAACTTCGTATAATGTATACTATACGAAGTTATCCTCG
AGCGGTACCGAAGTTCAGCTGGTTCAGTCTGGTGCTGAAGTTAAAAAACCAGGTGAATCTCTGAAAATCT
CTTGTAAAGGTTCTGGTTACATCTTCACCAACTACTGGATCGCTTGGGTTCGTCAGGTTCCAGGTAAAGG
TCTGGAATCTATGGGTATCATCTACCCAGGTGATTCTGATATCCGTTACTCTCCATCTTTCCAGGGTCAG
GTTACCATCTCTGCTGATAAATCTATCACCACCGCTTACCTGCAGTGGTCTTCTCTGAAAGCCTCGGACA
CCGCCATTTATTACTGTGCTCGTCACGATATCGAAGGTTTCGATTACTGGGGTCGTGGAACCCTGGTCAC
CGTCTCCTCAgctagcggcaaaccaatcccaaacccactgctgggc
> 5a - anifrolumab dLCDR1
(SEQ ID NO: 168)
cagttagataaaagaggcgcggcagcaagcggcgcgcatgccGAAATCGTTCTGACCCAGTCTCCAGGTA
CCCTGTCTCTGTCTCCAGGTGAACGTGCCACTCTGTCTTGTCGTGCTTCTagagaccatggccagtaagg
ccggtctctCTGGCTTGGTACCAGCAGAAACCAGGTCAGGCTCCACGTCTGCTGATCTACGGTGCTTCTT
CTCGTGCTACCGGTATCCCAGATCGTCTGTCTGGTTCTGGTTCTGGTACCGATTTCACCCTGACCATCAC
CCGTCTGGAACCAGAAGATTTCGCTGTTTACTACTGTCAGCAGTACGATTCTTCTGCTATCACCTTCGGT
GGTGGTACCAAAGTTGAAATCAAATCCGGAGGGTCGACCATAACTTCGTATAATGTATACTATACGAAGT
TATCCTCGAGCGGTACCGAAGTTCAGCTGGTTCAGTCTGGTGCTGAAGTTAAAAAACCAGGTGAATCTCT
GAAAATCTCTTGTAAAGGTTCTGGTTACATCTTCACCAACTACTGGATCGCTTGGGTTCGTCAGGTTCCA
GGTAAAGGTCTGGAATCTATGGGTATCATCTACCCAGGTGATTCTGATATCCGTTACTCTCCATCTTTCC
AGGGTCAGGTTACCATCTCTGCTGATAAATCTATCACCACCGCTTACCTGCAGTGGTCTTCTCTGAAAGC
CTCGGACACCGCCATTTATTACTGTGCTCGTCACGATATCGAAGGTTTCGATTACTGGGGTCGTGGAACC
CTGGTCACCGTCTCCTCAgctagcggcaaaccaatcccaaacccactgctgggc
> 5b - anifrolumab dLCDR2
(SEQ ID NO: 169)
cagttagataaaagaggcgcggcagcaagcggcgcgcatgccGAAATCGTTCTGACCCAGTCTCCAGGTA
CCCTGTCTCTGTCTCCAGGTGAACGTGCCACTCTGTCTTGTCGTGCTTCTCAGTCTGTTTCTTCTTCTTT
CCTGGCTTGGTACCAGCAGAAACCAGGTCAGGCTCCACGTCTGCTGATCTACagagaccatggccagtaa
ggccggtctctGGTATCCCAGATCGTCTGTCTGGTTCTGGTTCTGGTACCGATTTCACCCTGACCATCAC
CCGTCTGGAACCAGAAGATTTCGCTGTTTACTACTGTCAGCAGTACGATTCTTCTGCTATCACCTTCGGT
GGTGGTACCAAAGTTGAAATCAAATCCGGAGGGTCGACCATAACTTCGTATAATGTATACTATACGAAGT
TATCCTCGAGCGGTACCGAAGTTCAGCTGGTTCAGTCTGGTGCTGAAGTTAAAAAACCAGGTGAATCTCT
GAAAATCTCTTGTAAAGGTTCTGGTTACATCTTCACCAACTACTGGATCGCTTGGGTTCGTCAGGTTCCA
GGTAAAGGTCTGGAATCTATGGGTATCATCTACCCAGGTGATTCTGATATCCGTTACTCTCCATCTTTCC
AGGGTCAGGTTACCATCTCTGCTGATAAATCTATCACCACCGCTTACCTGCAGTGGTCTTCTCTGAAAGC
CTCGGACACCGCCATTTATTACTGTGCTCGTCACGATATCGAAGGTTTCGATTACTGGGGTCGTGGAACC
CTGGTCACCGTCTCCTCAgctagcggcaaaccaatcccaaacccactgctgggc
> 5c - anifrolumab dLCDR3
(SEQ ID NO: 170)
cagttagataaaagaggcgcggcagcaagcggcgcgcatgccGAAATCGTTCTGACCCAGTCTCCAGGTA
CCCTGTCTCTGTCTCCAGGTGAACGTGCCACTCTGTCTTGTCGTGCTTCTCAGTCTGTTTCTTCTTCTTT
CCTGGCTTGGTACCAGCAGAAACCAGGTCAGGCTCCACGTCTGCTGATCTACGGTGCTTCTTCTCGTGCT
ACCGGTATCCCAGATCGTCTGTCTGGTTCTGGTTCTGGTACCGATTTCACCCTGACCATCACCCGTCTGG
AACCAGAAGATTTCGCTGTTTACTACTGTagagaccatggccagtaaggccggtctctTTCGGTGGTGGT
ACCAAAGTTGAAATCAAATCCGGAGGGTCGACCATAACTTCGTATAATGTATACTATACGAAGTTATCCT
CGAGCGGTACCGAAGTTCAGCTGGTTCAGTCTGGTGCTGAAGTTAAAAAACCAGGTGAATCTCTGAAAAT
CTCTTGTAAAGGTTCTGGTTACATCTTCACCAACTACTGGATCGCTTGGGTTCGTCAGGTTCCAGGTAAA
GGTCTGGAATCTATGGGTATCATCTACCCAGGTGATTCTGATATCCGTTACTCTCCATCTTTCCAGGGTC
AGGTTACCATCTCTGCTGATAAATCTATCACCACCGCTTACCTGCAGTGGTCTTCTCTGAAAGCCTCGGA
CACCGCCATTTATTACTGTGCTCGTCACGATATCGAAGGTTTCGATTACTGGGGTCGTGGAACCCTGGTC
ACCGTCTCCTCAgctagcggcaaaccaatcccaaacccactgctgggc
> 5d - anifrolumab dHCDR1
(SEQ ID NO: 171)
cagttagataaaagaggcgcggcagcaagcggcgcgcatgccGAAATCGTTCTGACCCAGTCTCCAGGTA
CCCTGTCTCTGTCTCCAGGTGAACGTGCCACTCTGTCTTGTCGTGCTTCTCAGTCTGTTTCTTCTTCTTT
CCTGGCTTGGTACCAGCAGAAACCAGGTCAGGCTCCACGTCTGCTGATCTACGGTGCTTCTTCTCGTGCT
ACCGGTATCCCAGATCGTCTGTCTGGTTCTGGTTCTGGTACCGATTTCACCCTGACCATCACCCGTCTGG
AACCAGAAGATTTCGCTGTTTACTACTGTCAGCAGTACGATTCTTCTGCTATCACCTTCGGTGGTGGTAC
CAAAGTTGAAATCAAATCCGGAGGGTCGACCATAACTTCGTATAATGTATACTATACGAAGTTATCCTCG
AGCGGTACCGAAGTTCAGCTGGTTCAGTCTGGTGCTGAAGTTAAAAAACCAGGTGAATCTCTGAAAATCT
CTTGTAAAGGTTCTagagaccatggccagtaaggccggtctctATCGCTTGGGTTCGTCAGGTTCCAGGT
AAAGGTCTGGAATCTATGGGTATCATCTACCCAGGTGATTCTGATATCCGTTACTCTCCATCTTTCCAGG
GTCAGGTTACCATCTCTGCTGATAAATCTATCACCACCGCTTACCTGCAGTGGTCTTCTCTGAAAGCCTC
GGACACCGCCATTTATTACTGTGCTCGTCACGATATCGAAGGTTTCGATTACTGGGGTCGTGGAACCCTG
GTCACCGTCTCCTCAgctagcggcaaaccaatcccaaacccactgctgggc
> 5e - anifrolumab dHCDR2
(SEQ ID NO: 172)
cagttagataaaagaggcgcggcagcaagcggcgcgcatgccGAAATCGTTCTGACCCAGTCTCCAGGTA
CCCTGTCTCTGTCTCCAGGTGAACGTGCCACTCTGTCTTGTCGTGCTTCTCAGTCTGTTTCTTCTTCTTT
CCTGGCTTGGTACCAGCAGAAACCAGGTCAGGCTCCACGTCTGCTGATCTACGGTGCTTCTTCTCGTGCT
ACCGGTATCCCAGATCGTCTGTCTGGTTCTGGTTCTGGTACCGATTTCACCCTGACCATCACCCGTCTGG
AACCAGAAGATTTCGCTGTTTACTACTGTCAGCAGTACGATTCTTCTGCTATCACCTTCGGTGGTGGTAC
CAAAGTTGAAATCAAATCCGGAGGGTCGACCATAACTTCGTATAATGTATACTATACGAAGTTATCCTCG
AGCGGTACCGAAGTTCAGCTGGTTCAGTCTGGTGCTGAAGTTAAAAAACCAGGTGAATCTCTGAAAATCT
CTTGTAAAGGTTCTGGTTACATCTTCACCAACTACTGGATCGCTTGGGTTCGTCAGGTTCCAGGTAAAGG
TCTGGAATCTATGGGTATCagagaccatggccagtaaggccggtctctCGTTACTCTCCATCTTTCCAGG
GTCAGGTTACCATCTCTGCTGATAAATCTATCACCACCGCTTACCTGCAGTGGTCTTCTCTGAAAGCCTC
GACACCGCCATTTATTACTGTGCTCGTCACGATATCGAAGGTTTCGATTACTGGGGTCGTGGAACCCTG
GTCACCGTCTCCTCAgctagcggcaaaccaatcccaaacccactgctgggc
> 5f - anifrolumab dHCDR3
(SEQ ID NO: 173)
cagttagataaaagaggcgcggcagcaagcggcgcgcatgccGAAATCGTTCTGACCCAGTCTCCAGGTA
CCCTGTCTCTGTCTCCAGGTGAACGTGCCACTCTGTCTTGTCGTGCTTCTCAGTCTGTTTCTTCTTCTTT
CCTGGCTTGGTACCAGCAGAAACCAGGTCAGGCTCCACGTCTGCTGATCTACGGTGCTTCTTCTCGTGCT
ACCGGTATCCCAGATCGTCTGTCTGGTTCTGGTTCTGGTACCGATTTCACCCTGACCATCACCCGTCTGG
AACCAGAAGATTTCGCTGTTTACTACTGTCAGCAGTACGATTCTTCTGCTATCACCTTCGGTGGTGGTAC
CAAAGTTGAAATCAAATCCGGAGGGTCGACCATAACTTCGTATAATGTATACTATACGAAGTTATCCTCG
AGCGGTACCGAAGTTCAGCTGGTTCAGTCTGGTGCTGAAGTTAAAAAACCAGGTGAATCTCTGAAAATCT
CTTGTAAAGGTTCTGGTTACATCTTCACCAACTACTGGATCGCTTGGGTTCGTCAGGTTCCAGGTAAAGG
TCTGGAATCTATGGGTATCATCTACCCAGGTGATTCTGATATCCGTTACTCTCCATCTTTCCAGGGTCAG
GTTACCATCTCTGCTGATAAATCTATCACCACCGCTTACCTGCAGTGGTCTTCTCTGAAAGCCTCGGACA
CCGCCATTTATTACTGTagagaccatggccagtaaggccggtctctGGAACCCTGGTCACCGTCTCCTCA
gctagcggcaaaccaatcccaaacccactgctgggc
> 6 - evolocumab
(SEQ ID NO: 174)
cagttagataaaagaggcgcggcagcaagcggcgcgcatgccGAATCTGCTCTGACCCAGCCAGCTTCTG
TTTCTGGTTCTCCAGGTCAGTCTATCACCATCTCTTGTACCGGTACCTCTTCTGATGTTGGTGGTTACAA
CTCTGTTTCTTGGTACCAGCAGCACCCAGGTAAAGCTCCAAAACTGATGATCTACGAAGTTTCTAACCGT
CCATCTGGTGTTTCTAACCGTTTCTCTGGTTCTAAATCTGGTAACACCGCTTCTCTGACCATCTCTGGTC
TGCAGGCTGAAGATGAAGCTGATTACTACTGTAACTCTTACACCTCTACCTCTATGGTTTTCGGTGGTGG
TACCAAACTGACCGTTCTGTCCGGAGGGTCGACCATAACTTCGTATAATGTATACTATACGAAGTTATCC
TCGAGCGGTACCGAAGTTCAGCTGGTTCAGTCTGGTGCTGAAGTTAAAAAACCAGGTGCTTCTGTTAAAG
TTTCTTGTAAAGCTTCTGGTTACACCCTGACCTCTTACGGTATCTCTTGGGTTCGTCAGGCTCCAGGTCA
GGGTCTGGAATGGATGGGTTGGGTTTCTTTCTACAACGGTAACACCAACTACGCTCAGAAACTGCAGGGT
CGTGGTACCATGACCACCGATCCATCTACCTCTACCGCTTACATGGAACTGCGTTCTCTGCGTTCTGACG
ACACGGCCGTGTATTACTGTGCTCGTGGTTACGGTATGGATGTTTGGGGTCAGGGGACCACGGTCACCGT
CTCCTCAgctagcggcaaaccaatcccaaacccactgctgggc
> 6a - evolocumab dLCDR1
(SEQ ID NO: 175)
cagttagataaaagaggcgcggcagcaagcggcgcgcatgccGAATCTGCTCTGACCCAGCCAGCTTCTG
TTTCTGGTTCTCCAGGTCAGTCTATCACCATCTCTTGTACCGGTACCagagaccatggccagtaaggccg
gtctctGTTTCTTGGTACCAGCAGCACCCAGGTAAAGCTCCAAAACTGATGATCTACGAAGTTTCTAACC
GTCCATCTGGTGTTTCTAACCGTTTCTCTGGTTCTAAATCTGGTAACACCGCTTCTCTGACCATCTCTGG
TCTGCAGGCTGAAGATGAAGCTGATTACTACTGTAACTCTTACACCTCTACCTCTATGGTTTTCGGTGGT
GGTACCAAACTGACCGTTCTGTCCGGAGGGTCGACCATAACTTCGTATAATGTATACTATACGAAGTTAT
CCTCGAGCGGTACCGAAGTTCAGCTGGTTCAGTCTGGTGCTGAAGTTAAAAAACCAGGTGCTTCTGTTAA
AGTTTCTTGTAAAGCTTCTGGTTACACCCTGACCTCTTACGGTATCTCTTGGGTTCGTCAGGCTCCAGGT
CAGGGTCTGGAATGGATGGGTTGGGTTTCTTTCTACAACGGTAACACCAACTACGCTCAGAAACTGCAGG
GTCGTGGTACCATGACCACCGATCCATCTACCTCTACCGCTTACATGGAACTGCGTTCTCTGCGTTCTGA
CGACACGGCCGTGTATTACTGTGCTCGTGGTTACGGTATGGATGTTTGGGGTCAGGGGACCACGGTCACC
GTCTCCTCAgctagcggcaaaccaatcccaaacccactgctgggc
> 6b - evolocumab dLCDR2
(SEQ ID NO: 176)
cagttagataaaagaggcgcggcagcaagcggcgcgcatgccGAATCTGCTCTGACCCAGCCAGCTTCTG
TTTCTGGTTCTCCAGGTCAGTCTATCACCATCTCTTGTACCGGTACCTCTTCTGATGTTGGTGGTTACAA
CTCTGTTTCTTGGTACCAGCAGCACCCAGGTAAAGCTCCAAAACTGATGATCTACagagaccatggccag
taaggccggtctctGGTGTTTCTAACCGTTTCTCTGGTTCTAAATCTGGTAACACCGCTTCTCTGACCAT
CTCTGGTCTGCAGGCTGAAGATGAAGCTGATTACTACTGTAACTCTTACACCTCTACCTCTATGGTTTTC
GGTGGTGGTACCAAACTGACCGTTCTGTCCGGAGGGTCGACCATAACTTCGTATAATGTATACTATACGA
AGTTATCCTCGAGCGGTACCGAAGTTCAGCTGGTTCAGTCTGGTGCTGAAGTTAAAAAACCAGGTGCTTC
TGTTAAAGTTTCTTGTAAAGCTTCTGGTTACACCCTGACCTCTTACGGTATCTCTTGGGTTCGTCAGGCT
CCAGGTCAGGGTCTGGAATGGATGGGTTGGGTTTCTTTCTACAACGGTAACACCAACTACGCTCAGAAAC
TGCAGGGTCGTGGTACCATGACCACCGATCCATCTACCTCTACCGCTTACATGGAACTGCGTTCTCTGCG
TTCTGACGACACGGCCGTGTATTACTGTGCTCGTGGTTACGGTATGGATGTTTGGGGTCAGGGGACCACG
GTCACCGTCTCCTCAgctagcggcaaaccaatcccaaacccactgctgggc
> 6c - evolocumab dLCDR3
(SEQ ID NO: 177)
cagttagataaaagaggcgcggcagcaagcggcgcgcatgccGAATCTGCTCTGACCCAGCCAGCTTCTG
TTTCTGGTTCTCCAGGTCAGTCTATCACCATCTCTTGTACCGGTACCTCTTCTGATGTTGGTGGTTACAA
CTCTGTTTCTTGGTACCAGCAGCACCCAGGTAAAGCTCCAAAACTGATGATCTACGAAGTTTCTAACCGT
CCATCTGGTGTTTCTAACCGTTTCTCTGGTTCTAAATCTGGTAACACCGCTTCTCTGACCATCTCTGGTC
TGCAGGCTGAAGATGAAGCTGATTACTACTGTagagaccatggccagtaaggccggtctctTTCGGTGGT
GGTACCAAACTGACCGTTCTGTCCGGAGGGTCGACCATAACTTCGTATAATGTATACTATACGAAGTTAT
CCTCGAGCGGTACCGAAGTTCAGCTGGTTCAGTCTGGTGCTGAAGTTAAAAAACCAGGTGCTTCTGTTAA
AGTTTCTTGTAAAGCTTCTGGTTACACCCTGACCTCTTACGGTATCTCTTGGGTTCGTCAGGCTCCAGGT
CAGGGTCTGGAATGGATGGGTTGGGTTTCTTTCTACAACGGTAACACCAACTACGCTCAGAAACTGCAGG
GTCGTGGTACCATGACCACCGATCCATCTACCTCTACCGCTTACATGGAACTGCGTTCTCTGCGTTCTGA
CGACACGGCCGTGTATTACTGTGCTCGTGGTTACGGTATGGATGTTTGGGGTCAGGGGACCACGGTCACC
GTCTCCTCAgctagcggcaaaccaatcccaaacccactgctgggc
> 6d - evolocumab dHCDR1
(SEQ ID NO: 178)
cagttagataaaagaggcgcggcagcaagcggcgcgcatgccGAATCTGCTCTGACCCAGCCAGCTTCTG
TTTCTGGTTCTCCAGGTCAGTCTATCACCATCTCTTGTACCGGTACCTCTTCTGATGTTGGTGGTTACAA
CTCTGTTTCTTGGTACCAGCAGCACCCAGGTAAAGCTCCAAAACTGATGATCTACGAAGTTTCTAACCGT
CCATCTGGTGTTTCTAACCGTTTCTCTGGTTCTAAATCTGGTAACACCGCTTCTCTGACCATCTCTGGTC
TGCAGGCTGAAGATGAAGCTGATTACTACTGTAACTCTTACACCTCTACCTCTATGGTTTTCGGTGGTGG
TACCAAACTGACCGTTCTGTCCGGAGGGTCGACCATAACTTCGTATAATGTATACTATACGAAGTTATCC
TCGAGCGGTACCGAAGTTCAGCTGGTTCAGTCTGGTGCTGAAGTTAAAAAACCAGGTGCTTCTGTTAAAG
TTTCTTGTAAAGCTTCTagagaccatggccagtaaggccggtctctATCTCTTGGGTTCGTCAGGCTCCA
GGTCAGGGTCTGGAATGGATGGGTTGGGTTTCTTTCTACAACGGTAACACCAACTACGCTCAGAAACTGC
AGGGTCGTGGTACCATGACCACCGATCCATCTACCTCTACCGCTTACATGGAACTGCGTTCTCTGCGTTC
TGACGACACGGCCGTGTATTACTGTGCTCGTGGTTACGGTATGGATGTTTGGGGTCAGGGGACCACGGTC
ACCGTCTCCTCAgctagcggcaaaccaatcccaaacccactgctgggc
> 6e - evolocumab dHCDR2
(SEQ ID NO: 179)
cagttagataaaagaggcgcggcagcaagcggcgcgcatgccGAATCTGCTCTGACCCAGCCAGCTTCTG
TTTCTGGTTCTCCAGGTCAGTCTATCACCATCTCTTGTACCGGTACCTCTTCTGATGTTGGTGGTTACAA
CTCTGTTTCTTGGTACCAGCAGCACCCAGGTAAAGCTCCAAAACTGATGATCTACGAAGTTTCTAACCGT
CCATCTGGTGTTTCTAACCGTTTCTCTGGTTCTAAATCTGGTAACACCGCTTCTCTGACCATCTCTGGTC
TGCAGGCTGAAGATGAAGCTGATTACTACTGTAACTCTTACACCTCTACCTCTATGGTTTTCGGTGGTGG
TACCAAACTGACCGTTCTGTCCGGAGGGTCGACCATAACTTCGTATAATGTATACTATACGAAGTTATCC
TCGAGCGGTACCGAAGTTCAGCTGGTTCAGTCTGGTGCTGAAGTTAAAAAACCAGGTGCTTCTGTTAAAG
TTTCTTGTAAAGCTTCTGGTTACACCCTGACCTCTTACGGTATCTCTTGGGTTCGTCAGGCTCCAGGTCA
GGGTCTGGAATGGATGGGTTGGagagaccatggccagtaaggccggtctctAACTACGCTCAGAAACTGC
AGGGTCGTGGTACCATGACCACCGATCCATCTACCTCTACCGCTTACATGGAACTGCGTTCTCTGCGTTC
TGACGACACGGCCGTGTATTACTGTGCTCGTGGTTACGGTATGGATGTTTGGGGTCAGGGGACCACGGTC
ACCGTCTCCTCAgctagcggcaaaccaatcccaaacccactgctgggc
> 6f - evolocumab dHCDR3
(SEQ ID NO: 180)
cagttagataaaagaggcgcggcagcaagcggcgcgcatgccGAATCTGCTCTGACCCAGCCAGCTTCTG
TTTCTGGTTCTCCAGGTCAGTCTATCACCATCTCTTGTACCGGTACCTCTTCTGATGTTGGTGGTTACAA
CTCTGTTTCTTGGTACCAGCAGCACCCAGGTAAAGCTCCAAAACTGATGATCTACGAAGTTTCTAACCGT
CCATCTGGTGTTTCTAACCGTTTCTCTGGTTCTAAATCTGGTAACACCGCTTCTCTGACCATCTCTGGTC
TGCAGGCTGAAGATGAAGCTGATTACTACTGTAACTCTTACACCTCTACCTCTATGGTTTTCGGTGGTGG
TACCAAACTGACCGTTCTGTCCGGAGGGTCGACCATAACTTCGTATAATGTATACTATACGAAGTTATCC
TCGAGCGGTACCGAAGTTCAGCTGGTTCAGTCTGGTGCTGAAGTTAAAAAACCAGGTGCTTCTGTTAAAG
TTTCTTGTAAAGCTTCTGGTTACACCCTGACCTCTTACGGTATCTCTTGGGTTCGTCAGGCTCCAGGTCA
GGGTCTGGAATGGATGGGTTGGGTTTCTTTCTACAACGGTAACACCAACTACGCTCAGAAACTGCAGGGT
CGTGGTACCATGACCACCGATCCATCTACCTCTACCGCTTACATGGAACTGCGTTCTCTGCGTTCTGACG
ACACGGCCGTGTATTACTGTagagaccatggccagtaaggccggtctctGGGACCACGGTCACCGTCTCC
TCAgctagcggcaaaccaatcccaaacccactgctgggc

Sequences 1-6 refer to the coding sequence of the exemplary scaffolds as indicated and sequences 1a-1f, 2a-2f, 3a-3f, 4a-4f, 5a-5f, and 6a-6f refer to sequences in which cloning sites were inserted flanking the corresponding CDRs of each exemplary scaffold as indicated.

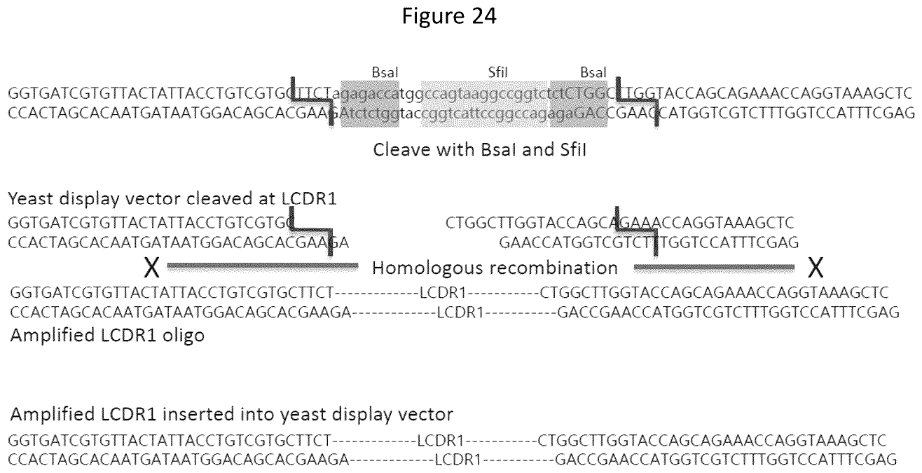

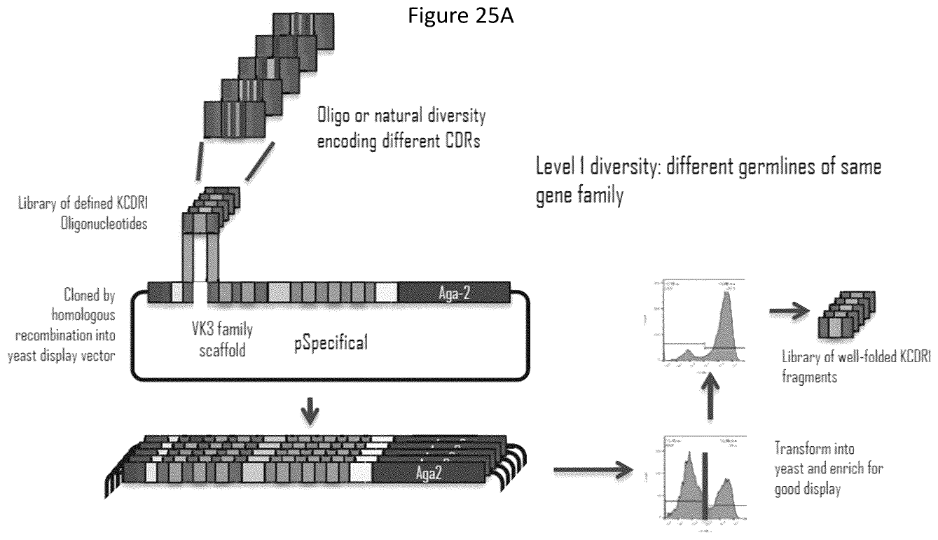

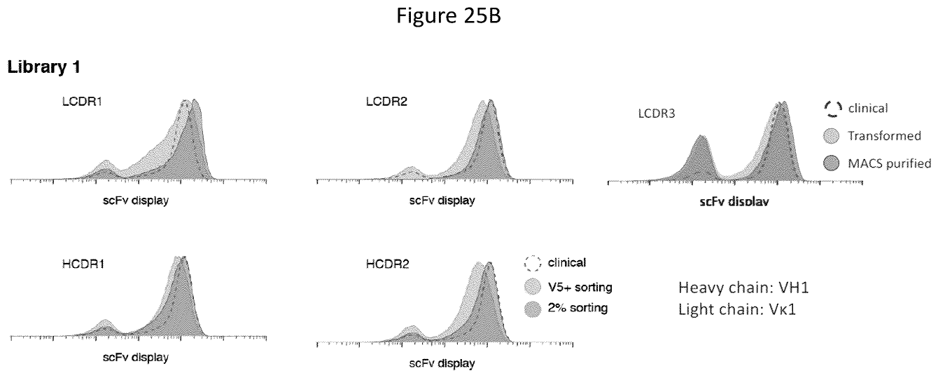

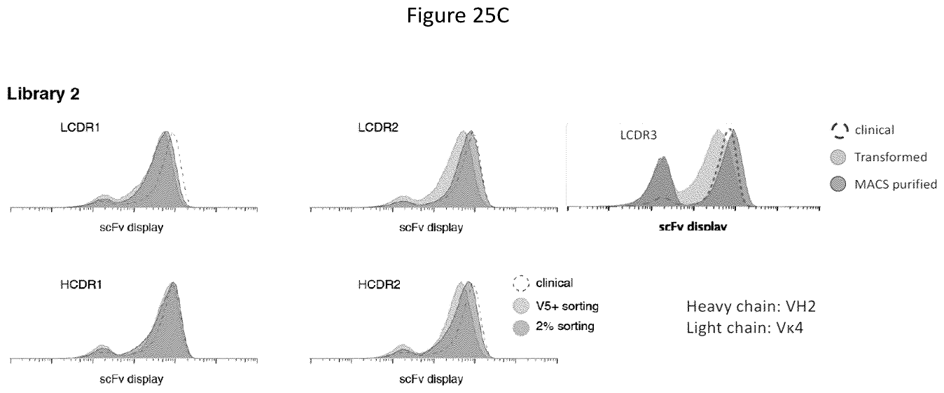

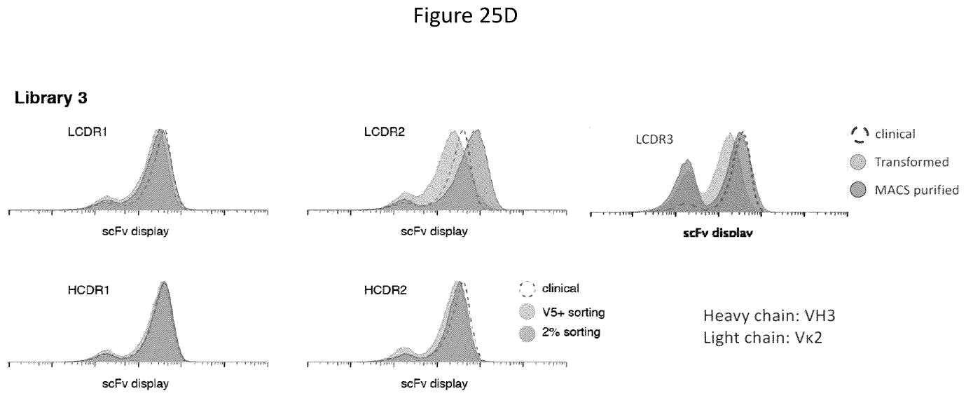

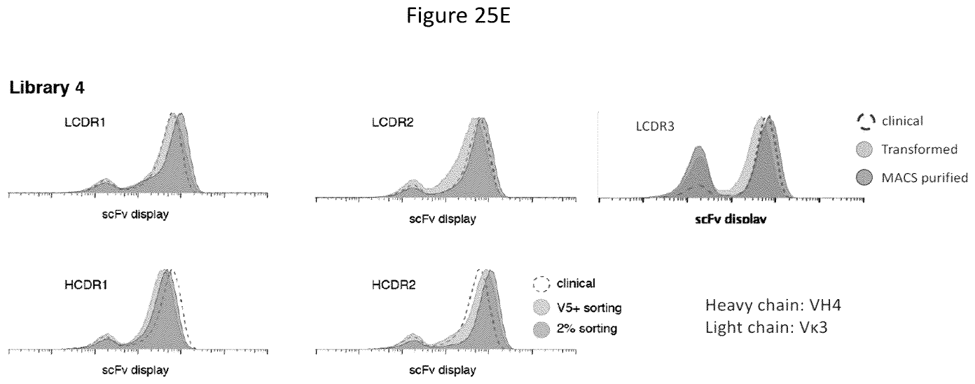

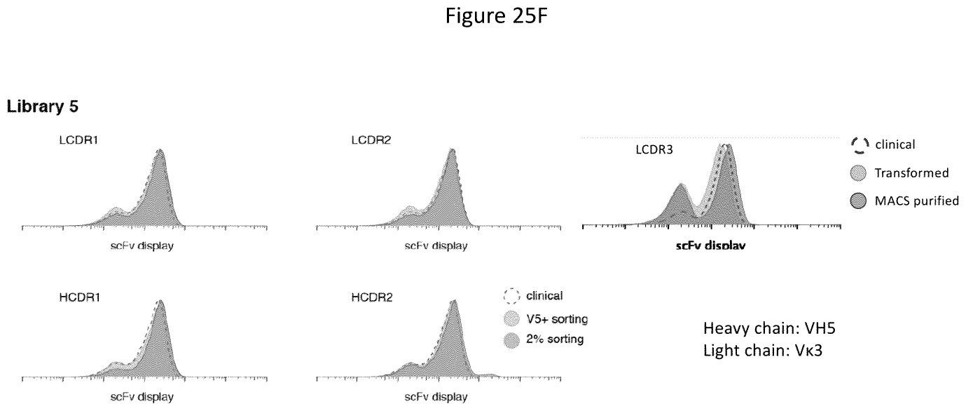

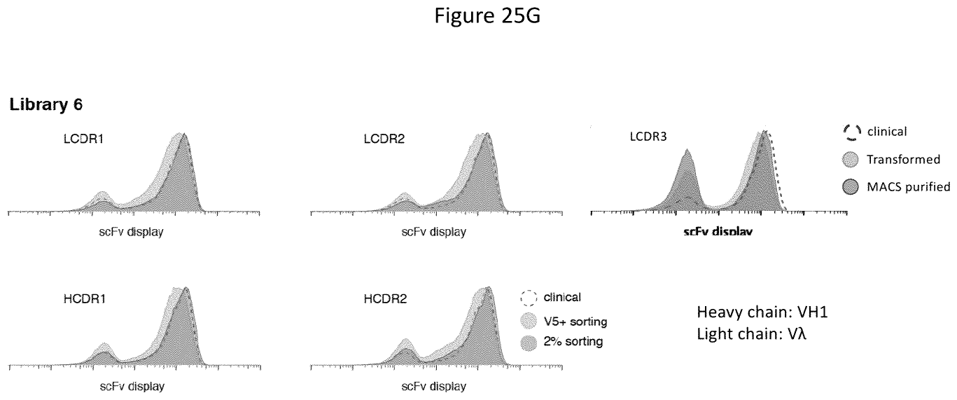

Using LCDR1 of library 1 as an example, the LCDR1 scaffold vector was digested with BsaI and Sfil leaving a gap at the site of LCDR1, as shown in FIG. 24. The cleaved vector and the collection of LCDR1 oligonucleotides were then transformed into yeast. Inside yeast cells, homologous recombination between the vector and the LCDR1 oligonucleotides results in insertion of the LCDR1 oligonucleotides into the LCDR1 scaffold vector. The entire population of LCDR1 yeast scaffold vectors carrying each of the LCDR1oligonucleotides identified above constitutes a LCDR1 yeast display library (FIG. 25). For the LCDR1 scaffold vector, all portions of the VH and VL domains are constant except for the LCDR1 that is assessed. Selection for functional LCDR1 was carried out by sorting for display (i.e., expression). After the display of the scFv is induced, the yeast cells are stained with fluorescent-labelled antibody that detects scFv display (using the mAb recognizing the SV5 tag). For CDR1-2 libraries, the populations are analyzed by flow cytometry and sorted by fluorescence activated cell sorting by gating the top 2% most fluorescent cells among the positive population—this ensures the enrichment for CDRs that promote high levels of display. In FIGS. 22A-L, the population after this stringent 2% sorting is indicated as

“Replicated natural after filtering”. At least a 10-fold number of cells is sorted as compared to the theoretical diversity to ensure recovery of all possible clones. For LCDR3, after scFv display induction and staining with fluorescent-labelled antibody that detects the scFv display (SV5), the positive population (scFv displaying) is purified using MACS (magnetic-activated cell sorting) employing magnetic nanoparticles that recognize the primary antibody (SV5) used. The higher the level of the scFv display, the higher the probability of the cell binding the nanoparticles—especially when competition is employed by having a number of cells that far exceeds the binding capacity of the nanoparticles, thus, enriching for well displaying sequences, analogously to fluorescence activated cell sorting of the most fluorescent 2% for the CDR1-2 described above. The MACS technique is preferentially employed for LCDR3 due to the capacity of purifying a large number of cells in a short span of time, since the theoretical diversity of the LCDR3 is several fold higher than CDR1-2.

Although scFvs are used in this example for yeast display, the format of CDR-specific scaffold vectors can be VH, VL, scFv, Fab or full-length immunoglobulin, the requirement being that display can be carried out. By sorting libraries of single CDRs cloned into well folded scaffolds, those CDRs that contain stop codons, frameshifts, or are poorly expressed or polyreactive may be eliminated. Effective display on the yeast surface has been previously correlated with improved stability and folding in diverse proteins (Cherf, G. M. and J. R. Cochran (2015). “Applications of Yeast Surface Display for Protein Engineering.” Methods Mol Biol 1319:155-175; Pavoor, T. V., et al., (2012). “An enhanced approach for engineering thermally stable proteins using yeast display.” Protein engineering, design & selection: PEDS 25(10):625-630; Pepper, L. R., et al., (2008). “A decade of yeast surface display technology: where are we now?” Comb Chem High Throughput Screen 11(2):127-134; Xu, L., et al., (2013). “Rapid optimization and prototyping for therapeutic antibody-like molecules.” MAbs 5(2):237-254.). After each CDR library is sorted, a collection of well expressed, non-polyreactive CDRs is obtained by isolating DNA from yeast cells expressing well folded CDRs. Schematic illustrations of how functional CDR libraries are cloned and sorted are shown in FIGS. 23-25. The non-filtered and filtered libraries for the 6 different scaffolds are show in FIGS. 25B-G: the populations are analyzed by flow cytometry and expression levels (x axis) are represented as a histogram. The analysis shows a clear improvement after enrichment (top 2% by fluorescence activated cell sorting for CDRs 1-2, and magnetic activated cell sorting for LCDR3), with most libraries showing display levels exceeding that of the original clinical candidate from which the scaffolds were generated.

In the examples provided here, we have sorted for improved expression levels. However, a similar approach can be taken using any selective method that distinguishes yeast displaying antibodies with desirable properties (e.g. high expression, low polyreactivity, good developability) from yeast displaying antibodies with undesirable properties (e.g. low expression, high polyreactivity, poor developability). For example, to select antibodies with reduced polyreactivity, clones displaying antibodies (scFvs, Fabs, IgGs, or other antibody fragment) that do not bind to polyspecificity reagents would be selected. Examples of such polyspecificity reagents include those described in (Hotzel, I. et al. A strategy for risk mitigation of antibodies with fast clearance. MAbs 4, 753-760, doi: 10.4161/mabs.22189 (2012); Xu, Y. et al. Addressing polyspecificity of antibodies selected from an in vitro yeast presentation system: a FACS-based, high-throughput selection and analytical tool. Protein Eng Des Sel 26, 663-670, doi:10.1093/protein/gzt047 (2013); Kelly, R. L. et al. Chaperone proteins as single component reagents to assess antibody nonspecificity. MAbs 9, 1036-1040, doi: 10.1080/19420862.2017.1356529(2017).)

### Example 8: Assembly of Full Length scFv Library

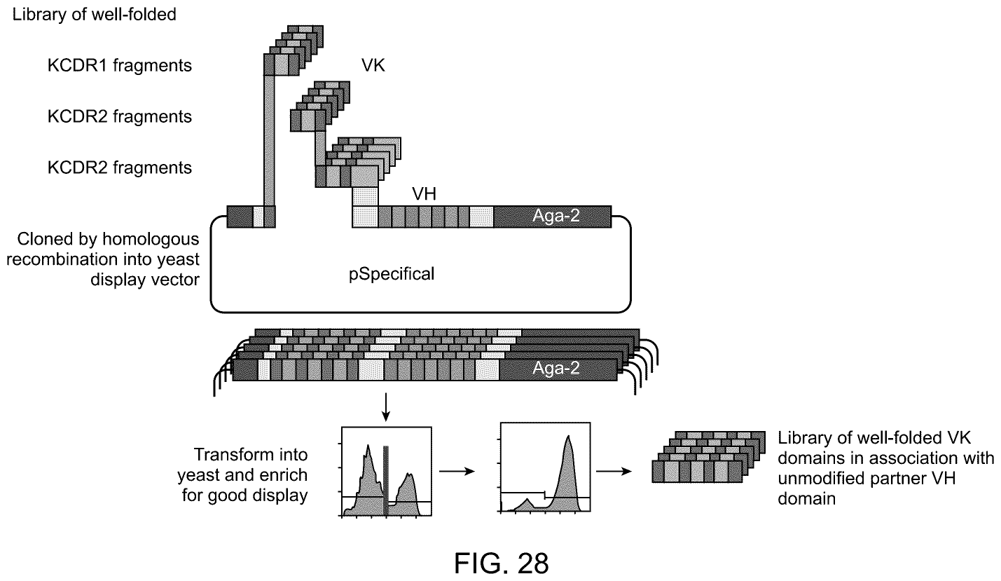

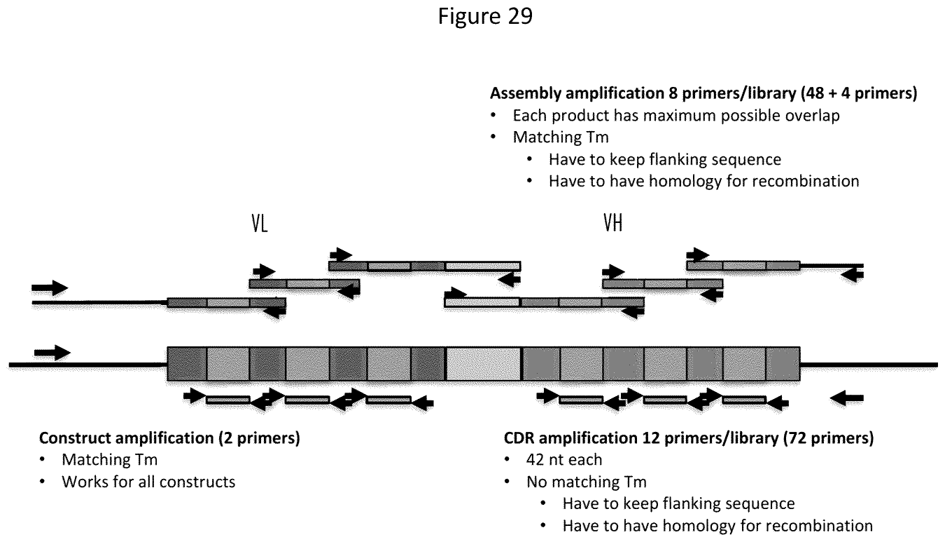

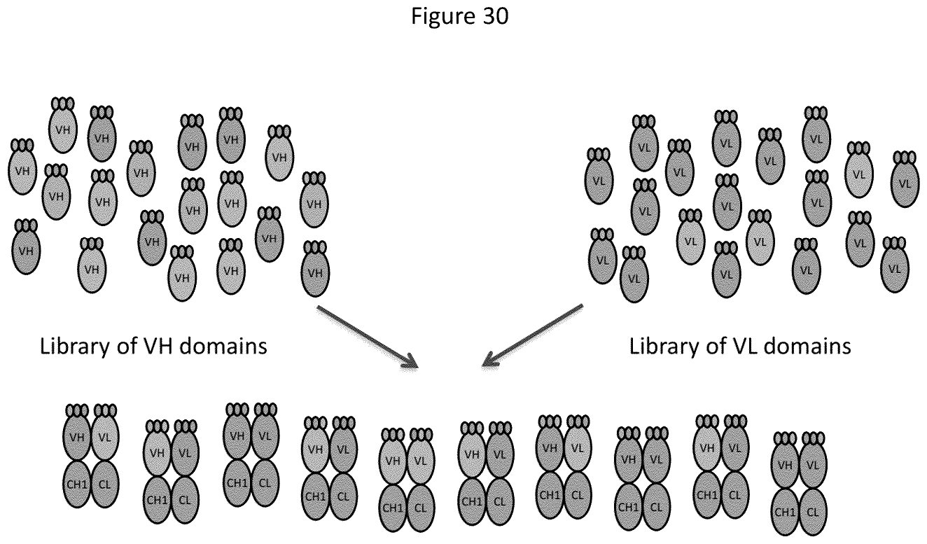

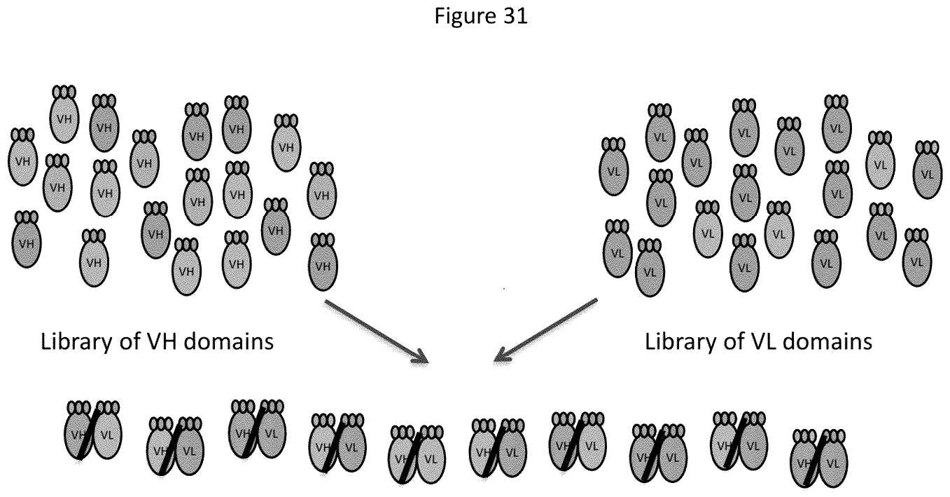

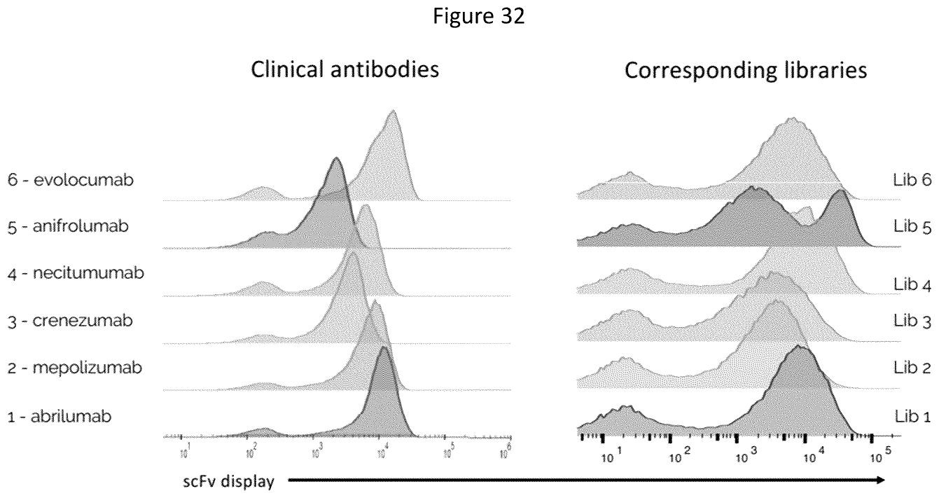

Once each individual CDR library was screened for expression and developability, the VH and VL CDRs were assembled into full length VH or VL domains. Individual CDRs in VL and VH were amplified with the flanking sequence using the primers described in Table 7 and Table 8. This was carried out as illustrated in FIGS. 29-31, with the VH and VL first assembled from their constituent CDR fragments and flanking framework regions, and then combined into complete scFvs or Fabs. An alternative approach would be to assemble each full length VH or VL within the context of their non-modified VL or VH partner chains as illustrated in FIGS. 26-28. This alternative approach would allow sorting (by FACS or MACS) for VH or VL libraries that are functional within the context of their unmodified partner chains. However, we found that the direct assembly of complete scFvs directly from libraries of filtered CDRs led to highly functional scFv libraries without the need for this intermediate step, as illustrated in FIG. 32, which shows that the peak display level of scFvs derived from the clinical antibodies used as scaffolds is similar to that for the corresponding libraries, except that the distribution of library display levels is broader than that of the clinical candidate antibodies used as scaffolds, and includes some scFvs that are displayed better than the parental clinical scaffold scFv. The functional antibody library can be assembled within the context of different display vectors, including phage, yeast or mammalian display vectors.

### Example 9: Cloning into a Phage Display Vector (pDAN5)

Once the scFv or Fab libraries were assembled, they were ligated into a phage display vector, such as pDAN5 to explore their functionality. This vector contains a cloning site upstream of the g3 of the filamentous phage, comprised by the restriction enzyme sites for BssHII and NheI. The scFv/Fabs created in Example 8 were amplified with flanking primers containing the BssHII restriction site upstream of the light chain and the NheI downstream of the heavy chain. The PCR product was then digested with the same enzymes to generate cohesive ends. The pDAN5 plasmid was cultivated in E. coli, extracted by alkaline lysis and purified by cesium chloride/ethidium bromide gradient. The plasmid was digested with the same enzymes and the backbone purified by agarose gel electrophoresis extraction followed by chromatography to remove contaminants. The backbone was ligated to the scFv/Fab library using T4 DNA ligase overnight at 16° C. The ligation was purified and electrotransformed into electrocompetent E. coli TG1 cells. The transformed cells were plated out on agar plates containing carbenicillin and glucose to select for bacteria that received the plasmid. Analysis of the scFv libraries by PacBio sequencing revealed >90% open reading frames (Table 9), and essentially no clone duplication (Table 10).

TABLE 9
Percentage of open reading frames in scFv libraries
as assessed by PacBio sequencing.

| | sequences | correct | | |
|---|---|---|---|---|
| Library | analyzed | frame | ORFs | ORF % |
| Lib1 | 6,510 | 6,103 | 6,050 | 93% |
| Lib2 | 5,699 | 5,403 | 5,355 | 94% |
| Lib3 | 7,012 | 6,623 | 6,583 | 94% |
| Lib4 | 9,168 | 8,660 | 8,613 | 94% |
| Lib5 | 7,640 | 7,282 | 7,242 | 95% |
| Lib6 | 5,378 | 5,027 | 5,005 | 93% |

TABLE 10
Analysis of library diversity by PacBio.

| | | Full-Length | | HCDR3 + LCDR3 | | HCDR3 | | LCDR3 | | HCDR2 | | LCDR2 | | HCDR1 | | LCDR1 | |
|---|---|---|---|---|---|---|---|---|---|---|---|---|---|---|---|---|---|
| | | Unique reads | | | | | | | | | | | | | | | |
| library | No. reads | No. | % | No. | % | No. | % | No. | % | No. | % | No. | % | No. | % | No. | % |
| 1 | 6000 | 5998 | 99.97 | 5998 | 99.97 | 5851 | 97.52 | 5679 | 94.65 | 1786 | 29.77 | 2224 | 37.07 | 2158 | 35.97 | 1328 | 22.13 |
| 2 | 5265 | 5258 | 99.87 | 5258 | 99.87 | 4740 | 90.03 | 4675 | 88.79 | 1927 | 36.60 | 1796 | 34.11 | 2267 | 43.06 | 481 | 9.14 |
| 3 | 6496 | 6493 | 99.95 | 6493 | 99.95 | 6300 | 96.98 | 5828 | 89.72 | 3077 | 47.37 | 1329 | 20.46 | 3676 | 56.59 | 438 | 6.74 |
| 4 | 8423 | 8420 | 99.96 | 8420 | 99.96 | 8137 | 96.60 | 7965 | 94.56 | 2538 | 30.13 | 1355 | 16.09 | 2525 | 29.98 | 1399 | 16.61 |
| 5 | 7030 | 7029 | 99.99 | 7015 | 99.79 | 6457 | 91.85 | 6263 | 89.09 | 1780 | 25.32 | 1873 | 26.64 | 2684 | 38.18 | 2090 | 29.73 |
| 6 | 4899 | 4898 | 99.98 | 4898 | 99.98 | 4735 | 96.65 | 4725 | 96.45 | 1639 | 33.46 | 1148 | 23.43 | 1833 | 37.42 | 1545 | 31.54 |

### Example 10: Creation of Bacteriophage Particles, Including Western Blot

The transformed bacteria were cultivated in a shaking flask containing liquid 2x YT media+carbenicillin+glucose (the glucose is to inhibit scFv/Fab expression) at 37° C. until an OD600 nm of 0.5 was reached. The bacteria were superinfected with M13KO7 helper phage (at a multiplicity of infection of 5) for 30 min at 37° C. without shaking. The bacteria were centrifuged, the media removed and replaced with 2xYT media+carbenicillin+kanamycin and cultivated for 16 h at 25° C. in a shaker incubator.

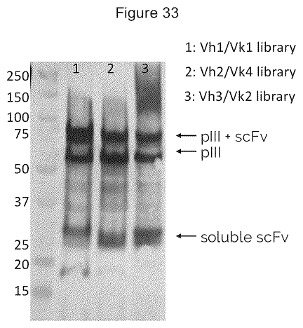

To recover the phage particles, the cultures were centrifuged to separate the bacteria and the supernatant, where the phage is found. The supernatant was mixed with a 20% PEG 8000+2.5 M NaCl solution at a 5:1 ratio. This causes the phage to precipitate, allowing them to be harvested by centrifugation. The supernatant was discarded and the phage pellet was resuspended in a PBS solution. The display of the scFv/Fab by the phage was assessed by SDS-PAGE+western blot using an antibody that specifically recognizes the expression tag (SV5) as show in FIG. 33.

### Example 11: Antibody Selection by Combined Phage and Yeast Display Using the Library

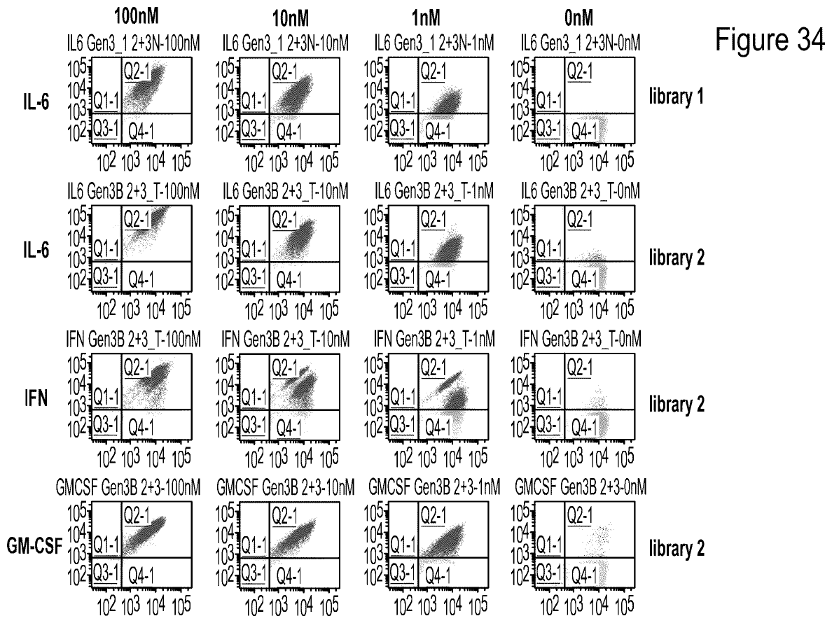

After construction and phage particle production, the library was screened against targets of interest. While selection can be carried out using phage display alone (Sblattero, D. & Bradbury, A. Exploiting recombination in single bacteria to make large phage antibody libraries. Nat Biotechnol 18, 75-80 (2000)), we prefer to combine phage and yeast display technologies (Ferrara, F. et al. Using phage and yeast display to select hundreds of monoclonal antibodies: application to antigen 85, a tuberculosis biomarker. PLoS One 7, e49535 (2012)). 10¹² phage particles displaying the scFv library were used in two rounds of selection against the biotinylated recombinant human antigens of clinical interest—interleukin 6, interferon alpha, and GM-CSF—using the Kingfisher magnetic bead system: 2×10⁷ streptavidin-conjugated magnetic beads (Dynabeads M-280) coated with the biotinylated proteins (100-400 nM) were washed, coated with the antigen, incubated with the phage particles and washed again to remove non-binders. Phage particles were then eluted by reducing the pH and infecting F′ pilus-carrying bacteria (Ominmax-2T1, Thermo Fisher Scientific). The phages were propagated, and the selection cycle reiterated. After two rounds of phage enrichment, the scFvs were PCR amplified and transferred to an N-terminal yeast display system by homologous recombination (pSpec yeast display vector), in which the scFv is displayed fused to the N terminus of Aga-2. The transformed yeast was then induced for scFv display by adding galactose to the culture media. The induced yeast minilibraries were then used for another two to three rounds of enrichment against the biotinylated recombinant human antigens by fluorescence activated cell sorting. Up to 10,000 yeast cells with positive antigen binding signal were sorted and propagated in each round. Target concentration in the first sorting round was 100 nM, reduced to 10 nM and then 1 nM. After these rounds of phage and yeast sorting enrichment the recovered populations was analyzed by flow cytometry to test for binding against the antigens in decreasing concentration of antigen and in the absence of the antigen to check for non-specific binding to secondary reagents (FIG. 34). Results show that the library can successfully yield high affinity binders to all antigens tested.

### Example 12: Affinity Determination of Selected Antibodies

Affinity determination of antibodies selected from the naïve library using the phage+yeast display protocol described in Example 11, was performed following the approaches described herein. Binding affinity of the antibody variants thus obtained to various targets (e.g., GM-CSF, IFN-a 2A and IL-6) was examined using a Carterra LSA machine. Briefly, supernatants from yeast expressing scFv-Fc fusions from selections against GM-CSF, IFN-a 2A and IL-6 were immobilized on a Carterra LSA HC200M chip with anti-human Fc. The chips were activated with 1:1:1 100 mM MES pH 5.5, 100 mM S-NHS, 400 mM EDC (all reconstituted in MES 5.5), and 100 μL of each were mixed in a vial immediately before running the assay. The polyclonal goat anti-human IgG was immobilized for 10-minute at 50 μg/mL followed by 7-minute deactivation with 1 M Ethanolamine pH 8.5.

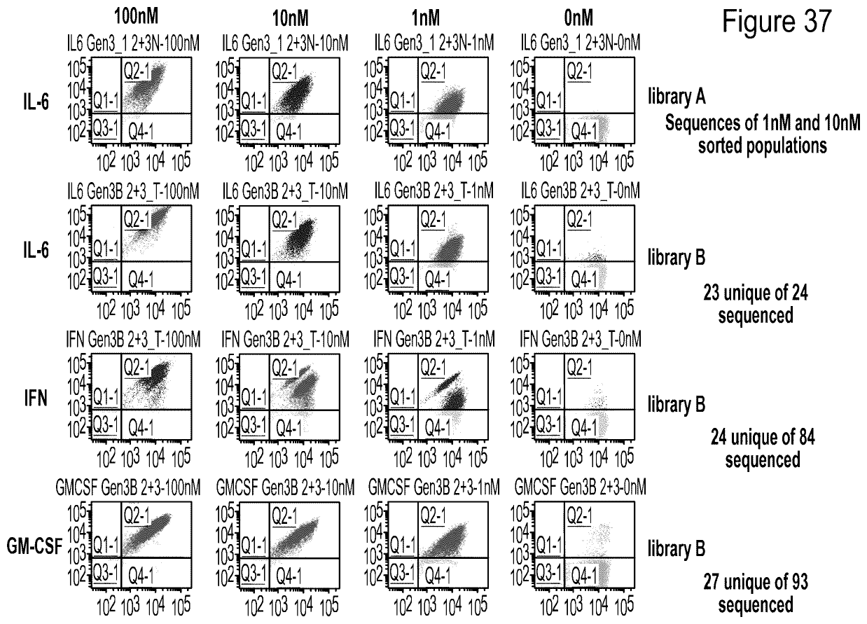

The scFv-Fc supernatants were diluted two or three fold into HBSTE buffer and cycled for 12 minutes across the anti-Fc surface. Antigens were tested in a three-fold dilution series starting at 6 nM for IFN-2A and 167 nM for IL-6/GM-SCF. The antigen samples were tested from lowest to highest concentration. FIG. 37.

Data was processed using a floated Rmax parameter for the IFN-2A and GM-SCF clones that did not dissociate fully between binding cycles; some of the data were also fit using a bulk shift parameter.

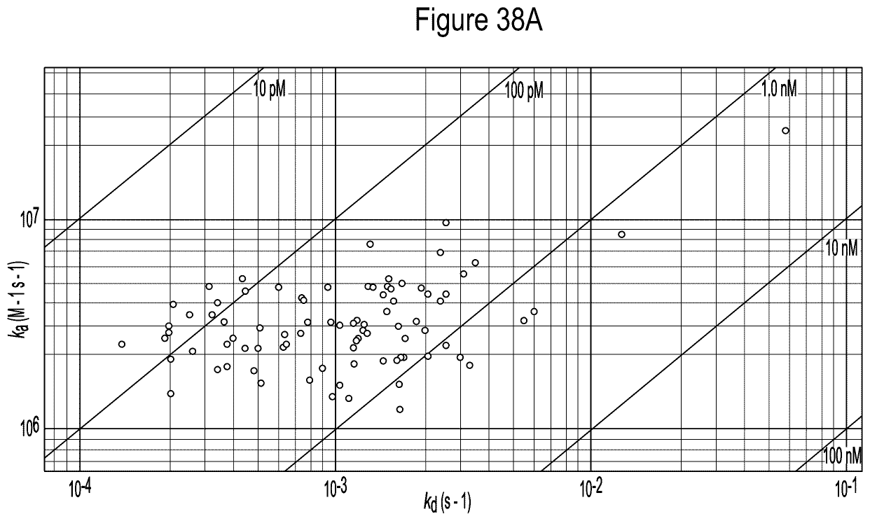

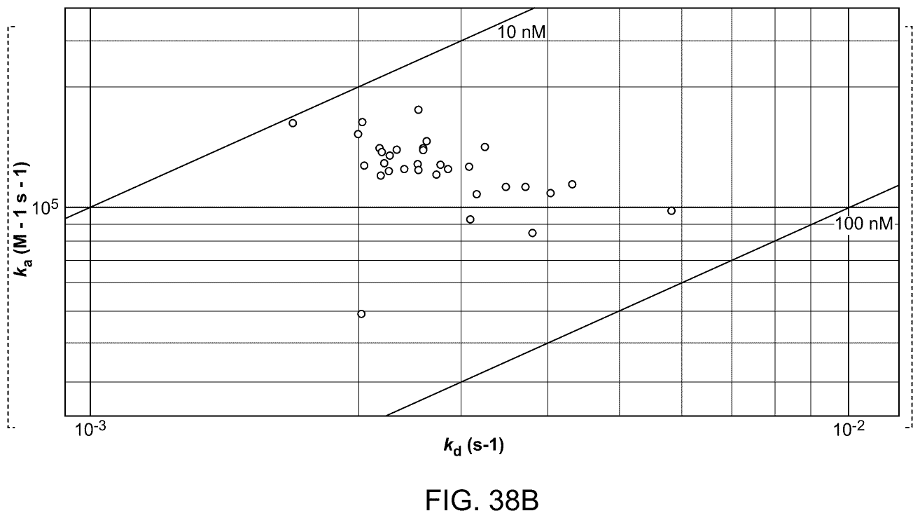

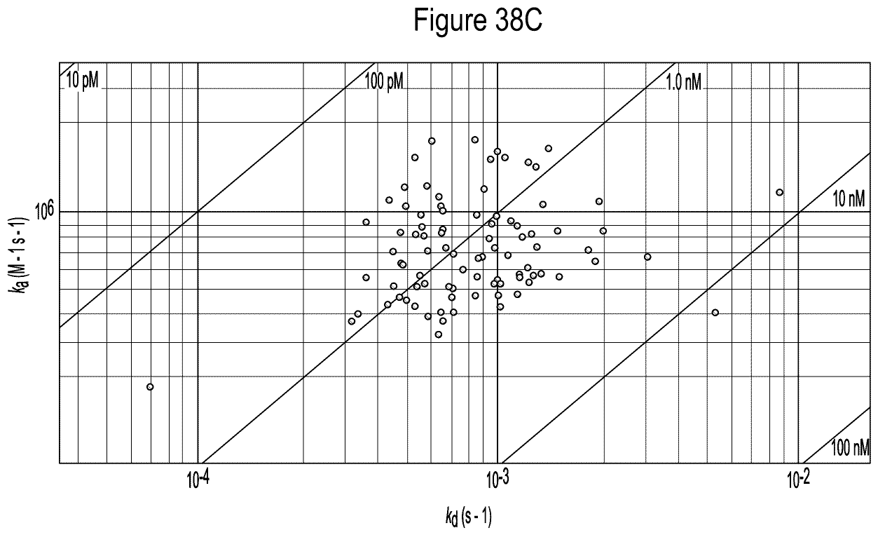

As shown in FIGS. 38A-38C, the affinities of antibodies selected directly from the library, constructed as described in Examples 1-10, are shown to be extremely potent, with many subnanomolar antibodies having been selected.

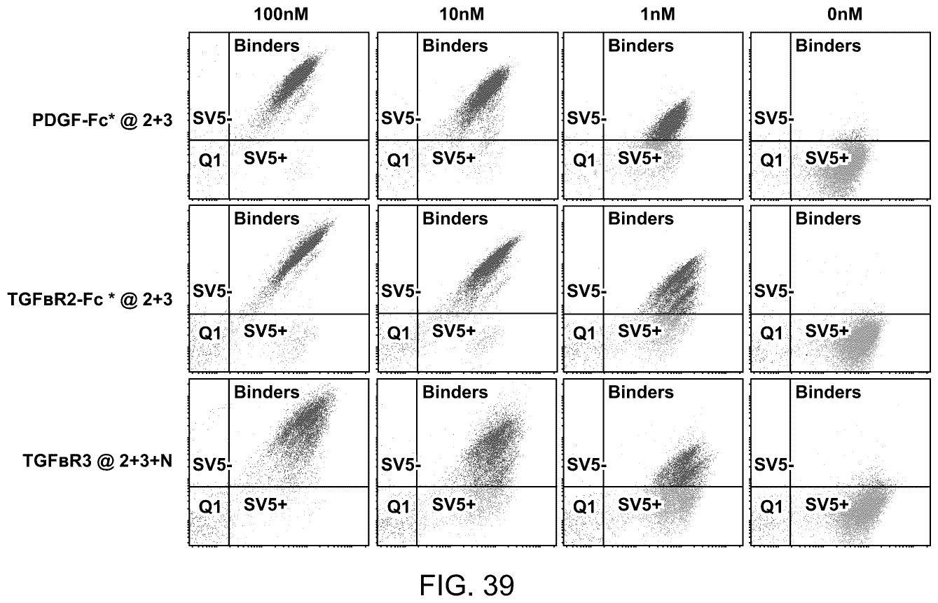

Antibodies to additional targets, including PDGF, TGFBR2, and TGFBR3, were explored using this approach and similar results were observed. FIG. 39.

### Example 13: Antibody Maturation

To select an optimized VL and VH pair and assemble the CDRs into a mature antibody, the following approach can be used. First, the VH is kept in unmodified form while the VLs in the LCDR libraries are shuffled. The remaining functional VLs are assembled with the unmodified VH and the formed antibodies are tested for functionality (FIG. 35). Likewise, the VL is kept unmodified and the VHs comprising synthetic HCDR1/2 and natural HCDR3 in the VH library is shuffled. Each of the remaining functional VH can be assembled with the unmodified VH and the newly formed antibodies are tested for their functions (FIG. 36).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one,

A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. An antibody library, comprising a first plurality of nucleic acids and a second plurality of nucleic acids,
wherein the first plurality of nucleic acids encodes a population of antibody heavy chain variable domains, which collectively comprise one or more heavy chain CDR1s, one or more heavy chain CDR2s, and one or more heavy chain CDR3s located at the CDR1 region, the CDR2 region, and the CDR3 region of an antibody heavy chain variable domain gene, respectively, wherein amino acid sequences of the one or more heavy chain CDR1s, the one or more heavy chain CDR2s, and the one or more heavy chain CDR3s are from naturally occurring human antibodies,
wherein the second plurality of nucleic acids encodes a population of light chain variable domains, which collectively comprise one or more light chain CDR1s, one or more light chain CDR2s, and one or more light chain CDR3s located at the CDR1 region, the CDR2 region, and the CDR3 region of an antibody light chain variable domain gene, respectively, wherein amino acid sequences of the one or more light chain CDR1s, the one or more light chain CDR2s, and the one or more light chain CDR3s, are from naturally occurring human antibodies,
wherein the one or more heavy chain CDR1s, the one or more heavy chain CDR2s, the one or more light chain CDR1s, the one or more light chain CDR2s, and the one or more light chain CDR3s are substantially free of sequence liabilities and non-functional members; and
wherein the antibody heavy chain variable domain gene and/or the antibody light chain variable domain gene are from a single therapeutic antibody or from a human, humanized, or partially humanized antibody germline sequence, wherein each framework region can contain up to five amino acid substitutions.

2. The antibody library of claim 1, wherein the nucleic acid sequences of the heavy chain CDR3s are obtained from human donor lymphocytes.

3. The antibody library of claim 1, wherein the antibody heavy chain variable domain framework 1, framework 2, framework 3, framework 4 are from a human or humanized VH3-23 (DP-47) sequence, wherein each framework region can contain up to 5 amino acid substitutions.

4. The antibody library of claim 1, wherein the antibody light chain variable domain framework 1, framework 2, framework 3, framework 4 are from a human or humanized kappa VK1-39 sequence, wherein each framework region can contain up to 5 amino acid substitutions.

5. The antibody library of claim 1, wherein the antibody light chain variable domain framework 1, framework 2, framework 3, framework 4 are from a single therapeutic antibody trastuzumab, wherein each framework region can contain up to 5 amino acid substitutions.

6. The antibody library of claim 1, wherein the antibody library is a fragment antigen-binding (Fab) library.

7. The antibody library of claim 1, wherein the antibody library is a single-chain variable fragment (scFv) library.

8. The antibody library of claim 1, wherein at least 80% of each of the one or more heavy chain CDR1s, the one or more heavy chain CDR2s, the one or more light chain CDR1s, the one or more light chain CDR2s, and the one or more light chain CDR3s are free of sequence liabilities.

9. The antibody library of claim 1, wherein at least 90% of each of the one or more heavy chain CDR1s, the one or more heavy chain CDR2s, the one or more heavy chain CDR3s, the one or more light chain CDR1s, the one or more light chain CDR2s, and the one or more light chain CDR3s are free of non-functional members.

10. The antibody library of claim 1, wherein the single therapeutic antibody is abituzumab, adalimumab, alemtuzumab, alirocumab, bapineuzumab, benralizumab, brodalumab, canakinumab, certolizumab, clazakizumab, dacetuzumab, daclizumab, daratumumab, eculizumab, efalizumab, elotuzumab, epratuzumab, farletuzumab, fasinumab, ficlatuzumab, fletikumab, fresolimumab, fulranumab, gevokizumab, ibalizumab, lintuzumab, matuzumab, mavrilimumab, mogamulizumab, motavizumab, natalizumab, nivolumab, obinutuzumab, ofatumumab, olokizumab, omalizumab, onartuzumab, otelixizumab, otlertuzumab, palivizumab, panitumumab, panobacumab, pertuzumab, pinatuzumab, polatuzumab, radretumab, ramucirumab, reslizumab, romosozumab, sarilumab, secukinumab, sifalimumab, tabalumab, tigatuzumab, tildrakizumab, tocilizumab, tovetumab, trastuzumab, vedolizumab, veltuzumab, zalutumumab, or zanolimumab.

11. The antibody library of claim 1, wherein the sequence liabilities are: (i) a glycosylation site, (ii) a deamidation site, (iii) an isomerization site, (iv) unpaired cysteine, (v) net charge greater than 1, (vi) a tripeptide motif containing at least two aromatic residues, (vii) a motif that promotes aggregation, (viii) a polyspecificity site; (ix) a protease sensitive site, (x) an integrin binding site, (xi) a lysine glycation site, (xii) a metal catalyzed fragmentation site, (xiii) a polyspecificity aggregation site; and/or (xiv) a streptavidin binding motif.

12. The antibody library of claim 11, wherein the glycosylation site of (i) comprises the motif NXS, NXT, or NXC, in which X represents any naturally-occurring amino acid residue except for proline; the deamidation site of (ii) comprises the motif of NG, NS, NT, NN, NA, NH, ND, GNF, GNY, GNT, or GNG; the isomerization site of (iii) comprises the motif of DT, DH, DS, DG, or DD; the tripeptide of (vi) is HYF or HWH; the motif that promotes aggregation of (vii) comprises the motif of FHW; the polyspecificity site of (viii) comprises the motif GG, GGG, RR, VG, W, WV, WW, WWW, YY, or WXW, in which X represents any amino acid residue; the protease cleavage site of (ix) comprises the motif of DX, in which X is P, G, S, V, Y, F, Q, K, L, or D; the integrin binding site of (x) comprises RGD, RYD, LDV, or KGD; the lysine glycation site of (xi) comprises KE, EK, or ED; the metal catalyzed fragmentation site of (xii) comprises the motif of HS, SH, KT, HXS, or SXH, in which X represents any amino acid residue; the polyspecificity aggregation site of (xiii) comprises a motif of X.sub.1X.sub.2X.sub.3, wherein each of X.sub.1, X.sub.2, and X.sub.3 independently is selected from the group consisting of F, I, L, V, Wand Y; and/or the streptavidin binding motif of (xiv) comprises the motif HPQ, EPDW (SEQ ID NO: 117), PWXWL (SEQ ID NO: 118), in which X represents any amino acid residue, GDWVFI (SEQ ID NO: 119), or PWPWLG (SEQ ID NO: 120).


SEQUENCE LISTING (298 sequences)

SEQ ID NO: 1 - DNA, 76 bp, synthetic construct
  misc_feature 1..76: note = Cloning Vector
  source 1..76: mol_type = other DNA
gtgttaccatcacctgtcgtgcttctagagaccatggccagtaaggccggtctctctggcttggtaccagcagaaa

SEQ ID NO: 2 - DNA, 76 bp, synthetic construct
  misc_feature 1..76: note = Cloning Vector
  source 1..76: mol_type = other DNA
cacaatggtagtggacagcacgaagatctctggtaccggtcattccggccagagagaccgaaccatggtcgtcttt

SEQ ID NO: 3 - protein, 24 aa, synthetic construct
  REGION 1..24: note = Cloning Vector
RVTITCRASRDHGQGRSLAWYQQK

SEQ ID NO: 4 - protein, 15 aa, Homo sapiens
  SITE 3: note = MISC_FEATURE - Xaa at position 3 is Met, Ile, or Leu
  SITE 4: note = MISC_FEATURE - Xaa at position 4 is Gly or Ala
  SITE 5: note = MISC_FEATURE - Xaa at position 5 is Trp, Arg, Glu, or His
  SITE 7: note = MISC_FEATURE - Xaa at position 7 is Ser, Asn, or Phe
  SITE 8: note = MISC_FEATURE - Xaa at position 8 is optional or is Gly, Thr, Val, Ala, or Pro
  SITE 9: note = MISC_FEATURE - Xaa at position 9 is Tyr, Asn, His, or Ser
  SITE 10: note = MISC_FEATURE - Xaa at position 10 is Asn, Ser, or Thr
  SITE 11: note = MISC_FEATURE - Xaa at position 11 is Gly or Asp
  SITE 12: note = MISC_FEATURE - Xaa at position 12 is Asn, Asp, Lys, Ala, Gly, or Glu
  SITE 13: note = MISC_FEATURE - Xaa at position 13 is Thr, Ala, Asn, or Lys
  SITE 14: note = MISC_FEATURE - Xaa at position 14 is Asn, Lys, Asp, or Ser
EWXXXIXXXXXXXXY

SEQ ID NO: 5 - protein, 15 aa, Homo sapiens
EWMGWISGYNGNTNY

SEQ ID NO: 6 - protein, 15 aa, Homo sapiens
EWMGWISTYNGNTNY

SEQ ID NO: 7 - protein, 15 aa, Homo sapiens
EWMGWISVYNGNTNY

SEQ ID NO: 8 - protein, 15 aa, Homo sapiens
EWMGWISAYNGNTKY

SEQ ID NO: 9 - protein, 15 aa, Homo sapiens
EWMGRISAYNGNTNY

SEQ ID NO: 10 - protein, 15 aa, Homo sapiens
EWMGWISAYNGDTNY

SEQ ID NO: 11 - protein, 15 aa, Homo sapiens
EWMGWISAYNGKTNY

SEQ ID NO: 12 - protein, 15 aa, Homo sapiens
EWMGWISPYNGNTNY

SEQ ID NO: 13 - protein, 15 aa, Homo sapiens
EWMGWINPNSGDTNY

SEQ ID NO: 14 - protein, 15 aa, Homo sapiens
EWMGWINPNSGATNY

SEQ ID NO: 15 - protein, 15 aa, Homo sapiens
EWMGWINPNSGGTDY

SEQ ID NO: 16 - protein, 15 aa, Homo sapiens
EWMGWINPNSGGTSY

SEQ ID NO: 17 - protein, 15 aa, Homo sapiens
EWMGWINPNSGGTKY

SEQ ID NO: 18 - protein, 15 aa, Homo sapiens
EWMGWINPNSGGANY

SEQ ID NO: 19 - protein, 15 aa, Homo sapiens
EWMGRINPNSGGTNY

SEQ ID NO: 20 - protein, 15 aa, Homo sapiens
EWMGWINPNNGGTNY

SEQ ID NO: 21 - protein, 15 aa, Homo sapiens
EWMGWINPNTGGTNY

SEQ ID NO: 22 - protein, 14 aa, Homo sapiens
EWIGEINHSGNTNY

SEQ ID NO: 23 - protein, 14 aa, Homo sapiens
EWLAHIFSNDENSY

SEQ ID NO: 24 - protein, 14 aa, Homo sapiens
EWLAHIFSNGEKSY

SEQ ID NO: 25 - skipped

SEQ ID NO: 26 - protein, 15 aa, Homo sapiens
EWMGVIYPGDSDTRY

SEQ ID NO: 27 - protein, 15 aa, Homo sapiens
EWMGLIYPGDSDTRY

SEQ ID NO: 28 - protein, 15 aa, Homo sapiens
EWMGIIFPGDSDTRY

SEQ ID NO: 29 - protein, 15 aa, Homo sapiens
EWMGIIYPGDSDIRY

SEQ ID NO: 30 - protein, 15 aa, Homo sapiens
EWMGIIYPGDSETRY

SEQ ID NO: 31 - protein, 15 aa, Homo sapiens
EWMGIIYPGDSDTSY

SEQ ID NO: 32 - protein, 15 aa, Homo sapiens
EWMGIIYPGDSDTKY

SEQ ID NO: 33 - protein, 15 aa, Homo sapiens
EWMGIIYPGDSDTTY

SEQ ID NO: 34 - protein, 15 aa, Homo sapiens
EWMGIIYPSDSDTRY

SEQ ID NO: 35 - protein, 15 aa, Homo sapiens
EWMGIIYPADSDTRY

SEQ ID NO: 36 - protein, 15 aa, Homo sapiens
EWMGIIYPDDSDTRY

SEQ ID NO: 37 - protein, 14 aa, Homo sapiens
EWLALIYWDGDKRY

SEQ ID NO: 38 - protein, 14 aa, Homo sapiens
EWLALVYWDDDKRY

SEQ ID NO: 39 - protein, 14 aa, Homo sapiens
EWLALIYWNDDKRY

SEQ ID NO: 40 - protein, 14 aa, Homo sapiens
EWLALIYWDDDRRY

SEQ ID NO: 41 - protein, 14 aa, Homo sapiens
EWLARIDWDDDKYY

SEQ ID NO: 42 - protein, 14 aa, Homo sapiens
EWLALIYWDDDKYY

SEQ ID NO: 43 - protein, 14 aa, Homo sapiens
EWLAVIYWDDDKRY

SEQ ID NO: 44 - protein, 14 aa, Homo sapiens
EWLAFIYWDDDKRY

SEQ ID NO: 45 - protein, 14 aa, Homo sapiens
EWLAIIYWDDDKRY

SEQ ID NO: 46 - skipped

SEQ ID NO: 47 - protein, 15 aa, Homo sapiens
EWMGWINPNRSGTSY

SEQ ID NO: 48 - protein, 15 aa, Homo sapiens
EWMGWINLNRSGTTY

SEQ ID NO: 49 - protein, 15 aa, Homo sapiens
EWMGRINPNRSDTNY

SEQ ID NO: 50 - protein, 15 aa, Homo sapiens
EWMGNISPGDPDTRY

SEQ ID NO: 51 - protein, 15 aa, Homo sapiens
EWMGKINRSGGSTSY

SEQ ID NO: 52 - protein, 15 aa, Homo sapiens
EWMGIINHSGGTTSY

SEQ ID NO: 53 - protein, 15 aa, Homo sapiens
EWMGIINLSSRFTSY

SEQ ID NO: 54 - protein, 15 aa, Homo sapiens
EWIGIINLSSGSTSY

SEQ ID NO: 55 - protein, 15 aa, Homo sapiens
EWMGISNLSGGSTSY

SEQ ID NO: 56 - protein, 15 aa, Homo sapiens
EWLGIINASGGSTRY

SEQ ID NO: 57 - protein, 15 aa, Homo sapiens
EWVGIINLTGGATRY

SEQ ID NO: 58 - protein, 15 aa, Homo sapiens
EWVSSINWSGGSTYY

SEQ ID NO: 59 - protein, 15 aa, Homo sapiens
EWVSTINESGGKTHY

SEQ ID NO: 60 - protein, 15 aa, Homo sapiens
EWVANISGGGGAIYY

SEQ ID NO: 61 - protein, 15 aa, Homo sapiens
EWVAYINRSGSTIYY

SEQ ID NO: 62 - protein, 15 aa, Homo sapiens
EWVDVIWYAGRNKSY

SEQ ID NO: 63 - protein, 15 aa, Homo sapiens
EWVAVISHDRSNKSY

SEQ ID NO: 64 - protein, 15 aa, Homo sapiens
EWMSWINASSGGTNY

SEQ ID NO: 65 - protein, 15 aa, Homo sapiens
EWMGRNITILGIANY

SEQ ID NO: 66 - protein, 15 aa, Homo sapiens
EWMGGNITIFGKANY

SEQ ID NO: 67 - skipped

SEQ ID NO: 68 - protein, 15 aa, Homo sapiens
EWMGIINPNSGCTNY

SEQ ID NO: 69 - protein, 15 aa, Homo sapiens
EWMGWINPNVCGTNY

SEQ ID NO: 70 - protein, 15 aa, Homo sapiens
EWMGMIYPGNCDTSY

SEQ ID NO: 71 - protein, 15 aa, Homo sapiens
EWMGIIYPGSCETKY

SEQ ID NO: 72 - protein, 15 aa, Homo sapiens
EWMGGIIPIFCTEYY

SEQ ID NO: 73 - protein, 15 aa, Homo sapiens
EWMGIICPGDAATRY

SEQ ID NO: 74 - protein, 15 aa, Homo sapiens
EWMGCISAYYGNPNY

SEQ ID NO: 75 - protein, 15 aa, Homo sapiens
EWVGVISHDGGNECY

SEQ ID NO: 76 - protein, 15 aa, Homo sapiens
EWMGCINAADGNTKY

SEQ ID NO: 77 - protein, 15 aa, Homo sapiens
EWMGCFEPKDGETIY

SEQ ID NO: 78 - protein, 14 aa, Homo sapiens
EWLAHICSNDGKRY

SEQ ID NO: 79 - protein, 14 aa, Homo sapiens
EWIGLINQCGSTNY

SEQ ID NO: 80 - protein, 14 aa, Homo sapiens
EWIGYIYYCGSPNY

SEQ ID NO: 81 - protein, 15 aa, Homo sapiens
EWLSYSSCSGTPIYY

SEQ ID NO: 82 - protein, 15 aa, Homo sapiens
EWVSYICGSSSTIYY

SEQ ID NO: 83 - protein, 15 aa, Homo sapiens
EWVSSISSCGSSTYY

SEQ ID NO: 84 - protein, 15 aa, Homo sapiens
EWVSYISSCGSTINY

SEQ ID NO: 85 - protein, 14 aa, Homo sapiens
EWVSIIYRCGTTYY

SEQ ID NO: 86 - protein, 14 aa, Homo sapiens
EWMGYIYCSSSANY

SEQ ID NO: 87 - protein, 15 aa, Homo sapiens
EWMGRIYPCDSYINY

SEQ ID NO: 88 - protein, 25 aa, Homo sapiens
AGPSITESHYCLDCAAKDYYYGLDV

SEQ ID NO: 89 - protein, 17 aa, Homo sapiens
AKDARDCLLCADWHFDL

SEQ ID NO: 90 - protein, 16 aa, Homo sapiens
AKFSGKDCSGTSCRDY

SEQ ID NO: 91 - protein, 18 aa, Homo sapiens
ARAPDCADADCHKGAFGY

SEQ ID NO: 92 - protein, 19 aa, Homo sapiens
ARDGGHGFCSSASCFGPDY

SEQ ID NO: 93 - protein, 16 aa, Homo sapiens
ARRGSCDYCGDFPWQY

SEQ ID NO: 94 - protein, 17 aa, Homo sapiens
ARSPSYICSGGTCVFDH

SEQ ID NO: 95 - protein, 18 aa, Homo sapiens
ARVGYCSSTSCNRGAFDI

SEQ ID NO: 96 - protein, 14 aa, Homo sapiens
VRGHCDGTTCSRAY

SEQ ID NO: 97 - protein, 17 aa, Homo sapiens
VRKGPSCPHCGDFHWQH

SEQ ID NO: 98 - protein, 20 aa, Homo sapiens
VRSVTPRYCGGGFCYGEFDY

SEQ ID NO: 99 - protein, 18 aa, Homo sapiens
VRTADCERDPCKGWVFPH

SEQ ID NO: 100 - protein, 18 aa, Homo sapiens
VTLPDLCPGDNCTYPDAS

SEQ ID NO: 101 - protein, 25 aa, Homo sapiens
VRGRSCCGGRRHCNGADCFNWDFQH

SEQ ID NO: 102 - protein, 37 aa, Homo sapiens
AKDLREDECEEWWSDYYDFGKQLPCRKSRGVAGIFDG

SEQ ID NO: 103 - DNA, 96 bp, synthetic construct
  misc_feature 1..96: note = Yeast Display Vector
  source 1..96: mol_type = other DNA
ggtgatcgtgttactattacctgtcgtgcttctagagaccatggccagtaaggccggtctctctggcttggtaccagcagaaaccaggtaaagctc

SEQ ID NO: 104 - DNA, 96 bp, synthetic construct
  misc_feature 1..96: note = Yeast Display Vector
  source 1..96: mol_type = other DNA
ccactagcacaatgataatggacagcacgaagatctctggtaccggtcattccggccagagagaccgaaccatggtcgtctttggtccatttcgag

SEQ ID NO: 105 - DNA, 29 bp, synthetic construct
  misc_feature 1..29: note = Yeast Display Vector
  source 1..29: mol_type = other DNA
ggtgatcgtgttactattacctgtcgtgc

SEQ ID NO: 106 - DNA, 34 bp, synthetic construct
  misc_feature 1..34: note = Yeast Display Vector
  source 1..34: mol_type = other DNA
ctggcttggtaccagcagaaaccaggtaaagctc

SEQ ID NO: 107 - DNA, 33 bp, synthetic construct
  misc_feature 1..33: note = Yeast Display Vector
  source 1..33: mol_type = other DNA
ccactagcacaatgataatggacagcacgaaga

SEQ ID NO: 108 - DNA, 30 bp, synthetic construct
  misc_feature 1..30: note = Yeast Display Vector
  source 1..30: mol_type = other DNA
gaaccatggtcgtctttggtccatttcgag

SEQ ID NO: 109 - DNA, 33 bp, synthetic construct
  misc_feature 1..33: note = Amplified LCDR1 Oligo
  source 1..33: mol_type = other DNA
ggtgatcgtgttactattacctgtcgtgcttct

SEQ ID NO: 110 - DNA, 34 bp, synthetic construct; identical to SEQ ID NO: 106
  misc_feature 1..34: note = Amplified LCDR1 Oligo
  source 1..34: mol_type = other DNA

SEQ ID NO: 111 - DNA, 33 bp, synthetic construct; identical to SEQ ID NO: 107
  misc_feature 1..33: note = Amplified LCDR1 Oligo
  source 1..33: mol_type = other DNA

SEQ ID NO: 112 - DNA, 34 bp, synthetic construct
  misc_feature 1..34: note = Amplified LCDR1 Oligo
  source 1..34: mol_type = other DNA
gaccgaaccatggtcgtctttggtccatttcgag

SEQ ID NO: 113 - DNA, 33 bp, synthetic construct; identical to SEQ ID NO: 109
  misc_feature 1..33: note = Cloned Construct
  source 1..33: mol_type = other DNA

SEQ ID NO: 114 - DNA, 34 bp, synthetic construct; identical to SEQ ID NO: 106
  misc_feature 1..34: note = Cloned Construct
  source 1..34: mol_type = other DNA

SEQ ID NO: 115 - DNA, 33 bp, synthetic construct; identical to SEQ ID NO: 107
  misc_feature 1..33: note = Cloned Construct
  source 1..33: mol_type = other DNA

SEQ ID NO: 116 - DNA, 34 bp, synthetic construct; identical to SEQ ID NO: 112
  misc_feature 1..34: note = Cloned Construct
  source 1..34: mol_type = other DNA

SEQ ID NO: 117 - protein, 4 aa, synthetic construct
  REGION 1..4: note = Streptavidin Binding Motif
EPDW

SEQ ID NO: 118 - protein, 5 aa, synthetic construct
  REGION 1..5: note = Streptavidin Binding Motif
  SITE 3: note = MISC_FEATURE - Xaa is any amino acid
PWXWL

SEQ ID NO: 119 - protein, 6 aa, synthetic construct
  REGION 1..6: note = Streptavidin Binding Motif
GDWVFI

SEQ ID NO: 120 - protein, 6 aa, synthetic construct
  REGION 1..6: note = Streptavidin Binding Motif
PWPWLG

SEQ ID NO: 121 - protein, 107 aa, Homo sapiens
DIQMTQSPSSVSASVGDRVTITCRASQGISSWLAWYQQKPGKAPKLLIYGASNLESGVPSRFSGSGSGTDFTLTISSLQPEDFANYYCQQANSFPWTFGGGTKVEIK

SEQ ID NO: 122 - protein, 21 aa, synthetic construct
  REGION 1..21: note = Linker Sequence
SGGSTITSYNVYYTKLSSSGT

SEQ ID NO: 123 - protein, 118 aa, Homo sapiens [sequence omitted]

SEQ ID NO: 124 - protein, 113 aa, Homo sapiens
DIVMTQSPDSLAVSLGERATINCKSSQSLLNSGNQKNYLAWYQQKPGQPPKLLIYGASTRESGVPDRFSGSGSGTDFTLTISSLQAEDVAVYYCQNVHSFPFTFGGGTKVEIK

SEQ ID NO: 125 - protein, 21 aa, synthetic construct
  REGION 1..21: note = Linker Sequence
SGGSTITSYNVYYTKLSSSGT

SEQ ID NO: 126 - protein, 118 aa, Homo sapiens
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGTLVTVSS

SEQ ID NO: 127 - protein, 112 aa, Homo sapiens
DIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGGGTKVEIK

SEQ ID NO: 128 - protein, 21 aa, synthetic construct
  REGION 1..21: note = Linker Sequence
SGGSTITSYNVYYTKLSSSGT

SEQ ID NO: 129 - protein, 112 aa, Homo sapiens
EVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSS

SEQ ID NO: 130 - protein, 107 aa, Homo sapiens
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKVEIK

SEQ ID NO: 131 - protein, 21 aa, synthetic construct
  REGION 1..21: note = Linker Sequence
SGGSTITSYNVYYTKLSSSGT

SEQ ID NO: 132 - protein, 121 aa, Homo sapiens [sequence omitted]

SEQ ID NO: 133 - protein, 108 aa, Homo sapiens
EIVLTQSPGTLSLSPGERATLSCRASQSVSSSFLAWYQQKPGQAPRLLIYGASSRATGIPDRLSGSGSGTDFTLTITRLEPEDFAVYYCQQYDSSAITFGGGTKVEIK

SEQ ID NO: 134 - protein, 21 aa, synthetic construct
  REGION 1..21: note = Linker Sequence
SGGSTITSYNVYYTKLSSSGT

SEQ ID NO: 135 - protein, 117 aa, Homo sapiens
EVQLVQSGAEVKKPGESLKISCKGSGYIFTNYWIAWVRQVPGKGLESMGIIYPGDSDIRYSPSFQGQVTISADKSITTAYLQWSSLKASDTAIYYCARHDIEGFDYWGRGTLVTVSS

SEQ ID NO: 136 - protein, 109 aa, Homo sapiens [sequence omitted]

SEQ ID NO: 137 - protein, 21 aa, synthetic construct
  REGION 1..21: note = Linker Sequence
SGGSTITSYNVYYTKLSSSGT

SEQ ID NO: 138 - protein, 114 aa, Homo sapiens
VQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 139 - DNA, 816 bp, synthetic construct
  misc_feature 1..816: note = Oligonucleotide
  source 1..816: mol_type = other DNA
cagttagataaaagaggcgcggcagcaagcggcgcgcatgccgatatccagatgacccagtctccatcttctgtttctgcttctgttggtgatcgtgttactattacctgtcgtgcttctcagggtatctcttcttggctggcttggtaccagcagaaaccaggtaaagctccaaaactgctgatctacggtgcttctaacctggaatctggtgttccatctcgtttctctggttctggttctggtaccgatttcaccctgaccatctcttctctgcagccagaagatttcgctaactactactgtcagcaggctaactctttcccatggaccttcggtggtggtaccaaagttgaaatcaaatccggagggtcgaccataacttcgtataatgtatactatacgaagttatcctcgagcggtacccaggttcagctggttcagtctggtgctgaagttaaaaaaccaggtgcttctgttaaagtttcttgtaaagtttctggttacaccctgtctgatctgtctatccactgggttcgtcaggctccaggtaaaggtctggaatggatgggtggtttcgatccacaggatggtgaaaccatctacgctcagaaattccagggtcgtgttaccatgaccgaagatacctctaccgataccgcttacatggaactgtcttctctgaaatctgaggacacggccgtgtattactgtgctaccggttcttcttcttcttggttcgatccatggggtcagggaaccctggtcaccgtctcctcagctagcggcaaaccaatcccaaacccactgctgggc

SEQ ID NO: 140 - DNA, 827 bp, synthetic construct
  misc_feature 1..827: note = Oligonucleotide
  source 1..827: mol_type = other DNA
cagttagataaaagaggcgcggcagcaagcggcgcgcatgccgatatccagatgacccagtctccatcttctgtttctgcttctgttggtgatcgtgttactattacctgtcgtgcttctagagaccatggccagtaaggccggtctctctggcttggtaccagcagaaaccaggtaaagctccaaaactgctgatctacggtgcttctaacctggaatctggtgttccatctcgtttctctggttctggttctggtaccgatttcaccctgaccatctcttctctgcagccagaagatttcgctaactactactgtcagcaggctaactctttcccatggaccttcggtggtggtaccaaagttgaaatcaaatccggagggtcgaccataacttcgtataatgtatactatacgaagttatcctcgagcggtacccaggttcagctggttcagtctggtgctgaagttaaaaaaccaggtgcttctgttaaagtttcttgtaaagtttctggttacaccctgtctgatctgtctatccactgggttcgtcaggctccaggtaaaggtctggaatggatgggtggtttcgatccacaggatggtgaaaccatctacgctcagaaattccagggtcgtgttaccatgaccgaagatacctctaccgataccgcttacatggaactgtcttctctgaaatctgaggacacggccgtgtattactgtgctaccggttcttcttcttcttggttcgatccatggggtcagggaaccctggtcaccgtctcctcagctagcggcaaaccaatcccaaacccactgctgggc

SEQ ID NO: 141 - DNA, 824 bp, synthetic construct
  misc_feature 1..824: note = Oligonucleotide
  source 1..824: mol_type = other DNA
cagttagataaaagaggcgcggcagcaagcggcgcgcatgccgatatccagatgacccagtctccatcttctgtttctgcttctgttggtgatcgtgttactattacctgtcgtgcttctcagggtatctcttcttggctggcttggtaccagcagaaaccaggtaaagctccaaaactgctgatctacagagaccatggccagtaaggccggtctctggtgttccatctcgtttctctggttctggttctggtaccgatttcaccctgaccatctcttctctgcagccagaagatttcgctaactactactgtcagcaggctaactctttcccatggaccttcggtggtggtaccaaagttgaaatcaaatccggagggtcgaccataacttcgtataatgtatactatacgaagttatcctcgagcggtacccaggttcagctggttcagtctggtgctgaagttaaaaaaccaggtgcttctgttaaagtttcttgtaaagtttctggttacaccctgtctgatctgtctatccactgggttcgtcaggctccaggtaaaggtctggaatggatgggtggtttcgatccacaggatggtgaaaccatctacgctcagaaattccagggtcgtgttaccatgaccgaagatacctctaccgataccgcttacatggaactgtcttctctgaaatctgaggacacggccgtgtattactgtgctaccggttcttcttcttcttggttcgatccatggggtcagggaaccctggtcaccgtctcctcagctagcggcaaaccaatcccaaacccactgctgggc

SEQ ID NO: 142 - DNA, 818 bp, synthetic construct
  misc_feature 1..818: note = Oligonucleotide
  source 1..818: mol_type = other DNA
cagttagataaaagaggcgcggcagcaagcggcgcgcatgccgatatccagatgacccagtctccatcttctgtttctgcttctgttggtgatcgtgttactattacctgtcgtgcttctcagggtatctcttcttggctggcttggtaccagcagaaaccaggtaaagctccaaaactgctgatctacggtgcttctaacctggaatctggtgttccatctcgtttctctggttctggttctggtaccgatttcaccctgaccatctcttctctgcagccagaagatttcgctaactactactgtagagaccatggccagtaaggccggtctctttcggtggtggtaccaaagttgaaatcaaatccggagggtcgaccataacttcgtataatgtatactatacgaagttatcctcgagcggtacccaggttcagctggttcagtctggtgctgaagttaaaaaaccaggtgcttctgttaaagtttcttgtaaagtttctggttacaccctgtctgatctgtctatccactgggttcgtcaggctccaggtaaaggtctggaatggatgggtggtttcgatccacaggatggtgaaaccatctacgctcagaaattccagggtcgtgttaccatgaccgaagatacctctaccgataccgcttacatggaactgtcttctctgaaatctgaggacacggccgtgtattactgtgctaccggttcttcttcttcttggttcgatccatggggtcagggaaccctggtcaccgtctcctcagctagcggcaaaccaatcccaaacccactgctgggc

SEQ ID NO: 143 - DNA, 821 bp, synthetic construct
  misc_feature 1..821: note = Oligonucleotide
  source 1..821: mol_type = other DNA
cagttagataaaagaggcgcggcagcaagcggcgcgcatgccgatatccagatgacccagtctccatcttctgtttctgcttctgttggtgatcgtgttactattacctgtcgtgcttctcagggtatctcttcttggctggcttggtaccagcagaaaccaggtaaagctccaaaactgctgatctacggtgcttctaacctggaatctggtgttccatctcgtttctctggttctggttctggtaccgatttcaccctgaccatctcttctctgcagccagaagatttcgctaactactactgtcagcaggctaactctttcccatggaccttcggtggtggtaccaaagttgaaatcaaatccggagggtcgaccataacttcgtataatgtatactatacgaagttatcctcgagcggtacccaggttcagctggttcagtctggtgctgaagttaaaaaaccaggtgcttctgttaaagtttcttgtaaagtttctagagaccatggccagtaaggccggtctctatccactgggttcgtcaggctccaggtaaaggtctggaatggatgggtggtttcgatccacaggatggtgaaaccatctacgctcagaaattccagggtcgtgttaccatgaccgaagatacctctaccgataccgcttacatggaactgtcttctctgaaatctgaggacacggccgtgtattactgtgctaccggttcttcttcttcttggttcgatccatggggtcagggaaccctggtcaccgtctcctcagctagcggcaaaccaatcccaaacccactgctgggc

SEQ ID NO: 144 - DNA, 821 bp, synthetic construct
  misc_feature 1..821: note = Oligonucleotide
  source 1..821: mol_type = other DNA
cagttagataaaagaggcgcggcagcaagcggcgcgcatgccgatatccagatgacccagtctccatcttctgtttctgcttctgttggtgatcgtgttactattacctgtcgtgcttctcagggtatctcttcttggctggcttggtaccagcagaaaccaggtaaagctccaaaactgctgatctacggtgcttctaacctggaatctggtgttccatctcgtttctctggttctggttctggtaccgatttcaccctgaccatctcttctctgcagccagaagatttcgctaactactactgtcagcaggctaactctttcccatggaccttcggtggtggtaccaaagttgaaatcaaatccggagggtcgaccataacttcgtataatgtatactatacgaagttatcctcgagcggtacccaggttcagctggttcagtctggtgctgaagttaaaaaaccaggtgcttctgttaaagtttcttgtaaagtttctggttacaccctgtctgatctgtctatccactgggttcgtcaggctccaggtaaaggtctggaatggatgggtggtagagaccatggccagtaaggccggtctctatctacgctcagaaattccagggtcgtgttaccatgaccgaagatacctctaccgataccgcttacatggaactgtcttctctgaaatctgaggacacggccgtgtattactgtgctaccggttcttcttcttcttggttcgatccatggggtcagggaaccctggtcaccgtctcctcagctagcggcaaaccaatcccaaacccactgctgggc

SEQ ID NO: 145 - DNA, 803 bp, synthetic construct
  misc_feature 1..803: note = Oligonucleotide
  source 1..803: mol_type = other DNA
cagttagataaaagaggcgcggcagcaagcggcgcgcatgccgatatccagatgacccagtctccatcttctgtttctgcttctgttggtgatcgtgttactattacctgtcgtgcttctcagggtatctcttcttggctggcttggtaccagcagaaaccaggtaaagctccaaaactgctgatctacggtgcttctaacctggaatctggtgttccatctcgtttctctggttctggttctggtaccgatttcaccctgaccatctcttctctgcagccagaagatttcgctaactactactgtcagcaggctaactctttcccatggaccttcggtggtggtaccaaagttgaaatcaaatccggagggtcgaccataacttcgtataatgtatactatacgaagttatcctcgagcggtacccaggttcagctggttcagtctggtgctgaagttaaaaaaccaggtgcttctgttaaagtttcttgtaaagtttctggttacaccctgtctgatctgtctatccactgggttcgtcaggctccaggtaaaggtctggaatggatgggtggtttcgatccacaggatggtgaaaccatctacgctcagaaattccagggtcgtgttaccatgaccgaagatacctctaccgataccgcttacatggaactgtcttctctgaaatctgaggacacggccgtgtattactgtagagaccatggccagtaaggccggtctctggaaccctggtcaccgtctcctcagctagcggcaaaccaatcccaaacccactgctgggc

SEQ ID NO: 146 - DNA, 834 bp, synthetic construct
  misc_feature 1..834: note = Oligonucleotide
  source 1..834: mol_type = other DNA
cagttagataaaagaggcgcggcagcaagcggcgcgcatgccgatatcgttatgacccagtctccagattctctggctgtttctctgggtgaacgtgctaccatcaactgcaaatcttctcagtctctgctgaactctggtaaccagaaaaactacctggcttggtaccagcagaaaccaggtcagccaccaaaactgctgatctacggtgcttctacccgtgaatctggtgttccagatcgtttctctggttctggttctggtaccgatttcaccctgaccatctcttctctgcaggctgaagatgttgctgtttactactgtcagaacgttcactctttcccattcaccttcggtggtggtaccaaagttgaaatcaaatccggagggtcgaccataacttcgtataatgtatactatacgaagttatcctcgagcggtacccaggttaccctgcgtgaatctggtccagctctggttaaaccaacccagaccctgaccctgacctgtaccgtttctggtttctctctgtctgcttactctgttaactggatccgtcagccaccaggtaaagctctggaatggctggctatgatctggggtgatggtaaaatcgtttacaactctgctctgaaatctcgtctgaccatctctaaagatacctctaaaaaccaggttgttctgaccatgaccaacatggatcctgtggacacagccacatattactgtgctggtgatggttactacccatacgctatggataactggggtcagggaaccctggtcaccgtctcctcagctagcggcaaaccaatcccaaacccactgctgggc

SEQ ID NO: 147 - DNA, 827 bp, synthetic construct
  misc_feature 1..827: note = Oligonucleotide
  source 1..827: mol_type = other DNA
cagttagataaaagaggcgcggcagcaagcggcgcgcatgccgatatcgttatgacccagtctccagattctctggctgtttctctgggtgaacgtgctaccatcaactgcaaatcttctagagaccatggccagtaaggccggtctctctggcttggtaccagcagaaaccaggtcagccaccaaaactgctgatctacggtgcttctacccgtgaatctggtgttccagatcgtttctctggttctggttctggtaccgatttcaccctgaccatctcttctctgcaggctgaagatgttgctgtttactactgtcagaacgttcactctttcccattcaccttcggtggtggtaccaaagttgaaatcaaatccggagggtcgaccataacttcgtataatgtatactatacgaagttatcctcgagcggtacccaggttaccctgcgtgaatctggtccagctctggttaaaccaacccagaccctgaccctgacctgtaccgtttctggtttctctctgtctgcttactctgttaactggatccgtcagccaccaggtaaagctctggaatggctggctatgatctggggtgatggtaaaatcgtttacaactctgctctgaaatctcgtctgaccatctctaaagatacctctaaaaaccaggttgttctgaccatgaccaacatggatcctgtggacacagccacatattactgtgctggtgatggttactacccatacgctatggataactggggtcagggaaccctggtcaccgtctcctcagctagcggcaaaccaatcccaaacccactgctgggc

SEQ ID NO: 148 - DNA, 842 bp, synthetic construct
  misc_feature 1..842: note = Oligonucleotide
  source 1..842: mol_type = other DNA
cagttagataaaagaggcgcggcagcaagcggcgcgcatgccgatatcgttatgacccagtctccagattctctggctgtttctctgggtgaacgtgctaccatcaactgcaaatcttctcagtctctgctgaactctggtaaccagaaaaactacctggcttggtaccagcagaaaccaggtcagccaccaaaactgctgatctacagagaccatggccagtaaggccggtctctggtgttccagatcgtttctctggttctggttctggtaccgatttcaccctgaccatctcttctctgcaggctgaagatgttgctgtttactactgtcagaacgttcactctttcccattcaccttcggtggtggtaccaaagttgaaatcaaatccggagggtcgaccataacttcgtataatgtatactatacgaagttatcctcgagcggtacccaggttaccctgcgtgaatctggtccagctctggttaaaccaacccagaccctgaccctgacctgtaccgtttctggtttctctctgtctgcttactctgttaactggatccgtcagccaccaggtaaagctctggaatggctggctatgatctggggtgatggtaaaatcgtttacaactctgctctgaaatctcgtctgaccatctctaaagatacctctaaaaaccaggttgttctgaccatgaccaacatggatcctgtggacacagccacatattactgtgctggtgatggttactacccatacgctatggataactggggtcagggaaccctggtcaccgtctcctcagctagcggcaaaccaatcccaaacccactgctgggc

SEQ ID NO: 149 - DNA, 836 bp, synthetic construct
  misc_feature 1..836: note = Oligonucleotide
  source 1..836: mol_type = other DNA
cagttagataaaagaggcgcggcagcaagcggcgcgcatgccgatatcgttatgacccagtctccagattctctggctgtttctctgggtgaacgtgctaccatcaactgcaaatcttctcagtctctgctgaactctggtaaccagaaaaactacctggcttggtaccagcagaaaccaggtcagccaccaaaactgctgatctacggtgcttctacccgtgaatctggtgttccagatcgtttctctggttctggttctggtaccgatttcaccctgaccatctcttctctgcaggctgaagatgttgctgtttactactgtagagaccatggccagtaaggccggtctctttcggtggtggtaccaaagttgaaatcaaatccggagggtcgaccataacttcgtataatgtatactatacgaagttatcctcgagcggtacccaggttaccctgcgtgaatctggtccagctctggttaaaccaacccagaccctgaccctgacctgtaccgtttctggtttctctctgtctgcttactctgttaactggatccgtcagccaccaggtaaagctctggaatggctggctatgatctggggtgatggtaaaatcgtttacaactctgctctgaaatctcgtctgaccatctctaaagatacctctaaaaaccaggttgttctgaccatgaccaacatggatcctgtggacacagccacatattactgtgctggtgatggttactacccatacgctatggataactggggtcagggaaccctggtcaccgtctcctcagctagcggcaaaccaatcccaaacccactgctgggc

SEQ ID NO: 150 - DNA, 837 bp, synthetic construct
  misc_feature 1..837: note = Oligonucleotide
  source 1..837: mol_type = other DNA
cagttagataaaagaggcgcggcagcaagcggcgcgcatgccgatatcgttatgacccagtctccagattctctggctgtttctctgggtgaacgtgctaccatcaactgcaaatcttctcagtctctgctgaactctggtaaccagaaaaactacctggcttggtaccagcagaaaccaggtcagccaccaaaactgctgatctacggtttctacccgtgaatctggtgttccagatcgtttctctggttctggttctggtaccgatttcaccctgaccatctcttctctgcaggctgaagatgttgctgtttactactgtcagaacgttcactctttcccattcaccttcggtggtggtaccaaagttgaaatcaaatccggagggtcgaccataacttcgtataatgtatactatacgaagttatcctcgagcggtacccaggttaccctgcgtgaatctggtccagctctggttaaaccaacccagaccctgaccctgacctgtaccgtttctagagaccatggccagtaaggccggtctctgttaactggatccgtcagccaccaggtaaagctctggaatggctggctatgatctggggtgatggtaaaatcgtttacaactctgctctgaaatctcgtctgaccatctctaaagatacctctaaaaaccaggttgttctgaccatgaccaacatggatcctgtggacacagccacatattactgtgctggtgatggttactacccatacgctatggataactggggtcagggaaccctggtcaccgtctcctcagctagcggcaaaccaatcccaaacccactgctgggc

SEQ ID NO: 151 - DNA, 845 bp, synthetic construct
  misc_feature 1..845: note = Oligonucleotide
  source 1..845: mol_type = other DNA
cagttagataaaagaggcgcggcagcaagcggcgcgcatgccgatatcgttatgacccagtctccagattctctggctgtttctctgggtgaacgtgctaccatcaactgcaaatcttctcagtctctgctgaactctggtaaccagaaaaactacctggcttggtaccagcagaaaccaggtcagccaccaaaactgctgatctacggtgcttctacccgtgaatctggtgttccagatcgtttctctggttctggttctggtaccgatttcaccctgaccatctcttctctgcaggctgaagatgttgctgtttactactgtcagaacgttcactctttcccattcaccttcggtggtggtaccaaagttgaaatcaaatccggagggtcgaccataacttcgtataatgtatactatacgaagttatcctcgagcggtacccaggttaccctgcgtgaatctggtccagctctggttaaaccaacccagaccctgaccctgacctgtaccgtttctggtttctctctgtctgcttactctgttaactggatccgtcagccaccaggtaaagctctggaatggctggctatgagagaccatggccagtaaggccggtctctatcgtttacaactctgctctgaaatctcgtctgaccatctctaaagatacctctaaaaaccaggttgttctgaccatgaccaacatggatcctgtggacacagccacatattactgtgctggtgatggttactacccatacgctatggataactggggtcagggaaccctggtcaccgtctcctcagctagcggcaaaccaatcccaaacccactgctgggc

SEQ ID NO: 152 - DNA, 818 bp, synthetic construct
  misc_feature 1..818: note = Oligonucleotide
  source 1..818: mol_type = other DNA
cagttagataaaagaggcgcggcagcaagcggcgcgcatgccgatatcgttatgacccagtctccagattctctggctgtttctctgggtgaacgtgctaccatcaactgcaaatcttctcagtctctgctgaactctggtaaccagaaaaactacctggcttggtaccagcagaaaccaggtcagccaccaaaactgctgatctacggtgcttctacccgtgaatctggtgttccagatcgtttctctggttctggttctggtaccgatttcaccctgaccatctcttctctgcaggctgaagatgttgctgtttactactgtcagaacgttcactctttcccattcaccttcggtggtggtaccaaagttgaaatcaaatccggagggtcgaccataacttcgtataatgtatactatacgaagttatcctcgagcggtacccaggttaccctgcgtgaatctggtccagctctggttaaaccaacccagaccctgaccctgacctgtaccgtttctggtttctctctgtctgcttactctgttaactggatccgtcagccaccaggtaaagctctggaatggctggctatgatctggggtgatggtaaaatcgtttacaactctgctctgaaatctcgtctgaccatctctaaagatacctctaaaaaccaggttgttctgaccatgaccaacatggatcctgtggacacagccacatattactgtagagaccatggccagtaaggccggtctctggaaccctggtcaccgtctcctcagctagcggcaaaccaatcccaaacccactgctgggc

SEQ ID NO: 153 - DNA, 813 bp, synthetic construct
  misc_feature 1..813: note = Oligonucleotide
  source 1..813: mol_type = other DNA
cagttagataaaagaggcgcggcagcaagcggcgcgcatgccgatatcgttatgacccagtctccactgtctctgccagttaccccaggtgaaccagcttctatttcttgtcgttcttctcagtctctggtttactctaacggtgatacctacctgcactggtacctgcagaaaccaggtcagtctccacagctgctgatctacaaagtttctaaccgtttctctggtgttccagatcgtttctctggttctggttctggtaccgatttcaccctgaaaatctctcgtgttgaagctgaagatgttggtgtttactactgttctcagtctacccacgttccatggaccttcggtggtggtaccaaagttgaaatcaaatccggagggtcgaccataacttcgtataatgtatactatacgaagttatcctcgagcggtaccgaagttcagctggttgaatctggtggtggtctggttcagccaggtggttctctgcgtctgtcttgtgctgcttctggtttcaccttctcttcttacggtatgtcttgggttcgtcaggctccaggtaaaggtctggaactggttgcttctatcaactctaacggtggttctacctactacccagattctgttaaaggtcgtttcaccatctctcgtgataacgctaaaaactctctgtacctgcagatgaactctctgcgtgccgaggacacggctgtgtattactgtgcttctggtgattactggggtcaggggaccacggtcaccgtctcctcagctagcggcaaaccaatcccaaacccactgctgggc

SEQ ID NO: 154 - DNA, 809 bp, synthetic construct
  misc_feature 1..809: note = Oligonucleotide
  source 1..809: mol_type = other DNA
cagttagataaaagaggcgcggcagcaagcggcgcgcatgccgatatcgttatgacccagtctccactgtctctgccagttaccccaggtgaaccagcttctatttcttgtcgttcttctagagaccatggccagtaaggccggtctctctgcactggtacctgcagaaaccaggtcagtctccacagctgctgatctacaaagtttctaaccgtttctctggtgttccagatcgtttctctggttctggttctggtaccgatttcaccctgaaaatctctcgtgttgaagctgaagatgttggtgtttactactgttctcagtctacccacgttccatggaccttcggtggtggtaccaaagttgaaatcaaatccggagggtcgaccataacttcgtataatgtatactatacgaagttatcctcgagcggtaccgaagttcagctggttgaatctggtggtggtctggttcagccaggtggttctctgcgtctgtcttgtgctgcttctggtttcaccttctcttcttacggtatgtcttgggttcgtcaggctccaggtaaaggtctggaactggttgcttctatcaactctaacggtggttctacctactacccagattctgttaaaggtcgtttcaccatctctcgtgataacgctaaaaactctctgtacctgcagatgaactctctgcgtgccgaggacacggctgtgtattactgtgcttctggtgattactggggtcaggggaccacggtcaccgtctcctcagctagcggcaaaccaatcccaaacccactgctgggc

SEQ ID NO: 155 - DNA, 821 bp, synthetic construct
  misc_feature 1..821: note = Oligonucleotide
  source 1..821: mol_type = other DNA
cagttagataaaagaggcgcggcagcaagcggcgcgcatgccgatatcgttatgacccagtctccactgtctctgccagttaccccaggtgaaccagcttctatttcttgtcgttcttctcagtctctggtttactctaacggtgatacctacctgcactggtacctgcagaaaccaggtcagtctccacagctgctgatctacagagaccatggccagtaaggccggtctctggtgttccagatcgtttctctggttctggttctggtaccgatttcaccctgaaaatctctcgtgttgaagctgaagatgttggtgtttactactgttctcagtctacccacgttccatggaccttcggtggtggtaccaaagttgaaatcaaatccggagggtcgaccataacttcgtataatgtatactatacgaagttatcctcgagcggtaccgaagttcagctggttgaatctggtggtggtctggttcagccaggtggttctctgcgtctgtcttgtgctgcttctggtttcaccttctcttcttacggtatgtcttgggttcgtcaggctccaggtaaaggtctggaactggttgcttctatcaactctaacggtggttctacctactacccagattctgttaaaggtcgtttcaccatctctcgtgataacgctaaaaactctctgtacctgcagatgaactctctgcgtgccgaggacacggctgtgtattactgtgcttctggtgattactggggtcaggggaccacggtcaccgtctcctcagctagcggcaaaccaatcccaaacccactgctgggc

SEQ ID NO: 156 - DNA, 815 bp, synthetic construct
  misc_feature 1..815: note = Oligonucleotide
  source 1..815: mol_type = other DNA
cagttagataaaagaggcgcggcagcaagcggcgcgcatgccgatatcgttatgacccagtctccactgtctctgccagttaccccaggtgaaccagcttctatttcttgtcgttcttctcagtctctggtttactctaacggtgatacctacctgcactggtacctgcagaaaccaggtcagtctccacagctgctgatctacaaagtttctaaccgtttctctggtgttccagatcgtttctctggttctggttctggtaccgatttcaccctgaaaatctctcgtgttgaagctgaagatgttggtgtttactactgtagagaccatggccagtaaggccggtctctttcggtggtggtaccaaagttgaaatcaaatccggagggtcgaccataacttcgtataatgtatactatacgaagttatcctcgagcggtaccgaagttcagctggttgaatctggtggtggtctggttcagccaggtggttctctgcgtctgtcttgtgctgcttctggtttcaccttctcttcttacggtatgtcttgggttcgtcaggctccaggtaaaggtctggaactggttgcttctatcaactctaacggtggttctacctactacccagattctgttaaaggtcgtttcaccatctctcgtgataacgctaaaaactctctgtacctgcagatgaactctctgcgtgccgaggacacggctgtgtattactgtgcttctggtgattactggggtcaggggaccacggtcaccgtctcctcagctagcggcaaaccaatcccaaacccactgctgggc

SEQ ID NO: 157 - DNA, 818 bp, synthetic construct
  misc_feature 1..818: note = Oligonucleotide
  source 1..818: mol_type = other DNA
cagttagataaaagaggcgcggcagcaagcggcgcgcatgccgatatcgttatgacccagtctccactgtctctgccagttaccccaggtgaaccagcttctatttcttgtcgttcttctcagtctctggtttactctaacggtgatacctacctgcactggtacctgcagaaaccaggtcagtctccacagctgctgatctacaaagtttctaaccgtttctctggtgttccagatcgtttctctggttctggttctggtaccgatttcaccctgaaaatctctcgtgttgaagctgaagatgttggtgtttactactgttctcagtctacccacgttccatggaccttcggtggtggtaccaaagttgaaatcaaatccggagggtcgaccataacttcgtataatgtatactatacgaagttatcctcgagcggtaccgaagttcagctggttgaatctggtggtggtctggttcagccaggtggttctctgcgtctgtcttgtgctgcttctagagaccatggccagtaaggccggtctctatgtcttgggttcgtcaggctccaggtaaaggtctggaactggttgcttctatcaactctaacggtggttctacctactacccagattctgttaaaggtcgtttcaccatctctcgtgataacgctaaaaactctctgtacctgcagatgaactctctgcgtgccgaggacacggctgtgtattactgtgcttctggtgattactggggtcaggggaccacggtcaccgtctcctcagctagcggcaaaccaatcccaaacccactgctgggc

SEQ ID NO: 158 - DNA, 818 bp, synthetic construct
  misc_feature 1..818: note = Oligonucleotide
  source 1..818: mol_type = other DNA
cagttagataaaagaggcgcggcagcaagcggcgcgcatgccgatatcgttatgacccagtctccactgtctctgccagttaccccaggtgaaccagcttctatttcttgtcgttcttctcagtctctggtttactctaacggtgatacctacctgcactggtacctgcagaaaccaggtcagtctccacagctgctgatctacaaagtttctaaccgtttctctggtgttccagatcgtttctctggttctggttctggtaccgatttcaccctgaaaatctctcgtgttgaagctgaagatgttggtgtttactactgttctcagtctacccacgttccatggaccttcggtggtggtaccaaagttgaaatcaaatccggagggtcgaccataacttcgtataatgtatactatacgaagttatcctcgagcggtaccgaagttcagctggttgaatctggtggtggtctggttcagccaggtggttctctgcgtctgtcttgtgctgcttctggtttcaccttctcttcttacggtatgtcttgggttcgtcaggctccaggtaaaggtctggaactggttgcttctagagaccatggccagtaaggccggtctcttactacccagattctgttaaaggtcgtttcaccatctctcgtgataacgctaaaaactctctgtacctgcagatgaactctctgcgtgccgaggacacggctgtgtattactgtgcttctggtgattactggggtcaggggaccacggtcaccgtctcctcagctagcggcaaaccaatcccaaacccactgctgggc

SEQ ID NO: 159 - DNA, 818 bp, synthetic construct
  misc_feature 1..818: note = Oligonucleotide
  source 1..818: mol_type = other DNA
cagttagataaaagaggcgcggcagcaagcggcgcgcatgccgatatcgttatgacccagtctccactgtctctgccagttaccccaggtgaaccagcttctatttcttgtcgttcttctcagtctctggtttactctaacggtgatacctacctgcactggtacctgcagaaaccaggtcagtctccacagctgctgatctacaaagtttctaaccgtttctctggtgttccagatcgtttctctggttctggttctggtaccgatttcaccctgaaaatctctcgtgttgaagctgaagatgttggtgtttactactgttctcagtctacccacgttccatggaccttcggtggtggtaccaaagttgaaatcaaatccggagggtcgaccataacttcgtataatgtatactatacgaagttatcctcgagcggtaccgaagttcagctggttgaatctggtggtggtctggttcagccaggtggttctctgcgtctgtcttgtgctgcttctggtttcaccttctcttcttacggtatgtcttgggttcgtcaggctccaggtaaaggtctggaactggttgcttctatcaactctaacggtggttctacctactacccagattctgttaaaggtcgtttcaccatctctcgtgataacgctaaaaactctctgtacctgcagatgaactctctgcgtgccgaggacacggctgtgtattactgtagagaccatggccagtaaggccggtctctgggaccacggtcaccgtctcctcagctagcggcaaaccaatcccaaacccactgctgggc

SEQ ID NO: 160 - DNA, 825 bp, synthetic construct
  misc_feature 1..825: note = Oligonucleotide
  source 1..825: mol_type = other DNA
cagttagataaaagaggcgcggcagcaagcggcgcgcatgccgaaatcgttatgacccagtctccagctaccctgtctctgtctccaggtgaacgtgccactctgtcttgtcgtgcttctcagtctgtttcttcttacctggcttggtaccagcagaaaccaggtcaggctccacgtctgctgatctacgatgcttctaaccgtgctaccggtatcccagctcgtttctctggttctggttctggtaccgatttcaccctgaccatctcttctctggaaccagaagatttcgctgtttactactgtcaccagtacggttctaccccactgaccttcggtggtggtaccaaagttgaaatcaaatccggagggtcgaccataacttcgtataatgtatactatacgaagttatcctcgagcggtacccaggttcagctgcaggaatctggtccaggtctggttaaaccatctcagaccctgtctctgacctgtaccgtttctggtggttctatctcttctggtgattactactggtcttggatccgtcagccaccaggtaaaggtctggaatggatcggttacatctactactctggttctaccgattacaacccatctctgaaatctcgtgttaccatgtctgttgatacctctaaaaaccagttctctctgaaagttaactctgttaccgccgcggacacggctgtgtattactgtgctcgtgtttctatcttcggtgttggtaccttcgattactggggtcagggaaccctggtcaccgtctcctcagctagcggcaaaccaatcccaaacccactgctgggc

SEQ ID NO: 161 - DNA, 836 bp, synthetic construct
  misc_feature 1..836: note = Oligonucleotide
  source 1..836: mol_type = other DNA
cagttagataaaagaggcgcggcagcaagcggcgcgcatgccgaaatcgttatgacccagtctccagctaccctgtctctgtctccaggtgaacgtgccactctgtcttgtcgtgcttctagagaccatggccagtaaggccggtctctctggcttggtaccagcagaaaccaggtcaggctccacgtctgctgatctacgatgcttctaaccgtgctaccggtatcccagctcgtttctctggttctggttctggtaccgatttcaccctgaccatctcttctctggaaccagaagatttcgctgtttactactgtcaccagtacggttctaccccactgaccttcggtggtggtaccaaagttgaaatcaaatccggagggtcgaccataacttcgtataatgtatactatacgaagttatcctcgagcggtacccaggttcagctgcaggaatctggtccaggtctggttaaaccatctcagaccctgtctctgacctgtaccgtttctggtggttctatctcttctggtgattactactggtcttggatccgtcagccaccaggtaaaggtctggaatggatcggttacatctactactctggttctaccgattacaacccatctctgaaatctcgtgttaccatgtctgttgatacctctaaaaaccagttctctctgaaagttaactctgttaccgccgcggacacggctgtgtattactgtgctcgtgtttctatcttcggtgttggtaccttcgattactggggtcagggaaccctggtcaccgtctcctcagctagcggcaaaccaatcccaaacccactgctgggc

SEQ ID NO: 162 - DNA, 833 bp, synthetic construct
  misc_feature 1..833: note = Oligonucleotide
  source 1..833: mol_type = other DNA
cagttagataaaagaggcgcggcagcaagcggcgcgcatgccgaaatcgttatgacccagtctccagctaccctgtctctgtctccaggtgaacgtgccactctgtcttgtcgtgcttctcagtctgtttcttcttacctggcttggtaccagcagaaaccaggtcaggctccacgtctgctgatctacagagaccatggccagtaaggccggtctctggtatcccagctcgtttctctggttctggttctggtaccgatttcaccctgaccatctcttctctggaaccagaagatttcgctgtttactactgtcaccagtacggttctaccccactgaccttcggtggtggtaccaaagttgaaatcaaatccggagggtcgaccataacttcgtataatgtatactatacgaagttatcctcgagcggtacccaggttcagctgcaggaatctggtccaggtctggttaaaccatctcagaccctgtctctgacctgtaccgtttctggtggttctatctcttctggtgattactactggtcttggatccgtcagccaccaggtaaaggtctggaatggatcggttacatctactactctggttctaccgattacaacccatctctgaaatctcgtgttaccatgtctgttgatacctctaaaaaccagttctctctgaaagttaactctgttaccgccgcggacacggctgtgtattactgtgctcgtgtttctatcttcggtgttggtaccttcgattactggggtcagggaaccctggtcaccgtctcctcagctagcggcaaaccaatcccaaacccactgctgggc

SEQ ID NO: 163 - DNA, 827 bp, synthetic construct
  misc_feature 1..827: note = Oligonucleotide
  source 1..827: mol_type = other DNA
cagttagataaaagaggcgcggcagcaagcggcgcgcatgccgaaatcgttatgacccagtctccagctaccctgtctctgtctccaggtgaacgtgccactctgtcttgtcgtgcttctcagtctgtttcttcttacctggcttggtaccagcagaaaccaggtcaggctccacgtctgctgatctacgatgcttctaaccgtgctaccggtatcccagctcgtttctctggttctggttctggtaccgatttcaccctgaccatctcttctctggaaccagaagatttcgctgtttactactgtagagaccatggccagtaaggccggtctctttcggtggtggtaccaaagttgaaatcaaatccggagggtcgaccataacttcgtataatgtatactatacgaagttatcctcgagcggtacccaggttcagctgcaggaatctggtccaggtctggttaaaccatctcagaccctgtctctgacctgtaccgtttctggtggttctatctcttctggtgattactactggtcttggatccgtcagccaccaggtaaaggtctggaatggatcggttacatctactactctggttctaccgattacaacccatctctgaaatctcgtgttaccatgtctgttgatacctctaaaaaccagttctctctgaaagttaactctgttaccgccgcggacacggctgtgtattactgtgctcgtgtttctatcttcggtgttggtaccttcgattactggggtcagggaaccctggtcaccgtctcctcagctagcggcaaaccaatcccaaacccactgctgggc

SEQ ID NO: 164 - DNA, 824 bp, synthetic construct
  misc_feature 1..824: note = Oligonucleotide
  source 1..824: mol_type = other DNA
cagttagataaaagaggcgcggcagcaagcggcgcgcatgccgaaatcgttatgacccagtctccagctaccctgtctctgtctccaggtgaacgtgccactctgtcttgtcgtgcttctcagtctgtttcttcttacctggcttggtaccagcagaaaccaggtcaggctccacgtctgctgatctacgatgcttctaaccgtgctaccggtatcccagctcgtttctctggttctggttctggtaccgatttcaccctgaccatctcttctctggaaccagaagatttcgctgtttactactgtcaccagtacggttctaccccactgaccttcggtggtggtaccaaagttgaaatcaaatccggagggtcgaccataacttcgtataatgtatactatacgaagttatcctcgagcggtacccaggttcagctgcaggaatctggtccaggtctggttaaaccatctcagaccctgtctctgacctgtaccgtttctagagaccatggccagtaaggccggtctcttggtcttggatccgtcagccaccaggtaaaggtctggaatggatcggttacatctactactctggttctaccgattacaacccatctctgaaatctcgtgttaccatgtctgttgatacctctaaaaaccagttctctctgaaagttaactctgttaccgccgcggacacggctgtgtattactgtgctcgtgtttctatcttcggtgttggtaccttcgattactggggtcagggaaccctggtcaccgtctcctcagctagcggcaaaccaatcccaaacccactgctgggc

SEQ ID NO: 165 - DNA, 833 bp, synthetic construct
  misc_feature 1..833: note = Oligonucleotide
  source 1..833: mol_type = other DNA
cagttagataaaagaggcgcggcagcaagcggcgcgcatgccgaaatcgttatgacccagtctccagctaccctgtctctgtctccaggtgaacgtgccactctgtcttgtcgtgcttctcagtctgtttcttcttacctggcttggtaccagcagaaaccaggtcaggctccacgtctgctgatctacgatgcttctaaccgtgctaccggtatcccagctcgtttctctggttctggttctggtaccgatttcaccctgaccatctcttctctggaaccagaagatttcgctgtttactactgtcaccagtacggttctaccccactgaccttcggtggtggtaccaaagttgaaatcaaatccggagggtcgaccataacttcgtataatgtatactatacgaagttatcctcgagcggtacccaggttcagctgcaggaatctggtccaggtctggttaaaccatctcagaccctgtctctgacctgtaccgtttctggtggttctatctcttctggtgattactactggtcttggatccgtcagccaccaggtaaaggtctggaatggatcggttacagagaccatggccagtaaggccggtctctgattacaacccatctctgaaatctcgtgttaccatgtctgttgatacctctaaaaaccagttctctctgaaagttaactctgttaccgccgcggacacggctgtgtattactgtgctcgtgtttctatcttcggtgttggtaccttcgattactggggtcagggaaccctggtcaccgtctcctcagctagcggcaaaccaatcccaaacccactgctgggc

SEQ ID NO: 166 - DNA, 806 bp, synthetic construct
  misc_feature 1..806: note = Oligonucleotide
  source 1..806: mol_type = other DNA
cagttagataaaagaggcgcggcagcaagcggcgcgcatgccgaaatcgttatgacccagtctccagctaccctgtctctgtctccaggtgaacgtgccactctgtcttgtcgtgcttctcagtctgtttcttcttacctggcttggtaccagcagaaaccaggtcaggctccacgtctgctgatctacgatgcttctaaccgtgctaccggtatcccagctcgtttctctggttctggttctggtaccgatttcaccctgaccatctcttctctggaaccagaagatttcgctgtttactactgtcaccagtacggttctaccccactgaccttcggtggtggtaccaaagttgaaatcaaatccggagggtcgaccataacttcgtataatgtatactatacgaagttatcctcgagcggtacccaggttcagctgcaggaatctggtccaggtctggttaaaccatctcagaccctgtctctgacctgtaccgtttctggtggttctatctcttctggtgattactactggtcttggatccgtcagccaccaggtaaaggtctggaatggatcggttacatctactactctggttctaccgattacaacccatctctgaaatctcgtgttaccatgtctgttgatacctctaaaaaccagttctctctgaaagttaactctgttaccgccgcggacacggctgtgtattactgtagagaccatggccagtaaggccggtctctggaaccctggtcaccgtctcctcagctagcggcaaaccaatcccaaacccactgctgggc

SEQ ID NO: 167 - DNA, 816 bp, synthetic construct
  misc_feature 1..816: note = Oligonucleotide
  source 1..816: mol_type = other DNA
cagttagataaaagaggcgcggcagcaagcggcgcgcatgccgaaatcgttctgacccagtctccaggtaccctgtctctgtctccaggtgaacgtgccactctgtcttgtcgtgcttctcagtctgtttcttcttctttcctggcttggtaccagcagaaaccaggtcaggctccacgtctgctgatctacggtgcttcttctcgtgctaccggtatcccagatcgtctgtctggttctggttctggtaccgatttcaccctgaccatcacccgtctggaaccagaagatttcgctgtttactactgtcagcagtacgattcttctgctatcaccttcggtggtggtaccaaagttgaaatcaaatccggagggtcgaccataacttcgtataatgtatactatacgaagttatcctcgagcggtaccgaagttcagctggttcagtctggtgctgaagttaaaaaaccaggtgaatctctgaaaatctcttgtaaaggttctggttacatcttcaccaactactggatcgcttgggttcgtcaggttccaggtaaaggtctggaatctatgggtatcatctacccaggtgattctgatatccgttactctccatctttccagggtcaggttaccatctctgctgataaatctatcaccaccgcttacctgcagtggtcttctctgaaagcctcggacaccgccatttattactgtgctcgtcacgatatcgaaggtttcgattactggggtcgtggaaccctggtcaccgtctcctcagctagcggcaaaccaatcccaaacccactgctgggc

SEQ ID NO: 168 - DNA, 824 bp, synthetic construct
  misc_feature 1..824: note = Oligonucleotide
  source 1..824: mol_type = other DNA
cagttagataaaagaggcgcggcagcaagcggcgcgcatgccgaaatcgttctgacccagtctccaggtaccctgtctctgtctccaggtgaacgtgccactctgtcttgtcgtgcttctagagaccatggccagtaaggccggtctctctggcttggtaccagcagaaaccaggtcaggctccacgtctgctgatctacggtgcttcttctcgtgctaccggtatcccagatcgtctgtctggttctggttctggtaccgatttcaccctgaccatcacccgtctggaaccagaagatttcgctgtttactactgtcagcagtacgattcttctgctatcaccttcggtggtggtaccaaagttgaaatcaaatccggagggtcgaccataacttcgtataatgtatactatacgaagttatcctcgagcggtaccgaagttcagctggttcagtctggtgctgaagttaaaaaaccaggtgaatctctgaaaatctcttgtaaaggttctggttacatcttcaccaactactggatcgcttgggttcgtcaggttccaggtaaaggtctggaatctatgggtatcatctacccaggtgattctgatatccgttactctccatctttccagggtcaggttaccatctctgctgataaatctatcaccaccgcttacctgcagtggtcttctctgaaagcctcggacaccgccatttattactgtgctcgtcacgatatcgaaggtttcgattactggggtcgtggaaccctggtcaccgtctcctcagctagcggcaaaccaatcccaaacccactgctgggc

SEQ ID NO: 169 - DNA, 824 bp, synthetic construct
  misc_feature 1..824: note = Oligonucleotide
  source 1..824: mol_type = other DNA
cagttagataaaagaggcgcggcagcaagcggcgcgcatgccgaaatcgttctgacccagtctccaggtaccctgtctctgtctccaggtgaacgtgccactctgtcttgtcgtgcttctcagtctgtttcttcttctttcctggcttggtaccagcagaaaccaggtcaggctccacgtctgctgatctacagagaccatggccagtaaggccggtctctggtatcccagatcgtctgtctggttctggttctggtaccgatttcaccctgaccatcacccgtctggaaccagaagatttcgctgtttactactgtcagcagtacgattcttctgctatcaccttcggtggtggtaccaaagttgaaatcaaatccggagggtcgaccataacttcgtataatgtatactatacgaagttatcctcgagcggtaccgaagttcagctggttcagtctggtgctgaagttaaaaaaccaggtgaatctctgaaaatctcttgtaaaggttctggttacatcttcaccaactactggatcgcttgggttcgtcaggttccaggtaaaggtctggaatctatgggtatcatctacccaggtgattctgatatccgttactctccatctttccagggtcaggttaccatctctgctgataaatctatcaccaccgcttacctgcagtggtcttctctgaaagcctcggacaccgccatttattactgtgctcgtcacgatatcgaaggtttcgattactggggtcgtggaaccctggtcaccgtctcctcagctagcggcaaaccaatcccaaacccactgctgggc

SEQ ID NO: 170 - DNA, 818 bp, synthetic construct
  misc_feature 1..818: note = Oligonucleotide
  source 1..818: mol_type = other DNA
cagttagataaaagaggcgcggcagcaagcggcgcgcatgccgaaatcgttctgacccagtctccaggtaccctgtctctgtctccaggtgaacgtgccactctgtcttgtcgtgcttctcagtctgtttcttcttctttcctggcttggtaccagcagaaaccaggtcaggctccacgtctgctgatctacggtgcttcttctcgtgctaccggtatcccagatcgtctgtctggttctggttctggtaccgatttcaccctgaccatcacccgtctggaaccagaagatttcgctgtttactactgtagagaccatggccagtaaggccggtctctttcggtggtggtaccaaagttgaaatcaaatccggagggtcgaccataacttcgtataatgtatactatacgaagttatcctcgagcggtaccgaagttcagctggttcagtctggtgctgaagttaaaaaaccaggtgaatctctgaaaatctcttgtaaaggttctggttacatcttcaccaactactggatcgcttgggttcgtcaggttccaggtaaaggtctggaatctatgggtatcatctacccaggtgattctgatatccgttactctccatctttccagggtcaggttaccatctctgctgataaatctatcaccaccgcttacctgcagtggtcttctctgaaagcctcggacaccgccatttattactgtgctcgtcacgatatcgaaggtttcgattactggggtcgtggaaccctggtcaccgtctcctcagctagcggcaaaccaatcccaaacccactgctgggc

SEQ ID NO: 171 - DNA, 821 bp, synthetic construct
  misc_feature 1..821: note = Oligonucleotide
  source 1..821: mol_type = other DNA
cagttagataaaagaggcgcggcagcaagcggcgcgcatgccgaaatcgttctgacccagtctccaggtaccctgtctctgtctccaggtgaacgtgccactctgtcttgtcgtgcttctcagtctgtttcttcttctttcctggcttggtaccagcagaaaccaggtcaggctccacgtctgctgatctacggtgcttcttctcgtgctaccggtatcccagatcgtctgtctggttctggttctggtaccgatttcaccctgaccatcacccgtctggaaccagaagatttcgctgtttactactgtcagcagtacgattcttctgctatcaccttcggtggtggtaccaaagttgaaatcaaatccggagggtcgaccataacttcgtataatgtatactatacgaagttatcctcgagcggtaccgaagttcagctggttcagtctggtgctgaagttaaaaaaccaggtgaatctctgaaaatctcttgtaaaggttctagagaccatggccagtaaggccggtctctatcgcttgggttcgtcaggttccaggtaaaggtctggaatctatgggtatcatctacccaggtgattctgatatccgttactctccatctttccagggtcaggttaccatctctgctgataaatctatcaccaccgcttacctgcagtggtcttctctgaaagcctcggacaccgccatttattactgtgctcgtcacgatatcgaaggtttcgattactggggtcgtggaaccctggtcaccgtctcctcagctagcggcaaaccaatcccaaacccactgctgggc

SEQ ID NO: 172 - DNA, 820 bp, synthetic construct
  misc_feature 1..820: note = Oligonucleotide
  source 1..820: mol_type = other DNA
cagttagataaaagaggcgcggcagcaagcggcgcgcatgccgaaatcgttctgacccagtctccaggtaccctgtctctgtctccaggtgaacgtgccactctgtcttgtcgtgcttctcagtctgtttcttcttctttcctggcttggtaccagcagaaaccaggtcaggctccacgtctgctgatctacggtgcttcttctcgtgctaccggtatcccagatcgtctgtctggttctggttctggtaccgatttcaccctgaccatcacccgtctggaaccagaagatttcgctgtttactactgtcagcagtacgattcttctgctatcaccttcggtggtggtaccaaagttgaaatcaaatccggagggtcgaccataacttcgtataatgtatactatacgaagttatcctcgagcggtaccgaagttcagctggttcagtctggtgctgaagttaaaaaaccaggtgaatctctgaaaatctcttgtaaaggttctggttacatcttcaccaactactggatcgcttgggttcgtcaggttccaggtaaaggtctggaatctatgggtatcagagaccatggccagtaaggccggtctctcgttactctccatctttccagggtcaggttaccatctctgctgataaatctatcaccaccgcttacctgcagtggtcttctctgaaagcctcgacaccgccatttattactgtgctcgtcacgatatcgaaggtttcgattactggggtcgtggaaccctggtcaccgtctcctcagctagcggcaaaccaatcccaaacccactgctgggc

SEQ ID NO: 173 - DNA, 806 bp, synthetic construct
  misc_feature 1..806: note = Oligonucleotide
  source 1..806: mol_type = other DNA
cagttagataaaagaggcgcggcagcaagcggcgcgcatgccgaaatcgttctgacccagtctccaggtaccctgtctctgtctccaggtgaacgtgccactctgtcttgtcgtgcttctcagtctgtttcttcttctttcctggcttggtaccagcagaaaccaggtcaggctccacgtctgctgatctacggtgcttcttctcgtgctaccggtatcccagatcgtctgtctggttctggttctggtaccgatttcaccctgaccatcacccgtctggaaccagaagatttcgctgtttactactgtcagcagtacgattcttctgctatcaccttcggtggtggtaccaaagttgaaatcaaatccggagggtcgaccataacttcgtataatgtatactatacgaagttatcctcgagcggtaccgaagttcagctggttcagtctggtgctgaagttaaaaaaccaggtgaatctctgaaaatctcttgtaaaggttctggttacatcttcaccaactactggatcgcttgggttcgtcaggttccaggtaaaggtctggaatctatgggtatcatctacccaggtgattctgatatccgttactctccatctttccagggtcaggttaccatctctgctgataaatctatcaccaccgcttacctgcagtggtcttctctgaaagcctcggacaccgccatttattactgtagagaccatggccagtaaggccggtctctggaaccctggtcaccgtctcctcagctagcggcaaaccaatcccaaacccactgctgggc

SEQ ID NO: 174 - DNA, 813 bp, synthetic construct
  misc_feature 1..813: note = Oligonucleotide
  source 1..813: mol_type = other DNA
cagttagataaaagaggcgcggcagcaagcggcgcgcatgccgaatctgctctgacccagccagcttctgtttctggttctccaggtcagtctatcaccatctcttgtaccggtacctcttctgatgttggtggttacaactctgtttcttggtaccagcagcacccaggtaaagctccaaaactgatgatctacgaagtttctaaccgtccatctggtgtttctaaccgtttctctggttctaaatctggtaacaccgcttctctgaccatctctggtctgcaggctgaagatgaagctgattactactgtaactcttacacctctacctctatggttttcggtggtggtaccaaactgaccgttctgtccggagggtcgaccataacttcgtataatgtatactatacgaagttatcctcgagcggtaccgaagttcagctggttcagtctggtgctgaagttaaaaaaccaggtgcttctgttaaagtttcttgtaaagcttctggttacaccctgacctcttacggtatctcttgggttcgtcaggctccaggtcagggtctggaatggatgggttgggtttctttctacaacggtaacaccaactacgctcagaaactgcagggtcgtggtaccatgaccaccgatccatctacctctaccgcttacatggaactgcgttctctgcgttctgacgacacggccgtgtattactgtgctcgtggttacggtatggatgtttggggtcaggggaccacggtcaccgtctcctcagctagcggcaaaccaatcccaaacccactgctgggc

SEQ ID NO: 175 - DNA, 815 bp, synthetic construct
  misc_feature 1..815: note = Oligonucleotide
  source 1..815: mol_type = other DNA
cagttagataaaagaggcgcggcagcaagcggcgcgcatgccgaatctgctctgacccagccagcttctgtttctggttctccaggtcagtctatcaccatctcttgtaccggtaccagagaccatggccagtaaggccggtctctgtttcttggtaccagcagcacccaggtaaagctccaaaactgatgatctacgaagtttctaaccgtccatctggtgtttctaaccgtttctctggttctaaatctggtaacaccgcttctctgaccatctctggtctgcaggctgaagatgaagctgattactactgtaactcttacacctctacctctatggttttcggtggtggtaccaaactgaccgttctgtccggagggtcgaccataacttcgtataatgtatactatacgaagttatcctcgagcggtaccgaagttcagctggttcagtctggtgctgaagttaaaaaaccaggtgcttctgttaaagtttcttgtaaagcttctggttacaccctgacctcttacggtatctcttgggttcgtcaggctccaggtcagggtctggaatggatgggttgggtttctttctacaacggtaacaccaactacgctcagaaactgcagggtcgtggtaccatgaccaccgatccatctacctctaccgcttacatggaactgcgttctctgcgttctgacgacacggccgtgtattactgtgctcgtggttacggtatggatgtttggggtcaggggaccacggtcaccgtctcctcagctagcggcaaaccaatcccaaacccactgctgggc

SEQ ID NO: 176 - DNA, 821 bp, synthetic construct
  misc_feature 1..821: note = Oligonucleotide
  source 1..821: mol_type = other DNA
cagttagataaaagaggcgcggcagcaagcggcgcgcatgccgaatctgctctgacccagccagcttctgtttctggttctccaggtcagtctatcaccatctcttgtaccggtacctcttctgatgttggtggttacaactctgtttcttggtaccagcagcacccaggtaaagctccaaaactgatgatctacagagaccatggccagtaaggccggtctctggtgtttctaaccgtttctctggttctaaatctggtaacaccgcttctctgaccatctctggtctgcaggctgaagatgaagctgattactactgtaactcttacacctctacctctatggttttcggtggtggtaccaaactgaccgttctgtccggagggtcgaccataacttcgtataatgtatactatacgaagttatcctcgagcggtaccgaagttcagctggttcagtctggtgctgaagttaaaaaaccaggtgcttctgttaaagtttcttgtaaagcttctggttacaccctgacctcttacggtatctcttgggttcgtcaggctccaggtcagggtctggaatggatgggttgggtttctttctacaacggtaacaccaactacgctcagaaactgcagggtcgtggtaccatgaccaccgatccatctacctctaccgcttacatggaactgcgttctctgcgttctgacgacacggccgtgtattactgtgctcgtggttacggtatggatgtttggggtcaggggaccacggtcaccgtctcctcagctagcggcaaaccaatcccaaacccactgctgggc

SEQ ID NO: 177 - DNA, 815 bp, synthetic construct
  misc_feature 1..815: note = Oligonucleotide
  source 1..815: mol_type = other DNA
cagttagataaaagaggcgcggcagcaagcggcgcgcatgccgaatctgctctgacccagccagcttctgtttctggttctccaggtcagtctatcaccatctcttgtaccggtacctcttctgatgttggtggttacaactctgtttcttggtaccagcagcacccaggtaaagctccaaaactgatgatctacgaagtttctaaccgtccatctggtgtttctaaccgtttctctggttctaaatctggtaacaccgcttctctgaccatctctggtctgcaggctgaagatgaagctgattactactgtagagaccatggccagtaaggccggtctctttcggtggtggtaccaaactgaccgttctgtccggagggtcgaccataacttcgtataatgtatactatacgaagttatcctcgagcggtaccgaagttcagctggttcagtctggtgctgaagttaaaaaaccaggtgcttctgttaaagtttcttgtaaagcttctggttacaccctgacctcttacggtatctcttgggttcgtcaggctccaggtcagggtctggaatggatgggttgggtttctttctacaacggtaacaccaactacgctcagaaactgcagggtcgtggtaccatgaccaccgatccatctacctctaccgcttacatggaactgcgttctctgcgttctgacgacacggccgtgtattactgtgctcgtggttacggtatggatgtttggggtcaggggaccacggtcaccgtctcctcagctagcggcaaaccaatcccaaacccactgctgggc

SEQ ID NO: 178 - DNA, 818 bp, synthetic construct
  misc_feature 1..818: note = Oligonucleotide
  source 1..818: mol_type = other DNA
cagttagataaaagaggcgcggcagcaagcggcgcgcatgccgaatctgctctgacccagccagcttctgtttctggttctccaggtcagtctatcaccatctcttgtaccggtacctcttctgatgttggtggttacaactctgtttcttggtaccagcagcacccaggtaaagctccaaaactgatgatctacgaagtttctaaccgtccatctggtgtttctaaccgtttctctggttctaaatctggtaacaccgcttctctgaccatctctggtctgcaggctgaagatgaagctgattactactgtaactcttacacctctacctctatggttttcggtggtggtaccaaactgaccgttctgtccggagggtcgaccataacttcgtataatgtatactatacgaagttatcctcgagcggtaccgaagttcagctggttcagtctggtgctgaagttaaaaaaccaggtgcttctgttaaagtttcttgtaaagcttctagagaccatggccagtaaggccggtctctatctcttgggttcgtcaggctccaggtcagggtctggaatggatgggttgggtttctttctacaacggtaacaccaactacgctcagaaactgcagggtcgtggtaccatgaccaccgatccatctacctctaccgcttacatggaactgcgttctctgcgttctgacgacacggccgtgtattactgtgctcgtggttacggtatggatgtttggggtcaggggaccacggtcaccgtctcctcagctagcggcaaaccaatcccaaacccactgctgggc

SEQ ID NO: 179 - DNA, 818 bp, synthetic construct
  misc_feature 1..818: note = Oligonucleotide
  source 1..818: mol_type = other DNA
cagttagataaaagaggcgcggcagcaagcggcgcgcatgccgaatctgctctgacccagccagcttctgtttctggttctccaggtcagtctatcaccatctcttgtaccggtacctcttctgatgttggtggttacaactctgtttcttggtaccagcagcacccaggtaaagctccaaaactgatgatctacgaagtttctaaccgtccatctggtgtttctaaccgtttctctggttctaaatctggtaacaccgcttctctgaccatctctggtctgcaggctgaagatgaagctgattactactgtaactcttacacctctacctctatggttttcggtggtggtaccaaactgaccgttctgtccggagggtcgaccataacttcgtataatgtatactatacgaagttatcctcgagcggtaccgaagttcagctggttcagtctggtgctgaagttaaaaaaccaggtgcttctgttaaagtttcttgtaaagcttctggttacaccctgacctcttacggtatctcttgggttcgtcaggctccaggtcagggtctggaatggatgggttggagagaccatggccagtaaggccggtctctaactacgctcagaaactgcagggtcgtggtaccatgaccaccgatccatctacctctaccgcttacatggaactgcgttctctgcgttctgacgacacggccgtgtattactgtgctcgtggttacggtatggatgtttggggtcaggggaccacggtcaccgtctcctcagctagcggcaaaccaatcccaaacccactgctgggc

SEQ ID NO: 180 - DNA, 809 bp, synthetic construct
  misc_feature 1..809: note = Oligonucleotide
  source 1..809: mol_type = other DNA
cagttagataaaagaggcgcggcagcaagcggcgcgcatgccgaatctgctctgacccagccagcttctgtttctggttctccaggtcagtctatcaccatctcttgtaccggtacctcttctgatgttggtggttacaactctgtttcttggtaccagcagcacccaggtaaagctccaaaactgatgatctacgaagtttctaaccgtccatctggtgtttctaaccgtttctctggttctaaatctggtaacaccgcttctctgaccatctctggtctgcaggctgaagatgaagctgattactactgtaactcttacacctctacctctatggttttcggtggtggtaccaaactgaccgttctgtccggagggtcgaccataacttcgtataatgtatactatacgaagttatcctcgagcggtaccgaagttcagctggttcagtctggtgctgaagttaaaaaaccaggtgcttctgttaaagtttcttgtaaagcttctggttacaccctgacctcttacggtatctcttgggttcgtcaggctccaggtcagggtctggaatggatgggttgggtttctttctacaacggtaacaccaactacgctcagaaactgcagggtcgtggtaccatgaccaccgatccatctacctctaccgcttacatggaactgcgttctctgcgttctgacgacacggccgtgtattactgtagagaccatggccagtaaggccggtctctgggaccacggtcaccgtctcctcagctagcggcaaaccaatcccaaacccactgctgggc

SEQ ID NO: 181 - DNA, 42 bp, synthetic construct
  misc_feature 1..42: note = Primer
  source 1..42: mol_type = other DNA
gcttctgttggtgatcgtgttactattacctgtcgtgcttct

SEQ ID NO: 182 - DNA, 42 bp, synthetic construct
  misc_feature 1..42: note = Primer
  source 1..42: mol_type = other DNA
gtttctctgggtgaacgtgctaccatcaactgcaaatcttct

SEQ ID NO: 183 - DNA, 42 bp, synthetic construct
  misc_feature 1..42: note = Primer
  source 1..42: mol_type = other DNA
gttaccccaggtgaaccagcttctatttcttgtcgttcttct

SEQ ID NO: 184 - DNA, 42 bp, synthetic construct
  misc_feature 1..42: note = Primer
  source 1..42: mol_type = other DNA
ctgtctccaggtgaacgtgccactctgtcttgtcgtgcttct

SEQ ID NO: 185 - DNA, 42 bp, synthetic construct; identical to SEQ ID NO: 184
  misc_feature 1..42: note = Primer
  source 1..42: mol_type = other DNA

SEQ ID NO: 186 - DNA, 42 bp, synthetic construct
  misc_feature 1..42: note = Primer
  source 1..42: mol_type = other DNA
ggttctccaggtcagtctatcaccatctcttgtaccggtacc

SEQ ID NO: 187 - DNA, 42 bp, synthetic construct
  misc_feature 1..42: note = Primer
  source 1..42: mol_type = other DNA
cagttttggagctttacctggtttctgctggtaccaagccag

SEQ ID NO: 188 - DNA, 42 bp, synthetic construct
  misc_feature 1..42: note = Primer
  source 1..42: mol_type = other DNA
cagttttggtggctgacctggtttctgctggtaccaagccag

SEQ ID NO: 189 - DNA, 42 bp, synthetic construct
  misc_feature 1..42: note = Primer
  source 1..42: mol_type = other DNA
cagctgtggagactgacctggtttctgcaggtaccagtgcag

SEQ ID NO: 190 - DNA, 42 bp, synthetic construct
  misc_feature 1..42: note = Primer
  source 1..42: mol_type = other DNA
cagacgtggagcctgacctggtttctgctggtaccaagccag

SEQ ID NO: 191 - DNA, 42 bp, synthetic construct; identical to SEQ ID NO: 190
  misc_feature 1..42: note = Primer
  source 1..42: mol_type = other DNA

SEQ ID NO: 192 - DNA, 42 bp, synthetic construct
  misc_feature 1..42: note = Primer
  source 1..42: mol_type = other DNA
cagttttggagctttacctgggtgctgctggtaccaagaaac

SEQ ID NO: 193 - DNA, 42 bp, synthetic construct
  misc_feature 1..42: note = Primer
  source 1..42: mol_type = other DNA
taccagcagaaaccaggtaaagctccaaaactgctgatctac

SEQ ID NO: 194 - DNA, 42 bp, synthetic construct
  misc_feature 1..42: note = Primer
  source 1..42: mol_type = other DNA
taccagcagaaaccaggtcagccaccaaaactgctgatctac

SEQ ID NO: 195 - DNA, 42 bp, synthetic construct
  misc_feature 1..42: note = Primer
  source 1..42: mol_type = other DNA
tacctgcagaaaccaggtcagtctccacagctgctgatctac

SEQ ID NO: 196 - DNA, 42 bp, synthetic construct
  misc_feature 1..42: note = Primer
  source 1..42: mol_type = other DNA
taccagcagaaaccaggtcaggctccacgtctgctgatctac

SEQ ID NO: 197 - DNA, 42 bp, synthetic construct; identical to SEQ ID NO: 196
  misc_feature 1..42: note = Primer
  source 1..42: mol_type = other DNA

SEQ ID NO: 198 - DNA, 42 bp, synthetic construct
  misc_feature 1..42: note = Primer
  source 1..42: mol_type = other DNA
taccagcagcacccaggtaaagctccaaaactgatgatctac

SEQ ID NO: 199 - DNA, 42 bp, synthetic construct
  misc_feature 1..42: note = Primer
  source 1..42: mol_type = other DNA
atcggtaccagaaccagaaccagagaaacgagatggaacacc

SEQ ID NO: 200 - DNA, 42 bp, synthetic construct
  misc_feature 1..42: note = Primer
  source 1..42: mol_type = other DNA
atcggtaccagaaccagaaccagagaaacgatctggaacacc

SEQ ID NO: 201 - DNA, 42 bp, synthetic construct; identical to SEQ ID NO: 200
  misc_feature 1..42: note = Primer
  source 1..42: mol_type = other DNA

SEQ ID NO: 202 - DNA, 42 bp, synthetic construct
  misc_feature 1..42: note = Primer
  source 1..42: mol_type = other DNA
atcggtaccagaaccagaaccagagaaacgagctgggatacc

SEQ ID NO: 203 - DNA, 42 bp, synthetic construct
  misc_feature 1..42: note = Primer
  source 1..42: mol_type = other DNA
atcggtaccagaaccagaaccagacagacgatctgggatacc

SEQ ID NO: 204 - DNA, 42 bp, synthetic construct
  misc_feature 1..42: note = Primer
  source 1..42: mol_type = other DNA
ggtgttaccagatttagaaccagagaaacggttagaaacacc

SEQ ID NO: 205 - DNA, 42 bp, synthetic construct
  misc_feature 1..42: note = Primer
  source 1..42: mol_type = other DNA
atctcttctctgcagccagaagatttcgctaactactactgt

SEQ ID NO: 206 - DNA, 42 bp, synthetic construct
  misc_feature 1..42: note = Primer
  source 1..42: mol_type = other DNA
atctcttctctgcaggctgaagatgttgctgtttactactgt

SEQ ID NO: 207 - DNA, 42 bp, synthetic construct
  misc_feature 1..42: note = Primer
  source 1..42: mol_type = other DNA
atctctcgtgttgaagctgaagatgttggtgtttactactgt

SEQ ID NO: 208 - DNA, 42 bp, synthetic construct
  misc_feature 1..42: note = Primer
  source 1..42: mol_type = other DNA
atctcttctctggaaccagaagatttcgctgtttactactgt

SEQ ID NO: 209 - DNA, 42 bp, synthetic construct
  misc_feature 1..42: note = Primer
  source 1..42: mol_type = other DNA
atcacccgtctggaaccagaagatttcgctgtttactactgt

SEQ ID NO: 210 - DNA, 42 bp, synthetic construct
  misc_feature 1..42: note = Primer
  source 1..42: mol_type = other DNA
atctctggtctgcaggctgaagatgaagctgattactactgt

SEQ ID NO: 211 - DNA, 42 bp, synthetic construct
  misc_feature 1..42: note = Primer
  source 1..42: mol_type = other DNA
cgaccctccggatttgatttcaactttggtaccaccaccgaa

SEQ ID NO: 212 - DNA, 42 bp, synthetic construct
  misc_feature 1..42: note = Primer
  source 1..42: mol_type = other DNA
cgaccctccggacagaacggtcagtttggtaccaccaccgaa

SEQ ID NO: 213 - DNA, 42 bp, synthetic construct
  misc_feature 1..42: note = Primer
  source 1..42: mol_type = other DNA
aaaaaaccaggtgcttctgttaaagtttcttgtaaagtttct

SEQ ID NO: 214 - DNA, 42 bp, synthetic construct
  misc_feature 1..42: note = Primer
  source 1..42: mol_type = other DNA
gttaaaccaacccagaccctgaccctgacctgtaccgtttct

SEQ ID NO: 215 - DNA, 42 bp, synthetic construct
  misc_feature 1..42: note = Primer
  source 1..42: mol_type = other DNA
gttcagccaggtggttctctgcgtctgtcttgtgctgcttct

SEQ ID NO: 216 - DNA, 42 bp, synthetic construct
  misc_feature 1..42: note = Primer
  source 1..42: mol_type = other DNA
gttaaaccatctcagaccctgtctctgacctgtaccgtttct

SEQ ID NO: 217 - DNA, 42 bp, synthetic construct
  misc_feature 1..42: note = Primer
  source 1..42: mol_type = other DNA
aaaaaaccaggtgaatctctgaaaatctcttgtaaaggttct

SEQ ID NO: 218 - DNA, 42 bp, synthetic construct
  misc_feature 1..42: note = Primer
  source 1..42: mol_type = other DNA
aaaaaaccaggtgcttctgttaaagtttcttgtaaagcttct

SEQ ID NO: 219 - DNA, 42 bp, synthetic construct
  misc_feature 1..42: note = Primer
  source 1..42: mol_type = other DNA
ccattccagacctttacctggagcctgacgaacccagtggat

SEQ ID NO: 220 - DNA, 42 bp, synthetic construct
  misc_feature 1..42: note = Primer
  source 1..42: mol_type = other DNA
ccattccagagctttacctggtggctgacggatccagttaac

SEQ ID NO: 221 - DNA, 42 bp, synthetic construct
  misc_feature 1..42: note = Primer
  source 1..42: mol_type = other DNA
cagttccagacctttacctggagcctgacgaacccaagacat

SEQ ID NO: 222 - DNA, 42 bp, synthetic construct
  misc_feature 1..42: note = Primer
  source 1..42: mol_type = other DNA
ccattccagacctttacctggtggctgacggatccaagacca

SEQ ID NO: 223 - DNA, 42 bp, synthetic construct
  misc_feature 1..42: note = Primer
  source 1..42: mol_type = other DNA
agattccagacctttacctggaacctgacgaacccaagcgat

SEQ ID NO: 224 - DNA, 42 bp, synthetic construct
  misc_feature 1..42: note = Primer
  source 1..42: mol_type = other DNA
ccattccagaccctgacctggagcctgacgaacccaagagat

SEQ ID NO: 225 - DNA, 42 bp, synthetic construct
  misc_feature 1..42: note = Primer
  source 1..42: mol_type = other DNA
gttcgtcaggctccaggtaaaggtctggaatggatgggtggt

SEQ ID NO: 226 - DNA, 42 bp, synthetic construct
  misc_feature 1..42: note = Primer
  source 1..42: mol_type = other DNA
atccgtcagccaccaggtaaagctctggaatggctggctatg

SEQ ID NO: 227 - DNA, 42 bp, synthetic construct
  misc_feature 1..42: note = Primer
  source 1..42: mol_type = other DNA
gttcgtcaggctccaggtaaaggtctggaactggttgcttct

SEQ ID NO: 228 - DNA, 42 bp, synthetic construct
  misc_feature 1..42: note = Primer
  source 1..42: mol_type = other DNA
atccgtcagccaccaggtaaaggtctggaatggatcggttac

SEQ ID NO: 229 - DNA, 42 bp, synthetic construct
  misc_feature 1..42: note = Primer
  source 1..42: mol_type = other DNA
gttcgtcaggttccaggtaaaggtctggaatctatgggtatc

SEQ ID NO: 230 - DNA, 42 bp, synthetic construct
  misc_feature 1..42: note = Primer
  source 1..42: mol_type = other DNA
gttcgtcaggctccaggtcagggtctggaatggatgggttgg

SEQ ID NO: 231 - DNA, 42 bp, synthetic construct
  misc_feature 1..42: note = Primer
  source 1..42: mol_type = other DNA
ttcggtcatggtaacacgaccctggaatttctgagcgtagat

SEQ ID NO: 232 - DNA, 42 bp, synthetic construct
  misc_feature 1..42: note = Primer
  source 1..42: mol_type = other DNA
agagatggtcagacgagatttcagagcagagttgtaaacgat

SEQ ID NO: 233 - DNA, 42 bp, synthetic construct
  misc_feature 1..42: note = Primer
  source 1..42: mol_type = other DNA
acgagagatggtgaaacgacctttaacagaatctgggtagta

SEQ ID NO: 234 - DNA, 42 bp, synthetic construct
  misc_feature 1..42: note = Primer
  source 1..42: mol_type = other DNA
aacagacatggtaacacgagatttcagagatgggttgtaatc

SEQ ID NO: 235 - DNA, 42 bp, synthetic construct
  misc_feature 1..42: note = Primer
  source 1..42: mol_type = other DNA
agcagagatggtaacctgaccctggaaagatggagagtaacg

SEQ ID NO: 236 - DNA, 42 bp, synthetic construct
  misc_feature 1..42: note = Primer
  source 1..42: mol_type = other DNA
ggtggtcatggtaccacgaccctgcagtttctgagcgtagtt

SEQ ID NO: 237 - DNA, 42 bp, synthetic construct
  misc_feature 1..42: note = Primer
  source 1..42: mol_type = other DNA
ctgtcttctctgaaatctgaggacacggccgtgtattactgt

SEQ ID NO: 238 - DNA, 42 bp, synthetic construct
  misc_feature 1..42: note = Primer
  source 1..42: mol_type = other DNA
atgaccaacatggatcctgtggacacagccacatattactgt

SEQ ID NO: 239 - DNA, 42 bp, synthetic construct
  misc_feature 1..42: note = Primer
  source 1..42: mol_type = other DNA
atgaactctctgcgtgccgaggacacggctgtgtattactgt

SEQ ID NO: 240 - DNA, 42 bp, synthetic construct
  misc_feature 1..42: note = Primer
  source 1..42: mol_type = other DNA
gttaactctgttaccgccgcggacacggctgtgtattactgt

SEQ ID NO: 241 - DNA, 42 bp, synthetic construct
  misc_feature 1..42: note = Primer
  source 1..42: mol_type = other DNA
tggtcttctctgaaagcctcggacaccgccatttattactgt

SEQ ID NO: 242 - DNA, 42 bp, synthetic construct
  misc_feature 1..42: note = Primer
  source 1..42: mol_type = other DNA
ctgcgttctctgcgttctgacgacacggccgtgtattactgt

SEQ ID NO: 243 - DNA, 42 bp, synthetic construct
  misc_feature 1..42: note = Primer
  source 1..42: mol_type = other DNA
gattggtttgccgctagctgaggagacggtgaccagggttcc

SEQ ID NO: 244 - DNA, 42 bp, synthetic construct
  misc_feature 1..42: note = Primer
  source 1..42: mol_type = other DNA
gattggtttgccgctagctgaggagacggtgaccgtggtccc

SEQ ID NO: 245 - DNA, 24 bp, synthetic construct
  misc_feature 1..24: note = Primer
  source 1..24: mol_type = other DNA
gtagatcagcagttttggagcttt

SEQ ID NO: 246 - DNA, 21 bp, synthetic construct
  misc_feature 1..21: note = Primer
  source 1..21: mol_type = other DNA
gtagatcagcagttttggtgg

SEQ ID NO: 247 - DNA, 21 bp, synthetic construct
  misc_feature 1..21: note = Primer
  source 1..21: mol_type = other DNA
gtagatcagcagctgtggaga

SEQ ID NO: 248 - DNA, 20 bp, synthetic construct
  misc_feature 1..20: note = Primer
  source 1..20: mol_type = other DNA
gtagatcagcagacgtggag

SEQ ID NO: 249 - DNA, 20 bp, synthetic construct
  misc_feature 1..20: note = Primer
  source 1..20: mol_type = other DNA
gtagatcagcagacgtggag

SEQ ID NO: 250 - DNA, 25 bp, synthetic construct
  misc_feature 1..25: note = Primer
  source 1..25: mol_type = other DNA
gtagatcatcagttttggagcttta

SEQ ID NO: 251 - DNA, 21 bp, synthetic construct
  misc_feature 1..21: note = Primer
  source 1..21: mol_type = other DNA
ctggcttggtaccagcagaaa

SEQ ID NO: 252 - DNA, 21 bp, synthetic construct
  misc_feature 1..21: note = Primer
  source 1..21: mol_type = other DNA
ctggcttggtaccagcagaaa

SEQ ID NO: 253 - DNA, 21 bp, synthetic construct
  misc_feature 1..21: note = Primer
  source 1..21: mol_type = other DNA
ctgcactggtacctgcagaaa

SEQ ID NO: 254 - DNA, 21 bp, synthetic construct
  misc_feature 1..21: note = Primer
  source 1..21: mol_type = other DNA
ctggcttggtaccagcagaaa

SEQ ID NO: 255 - DNA, 21 bp, synthetic construct
  misc_feature 1..21: note = Primer
  source 1..21: mol_type = other DNA
ctggcttggtaccagcagaaa

SEQ ID NO: 256 - DNA, 21 bp, synthetic construct
  misc_feature 1..21: note = Primer
  source 1..21: mol_type = other DNA
gtttcttggtaccagcagcac

SEQ ID NO: 257 - DNA, 25 bp, synthetic construct
  misc_feature 1..25: note = Primer
  source 1..25: mol_type = other DNA
acagtagtagttagcgaaatcttct

SEQ ID NO: 258 - DNA, 25 bp, synthetic construct
  misc_feature 1..25: note = Primer
  source 1..25: mol_type = other DNA
acagtagtaaacagcaacatcttca

SEQ ID NO: 259 - DNA, 25 bp, synthetic construct
  misc_feature 1..25: note = Primer
  source 1..25: mol_type = other DNA
acagtagtaaacaccaacatcttca

SEQ ID NO: 260 - DNA, 25 bp, synthetic construct
  misc_feature 1..25: note = Primer
  source 1..25: mol_type = other DNA
acagtagtaaacagcgaaatcttct

SEQ ID NO: 261 - DNA, 25 bp, synthetic construct
  misc_feature 1..25: note = Primer
  source 1..25: mol_type = other DNA
acagtagtaaacagcgaaatcttct

SEQ ID NO: 262 - DNA, 25 bp, synthetic construct
  misc_feature 1..25: note = Primer
  source 1..25: mol_type = other DNA
acagtagtaatcagcttcatcttca

SEQ ID NO: 263 - DNA, 21 bp, synthetic construct
  misc_feature 1..21: note = Primer
  source 1..21: mol_type = other DNA
ggtgttccatctcgtttctct

SEQ ID NO: 264 - DNA, 21 bp, synthetic construct
  misc_feature 1..21: note = Primer
  source 1..21: mol_type = other DNA
ggtgttccagatcgtttctct

SEQ ID NO: 265 - DNA, 21 bp, synthetic construct
  misc_feature 1..21: note = Primer
  source 1..21: mol_type = other DNA
ggtgttccagatcgtttctct

SEQ ID NO: 266 - DNA, 21 bp, synthetic construct
  misc_feature 1..21: note = Primer
  source 1..21: mol_type = other DNA
ggtatcccagctcgtttctct

SEQ ID NO: 267 - DNA, 21 bp, synthetic construct
  misc_feature 1..21: note = Primer
  source 1..21: mol_type = other DNA
ggtatcccagatcgtctgtct

SEQ ID NO: 268 - DNA, 22 bp, synthetic construct
  misc_feature 1..22: note = Primer
  source 1..22: mol_type = other DNA
ggtgtttctaaccgtttctctg

SEQ ID NO: 269 - DNA, 20 bp, synthetic construct
  misc_feature 1..20: note = Primer
  source 1..20: mol_type = other DNA
accacccatccattccagac

SEQ ID NO: 270 - DNA, 20 bp, synthetic construct
  misc_feature 1..20: note = Primer
  source 1..20: mol_type = other DNA
catagccagccattccagag

SEQ ID NO: 271 - DNA, 21 bp, synthetic construct
  misc_feature 1..21: note = Primer
  source 1..21: mol_type = other DNA
agaagcaaccagttccagacc

SEQ ID NO: 272 - DNA, 21 bp, synthetic construct
  misc_feature 1..21: note = Primer
  source 1..21: mol_type = other DNA
gtaaccgatccattccagacc

SEQ ID NO: 273 - DNA, 24 bp, synthetic construct
  misc_feature 1..24: note = Primer
  source 1..24: mol_type = other DNA
gatacccatagattccagaccttt

SEQ ID NO: 274 - DNA, 20 bp, synthetic construct
  misc_feature 1..20: note = Primer
  source 1..20: mol_type = other DNA
ccaacccatccattccagac

SEQ ID NO: 275 - DNA, 19 bp, synthetic construct
  misc_feature 1..19: note = Primer
  source 1..19: mol_type = other DNA
atccactgggttcgtcagg

SEQ ID NO: 276 - DNA, 21 bp, synthetic construct
  misc_feature 1..21: note = Primer
  source 1..21: mol_type = other DNA
gttaactggatccgtcagcca

SEQ ID NO: 277 - DNA, 21 bp, synthetic construct
  misc_feature 1..21: note = Primer
  source 1..21: mol_type = other DNA
atgtcttgggttcgtcaggct

SEQ ID NO: 278 - DNA, 19 bp, synthetic construct
  misc_feature 1..19: note = Primer
  source 1..19: mol_type = other DNA
tggtcttggatccgtcagc

SEQ ID NO: 279 - DNA, 21 bp, synthetic construct
  misc_feature 1..21: note = Primer
  source 1..21: mol_type = other DNA
atcgcttgggttcgtcaggtt

SEQ ID NO: 280 - DNA, 21 bp, synthetic construct
  misc_feature 1..21: note = Primer
  source 1..21: mol_type = other DNA
atctcttgggttcgtcaggct

SEQ ID NO: 281 - DNA, 21 bp, synthetic construct
  misc_feature 1..21: note = Primer
  source 1..21: mol_type = other DNA
acagtaatacacggccgtgtc

SEQ ID NO: 282 - DNA, 23 bp, synthetic construct
  misc_feature 1..23: note = Primer
  source 1..23: mol_type = other DNA
acagtaatatgtggctgtgtcca

SEQ ID NO: 283 - DNA, 21 bp, synthetic construct
  misc_feature 1..21: note = Primer
  source 1..21: mol_type = other DNA
acagtaatacacagccgtgtc

SEQ ID NO: 284 - DNA, 21 bp, synthetic construct
  misc_feature 1..21: note = Primer
  source 1..21: mol_type = other DNA
acagtaatacacagccgtgtc

SEQ ID NO: 285 - DNA, 22 bp, synthetic construct
  misc_feature 1..22: note = Primer
  source 1..22: mol_type = other DNA
acagtaataaatggcggtgtcc

SEQ ID NO: 286 - DNA, 21 bp, synthetic construct
  misc_feature 1..21: note = Primer
  source 1..21: mol_type = other DNA
acagtaatacacggccgtgtc

SEQ ID NO: 287 - DNA, 22 bp, synthetic construct
  misc_feature 1..22: note = Primer
  source 1..22: mol_type = other DNA
atctacgctcagaaattccagg

SEQ ID NO: 288 - DNA, 24 bp, synthetic construct
  misc_feature 1..24: note = Primer
  source 1..24: mol_type = other DNA
gtttacaactctgctctgaaatct

SEQ ID NO: 289 - DNA, 24 bp, synthetic construct
  misc_feature 1..24: note = Primer
  source 1..24: mol_type = other DNA
tactacccagattctgttaaaggt

SEQ ID NO: 290 - DNA, 24 bp, synthetic construct
  misc_feature 1..24: note = Primer
  source 1..24: mol_type = other DNA
gattacaacccatctctgaaatct

SEQ ID NO: 291 - DNA, 21 bp, synthetic construct
  misc_feature 1..21: note = Primer
  source 1..21: mol_type = other DNA
cgttactctccatctttccag

SEQ ID NO: 292 - DNA, 21 bp, synthetic construct
  misc_feature 1..21: note = Primer
  source 1..21: mol_type = other DNA
aactacgctcagaaactgcag

SEQ ID NO: 293 - DNA, 23 bp, synthetic construct
  misc_feature 1..23: note = Primer
  source 1..23: mol_type = other DNA
cggattgtcttcaaccaacacaa

SEQ ID NO: 294 - DNA, 20 bp, synthetic construct
  misc_feature 1..20: note = Primer
  source 1..20: mol_type = other DNA
ctcctcctgttgaatccagg

SEQ ID NO: 295 - DNA, 21 bp, synthetic construct
  misc_feature 1..21: note = Primer
  source 1..21: mol_type = other DNA
cagttagataaaagaggcgcg

SEQ ID NO: 296 - DNA, 18 bp, synthetic construct
  misc_feature 1..18: note = Primer
  source 1..18: mol_type = other DNA
gcccagcagtgggtttgg

SEQ ID NO: 297 - DNA, 19 bp, synthetic construct
  misc_feature 1..19: note = Primer
  source 1..19: mol_type = other DNA
tccggagggtcgaccataa

SEQ ID NO: 298 - DNA, 21 bp, synthetic construct
  misc_feature 1..21: note = Primer
  source 1..21: mol_type = other DNA
ggtaccgctcgaggataactt